(19) United States
(12) Patent Application Publication
(10) Pub. No.: US 2023/0331809 A1
(43) Pub. Date: Oct. 19, 2023

(54) FUSION PROTEINS COMPRISING A LIGAND-RECEPTOR PAIR AND A BIOLOGICALLY FUNCTIONAL PROTEIN

(71) Applicant: ZYMEWORKS INC., Vancouver (CA)

(72) Inventors: Surjit Bhimarao Dixit, Richmond (CA); Gesa Volkers, Vancouver (CA); Florian Heinkel, Vancouver (CA); Eric ESCOBAR-CABRERA, Burnaby (CA); Thomas Spreter Von Kreudenstein, Vancouver (CA); Anna Von Rossum, Vancouver (CA)

(21) Appl. No.: 18/017,319

(22) PCT Filed: Jul. 20, 2021
(86) PCT No.: PCT/CA2021/051006

§ 371 Date: Jan. 20, 2023

Related U.S. Application Data
(60) Provisional application No. 63/172,626, filed on Apr. 8, 2021.
(60) Provisional application No. 63/054,180, filed on Jul. 20, 2020.

(51) Int. Cl. C07K 14/705; C07K 16/28

(52) U.S. Cl. CPC C07K 14/70521; C07K 16/2809; C07K 16/2863; C07K 2317/52; C07K 2317/55; C07K 2317/565; C07K 2317/732; C07K 2319/30

(57) ABSTRACT

The present disclosure provides fusion proteins with a multifunctional biologic design for programmed target engagement. In certain embodiments, the fusion proteins described herein provide for concurrent target antigen engagement and immune checkpoint or costimulatory receptor targeting. In certain aspects, the fusion protein is masked from presenting any on-target off-tissue action (i.e., toxicity) associated with target engagements. In certain embodiments, the fusion proteins provide a masked antigen binding domain as well as a masked immunomodulatory target binding domain, such that the programmed activation of one binding functionality results in the activation of the other binding functionality as well, thereby yielding a bispecific molecule. Thus, the disclosure also provides for methods of masking and conditional activation of antigen binding domains in specific target tissue setting and targeting and activation of immunomodulatory targets without severe adverse toxicity effects.

### BACKGROUND

With the development of monoclonal antibodies and other biologics as drugs, highly specific and targeted therapeutic agents can be designed. The use of these agents, however, is often impeded by the fact that most molecular targets that could mark a diseased cell such as cancer can also appear in non-diseased (normal) cells in the body of the patient, albeit with some degree of differential expression. As a result, active targeted biomolecules upon use as therapeutic agents can show unintended activity at locations outside of where they are expected to act for therapeutic benefit, and this could result in potential toxicity and undesirable side effects. This is referred to as on-target off-tumor (also known as on-target off-tissue) action and impacts the dosing regimen as well as balance between efficacy and toxicity of the drug. The on-target off-tumor action could lead to the unintended uptake and accelerated clearance of the therapeutic agent by non-diseased cells, resulting in an unfavorable pharmacokinetic profile of the therapeutic, also referred to as target mediated drug disposition (TMDD). Thus, beyond high specificity for the molecular target, these challenges call for features in the therapeutic design which allows for the conditional and localized action of the therapeutic agent on the diseased cell/tissue while avoiding impact of the drug on the same target expressed off-tumor tissue.

Targeting immune checkpoint pathways, via either positive or negative costimulatory molecules, can provide durable treatment responses with the active engagement of the patient’s immune system. Unfortunately, the checkpoint pathway targeting therapies can also suffer from issues of target mediated drug toxicity and clearance challenges. There is also a growing realization that when co-targeting more than one of these checkpoints and /or costimulatory pathways or when these checkpoint targets are combined with other non-immune related targets and therapies there can be a more effective revitalization of the immune response. Hence, there is great interest to design therapeutic strategies involving checkpoint targets, but the issues related to immune related adverse events (irAE’s), i.e., toxicity and clearance, remain a challenge. A design that provides conditional engagement of a therapeutic agent could provide for a less toxic and more effective solution for targeting of immunomodulatory molecules.

### SUMMARY OF INVENTION

Described herein is a fusion protein comprising a biologically functional protein, a ligand-receptor pair, a first peptidic linker and a second peptidic linker; wherein the biologically functional protein comprises at least a first polypeptide and a second polypeptide; and the ligand-receptor pair comprises an extracellular portion of an immunoglobulin superfamily (IgSF) receptor and its cognate ligand or a receptor-binding fragment thereof; wherein the ligand is fused to a terminus of the first polypeptide via the first peptidic linker; the receptor is fused to the same respective terminus of the second polypeptide via the second peptidic linker; and the first and second peptidic linkers are of sufficient length to allow pairing of the ligand and receptor. In some embodiments, at least one of the first and second peptidic linkers comprises a protease cleavage site. In certain embodiments the ligand is fused to the N-terminus of the first polypeptide via the first peptidic linker, and the receptor is fused to the N-terminus of the second polypeptide via the second peptidic linker.

In certain embodiments, the biologically functional protein comprises an antibody or antigen-binding antibody fragment. In certain embodiments, the biologically functional protein consists of a polypeptide scaffold. In certain embodiments the polypeptide scaffold is a dimeric Fc region, wherein the first polypeptide consists of a first Fc polypeptide and the second polypeptide consists of a second Fc polypeptide, the first and second Fc polypeptides forming the dimeric Fc region. In certain embodiments, the biologically functional protein comprises a polypeptide scaffold.

In certain embodiments, the polypeptide scaffold comprises a dimeric Fc region. In certain embodiments, the dimeric Fc region is a heterodimeric Fc. In certain embodiments, the at least one of the ligand or the receptor in the ligand-receptor pair is capable of binding to an immunomodulatory target.

In some embodiments, the ligand receptor pair is involved in a cellular response selected from the group consisting of: modulation of an immune checkpoint, modulation of immune cell activity, modulation of T-cell receptor signaling, modulation of T-cell dependent cytotoxicity (TDCC), modulation of antibody-dependent cellular phagocytosis (ADCP) and modulation of antibody-dependent cellular cytotoxicity (ADCC). In some embodiments, the receptor comprises one or more mutations that increase or decrease binding affinity of the receptor for its cognate ligand as compared to a wild-type receptor.

In some embodiments, the ligand comprises one or more mutations that increase or decrease binding affinity of the ligand for its cognate receptor as compared to a wild-type ligand. In certain embodiments, the ligand-receptor pair is selected from the group consisting of: PD1-PDL1, PD1-PDL2, CTLA4-CD80, CD28-CD80, CD28-CD86, CTLA4-CD86, PDL1-CD80, ICOS-ICOSL, NCRSRLG1-NKp30 and CD47-SIRPa. In certain embodiments, the ligand-receptor pair is PD1-PDL1. In certain embodiments, the ligand PDL1 comprises an amino acid sequence according to SEQ ID NO: 8. In certain embodiments, the receptor PD1 comprises an amino acid sequence according to SEQ ID NO: 9.

In certain embodiments, the ligand-receptor pair is CTLA4-CD80. In certain embodiments, the ligand CD80 comprises an amino acid sequence according to SEQ ID NO: 25, SEQ ID NO: 185, SEQ ID NO: 187 or SEQ ID NO: 189. In certain embodiments, the receptor CTLA4 comprises an amino acid sequence according to SEQ ID NO: 26.

In certain embodiments, the receptor and the ligand are fused to the respective N- termini of the first and second polypeptides. In certain embodiments, the one of the first or second peptidic linkers comprises more than one protease cleavage site. In certain embodiments, the one of the peptidic linkers fused to the ligand or the receptor is engineered to comprise one or more additional protease cleavage sites, and wherein the one or more protease cleavage sites in the ligand or the receptor and the protease cleavage site in the first or second peptidic linker are cleavable by the same protease or a different protease.

In certain embodiments, the protease is selected from the group consisting of: a serine protease, MMP1, MMP2, MMP3, MMP7, MMP8, MMP9, MW10, MMP11, MMP12, MMP13, MMP14, MMP15, MMP16, MMP17, MMP18 (collagenase 4), MMP19, MMP20, MMP21, an adamalysin, a serralysin, an astacin, caspase 1, caspase 2, caspase 3, caspase 4, caspase 5, caspase 6, caspase 7, caspase 8, caspase 9, caspase 10, caspase 11, caspase 12, caspase 13, caspase 14, cathepsin A, cathepsin B, cathepsin D, cathepsin E, cathepsin K, cathepsin S, granzyme B, guanidinobenzoatase (GB), hepsin, elastase, legumain, matriptase, matriptase 2, meprin, neurosin, MT-SP1, neprilysin, plasmin, PSA, PSMA, TACE, TMPRSS3, TMPRSS4, uPA, calpain, FAP and KLK. In certain embodiments, the protease is uPA or matriptase.

In certain embodiments, the peptidic linker is 3-50 or 5-20 amino acids in length. In certain embodiments, the one of the first or second peptidic linkers does not have a protease cleavage site. In certain embodiments, the peptidic linker is a (GlyₙSer) linker, wherein the (GlyₙSer) linker comprises an amino acid sequence selected from the group consisting of (Gly₃Ser)ₙ(Gly₄Ser)₁, (Gly₃Ser)₁(Gly₄Ser)ₙ (Gly₃Ser)ₙ(Gly₄Ser)ₙ, and (Gly₄Ser)ₙ, wherein n is an integer of 1 to 5. In certain embodiments, the peptidic linker is an (EAAAK)ₙ linker, wherein n is an integer between 1 and 5. In certain embodiments, the peptidic linker comprises the amino acid sequence EAAAKEAAAK (SEQ ID. NO: 38). In certain embodiments, the peptidic linker is a polyproline linker, optionally PPP or PPPP. In certain embodiments, the peptidic linker comprises an immunoglobulin hinge region sequence comprising an amino acid sequence having up to a 30 percent difference in amino acid sequence identity compared to a wild type immunoglobulin hinge region amino acid sequence. In certain embodiments, the peptidic linker comprises a protease cleavage site comprising the amino acid sequence MSGRSANA (SEQ ID NO: 28).

Also described herein is a fusion protein comprising a Fab region and an Fc region; wherein the Fab region comprises a VH polypeptide and a VL polypeptide that form an antigen-binding domain, and a ligand-receptor pair comprising an extracellular portion of an immunoglobulin superfamily receptor and its cognate ligand or a receptor-binding fragment thereof; wherein the ligand is fused to the N-terminus of one of the VH or VL polypeptides via a first peptidic linker and the receptor is fused to the N-terminus of the other VH or VL polypeptide via a second peptidic linker; wherein first and second peptidic linkers are of sufficient length to allow pairing of the ligand and receptor; wherein at least one of the first and second peptidic linkers comprises a protease cleavage site; and wherein the ligand-receptor pair sterically hinders binding of the antigen-binding domain to its cognate antigen.

In some embodiments, the at least one of the first and second polypeptides comprise a first VH polypeptide and a first VL polypeptide, the first VH and VL polypeptides forming a first antigen-binding domain of the antibody, wherein the ligand is fused to one of the first VH or VL polypeptides via the first peptidic linker and the receptor is fused to the other of the first VH or VL polypeptides via the second peptidic linker, and wherein the ligand-receptor pair sterically hinders binding of the first antigen-binding domain to its cognate antigen. In certain embodiments, the first and second polypeptides further comprise a dimeric Fc. In certain embodiments, the dimeric Fc region is a heterodimeric Fc.

In certain embodiments, the fusion protein comprises, from N terminus to C terminus, Ligand-Linker-VL, Receptor-Linker-VL, Ligand-Linker-VH, or Receptor-Linker-VH.

In certain embodiments, the fusion protein comprises from N terminus to C terminus, Ligand-cleavable Linker-VL, Receptor- cleavable Linker-VL, Ligand- cleavable Linker-VH, or Receptor- cleavable Linker-VH.

In certain embodiments, the fusion protein comprises from N terminus to C terminus, Ligand-linker (SEQ ID NO:114)-VL, Receptor-linker (SEQ ID NO:114)-VL, Ligand-linker (SEQ ID NO: 14)-VH, or Receptor-linker (SEQ ID NO: 14)-VH.

In certain embodiments, the fusion protein comprises from N terminus to C terminus, Ligand-linker (SEQ ID NO: 145)-VL, Receptor-linker (SEQ ID NO: 145)- VL, Ligand-linker (SEQ ID NO: 145)-VH, or Receptor-linker (SEQ ID NO: 145)-VH.

In certain embodiments, the fusion protein comprises from N terminus to C terminus, Ligand-linker (SEQ ID NO: 147)-VL, Receptor-linker (SEQ ID NO: 147)- VL, Ligand-linker (SEQ ID NO: 147)-VH, or Receptor-linker (SEQ ID NO: 147)-VH.

In certain embodiments, the fusion protein comprises from N terminus to C terminus, Ligand-linker (SEQ ID NO: 154)-VL, Receptor-linker (SEQ ID NO: 154)- VL, Ligand-linker (SEQ ID NO: 154)-VH, or Receptor-linker (SEQ ID NO: 154)-VH.

In certain embodiments, the fusion protein comprises from N terminus to C terminus, Ligand-linker (SEQ ID NO:203)-VL, Receptor-linker (SEQ ID NO:203)-VL, Ligand-linker (SEQ ID NO:203)-VH, or Receptor-linker (SEQ ID NO:203)-VH.

In certain embodiments, the at least one of the ligand or the receptor of the ligand-receptor pair is capable of binding to an immunomodulatory target. In certain embodiments, the ligand receptor pair is involved in a cellular response selected from the group consisting of: modulation of an immune checkpoint, modulation of immune cell activity, modulation of T-cell receptor signaling, modulation of T-cell dependent cytotoxicity (TDCC), modulation of antibody-dependent cellular phagocytosis (ADCP) and modulation of antibody-dependent cellular cytotoxicity (ADCC).

In certain embodiments, the receptor comprises one or more mutations that increase or decrease binding affinity of the receptor for its cognate ligand as compared to a wild-type receptor. In certain embodiments, the ligand comprises one or more mutations that increase or decrease binding affinity of the ligand for its cognate receptor as compared to a wild-type ligand. In certain embodiments, the ligand-receptor pair is selected from the group consisting of: PD1-PDL1, PD1-PDL2, CTLA4-CD80, CD28-CD80, CD28-CD86, CTLA4-CD86, PDL1-CD80, ICOS-ICOSL, NCRSRLG1-NKp30 and CD47-SIRPa. In certain embodiments, the ligand-receptor pair is PD1-PDL1. In certain embodiments, the ligand PDL1 comprises an amino acid sequence according to SEQ ID NO: 8. In certain embodiments, the receptor PD1 comprises an amino acid sequence according to SEQ ID NO: 9. In certain embodiments, the ligand-receptor pair is CTLA4-CD80. In certain embodiments, the ligand CD80 comprises an amino acid sequence according to SEQ ID NO: 25. In certain embodiments, the receptor CTLA4 comprises an amino acid sequence according to SEQ ID NO: 26.

In some embodiments, the receptor and the ligand are fused to the respective N- termini of the first and second polypeptides. In certain embodiments, one of the first or second peptidic linkers comprises more than one protease cleavage site. In certain embodiments, one of the ligand or the receptor is engineered to comprise one or more additional protease cleavage sites, and wherein the one or more protease cleavage sites in the ligand or the receptor and the protease cleavage site in the first or second peptidic linker are cleavable by the same protease or by different proteases.

In certain embodiments, the protease is selected from the group consisting of: a serine protease, MMP1, MMP2, MMP3, MMP7, MMP8, MMP9, MMP10, MMP 11, MMP12, MMP13, MMP14, MMP15, MMP16, MMP17, MMP18 (collagenase 4), MMP19, MMP20, MMP21, an adamalysin, a serralysin, an astacin, caspase 1, caspase 2, caspase 3, caspase 4, caspase 5, caspase 6, caspase 7, caspase 8, caspase 9, caspase 10, caspase 11, caspase 12, caspase 13, caspase 14, cathepsin A, cathepsin B, cathepsin D, cathepsin E, cathepsin K, cathepsin S, granzyme B, guanidinobenzoatase (GB), hepsin, elastase, legumain, matriptase, matriptase 2, meprin, neurosin, MT-SP1, neprilysin, plasmin, PSA, PSMA, TACE, TMPRSS3, TMPRSS4, uPA, calpain, FAP and KLK. In certain embodiments, the protease is uPA or matriptase. In certain embodiments, the peptidic linker is 3-50 or 5-20 amino acids in length. In certain embodiments, the one of the first or second peptidic linkers does not have a protease cleavage site. In certain embodiments, the peptidic linker is a (GlyₙSer) linker, wherein the (GlyₙSer) linker comprises an amino acid sequence selected from the group consisting of (Gly₃Ser)ₙ(Gly₄Ser)₁, (Gly₃Ser)₁Gly₄Ser)ₙ, (Gly₃Ser)ₙ(Gly₄Ser)ₙ, and (Gly₄Ser)ₙ, wherein n is an integer of 1 to 5. In certain embodiments, the peptidic linker an (EAAAK)ₙ linker, wherein n is an integer between 1 and 5. In certain embodiments, the peptidic linker that does not have a protease cleavage site comprises the amino acid sequence EAAAKEAAAK (SEQ ID. NO: 38). In certain embodiments, the peptidic linker is a polyproline linker, optionally PPP or PPPP. In certain embodiments the linker is glycine (G) proline (P) polypeptide linker, optionally GPPPG, GGPPPGG, GPPPPG or GGPPPGG. In certain embodiments, the peptidic linker comprises an immunoglobulin hinge region sequence comprising an amino acid sequence having up to a 30 percent difference in amino acid sequence identity compared to a wild type immunoglobulin hinge region amino acid sequence. In certain embodiments, the peptidic linker comprising a protease cleavage site comprises the amino acid sequence MSGRSANA (SEQ ID NO: 28).

In certain embodiments, binding of the first antigen-binding domain to its cognate antigen is reduced by 10-fold or more as compared to a parental antigen-binding domain that is not fused to the ligand-receptor pair. In certain embodiments, cleavage of the protease cleavage site in a cellular environment releases one member of the ligand-receptor pair from the fusion protein, thereby allowing the antigen-binding domain to bind its cognate antigen.

In certain embodiments, the first antigen-binding domain is a Fab. In certain embodiments, the first antigen-binding domain binds an antigen that is expressed on a cancer cell or an immune cell. In certain embodiments, the first antigen-binding domain binds an antigen that is expressed on a T-cell. In certain embodiments, the first antigen binding domain binds to a tumor-associated antigen (TAA). In certain embodiments, the first antigen-binding domain binds to an antigen selected from the group consisting of: Cluster of Differentiation 3 (CD3), Human Epidermal Growth Factor Receptor 2 (HER2), Epidermal Growth Factor Receptor (EGFR), Mesothelin (MSLN), Tissue Factor (TF), Cluster of Differentiation 19 (CD19), tyrosine-protein kinase Met (c-Met), Cluster of Differentiation 40 (CD40) and Cadherin 3 (CDH3).

In certain embodiments, the antibody or antibody fragment comprises a second antigen binding domain comprising a second VH polypeptide and a second VL polypeptide. In certain embodiments, the fusion protein comprises a second ligand-receptor pair, wherein the ligand of the second ligand-receptor pair is fused to one of the second VH or VL polypeptides via a third peptidic linker and the receptor of the second ligand-receptor pair is fused to the other of the second VH or VL polypeptides via a fourth peptidic linker, wherein at least one of the third and fourth peptidic linkers comprise a protease cleavage site, and wherein the ligand-receptor pair sterically hinders binding of the second antigen-binding domain to its cognate antigen. In certain embodiments, the fusion protein binds to two distinct antigens. In certain embodiments, one antigen is an antigen expressed by T cells and the other antigen is an antigen expressed by cancer cells. In certain embodiments, the fusion protein binds to CD3 and HER2.

Also described herein is a fusion protein comprising an Fc region comprising a first Fc polypeptide and a second Fc polypeptide, and a ligand-receptor pair comprising an extracellular portion of an immunoglobulin superfamily receptor and its cognate ligand or a receptor-binding fragment thereof; wherein the ligand is fused to a terminus of the first Fc polypeptide via a first peptidic linker and the receptor is fused to the same respective terminus of the second Fc polypeptide via a second peptidic linker; wherein the first and second peptidic linkers are of sufficient length to allow pairing of the ligand and receptor; and wherein at least one of the first and second peptidic linkers comprises a protease cleavage site.

### BRIEF DESCRIPTION OF FIGURES

These and other features, aspects, and advantages of the present invention will become better understood with regard to the following description, and accompanying drawings, where:

FIG. 1(A) shows a schematic drawing of the structure of certain fusion proteins described herein. By fusing PD-1 (checkered) and PD-L1 (striped) to the N termini of heavy and light chain, respectively, the paratope of a Fab (grey) can be sterically blocked by the Ig superfamily heterodimer that is formed between the two. Upon removal of one side of this mask via the TME-specific, proteolytic cleavage (bolt) of one of the linkers that is introduced between the masking domain and the Fab, part of the mask can be released and binding to the target can be restored. Furthermore, the part of the mask that remains covalently attached to the Fab adds functionality by binding to its immunomodulatory partner. FIG. 1(B) shows a schematic of an antibody with two Fab arms that are masked using IgSF domain pairs attached N-terminally with TME protease cleavable or uncleavable linkers. Fab paratopes a-TAA 1 and a-TAA 2 may be the same or different and IgSF pairs 1:2 and 3:4 may be the same or different. FIG. 1(C) shows a schematic of a Fab x scFv construct with a Fab arm specific for target 1 and an scFv arm specific for target 2. The Fab arm and binding to target 1 is masked by a IgSF domain pair attached to the N-termini using TME protease cleavable or uncleavable linkers.

FIG. 2 shows a schematic drawing of a modified bispecific CD3 × Her2 Fab × scFv Fc fusion protein described herein. One arm of the antibody-like molecule contains the anti CD3 Fab that is blocked by a PD-1/PD-L1 mask, while the other arm contains an anti-Her2 scFv.

FIG. 3 shows UPLC-SEC chromatograms and non-reducing and reducing CE-SDS profiles for representative bispecific CD3 × Her2 Fab × scFv Fc variants. (A) UPLC-SEC chromatogram of unmasked variant 30421, (B) non-reducing (left) and reducing (right) CE-SDS profiles of unmasked variant 30421, (C) UPLC-SEC chromatogram of masked, uncleavable variant 30423, (D) non-reducing (left) and reducing (right) CE-SDS profiles of masked, uncleavable variant 30423, (E) UPLC-SEC chromatogram of masked, light-chain-cleavable variant 30430, (F) non-reducing (left) and reducing (right) CE-SDS profiles of masked, light-chain-cleavable variant 30430, (G) UPLC-SEC chromatogram of masked, heavy-chain-cleavable variant 30436, (H) non-reducing (left) and reducing (right) CE-SDS profiles of masked, heavy-chain-cleavable variant 30436.

FIG. 4 shows an overlay of DSC thermograms for unmodified (30421) and PD-1:PD-L1 masked variants (30430, 30436) of the investigated CD3 × Her2 Fab × scFv Fc system.

FIG. 5 shows reducing CE-SDS profiles of representative variants without (-uPa) and with uPa treatment (+uPa) for 24 h at 37° C. at a 1:50 uPa:variant ratio. Profiles for unmasked (30421), masked but uncleavable (30423), and masked cleavable variants (30430, 30436, 31934) are shown.

FIG. 6 shows native binding results of CD3 targeted variants to Jurkat cells as determined by ELISA. Results are shown for an unmasked variant (30421), constructs with only the PD-L1 or PD-1 moiety attached (31929, 31931), and variants with a full, uncleavable mask (30423) or with a full mask and a cleavable PD-L1 or PD-1 moiety (30430, 30436). For samples of variants 30423, 30430, 30436, uPa untreated (-uPa) and treated (+uPa) samples were tested.

FIG. 7 shows cell killing of JIMT-1 tumor cells by Pan T-cells as determined in a TDCC assay after treatment with engineered variants cross-linking T-cells and tumor cells. Results are shown for an unmasked variant (30421), a variant with only the PD-1 moiety attached to the heavy chain (31929), and variants with a full, uncleavable mask (30423) or with a full mask and a cleavable PD-L1 moiety on the light chain (30430). For variant 30430 uPa untreated (-uPa) and treated (+uPa) samples were tested. An irrelevant anti-RSV antibody (22277) was used as a negative control.

FIG. 8 shows results of a native binding study by flow cytometry of select CD3 targeted variants to (A) PD-L1 transfected and (B) PD-1 transfected CHO-S cells. Results are shown for an unmasked variant (30421), constructs with only the PD-L1 or PD-1 moiety attached (31929, 31931), and variants with a full, uncleavable mask (30423, 30426) or with a full mask and a cleavable PD-L1 or PD-1 moiety (30430, 30436). An Fc-fusion of the affinity-matured PD-1 moiety is also included (31829). For samples of variants 30423, 30426, 30430, 30436, uPa untreated (-uPa) and treated (+uPa) samples were tested.

FIG. 9 shows a schematic of a hybrid PD-1/PD-L1 Reporter Gene Assay probing cross-linking of T-cells and JIMT-1 cells and blockade of the PD-1:PD-L1 checkpoint engagement (A) as well as the analysis of the same (B). Results are shown for an unmasked variant (30421) and the same unmasked variant in combination with an excess of anti-PD-L1 antibody (30421 + 150 nM anti-PD-L1). A construct with only the PD-1 moiety attached to the heavy chain (31929), and variants with a full, uncleavable mask (30423) or with a full mask and a cleavable PD-L1 moiety on the light chain (30430) were also investigated. For variant 30430, uPa untreated (-uPa) and treated (+uPa) samples were tested. An irrelevant anti-RSV antibody (22277) was used as a negative control. Measurements were performed in triplicate and error bars reflecting standard deviation are shown.

FIG. 10 is a drawing representative of a modified monospecific, bivalent fusion protein targeted against tumor associated antigens (TAA). The paratope of the Fab is sterically blocked by the PD-1/PD-L1 mask.

FIG. 11 shows UPLC-SEC chromatograms (A-J) and non-reducing SDS-PAGE (K) or non-reducing and reducing CE-SDS profiles (L) of masked fusion proteins targeted against EGFR, MSLN, TF, CD19, cMet, CDH3. For all fusion proteins, data for uncleavable variants is shown (31722, 31728, 31736, 31732, 28647, 28662), while for EGFR, MSLN, TF and CD19, samples of cleavable variants are also included (31723, 31729, 31737, 31733).

FIG. 12 shows reducing SDS-PAGE profiles of representative fusion proteins targeted against (A) EGFR, (B) MSLN, (C) TF, (D) CD19. Untreated (-uPa) and uPa-treated (+uPa) samples are investigated. For each system, data for a uPa-uncleavable variant (31722, 31728, 31736, 31732) and a variant with a u-Pa cleavage sequence between the VL and the PD-L1 moiety (31723, 31729, 31737, 31733) is shown.

FIG. 13 Shows flow cytometry native binding results for select fusion proteins targeted against different antigens to the following cell lines expressing that antigen: (A) EGFR on MDA-MB-468, (B) MSLN on OVCAR3, (C) TF on MDA-MB-231, (D) CD19 on Raji, (E) cMet on EBC1, (F) CDH3 on JIMT1. For all systems, data for uncleavable variants is shown (31722, 31728, 31736, 31732, 28647, 28662), while for EGFR, MSLN, TF and CD19, samples of cleavable variants are also included (31723, 31729, 31737, 31733) and tested without (-uPa) and with uPa-processing (+uPa). For all systems an unmodified control (32474, 16427 16417, 6323, 4372, 17606, 17214) is also included as well as an irrelevant control for cMet and CDH3 (22277). Where available (EGFR, MSLN, TF) data from SPR are included for comparison.

FIG. 14 shows results from a growth inhibition study of NCI-H292 cells treated with EGFR-targeted variants. Data is shown for unmasked (32474) and PD-1:PD-L masked variants. The masked variants include an uncleavable form (31722) as well as one with a cleavable PD-L1 moiety on the light chain (31723). An irrelevant control (22277) is also included. For all variants, samples are tested with (-uPa) and without (+uPa) treatment. The error bars reflect the standard deviation of triplicate measurements.

FIG. 15 shows a schematic drawing of a modified bispecific CD3 x Her2 Fab x scFv Fc variant that was investigated here. One arm of the fusion protein contains the anti CD3 Fab that is blocked by a CD80/CTLA4 mask, while the other arm contains an anti-Her2 scFv.

FIG. 16 shows UPLC-SEC chromatogram and non-reducing and reducing CE-SDS profiles of variant 30444. (A) UPLC-SEC chromatogram of masked, light-chain-cleavable variant 30444, (B) non-reducing (left) and reducing (right) CE-SDS profiles of masked, light-chain-cleavable variant 30444, (C) non-reducing (left) and reducing (right) CE-SDS profiles of masked, light-chain-cleavable variant 30444, (D-F) UPLC-SEC chromatograms of masked, light-chain-cleavable variants 33525, 33526, 33527 after protein A purification.

FIG. 17 shows reducing CE-SDS profiles of variant 30444 without (-uPa) and with uPa treatment (+uPa).

FIG. 18 shows native binding results of CD3 targeted variants to Jurkat cells as determined by ELISA. Results are shown for an unmasked variant (30421), a variant with a full PD-1/PD-L1-based mask and a cleavable PD-L1 moiety (30430) and a variant with a full CD80/CTLA4-based mask and a cleavable CTLA4 moiety (30444). For samples of variants 30430 and 30444, uPa untreated (-uPa) and treated (+uPa) samples were tested.

FIG. 19 shows a schematic of IgVs of an immunomodulator pair (e.g. PD-1:PD-L1) fused via the hinge to a heterodimeric IgG Fc. Cleavage of one of the two linkers by a TME-associated protease such as uPa releases one moiety (e.g. PD-L1) and leaves the one with the desired function (e.g. PD-1) still attached to the Fc and available to bind to its partner on cells. In the case of PD-1, it is able to bind PD-L1 on target cells and inhibit checkpoint function.

FIG. 20 shows (A-C) UPLC-SEC chromatograms and (D) non-reducing and reducing CE-SDS profiles of CD40-targeted variants. (E) Reducing CE-SDS, (F) flow cytometry binding data and (G) results from a CD40 RGA assay are also shown for the same variants without (-uPa) and with (+uPa) treatment with uPa. Test articles include an unmasked variant (32477), a variant with an uncleavable PD-1/PD-L1-based mask (32478) and one with a PD-1/PD-L1-based mask in which the PD-L1 moiety can be removed by cleavage with uPa (32479). In the functional investigation via RGA assay (G), the native CD40 binding partner CD40L and an irrelevant control (v22277) are also included. Data for the CD40 RGA assay is summarized in the table in (H).

FIG. 21 (A) PD1 and PDL1 are comprised of immunoglobulin domains that form a complex. In the image, the binding Fab is docked with the PD1-PDL1 complex on the paratope end. Linking the PD1 and PDL1 to the VH and VL chains with appropriate linker could block antigen binding. (B) Structures of other exemplary immunomodulator pairs that could serve as masks: PD-1/PD-L1 (PDB:4ZQK), PD-1/PD-L2 (PDB: 3BP5), CTLA4/CD86 (PDB:1I85), NCRSRLG1/NKp30 (PDB: 3PV6), SIRPa/CD47 (PDB:4KJY), CTLA4/CD80 (PDB: 1I8L).

FIG. 22 shows native binding results of CD3 targeted variants to Pan T-cells as determined by flow cytometry. Results are shown for an unmasked variant (30421), an anti-CD3 one-armed antibody (18560), a construct with only the PD-1 moiety attached (31929), and variants with a full, uncleavable mask (30423) or with a full mask and a cleavable PD-L1 moiety (30430, 30436). For samples of variants 30423, 30430, uPa untreated (-uPa) and treated (+uPa) samples were tested. Data is also shown for an irrelevant control (22277).

FIGS. 23 A and B show cell killing of HCC1954, JIMT-1, HCC827 and MCF-7 tumor cells by Pan T-cells as determined in two repeats of a TDCC assay after treatment with engineered variants cross-linking T-cells and tumor cells. Results are shown for an unmasked variant (30421) as well as a combination of unmasked variant with saturating amounts of an anti-PD-L1 antibody (30421 + 120 nM atezolizumab), a variant with only the PD-1 moiety attached to the heavy chain (31929), and variants with a full, uncleavable mask (30423) or with a full mask and a cleavable PD-L1 moiety on the light chain (30430). For variants 30430 and 30423, uPa untreated (-uPa) and treated (+uPa) samples were tested. An irrelevant anti-RSV antibody (22277) was used as a negative control.

FIG. 24 shows IFNγ release of Pan T-cells as determined in two repeats of a TDCC assay with HCC1954, JIMT-1, HCC827 and MCF-7cancer cells after treatment with engineered variants cross-linking T-cells and tumor cells. Results are shown for an unmasked variant (30421) as well as a combination of unmasked variant with saturating amounts of an anti-PD-L1 antibody (30421 + 120 nM atezolizumab), a variant with only the PD-1 moiety attached to the heavy chain (31929), and variants with a full, uncleavable mask (30423) or with a full mask and a cleavable PD-L1 moiety on the light chain (30430). For variants 30430 and 30423, uPa untreated (-uPa) and treated (+uPa) samples were tested. An irrelevant anti-RSV antibody (22277) was used as a negative control.

FIG. 25 shows the receptor number per cell of Her2 and PD-L1 for a set of cancer cell lines used in TDCC and RGA assays as determined by flow cytometry.

FIGS. 26 A to D show results from a hybrid PD-1/PD-L1 Reporter Gene Assay probing cross-linking of T-cells four different cancer cell lines (HCC1954, JIMT-1, HCC827, MCF-7) and blockade of the PD-1:PD-L1 checkpoint engagement. Results are shown for an unmasked variant (30421) as well as a combination of unmasked variant with saturating amounts of an anti-PD-L1 antibody (30421 + 150 nM atezolizumab), a variant with only the PD-1 moiety attached to the heavy chain (31929), and variants with a full, uncleavable mask (30423) or with a full mask and a cleavable PD-L1 moiety on the light chain (30430). For variant 30430, uPa untreated (-uPa) and treated (+uPa) samples were tested. An irrelevant anti-RSV antibody (22277) was used as a negative control.

FIG. 27 is a drawing representative of a modified monospecific, bivalent fusion protein targeting EGFR (a-EGFR). The paratope of the Fab is sterically blocked by the SIRPα/CD47 mask.

FIG. 28 shows (A) a UPLC-SEC chromatogram and (B) non-reducing and reducing CE-SDS profiles of an EGFR-targeted, SIRPα/CD47-masked, fully cleavable variant (34164). (C) Reducing CE-SDS are also shown for the same variant without (-uPa) and with (+uPa) treatment with uPa.

FIG. 29 shows results from a native binding assay by high content analysis to EGFR positive H292 cells. Test articles include an unmasked, EGFR-targeted control (v32474), an EGFR-targeted, SIRPα/CD47-masked, fully cleavable variant (34164) without (-uPa) and with (+uPa) treatment with uPa and an irrelevant control (v22277).

FIG. 30 shows (A) data from a single titration point (1 nM) in a flow cytometry binding experiment to Her2+/PD-L1+ JIMT-1 cells as well as (B) data from a bridging experiment of human Pan T-cells and Her2+/PD-L1+ JIMT-1 cells. Data is shown for a trispecific variant with only the PD-1 moiety attached to the heavy chain (v31929) as well as bispecific variants in the same format but incapable of binding to either PD-L1 or Her2 (v32497 and v33551, respectively). Data for an irrelevant control (v22277) is included in the bridging assay (B).

FIG. 31 shows the mechanism of T-cell recruitment and activation of a PD-1:PD-L1 masked CD3 × Her2 Fab × scFv Fc variant. (A) The therapeutic antibody gets directed to the tumor microenvironment (TME) via TAA binding. (B) The PD-L1 moiety of the mask gets released via cleavage of a TME specific protease. (C) The activated therapeutic engages and activates a T-cell for tumor cell killing via the unmasked a-CD3 paratope and inhibits checkpoint activity by binding to PD-L1 on the tumor cell.

FIG. 32 shows native binding results of CD3 targeted variants to Pan T-cells as determined by flow cytometry. Results are shown for an unmasked variant (30421), a construct with only the PD-1 moiety attached (31929) and a variant with a non-functional PD-1 domain appended to the heavy chain (32497). Data is also shown for an irrelevant control (22277).

FIG. 33 shows cell killing of JIMT-1 tumor cells by Pan T-cells as determined in a TDCC assay after treatment with engineered variants cross-linking T-cells and tumor cells. Results are shown for an unmasked variant (30421), a variant with only the PD-1 moiety attached to the heavy chain (31929) and a variant with a non-functional PD-1 domain appended to the heavy chain (32497).

### DETAILED DESCRIPTION

### Definitions

The terms used in the claims and specification are defined briefly here and, in more detail, below.

“Fusion protein” refers to a protein that comprises more than one polypeptide region or domain linked to each other, e.g., by peptide bonds. Accordingly, “fused” as used herein, refers to polypeptide sequences linked to one another through a peptide bond. Examples include antibodies or scaffolds fused to immunomodulatory ligand/receptor pairs. Fusion proteins described herein are sometimes referred to as “variants” or “constructs”.

“Biologically functional protein” broadly refers to a polypeptide or protein that has a biological function, e.g., an antibody, e.g., a dimeric Fc.

“Ligand-receptor pairs” refers to a receptor polypeptide and a ligand polypeptide that specifically bind to one another. Examples include PD-1-PD-L1, CTLA4-CD80 or CD28-CD80.

“Receptor-binding fragment”, refers to any polypeptide that binds specifically to the receptor of the ligand-receptor pair. A receptor binding fragment can be naturally occurring or non-naturally occurring.

An “immunomodulatory” molecule refers to a molecule having the ability either directly or indirectly to modulate an immune response, e.g., upregulation or downregulation of an immune response, and/or immune cell activity.

“Peptidic linker” refers to a peptide that joins or links other peptides or polypeptides.

The terms “Fc region,” “Fc” and “Fc domain” are used interchangeably herein and refer to a C-terminal region of an immunoglobulin heavy chain that contains at least a portion of a constant region.

“Bispecific” refers to a biologically functional protein that can bind specifically two distinct epitopes.

“Multispecific” refers to a biologically functional protein that can bind specifically to at two or more distinct target molecules or epitopes.

“Masked” refers to a polypeptide domain, e.g., an antigen-binding domain of an antibody, that is sterically hindered from binding to a target sequence, or a ligand that is sterically hindered from binding to its cognate binding partner, e.g., its receptor.

“Protease-activated” or “protease-cleaved” or “cleaved” refers to a fusion protein comprising a protease cleavage site after it has been cleaved by a protease.

“Protease cleavage site” refers to an amino acid sequence within a fusion protein that contains a protease recognition sequence and is cleaved by a protease.

“Immune checkpoint” refers to a regulatory pathway of the immune system that regulates the immune system activation.

“Specifically binds” (and grammatical variations thereof) when referring to binding of a particular antigen, epitope, ligand or receptor, means binding that is measurably different from a non-specific interaction.

As described in more detail below, “mammal” includes both humans and non-humans and include, but is not limited to, humans, non-human primates, canines, felines, murines, bovines, equines, and porcines.

It must be noted that, as used in the specification and the appended claims, the singular forms “a,” “an” and “the” include plural referents unless the context clearly dictates otherwise.

Abbreviations used in this application include the following: PD-1 (Programmed Cell Death Protein 1); PDL-1 (Programmed death-ligand 1); CD3 (Cluster of Differentiation 3); CTLA4 (Cytotoxic T-lymphocyte-Associated Protein 4 or Cluster of Differentiation 152); CD80 (Cluster of Differentiation 80); CD28 (Cluster of Differentiation 28); CD86 (Cluster of Differentiation 86); ICOS (Inducible T Cell Costimulator); ICOSL (Inducible T Cell Costimulator Ligand); CD47 (Cluster of Differentiation 47); SIRPA (Signal-Regulatory Protein Alpha), HHLA2 (Human endogenous retro virus-H Long repeat-associating 2), NKp30 (Natural Killer cell Receptor 3), NCR3LG1(Natural Killer Cell Cytotoxicity Receptor 3 Ligand 1), HHLA2 (HERV-H LTR-associating 2), VISTA (V-domain Ig Suppressor of T cell Activation) VTCN1 (V-set domain-containing T-cell activation inhibitor 1), CD276 (Cluster of Differentiation 276), Human Epidermal Growth Factor Receptor 2 (HER2), Epidermal Growth Factor Receptor (EGFR), Mesothelin (MSLN), Tissue Factor (TF), Cluster of Differentiation 19 (CD19), tyrosine-protein kinase Met (c-Met), and Cadherin 3 (CDH3).

As used herein, the term “about” refers to an approximately +/-10% variation from a given value. It is to be understood that such a variation is always included in any given value provided herein, whether or not it is specifically referred to.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

It is contemplated that any embodiment discussed herein can be implemented with respect to any method, use or composition disclosed herein, and vice versa.

It is also to be understood that the positive recitation of a feature in one embodiment, serves as a basis for excluding the feature in another embodiment. In particular, where a list of options is presented for a given embodiment or claim, it is to be understood that one or more option can be deleted from the list and the shortened list can form an alternative embodiment, whether or not such an alternative embodiment is specifically referred to.

Various amino acid sequences and sequences of clones referred to herein are found in Table AA.

### Fusion Proteins

Disclosed herein are fusion proteins comprising a biologically functional protein, e.g., an antibody or a polypeptide scaffold, fused to a ligand-receptor pair. In the fusion proteins according to the present disclosure, the biologically functional protein comprises at least a first polypeptide and a second polypeptide and the ligand is fused to a terminus of one of the polypeptides via a first peptidic linker and the receptor is fused to the same respective terminus of the other polypeptide via a second peptidic linker. In some embodiments at least one of the first and second peptidic linkers comprises a cleavage site for a protease that naturally occurs in a target cellular environment, e.g., in a tumor microenvironment. Also disclosed are methods of using the fusion proteins disclosed herein.

The fusion proteins according to the present disclosure are masked to decrease any on-target off-tissue (e.g., off-tumor) action (i.e., toxicity) associated with target engagements. Cleavage of the peptidic linker(s) comprising the protease cleavage site in the target cellular environment results in unmasking of the fusion protein. In certain embodiments, the fusion proteins according to the present disclosure comprise a polypeptide scaffold fused to the ligand-receptor pair. In this context, the fusion protein is masked in that each of the ligand and receptor of the ligand-receptor pair are hindered from engaging a native cognate receptor or ligand through their association with each other. Cleavage of the peptidic linker(s) comprising the protease cleavage site in the target cellular environment results in unmasking of the fusion protein by releasing one member of the ligand-receptor pair from the fusion protein, thereby allowing the other member of the ligand-receptor pair to bind its cognate partner. Thus, in certain embodiments, the present disclosure provides a biologic design for programmed checkpoint or costimulatory receptor targeting.

In certain embodiments, the fusion proteins according to the present disclosure comprise an antibody or antigen-binding antibody fragment comprising an antigen-binding domain fused to the ligand-receptor pair. In this context, the fusion protein is masked in that the ligand-receptor pair sterically hinders the antigen-binding domain from binding to its cognate antigen. The fusion protein is further masked in that each of the ligand and receptor of the ligand-receptor pair are hindered from engaging a native cognate receptor or ligand through their association with each other. Cleavage of the peptidic linker(s) comprising the protease cleavage site in the target cellular environment results in unmasking of the fusion protein by releasing one member of the ligand-receptor pair from the fusion protein, thereby allowing both the other member of the ligand-receptor pair to bind its cognate partner and the antigen-binding domain to bind its cognate antigen. Thus, in certain embodiments, the present disclosure provides a multifunctional biologic design for programmed target antigen engagement and concurrent checkpoint or costimulatory receptor targeting. In certain aspects, the design of the fusion proteins described herein decreases target mediated drug disposition. In certain embodiments, the fusion proteins provide a masked antigen binding domain, e.g., a biologically functional protein, as well as a masked immunomodulatory target binding domain, e.g., a ligand-receptor pair, such that the programmed activation of one binding functionality results in the activation of the other binding functionality as well, thereby yielding a bifunctional molecule. Thus, in certain embodiments, the disclosure provides for methods of masking and conditional activation of antigen binding domains in a specific target tissue setting, as well as targeting and activation of immunomodulatory targets with reduced adverse toxicity effects.

### Ligand-Receptor Pairs

Described herein are fusion proteins each comprising a ligand-receptor pair. In certain aspects, the ligand receptor pair is an immunomodulatory pair of ligand-receptor domains belonging to the Immunoglobulin Superfamily (IgSF) (Natarajan, Kannan; Mage, Michael G; and Margulies, David H (April 2015) Immunoglobulin Superfamily. In: eLS. John Wiley & Sons, Ltd: Chichester., A F Williams 1, A N Barclay (1988) The Immunoglobulin Superfamily--Domains for Cell Surface Recognition Annu Rev Immunol 6:381-405).

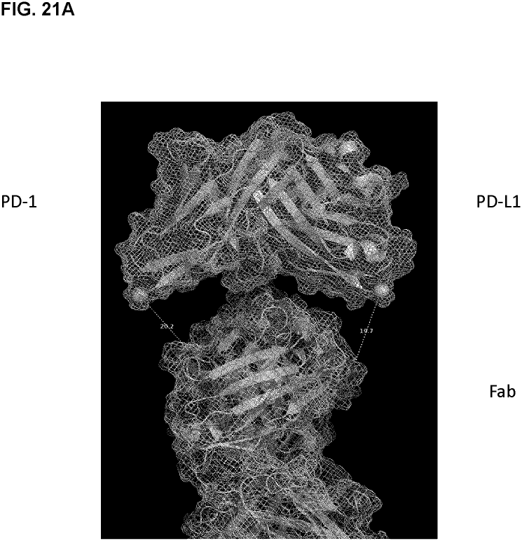

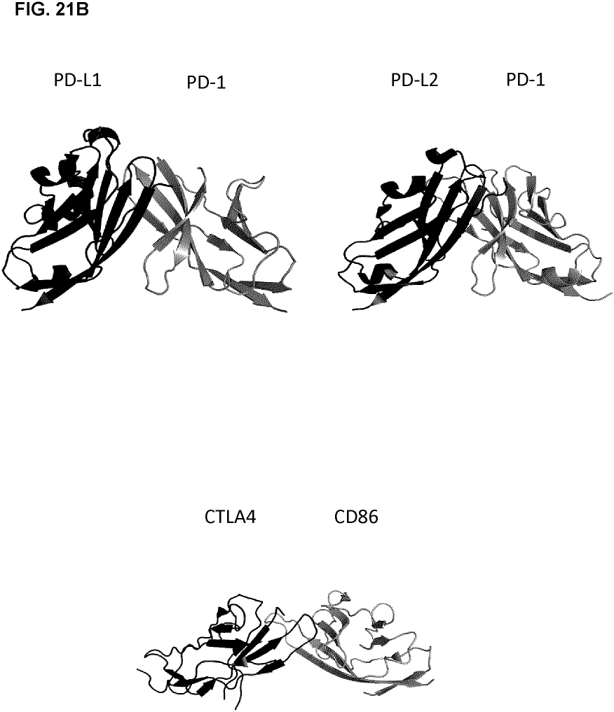

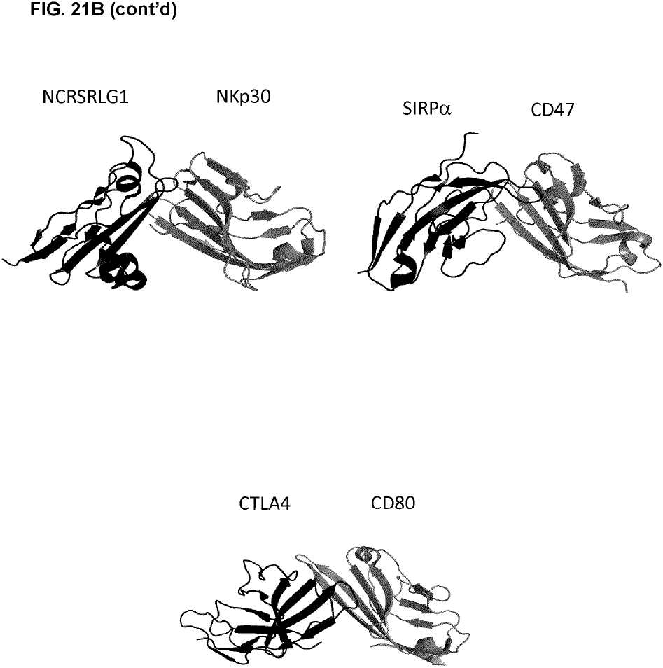

The Immunoglobulin Superfamily (IgSF) classifies a commonly found domain in proteins that is based on the core Immunoglobulin (Ig) fold. This Ig-fold consists of a beta-sandwich that is made up of a total of 7 antiparallel beta-strands that are arranged in two beta-sheets of 3 and 4 strands (FIG. 34A). The two beta-sandwiches are interconnected via a disulfide bridge between strands B and F. A structural motif commonly identified in Ig-folds is the “Greek Key” motif. Common sub-groups of the IgSF are IgV, IgC1 and IgC2 domains. Members are identified based on common structural features and the arrangement of the beta-strands. While IgC domains comprise 7 beta-strands arranged in two sheets of 3 and 4 strands (FIG. 34B), IgV domains comprise 9 beta-strands arranged in two sheets of 4 and 5 strands (FIGS. 34C,D). IgC1 and IgC2 differ in the structural arrangement of the strands. IgSF domains can be found in a wide variety of biologically important proteins including antigen receptors, immunoglobulins and immunomodulatory receptors. Surface exposed residues of the core beta sandwich as well as the loops connecting the beta strands can serve as interaction interfaces for antigen recognition, other structural domains in a tertiary/quarternary assembly or a receptor/ligand pair. As the antigen recognition site of immunoglobulins (the VH-VL pair in an antibody such as IgG1) comprises a dimer of two IgV domains, a dimer of either IgSF or IgV domains is structurally compatible to form a steric mask for that antigen recognition site if attached covalently to the N-termini of the antibody (FIG. 21).

In certain embodiments, the ligand-receptor pair is immunomodulatory, e.g., is an immune checkpoint, causes immune cell effector function modulation, modulation of T-cell receptor signaling, modulates interactions between antigen-presenting cells and effector cells or combinations thereof. In certain embodiments, the ligand-receptor pair comprises an extracellular portion of an IgSF receptor and its cognate ligand, or a receptor-binding fragment thereof. A receptor-binding fragment refers to any polypeptide that binds specifically to the receptor of the ligand-receptor pair, and can be naturally occurring or non-naturally occurring. “Naturally occurring,” as used herein and as applied to an object, refers to the fact that an object can be found in nature. For example, a polypeptide or polynucleotide sequence that is present in an organism that can be isolated from a source in nature and which has not been intentionally modified by man in the laboratory, is naturally occurring. In certain embodiments, the ligand-receptor pairs may be two interacting protein domains that belong to the immunoglobulin domain superfamily. “Non-naturally occurring”, as used herein, refers to an engineered polypeptide sequence with structural similarity to IgSF such as a mutant of a naturally occurring protein.

In certain embodiments, the disclosure herein relates to the use of an immunomodulatory pair of ligand-receptor domains belonging to the IgSF as a mask of an antibody or antibody fragment, thereby hindering target antigen binding. Examples of immunomodulatory pairs of ligand-receptor domains belonging to the Immunoglobulin Superfamily include, but are not limited to, pairs of the B7/CD28 families (such as PD1-PDL1, PD1-PDL2, CTLA4-CD80, CD28-CD80, CD28-CD86, CTLA4-CD86, PDL1-CD80, and ICOS-ICOSL, NCR3LG1-NKp30, HHLA2-CD28H and CD47-SIRPα. CD80 (also known as B7-1), CD86 (B7-2), PDL1 (B7-H1), ICOSL (B7-H2), PDL2 (B7-DC), CD276 (B7-H3), VTCN1 (B7-H4), VISTA (B7-H5), NCR3LG1 (B7-H6), HHLA2 (B7-H7) belong to the B7 family. The B7 family of proteins is typically considered the ligand and pair with members of the CD28 family which comprises CD28, CTLA4, CD28H, NKp30, PD1 and ICOS. (S.M.West and X.A. Deng. Considering B7-CD28 as a family through sequence and structure. Exp Biol Med (Maywood) 2019; 244(17): 1577-1583; doi: 10.1177/1535370219855970).

In certain embodiments, the ligand-receptor pair comprises a member of the IgSF B7/CD28 family. In certain embodiments, the ligand and the receptor comprise an extracellular portion of an immunoglobulin superfamily (IgSF) polypeptide. In certain embodiments, the ligand and the receptor comprise extracellular portions of an IgSF immunoglobulin variable (IgV) polypeptide. In certain embodiments, the ligand is a member of the IgSF B7 family and the receptor is a member of the IgSF CD28 family.

In certain embodiments, the ligand-receptor pair comprises a leukocyte costimulatory receptor. Examples of leukocyte costimulatory receptors that belong to the B7/CD28 family include ICOS (also known as CD278) and CD28. Examples of co-stimulatory ligand-receptor pairs include CD80:CD28, CD86:CD28 and ICOS:ICOSL (ICOS ligand). Examples of coinhibitory ligand-receptor pairs include PD1-PDL1, PD1-PDL2, CTLA4-CD80, CTLA4-CD86, PDL1-CD80 and CD47-SIRPα.. When linked to the N-terminus of a Fab, our results described herein indicate that they occlude access to the CDRs and hence block binding to antigens (FIG. 21A).

Other members of this large IgSFcan be used in a similar manner and carry immune modulating function. FIG. 21B shows a representation of known structures of known B7-CD28 members. The size and orientation of the domains of the other pairs is quite similar to that of PD-1 and PD-L1, and hence they may be used for binding or functional blockade similar to the PD-1/PD-L1 receptor-ligand pair.

The concept of a functional mask extends beyond members of the B7 -family. For example, FIG. 21B shows a representation of the structure of SIRPα/CD47, another ligand receptor pair with domains belonging to the IgSF, which shows good spatial compatibility to be situated at the N-terminus of a Fab and block binding. A number of therapeutic candidates are evaluating the use of antagonists in this axis to increase phagocytosis of cancer cells, making them good candidates for functional masks. (Murata Y, Saito Y, Kotani T, Matozaki T. (2018) CD47-signal regulatory protein α signaling system and its application to cancer immunotherapy. Cancer Sci. 2018 Aug; 109(8):2349-2357).

In certain embodiments, the affinity of the ligand-receptor domains in the ligand-receptor pair of the fusion protein is altered as compared to the wild-type ligand and receptor. In certain embodiments, one or both of the ligand-receptor domains in the masking pair is engineered, so as the ligand and receptor comprise sequences that are distinct from the wild-type ligand or receptor. In certain embodiments, the ligand comprises one or more mutations that increase binding affinity of the ligand for its cognate receptor. In certain embodiments, the relative binding affinity of the ligand of the ligand-receptor pair compared to a wild-type ligand is greater than 1, 1.5, 2, 2.5 3, 5, 10, 20, 30, 40, 50, 100, 500, 1000, 5,000, 10,000, 50,000 or 100,000 -fold that of the wild-type ligand to its naturally occurring, cognate receptor.

In certain embodiments, the receptor comprises one or more mutations that increase binding affinity of the receptor for its cognate ligand. In certain embodiments, the relative binding affinity of the receptor of the ligand-receptor pair compared to a wild-type receptor is greater than 1, 1.5, 2, 2.5 3, 5, 10, 20, 30, 40, 50, 100, 500, 1000, 5,000, 10,000, or 100,000 -fold that of the wild-type receptor to its naturally occurring, cognate ligand.

In certain embodiments, the ligand comprises one or more mutations that decrease binding affinity of the ligand for its cognate receptor. In certain embodiments, the relative binding affinity of the ligand of the ligand-receptor pair compared to a wild-type ligand is greater than 1, 1.5, 2, 2.5 3, 5, 10, 20, 30, 40, 50, 100, 500, 1000, 5,000, 10,000, 50,000 or 100,000 -fold lower than that of the wild-type ligand to its naturally occurring, cognate receptor.

In certain embodiments, the receptor comprises one or more mutations that decrease binding affinity of the receptor for its cognate ligand. In certain embodiments, the relative binding affinity of the receptor of the ligand-receptor pair compared to a wild-type receptor is greater than 1, 1.5, 2, 2.5 3, 5, 10, 20, 30, 40, 50, 100, 500, 1000, 5,000, 10,000, or 100,000 -fold less than that of the wild-type receptor to its naturally occurring, cognate ligand.

The ligand-receptor pair can be, e.g., the IgV domains of PD-L1 (Uniprot ID Q9NZQ7, 33-146) and PD-1 (Uniprot ID Q15116, 18-132) In some embodiments, the ligand is PD-L1 and has, e.g., an amino acid sequence corresponding to SEQ ID NO: 8 or SEQ ID NO: 10. In certain embodiments, the PD-L1 has an amino acid sequence that is substantially identical to SEQ ID NO: 8. In certain embodiments, the PD-L1 has an amino acid sequence that is about 80%, about 85%, about 90%, or about 95% identical to SEQ ID NO: 8. In certain embodiments, the PD-L1 has an amino acid sequence that is about 96%, 97%, 98%, or 99% identical to SEQ ID NO: 8. Any PD-L1 variant, e.g. high affinity variants, known in the art can be used, for example those provided in Z. Laing et al., High-affinity human PD-L1 variants attenuate the suppression of T cell activation; Oncotarget 8, 88360-88375 (2017) or WO2018/170021A1. In certain embodiments, the receptor is a high affinity PD-L1 variant. In some embodiments, the receptor is a high affinity PD-L1 variant having an amino acid sequence corresponding to SEQ ID NO: 10 or an amino acid sequence substantially identical to SEQ ID NO: 10.

In some embodiments, the receptor is PD-1 and has, e.g., an amino acid sequence corresponding to SEQ ID NO: 7 or 11. In certain embodiments, the PD-1 has an amino acid sequence that is substantially identical to SEQ ID NO: 7 or 11. In certain embodiments, the PD-1 has an amino acid sequence that is about 80%, about 85%, about 90%, or about 95% identical to SEQ ID NO: 7 or 11. In certain embodiments, the PD-1 has an amino acid sequence that is about 96%, 97%, 98%, or 99% identical to SEQ ID NO: 7 or 11. Any PD-1 variant, e.g. high affinity variants, known in the art can be used, for example those provided in R. L. Maute et al., Engineering high-affinity PD-1 variants for optimized immunotherapy and immuno-PET imaging. Proc Natl Acad Sci USA 112, E6506-6514 (2015), WO2016/022994A2 or E. Lazar-Molnar et al., Structure-guided development of a high affinity human Programmed Cell Death-1: Implications for tumor immunotherapy EBIOMedicine 17. 30-44 (2017) and WO2019/241758A1.

In certain embodiments, the receptor is a high affinity PD-1 variant. In some embodiments, the receptor is a high affinity PD-1 variant having an amino acid sequence corresponding to SEQ ID NO: 9 or an amino acid sequence substantially identical to SEQ ID NO: 9.

In certain some embodiments, the ligand is CD80 and has, e.g., an amino acid sequence corresponding to SEQ ID NO: 25. In certain embodiments, the CD80 has an amino acid sequence that is substantially identical to SEQ ID NO: 25. In certain embodiments, the CD80 has an amino acid sequence that is about 80%, about 85%, about 90%, or about 95% identical to SEQ ID NO: 25. In certain embodiments, the CD80 has an amino acid sequence that is about 96%, 97%, 98%, or 99% identical to SEQ ID NO: 25. In some embodiments, the CD80 has an amino acid sequence that is substantially identical to SEQ ID NO: 185, SEQ ID NO: 187 or SEQ ID NO: 189. In certain embodiments, the CD80 has an amino acid sequence that is about 96%, 97%, 98%, or 99% identical to SEQ ID NO: 185, SEQ ID NO: 187 or SEQ ID NO: 189. In certain embodiments, the CD80 has mutations that increase its affinity for its receptor or decrease its propensity to form homodimers during preparation. In certain embodiments, the CD80 has an amino acid sequence corresponding to SEQ ID NO: 25 with one of the following sets of mutations: (a) H18Y, A26E, E35D, M47S, I61S and D90G; (b) E35D, M47S, N48K, I61S, K89N; (c) E35D, D46V, M47S, I61S, D90G, K93E; or (d) H18Y, A26E, E35D, M47S, I61S, V68M, A71G, D90G.

In certain embodiments, the ligand is PD-L2 and has, e.g. an amino acid sequence corresponding to SEQ ID NO: 250. In certain embodiments, the PD-L2 has an amino acid sequence that is substantially identical to SEQ ID NO: 250. In certain embodiments, the PD-L2 has an amino acid sequence that is about 80%, about 85%, about 90%, or about 95% identical to SEQ ID NO: 250. In certain embodiments, the PD-L2 has an amino acid sequence that is about 96%, 97%, 98%, or 99% identical to SEQ ID NO: 250.

In certain embodiments, the ligand is CD86 and has, e.g. an amino acid sequence corresponding to SEQ ID NO: 248. In certain embodiments, the CD86 has an amino acid sequence that is substantially identical to SEQ ID NO: 248. In certain embodiments, the CD86 has an amino acid sequence that is about 80%, about 85%, about 90%, or about 95% identical to SEQ ID NO: 248. In certain embodiments, the CD86 has an amino acid sequence that is about 96%, 97%, 98%, or 99% identical to SEQ ID NO: 248.

In certain embodiments, the ligand is ICOSL and has, e.g. an amino acid sequence corresponding to SEQ ID NO: 256. In certain embodiments, the ICOSL has an amino acid sequence that is substantially identical to SEQ ID NO: 256. In certain embodiments, the ICOSL has an amino acid sequence that is about 80%, about 85%, about 90%, or about 95% identical to SEQ ID NO: 256. In certain embodiments, the ICOSL has an amino acid sequence that is about 96%, 97%, 98%, or 99% identical to SEQ ID NO: 256.

In certain embodiments, the ligand is CD276 and has, e.g. an amino acid sequence corresponding to SEQ ID NO: 258. In certain embodiments, the CD276 has an amino acid sequence that is substantially identical to SEQ ID NO: 258. In certain embodiments, the CD276 has an amino acid sequence that is about 80%, about 85%, about 90%, or about 95% identical to SEQ ID NO: 258. In certain embodiments, the CD276 has an amino acid sequence that is about 96%, 97%, 98%, or 99% identical to SEQ ID NO: 258.

In certain embodiments, the ligand is VTCN1 and has, e.g. an amino acid sequence corresponding to SEQ ID NO: 259. In certain embodiments, the VTCN1 has an amino acid sequence that is substantially identical to SEQ ID NO: 259. In certain embodiments, the VTCN1 has an amino acid sequence that is about 80%, about 85%, about 90%, or about 95% identical to SEQ ID NO: 259. In certain embodiments, the VTCN1 has an amino acid sequence that is about 96%, 97%, 98%, or 99% identical to SEQ ID NO: 259.

In certain embodiments, the ligand is VISTA and has, e.g. an amino acid sequence corresponding to SEQ ID NO: 260. In certain embodiments, the VISTA has an amino acid sequence that is substantially identical to SEQ ID NO: 260. In certain embodiments, the VISTA has an amino acid sequence that is about 80%, about 85%, about 90%, or about 95% identical to SEQ ID NO: 260. In certain embodiments, the VISTA has an amino acid sequence that is about 96%, 97%, 98%, or 99% identical to SEQ ID NO: 260.

In certain embodiments, the ligand is HHLA2 and has, e.g. an amino acid sequence corresponding to SEQ ID NO: 262. In certain embodiments, the HHLA2 has an amino acid sequence that is substantially identical to SEQ ID NO: 262. In certain embodiments, the HHLA2 has an amino acid sequence that is about 80%, about 85%, about 90%, or about 95% identical to SEQ ID NO: 262. In certain embodiments, the HHLA2 has an amino acid sequence that is about 96%, 97%, 98%, or 99% identical to SEQ ID NO: 262.

In certain embodiments, the ligand is SIRPα and has, e.g. an amino acid sequence corresponding to SEQ ID NO: 255. In certain embodiments, the SIRPα has an amino acid sequence that is substantially identical to SEQ ID NO: 255. In certain embodiments, the SIRPα has an amino acid sequence that is about 80%, about 85%, about 90%, or about 95% identical to SEQ ID NO: 255. In certain embodiments, the SIRPα has an amino acid sequence that is about 96%, 97%, 98%, or 99% identical to SEQ ID NO: 255.

In some embodiments, the receptor is CTLA4 and has, e.g., an amino acid sequence corresponding to SEQ ID NO: 26. In certain embodiments, the CTLA4 has an amino acid sequence that is substantially identical to SEQ ID NO: 26. In certain embodiments, the CTLA4 has an amino acid sequence that is about 80%, about 85%, about 90%, or about 95% identical to SEQ ID NO: 26. In certain embodiments, the CTLA4 has an amino acid sequence that is about 96%, 97%, 98%, or 99% identical to SEQ ID NO: 26.

In some embodiments, the receptor is CD28 and has, e.g., an amino acid sequence corresponding to SEQ ID NO: 253. In certain embodiments, the CD28 has an amino acid sequence that is substantially identical to SEQ ID NO: 253. In certain embodiments, the CD28 has an amino acid sequence that is about 80%, about 85%, about 90%, or about 95% identical to SEQ ID NO: 253. In certain embodiments, the CD28 has an amino acid sequence that is about 96%, 97%, 98%, or 99% identical to SEQ ID NO: 253.

In some embodiments, the receptor is CD28H and has, e.g., an amino acid sequence corresponding to SEQ ID NO: 263. In certain embodiments, the CD28H has an amino acid sequence that is substantially identical to SEQ ID NO: 263. In certain embodiments, the CD28H has an amino acid sequence that is about 80%, about 85%, about 90%, or about 95% identical to SEQ ID NO: 263. In certain embodiments, the CD28H has an amino acid sequence that is about 96%, 97%, 98%, or 99% identical to SEQ ID NO: 263.

In some embodiments, the receptor is NKp30 and has, e.g., an amino acid sequence corresponding to SEQ ID NO: 264. In certain embodiments, the NKp30 has an amino acid sequence that is substantially identical to SEQ ID NO: 264. In certain embodiments, the NKp30 has an amino acid sequence that is about 80%, about 85%, about 90%, or about 95% identical to SEQ ID NO: 264. In certain embodiments, the NKp30 has an amino acid sequence that is about 96%, 97%, 98%, or 99% identical to SEQ ID NO: 264.

In some embodiments, the receptor is ICOS and has, e.g., an amino acid sequence corresponding to SEQ ID NO: 257. In certain embodiments, the ICOS has an amino acid sequence that is substantially identical to SEQ ID NO: 257. In certain embodiments, the ICOS has an amino acid sequence that is about 80%, about 85%, about 90%, or about 95% identical to SEQ ID NO: 257. In certain embodiments, the ICOS has an amino acid sequence that is about 96%, 97%, 98%, or 99% identical to SEQ ID NO: 257.

In certain embodiments, the IgSF ligand and/or receptor has an immunoglobulin variable domain (IgV) like structure. The amino acid sequences of some exemplary naturally occurring IgV domain receptors and ligands described herein are shown in Table CC.

In certain embodiments, engineered non-naturally occurring but pairing ligands and/or receptors of the ligand-receptor pairs comprise an immunoglobulin domain with at least one of the domains with affinity for a naturally occurring immunomodulatory receptor.

In certain embodiments, the immunomodulatory ligand-receptor pairs are selected to function as antagonists or agonists of their cognate target pair. In certain embodiments, the immunomodulatory ligand-receptor pairs are selected to function as antagonist or agonist of their cognate target pair in a tumor environment. In certain embodiments, one or both the ligand or receptor of the ligand-receptor pair are designed to play a functional role following activation by protease cleavage.

### Fusion Protein Formats

The fusion proteins described herein can be in a number of different formats. The fusion proteins can be considered to have a modular architecture that includes at least a ligand receptor pair, wherein each of the ligand and receptor are fused to a biologically functional protein via peptidic linkers. The biologically functional protein, in turn, comprises at least a first and a second polypeptide. For example, either the N-terminus or C-terminus of the ligand or receptor of the ligand-receptor pair can be fused to the first and second polypeptides of the biologically functional protein, e.g., via a peptidic linker. The ligand is fused to the first polypeptide and the receptor is fused to the same respective terminus of the second polypeptide. The term, “same respective terminus”, when describing a ligand-receptor pair being fused to polypeptides, refers to the ligand and the receptor each being fused to either the N-termini of the first and second polypeptide or to the C-termini of the first and second polypeptide. Thus, in certain embodiments, the ligand is fused to the N-terminus of a first polypeptide via a first peptidic linker, and the receptor is fused to the N-terminus of a second polypeptide via a second peptidic linker. In certain embodiments, the ligand is fused to the C-terminus of a first polypeptide via a first peptidic linker, and the receptor is fused to the C-terminus of a second polypeptide via a second peptidic linker. The ligand and receptor may be fused via their C-termini or their N-termini. Both the ligand and receptor may be fused via their N- or C-termini or one of the ligand or receptor may be fused via its N-terminus while the other of the ligand or receptor is fused via its C-terminus.

In certain embodiments, the N-terminus of the ligand is fused to the N-terminus of a first polypeptide via a first peptidic linker, and the N-terminus of the receptor is fused to the N-terminus of a second polypeptide via a second peptidic linker. In certain embodiments, the C-terminus of the ligand is fused to the C-terminus of a first polypeptide via a first peptidic linker, and the C-terminus of the receptor is fused to a second polypeptide via a second peptidic linker.

In certain embodiments, the ligand is fused to a terminus of the first polypeptide of the biologically functional protein via a first peptidic linker that comprises a protease cleavage site. In certain embodiments, the receptor is fused to a terminus of the second polypeptide of the biologically functional protein via a second peptidic linker that comprises a protease cleavage site. In certain embodiments, the ligand is fused to a terminus of the first polypeptide of the biologically functional protein via a first peptidic linker that comprises a protease cleavage site, and the receptor is fused to a terminus of the second polypeptide of the biologically functional protein via a second peptidic linker that comprises a protease cleavage site. When both the first and second peptidic linkers comprise protease cleavage sites, the protease cleavage sites may be cleavable by the same protease or they may be cleavable by different proteases.

In certain embodiments, the ligand is fused to a terminus of the first polypeptide of the biologically functional protein via a first peptidic linker that comprises a protease cleavage site and the ligand is engineered to include an internal protease cleavage site which may be the same or different to the cleavage site in the first peptidic linker. In certain embodiments, the receptor is fused to a terminus of the second polypeptide of the biologically functional protein via a second peptidic linker that comprises a protease cleavage site and the receptor is engineered to include an internal protease cleavage site which may be the same or different to the cleavage site in the first peptidic linker. Including protease cleavage sites in both the peptidic linker and the member of the ligand-receptor pair that is joined to the biologically functional protein by the linker allows for cleavage and inactivation of that member of the ligand-receptor pair in the target cellular environment, while the member of the ligand-receptor pair that is still fused to the biologically active protein is unmasked (i.e., conditionally activated).

In certain embodiments, the fusion protein is conjugated to another therapeutic and/or diagnostic moiety, for example, a chemotherapeutic agent, or a radioisotope.

### Biologically Functional Proteins

The biologically functional protein can function as a scaffold and/or comprise a binding domain. Examples of polypeptide scaffolds include immunoglobulin Fc regions, albumin, albumin analogs and derivatives, toxins, cytokines, chemokines, growth factors and protein pairs such as leucine zipper domains. In certain embodiments, the biologically functional protein comprises a label, a drug, or combinations thereof. Any label known in the art suitable for detection of the fusion proteins described herein can be used. The biologically functional protein can comprise any drug, toxin or chemical known in the art to be capable of conjugation to a protein and to achieve a desired biological result.

In certain embodiments, the biologically functional proteins of the fusion proteins described herein comprise at least one antigen-binding domain. The binding domains can be, for example, immunoglobulin-based binding domains or non-immunoglobulin-based antibody mimetics, or other polypeptides or small molecules capable of specifically binding to their target, for example, a natural or engineered ligand. Non-immunoglobulin-based antibody mimetic formats include, for example, anticalins, fynomers, affimers, alphabodies, DARPins, and avimers.

The fusion proteins described herein include a biologically functional protein. Examples of biologically functional proteins include but are not limited to antibodies, e.g., polypeptides with antigen binding domains, and polypeptide scaffolds, e.g., a dimeric Fc. Thus, in certain embodiments, the first and second polypeptides of the biologically functional proteins are polypeptides comprising variable and/or constant domains of antibodies, or other domains conferring an antigen binding function or a scaffolding function to the fusion protein.

### Antibodies

In certain embodiments, the biologically functional protein is an antibody, i.e., immunoglobin. Antibodies according to the present disclosure can take on various formats as described herein, including antibody fragments. Thus, in certain embodiments, the biologically functional protein is an antibody fragment. The terms “antibody” and “immunoglobulin” are used interchangeably herein to refer to a polypeptide encoded by an immunoglobulin gene or genes, or a modified version of an immunoglobulin gene, which polypeptide specifically binds to an antigen.

Specific binding can be measured, for example, through an enzyme-linked immunosorbent assay (ELISA), a surface plasmon resonance (SPR) technique (employing, for example, a BIAcore instrument) (Liljeblad et al., 2000, Glyco J, 17:323-329), or a traditional binding assay (Heeley, 2002, Endocr Res, 28:217-229). In certain embodiments, specific binding is defined as the extent of binding to an unrelated protein being less than about 10% of the binding to the target antigen as measured by SPR, for example. In certain embodiments, specific binding of an antibody or antibody fragment for a particular antigen or an epitope is defined by a dissociation constant (K_{D}) of ≤1 µM, for example, <100 nM, <10 nM, ≤1 nM, ≤0.1 nM, <0.01 nM, or ≤ 0.001 nM. In certain embodiments, specific binding of an antibody or antibody fragment for a particular antigen or an epitope is defined by a dissociation constant (K_{D}) of 10⁻⁶ M or less, for example, 10⁻⁷ M or less, or 10⁻⁸ M or less. In some embodiments, specific binding of an antibody or antibody fragment for a particular antigen or an epitope is defined by a dissociation constant (K_{D}) between 10⁻⁶ M and 10⁻¹³ M, for example, between 10⁻⁷ M and 10⁻¹³ M, between 10⁻⁸ M and 10⁻¹³ M, or between 10⁻⁹ M and 10⁻¹³ M.

A traditional immunoglobulin structural unit is typically composed of two pairs of polypeptide chains, each pair having one “light” chain (about 25 kD) and one “heavy” chain (about 50-70 kD). Light chains are classified as either kappa or lambda. The “class” of an immunoglobulin refers to the type of constant domain possessed by its heavy chain. There are five major classes of antibodies: IgA, IgD, IgE, IgG and IgM, and several of these can be further divided into subclasses (isotypes), for example, IgG1, IgG2, IgG3, IgG4, IgA1 and IgA2. The heavy chain constant domains that correspond to the different classes of immunoglobulins are called alpha (α), delta (δ), epsilon (ε), gamma (γ) and mu (µ), respectively.

In certain embodiments, the antibodies described herein are based on an IgG class immunoglobulin, for example, an IgG1, IgG2, IgG3 or IgG4 immunoglobulin. In some embodiments, the antibodies described herein are based on an IgG1, IgG2 or IgG4 immunoglobulin. In some embodiments, the antibodies described herein are based on an IgG1 immunoglobulin. In the context of the present disclosure, when an antibody is based on a specified immunoglobulin isotype, it is meant that the antibody comprises all or a portion of the constant region of the specified immunoglobulin isotype. It is to be understood that the antibody can also comprise hybrids of isotypes and/or subclasses in some embodiments.

The N-terminal domain of each polypeptide chain of an immunoglobulin defines a variable region of about 100 to 110 or more amino acids in length that is primarily responsible for antigen recognition. The terms variable light chain (VL) and variable heavy chain (VH) refer to these domains in the light and heavy chain respectively.

Accordingly, it can be seen that immunoglobulins comprise different domains within the heavy and light chains. Such domains can be overlapping and include, the Fc domain (or Fc region), the CH1 domain, the CH2 domain, the CH3 domain, the hinge domain, the heavy constant domain (CH1-hinge-Fc or CH1-hinge-CH2-CH3), the variable heavy domain (VH), the variable light domain (VL) and the light constant domain (CL). The “Fc domain” includes the CH2 and CH3 domains, and optionally a hinge domain (or hinge region).

In each of the VH and VL domains of an immunoglobulin are three loops which are hypervariable in sequence and form an antigen-binding site. Each of these loops is referred to as a “hypervariable region” or “HVR.” The terms hypervariable region (HVR) and complementarity determining region (CDR) are used herein interchangeably in reference to the portions of the variable region that form the antigen-binding domain. With the exception of CDR1 in VH, CDRs generally comprise the amino acid residues that form the hypervariable loops. The VH and VL domains consist of relatively invariant stretches called framework regions (FRs) of between about 15 to 30 amino acids in length separated by the shorter CDRs, which are each typically between about 5 and 15 amino acids in length, although can occasionally be longer or shorter. The three CDRs and four FRs that make up each VH and VL domain are arranged from N- to C-terminus as follows: FR1-CDR1-FR2-CDR2-FR3-CDR3-FR4.

A number of different definitions of the CDR regions are in common use, including those described by Kabat et al. (1983, Sequences of Proteins of Immunological Interest, NIH Publication No. 369-847, Bethesda, MD), by Chothia et al. (1987, J Mol Biol, 196:901-917), as well as the IMGT, AbM and Contact definitions. These different definitions include overlapping or subsets of amino acid residues when compared against each other. By way of example, CDR definitions according to Kabat, Chothia, IMGT, AbM and Contact are provided in Table 1 below. Accordingly, as would be readily apparent to one skilled in the art, the exact numbering and placement of CDRs can differ based on the numbering system employed. However, it is to be understood that the disclosure herein of a variable heavy domain (VH) includes the disclosure of the associated (inherent) heavy chain CDRs (HCDRs) as defined by any of the known numbering systems. Similarly, disclosure herein of a variable light domain (VL) includes the disclosure of the associated (inherent) heavy chain CDRs (HCDRs) as defined by any of the known numbering systems.

TABLE 1

Common CDR Definitions¹

| Definition | Heavy Chain | | | Light Chain | | |
|---|---|---|---|---|---|---|
| | CDR1² | CDR2 | CDR3 | CDR1 | CDR2 | CDR3 |
| Kabat | H31-H35 | H50-H65 | H95-H102 | L24-L34 | L50-L56 | L89-L97 |
| Chothia | H26-H32, H33 or H34 | H52-H56 | H95-H102 | L24-L34 | L50-L56 | L89-L97 |
| IMGT | H26-H33, H34, H35, H35A or H35B | H51-H57 | H93-102 | L27-L32 | L50-L52 | L89-L97 |
| AbM | H26-H35B | H50-H58 | H95-H102 | L24-L34 | L50-L56 | L89-L97 |
| Contact | H30-H35B | H47-H58 | H95-H101 | L30-L36 | L46-L55 | L89-L96 |

¹ Either the Kabat or Chothia numbering system can be used for HCDR2, HCDR3 and the light chain CDRs for all definitions except Contact, which uses Chothia numbering
² Using Kabat numbering. The position in the Kabat numbering scheme that demarcates the end of the Chothia and IMGT CDR-H1 loop varies depending on the length of the loop due to the placement of insertions outside of those CDR definitions at positions 35A and 35B in Kabat. The IMGT and Chothia CDR-H1 loop can be unambiguously defined using Chothia numbering. CDR-H1 definitions using Chothia numbering are: Kabat H31-H35, Chothia H26-H32, AbM H26-H35, IMGT H26-H33, Contact H30-H35.

One skilled in the art will appreciate that a limited number of amino acid substitutions can be introduced into the CDR sequences or to the VH or VL sequences of known antibodies without the antibody losing its ability to bind its target. Candidate amino acid substitutions can be identified by computer modeling or by techniques such as alanine scanning as described above, with the resulting variants being tested for binding activity by standard techniques. For example, in certain embodiments the EGFR binding domain comprised by the fusion protein comprises a set of CDRs (i.e., heavy chain CDR1, CDR2 and CDR3, and light chain CDR1, CDR2 and CDR3) that have 90% or greater, 95% or greater, 98% or greater, 99% or greater, or 100% sequence identity to a set of CDRs from cetuximab or panitumumab, wherein the binding domain retains the ability to bind EGFR. In certain embodiments, the EGFR binding domain comprised by the fusion protein comprises a variant of these CDR sequences comprising between 1 and 10 amino acid substitutions across the three CDRs (that is, the CDRs can be modified by including up to 10 amino acid substitutions with any combination of CDRs being modified), for example, between 1 and 7 amino acid substitutions, between 1 and 5 amino acid substitutions, between 1 and 4 amino acid substitutions, between 1 and 3 amino acid substitutions, between 1 and 2 amino acid substitutions, or 1 amino acid substitution, across the CDRs, wherein the variant retains the ability to bind EGFR. Typically, such amino acid substitutions will be conservative amino acid substitutions such as those outlined in Column 1 or Column 2 of Table 4 below.

In certain embodiments, the antibodies described herein comprise at least one immunoglobulin domain from a mammalian immunoglobulin, such as a bovine immunoglobulin, a human immunoglobulin, a camelid immunoglobulin, a rat immunoglobulin or a mouse immunoglobulin. In some embodiments, a biologically functional protein can be a chimeric antibody and comprises two or more immunoglobulin domains, in which at least one domain is from a first mammalian immunoglobulin, for example a human immunoglobulin, and at least a second domain is from a second mammalian immunoglobulin, for example, a mouse or rat immunoglobulin. In some embodiments, the biologically functional protein comprises at least one immunoglobulin constant domain from a human immunoglobulin.

One skilled in the art will understand that these domains can be combined in various ways to provide an antibody having different formats, including multispecific antibodies of different formats. These formats are based generally on antibody formats known in the art (see, for example, review by Brinkmann & Kontermann, 2017, MABS, 9(2):182-212, and Müller & Kontermann, “Bispecific Antibodies” in Handbook of Therapeutic Antibodies, Wiley-VCH Verlag GmbH & Co. (2014)).

The antibodies of the biologically functional proteins described herein can have different valencies. In certain embodiments, the biologically functional protein comprises a single antigen binding domain. In certain embodiments, the biologically functional protein comprises two or more antigen binding domains. In certain embodiments, the biologically functional protein comprises an antibody that has different valencies and specificities. A “bispecific antibody” as used herein, comprises two binding domains. In certain embodiments, each of the two binding domains has a unique binding specificity. A “multispecific antibody” as used herein, comprises two or more binding domains. In certain embodiments, each of the two or more binding domains has a unique binding specificity. In some embodiments, at least two of the two or more binding domains have unique binding specificities. For example, the antibody can be bivalent and bispecific, or can be bivalent and have a single specificity. Alternatively, the antibody can be trivalent and bispecific, that is the antibody comprises three binding domains. The antibody can also be bispecific and tetravalent, that is the antibody comprises four binding domains. Other valencies are also possible.

When the antibody comprises two binding domains that bind to the same target molecule, the binding domains can bind to the same epitope on the target molecule or they can bind to different epitopes on the target molecule. In some embodiments, the antibody comprises two binding domains that bind to different epitopes on the target molecule. The term “biparatopic” can be used to refer to an antibody which comprises two binding domains that bind to different epitopes on the same target molecule (antigen). A biparatopic antibody can bind to a single antigen molecule through the two different epitopes, or it can bind to two separate antigen molecules, each through a different epitope.

In certain embodiments, the antibody is biparatopic and bispecific in that it comprises a first binding domain and a second binding domain, each of which binds to a different epitope on the first target molecule, and a third binding domain that binds to the second target molecule. Alternatively, a bispecific biparatopic antibody can comprise a first binding domain and a second binding domain, each binding to a different epitope on the first target molecule, and a third binding domain and a fourth binding domain, each binding to a different epitope on the second target molecule.

In some embodiments, the antibody further comprises a scaffold and the binding domains are operably linked to the scaffold. “Operably linked,” as used herein, means that the components described are in a relationship permitting each of them to function in their intended manner. The binding domains can be directly or indirectly linked to the scaffold. By indirectly linked, it is meant that a given binding domain is linked to the scaffold via another component, for example, a linker or one of the other binding domains. Various formats for fusion proteins that comprise a scaffold are described in more detail below.

### Antigen Binding Domain Formats

In some embodiments, the fusion proteins described herein include an antibody having at least one antigen binding domain that is an antibody fragment, such as a Fab, a Fab′, a single chain Fab (scFab), a single chain Fv (scFv) or a single domain antibody (sdAb).

A “Fab” or “Fab fragment” contains the constant domain (CL) of the light chain and the first constant domain (CH1) of the heavy chain along with the variable domains VL and VH on the light and heavy chains, respectively, which comprise the CDRs. A Fab′ or Fab′ fragment differs from a Fab fragment by the addition of a few amino acid residues at the C-terminus of the heavy chain CH1 domain, including one or more cysteine residues from the hinge region.

A Fab fragment can comprise two separate polypeptide chains (a light chain and a heavy chain) or it can be a single chain Fab. A single chain Fab is a Fab molecule in which the Fab light chain and the Fab heavy chain are connected by a peptide linker to form a single peptide chain. Typically, the C-terminus of the Fab light chain is connected to the N-terminus of the Fab heavy chain in the single-chain Fab molecule, however, other formats are also possible.

An “scFv” includes a heavy chain variable domain (VH) and a light chain variable domain (VL) of an antibody in a single polypeptide chain. The scFv can optionally comprise a polypeptide linker between the VH and VL domains which can assist the scFv in forming a desired structure for antigen binding. An scFv can include a VL connected from its C-terminus to the N-terminus of a VH by a linker, i.e., VL-Linker-VH, or alternately, an scFv can comprise a VH connected through its C-terminus to the N-terminus of a VL by a linker, i.e., VH-Linker-VL. For a review of scFvs see Pluckthun in The Pharmacology of Monoclonal Antibodies, vol. 113, Rosenburg and Moore eds., Springer-Verlag, New York, pp. 269-315 (1994).

The term “sdAb” refers to a single immunoglobulin domain. An sdAb can be, for example, of camelid origin. Camelid antibodies lack light chains and their antigen-binding sites consist of a single domain, termed a “VHH.” An sdAb comprises three CDR/hypervariable loops that form the antigen-binding site: CDR1, CDR2 and CDR3. sdAbs are fairly stable and easy to express, for example, as a fusion with the Fc chain of an antibody (see, for example, Harmsen & De Haard, 2007, Appl. Microbiol Biotechnol. 77(1):13-22).

In some embodiments, one or more of the binding domains comprised by the antibody can be a natural or engineered ligand for the target receptor, or a functional fragment of such a ligand, i.e., a fragment capable of specifically binding to the target receptor.

The antigen binding domains can be in the form of combinations of individual scFvs, Fabs, sdAbs. For example, when the binding domains are in the form of scFvs, formats such as tandem scFv ((scFv)₂ or taFv) or triplebody (3 scFvs) can be constructed, in which the scFvs are connected together by a flexible linker. scFvs can also be used to construct diabody, triabody and tetrabody (tandem diabodies or TandAbs) formats, which comprise 2, 3 and 4 scFvs, respectively, connected by a short linker. The restricted length of the linker (usually about 5 amino acids in length) results in dimerization of the scFvs in a head-to-tail manner. In any of the preceding formats, the scFvs can be further stabilized by inclusion of an interdomain disulfide bond. For example, a disulfide bond can be introduced between VL and VH through introduction of an additional cysteine residue in each chain (for example, at position 44 in VH and 100 in VL) (see, for example, Fitzgerald et al., 1997, Protein Engineering, 10:1221-1225) or a disulfide bond can be introduced between two VHs to provide an antigen binding domain having a DART format (see, for example, Johnson et al., 2010, J Mol. Biol., 399:436-449).

Similarly, formats comprising two or more sdAbs, such as VHs or VHHs, connected together through a suitable linker can be used for the biologically functional protein. Other examples of antibody formats that lack a scaffold include those based on Fab fragments, for example, Fab₂, F(ab′)₂ and F(ab′)₃ formats, in which the Fab fragments are connected through a linker or an IgG hinge region.

Combinations of antigen binding domains in different forms can also be employed to generate alternative formats. For example, an scFv or a sdAb can be fused to the C-terminus of either or both of the light and heavy chain of a Fab fragment resulting in a bivalent (Fab-scFv) or (Fab-sdAb) or trivalent (Fab-(scFv)₂ or Fab-(sdAb)₂). Similarly, one or two scFvs or sdAbs can be fused at the hinge region of a F(ab′) fragment to produce a tri-or tetravalent F(ab′)₂-scFv/sdAb. The binding domains can be in one or a combination of the forms described above (for example, scFvs, Fabs and/or sdAbs, or ligand-based binding domains).

In certain specific embodiments, the biologically functional protein comprises a bispecific antibody that binds an immune cell antigen, e.g., CD3, and a tumor associated antigen (TAA), e.g., HER2. In certain more specific embodiments, the biologically functional protein comprises a bi-specific antibody with a Fab-scFv format wherein the Fab binds an immune cell antigen and the scFv binds a TAA. In certain more specific embodiments, the biologically functional protein comprises a bi-specific antibody with a Fab-scFv format wherein the Fab binds CD3 and the scFv binds HER2. In some embodiments, the biologically functional protein comprises a bi-specific antibody with a Fab-Fab format wherein one Fab binds CD3 and the other Fab binds HER2.

In certain embodiments, the biologically functional protein comprises two or more antigen binding domains operably linked to a heterodimeric Fc. In this context, the biologically functional protein can be bivalent, trivalent or tetravalent. Non-limiting examples of formats are described below. Other configurations are known in the art (see, for example, Spiess et al., 2015, Mol Immunol., 67:95-106).

Exemplary configurations for a biologically functional protein comprising two binding domains operably linked to a heterodimeric Fc, i.e., a bivalent antibody, include, but are not limited to: a) mAb format in which the first binding domain is a Fab that is operably linked to the N-terminus of the first Fc polypeptide of the heterodimeric Fc and the second binding domain is a Fab that is operably linked to the N-terminus of the second Fc polypeptide; b) hybrid format in which the first binding domain is an scFv that is operably linked to the N-terminus of one Fc polypeptide of the heterodimeric Fc and the second binding domain is a Fab that is operably linked to the N-terminus of the other Fc polypeptide, and c) dual scFv format in which the first binding domain is an scFv that is operably linked to the N-terminus of the first Fc polypeptide of the heterodimeric Fc and the second binding domain is an scFv that is operably linked to the N-terminus of the second Fc polypeptide.

Other examples include antibodies comprising one binding domain (either first or second) as a Fab or an scFv operably linked to the N-terminus of the first Fc polypeptide and the other binding domain as a Fab or an scFv operably linked to the C-terminus of the second Fc polypeptide.

Exemplary configurations for a multispecific antibody comprising three binding domains operably linked to a heterodimeric Fc (i.e. a trivalent antibody) include, but are not limited to:
A) mAb-Fv format in which the first binding domain is a Fab that is operably linked to the N-terminus of the first Fc polypeptide of the heterodimeric Fc and the second binding domain is a Fab that is operably linked to the N-terminus of the second Fc polypeptide, with the third binding domain being made up of a VH domain attached to the C-terminus of one Fc polypeptide and a VL domain attached to the C-terminus of the other Fc polypeptide;
B) mAb-scFv format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is an scFv operably linked to the C-terminus of either the first or the second Fc polypeptide;
C) scFv-mAb format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is an scFv operably linked to the N-terminus of either the first or the second binding domain;
D) central scFv format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the first binding domain (scFv);
E) Fab-hybrid format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the N-terminus of the first or second binding domain;
F) scFv-hybrid format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is an scFv that is operably linked to the N-terminus of the first or second binding domain;
G) hybrid-scFv format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is an scFv that is operably linked to the C-terminus of either the first or the second Fc polypeptide;
H) hybrid-Fab format in which the first binding domain is an scFv that is operably linked to the N-terminus of one Fc polypeptide of the heterodimeric Fc, the second binding domain is a Fab that is operably linked to the N-terminus of the other Fc polypeptide, and the third binding domain is a Fab that is operably linked to the C-terminus of either the first or the second Fc polypeptide; and
I) Fab-mAb format in which the first binding domain is a Fab that is operably linked to the N-terminus of the first Fc polypeptide of the heterodimeric Fc, the second binding domain is a Fab that is operably linked to the N-terminus of the second Fc polypeptide and the third binding domain is a Fab operably linked to the N-terminus of either the first or the second binding domain.

Exemplary configurations for a multispecific antibody comprising four binding domains operably linked to a heterodimeric Fc, i.e., a tetravalent antibody, include, but are not limited to: i) central-scFv2 format in which the first binding domain is an scFv that is operably linked to the N-terminus of one Fc polypeptide of the heterodimeric Fc, the second binding domain is an scFv that is operably linked to the N-terminus of the other Fc polypeptide, the third binding domain is a Fab that is operably linked to one of the scFvs and the fourth binding domain is a Fab that is operably linked to the other scFv, and ii) dual variable domain format in which the first binding domain is a Fab that is operably linked to the N-terminus of one Fc polypeptide of the heterodimeric Fc, the second binding domain is a Fab that is operably linked to the N-terminus of the other Fc polypeptide, the third binding domain is an scFv that is operably linked to one of the Fabs and the fourth binding domain is an scFv that is operably linked to the other Fab.

The antibodies of the biologically functional proteins described herein can comprise a label, a drug, or combinations thereof. Any label known in the art suitable for detection of the fusion proteins described herein can be used. Antibody drug conjugates are described in more detail below.

In certain embodiments, the antigen binding domains of the antibodies of the biologically functional protein described herein bind to the same antigen on the same cell. In certain embodiments, the antigen binding domains bind to more than one antigen on the same cell. In certain embodiments, the antigen binding domains bind to more than one antigen, wherein at least one antigen is on a different cell than another antigen. In certain embodiments, the antigen binding domain(s) of the antibody bind to a tumor cell or an immune cell. In certain embodiments, the antigen binding domains of the antibody bind to a tumor cell and an immune cell.

### Chimeric and Humanized and Variant Antibodies

In some embodiments, the antibodies can be derived from immunoglobulins that are from different species, for example, the antibody can be a chimeric antibody or a humanized antibody. A “chimeric antibody” refers to an antibody that typically comprises at least one variable domain from a rodent antibody (usually a murine antibody) and at least one constant domain from a human antibody. A “humanized antibody” is a type of chimeric antibody that contains minimal sequence derived from a non-human antibody.

The human constant domain of a chimeric antibody need not be of the same isotype as the non-human constant domain it replaces. Chimeric antibodies are discussed, for example, in Morrison et al., 1984, Proc. Natl. Acad. Sci. USA, 81:6851-55, and U.S. Pat. No. 4,816,567. Generally, humanized antibodies are human immunoglobulins (recipient antibody) in which residues from a hypervariable region of the recipient are replaced by residues from a hypervariable region of a non-human species (donor antibody), such as mouse, rat, rabbit, or non-human primate, having the desired specificity and affinity for a target antigen. This technique for creating humanized antibodies is often referred to as “CDR grafting.” “Chimeric antibody” and “humanized antibody” both refer generally to antibodies that combine immunoglobulin regions or domains from more than one species.

In some instances, additional modifications are made to further refine antibody performance. For example, framework region (FR) residues of the human immunoglobulin are replaced by corresponding non-human residues, or the humanized antibodies can comprise residues that are not found in either the recipient antibody or the donor antibody. In general, a variable domain in a humanized antibody will comprise all or substantially all of the hypervariable regions from a non-human immunoglobulin and all or substantially all of the FRs from a human immunoglobulin sequence. Humanized antibodies are described in more detail in Jones, et al., 1986, Nature, 321:522-525; Riechmann, et al., 1988, Nature, 332:323-329 and Presta, 1992, Curr. Op. Struct. Biol., 2:593-596, for example.

A number of approaches are known in the art for selecting the most appropriate human frameworks in which to graft the non-human CDRs. Early approaches used a limited subset of well-characterized human antibodies, irrespective of the sequence identity to the non-human antibody providing the CDRs (the “fixed frameworks” approach). More recent approaches have employed variable regions with high amino acid sequence identity to the variable regions of the non-human antibody providing the CDRs (“homology matching” or “best-fit” approach). An alternative approach is to select fragments of the framework sequences within each light or heavy chain variable region from several different human antibodies. CDR-grafting can in some cases result in a partial or complete loss of affinity of the grafted molecule for its target antigen. In such cases, affinity can be restored by back-mutating some of the residues of human origin to the corresponding non-human ones. Methods for preparing humanized antibodies by these approaches are well-known in the art (see, for example, Tsurushita & Vasquez, 2004, Humanization of Monoclonal Antibodies, Molecular Biology of B Cells, 533-545, Elsevier Science (USA); Jones et al., 1986, Nature, 321:522-525; Riechmann et al., 1988, Nature, 332:323-329; Presta et al., 1997, Cancer Res, 57(20):4593-4599).

Alternatively, or in addition to, these traditional approaches, more recent technologies can be employed to further reduce the immunogenicity of a CDR-grafted humanized antibody. For example, frameworks based on human germline sequences or consensus sequences can be employed as acceptor human frameworks rather than human frameworks with somatic mutation(s). Another technique that aims to reduce the potential immunogenicity of non-human CDRs is to graft only specificity-determining residues (SDRs). In this approach, only the minimum CDR residues required for antigen-binding activity (the “SDRs”) are grafted into a human germline framework. This method improves the “humanness” (i.e. the similarity to human germline sequence) of the humanized antibody and thus helps reduce the risk of immunogenicity of the variable region. These techniques have been described in various publications (see, for example, Almagro & Fransson, 2008, Front Biosci, 13:1619-1633; Tan, et al., 2002, J Immunol, 169:1119-1125; Hwang, et al., 2005, Methods, 36:35-42; Pelat, et al., 2008, J Mol Biol, 384:1400-1407; Tamura, et al., 2000, J Immunol, 164:1432-1441; Gonzales, et al., 2004, Mol Immunol, 1:863-872, and Kashmiri, et al., 2005, Methods, 36:25-34).

In certain embodiments, the antibody comprises humanized antibody sequences, for example, one or more humanized variable domains. In some embodiments, the antibody is a humanized antibody.

In certain embodiments, an antigen binding domain comprised by the fusion protein is a substitutional variant of a known antibody that comprises one or more amino acid substitutions in the CDRs of the parent antibody. In certain embodiments, the substitution variant has modifications (for example, improvements) in certain biological properties relative to the parent antibody. For example, the substitution variant can have increased affinity for the target protein or it can have reduced immunogenicity. In some embodiments, the substitution variant substantially retains certain biological properties of the parent antibody.

CDR hotspots are residues encoded by codons that undergo mutation at high frequency during the somatic maturation process (see, for example, Chowdhury, 2008, Methods Mol. Biol., 207:179-196). Affinity maturation by constructing and reselecting from secondary libraries has been described (see, for example., Hoogenboom et al. in Methods in Molecular Biology, 178:1-37, O’Brien et al., ed., Human Press, Totowa, N.J. (2001)).

Methods of affinity maturation are well known in the art. For example, diversity can be introduced into the variable genes chosen for maturation by various techniques including, for example, error-prone PCR, chain shuffling or oligonucleotide-directed mutagenesis. A secondary library is then created, and this library is screened to identify any antibody variants with the desired affinity. Another method to introduce diversity involves CDR-directed approaches, in which several CDR residues (for example, 2, 3, 4 or more residues at a time) are randomized. CDR3 of either or both of the heavy or light chain is often targeted for CDR-directed approaches. CDR residues involved in antigen binding can be identified for example using alanine scanning mutagenesis (see, for example, Cunningham and Wells, 1989, Science, 244:1081-1085) or by computer modeling using a crystal structure of an antigen-antibody complex to identify contact points between the antibody and antigen.

In certain embodiments, a substitution variant comprises one or more substitutions within one or more CDRs provided that the substitutions do not substantially reduce the ability of the binding domain to bind its target antigen. For example, a substitution variant can comprise one or more conservative substitutions as described herein within one or more CDRs that do not substantially reduce binding affinity. In some embodiments, a substitution variant comprises one or more amino acid substitutions within the CDRs that do not involve the antigen-contacting amino acids. In some embodiments, a substitution variant comprises a variant VH or VL sequence in which each CDR either is unaltered or contains no more than one, two or three amino acid substitutions.

### Glycosylation Variants

In certain embodiments, the fusion proteins described herein comprise a biologically functional protein based on an IgG Fc in which native glycosylation has been modified. As is known in the art, glycosylation of an Fc can be modified to increase or decrease effector function.

For example, mutation of the conserved asparagine residue at position 297 to alanine, glutamine, lysine or histidine (i.e. N297A, Q, K or H) results in an aglycoslated Fc that lacks all effector function (Bolt et al., 1993, Eur. J. Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa(see, for example, Shields et al., 2002, J Biol Chem., 277:26733-26740, and Niwa et al., 2005, J. Immunol. Methods, 306:151-160). Such low fucose antibodies can be produced, for example in knockout Chinese hamster ovary (CHO) cells lacking fucosyltransferase (FUT8) (Yamane-Ohnuki et al., 2004, Biotechnol. Bioeng., 87:614-622), in the variant CHO cell line, Lec 13, that has a reduced ability to attach fucose to N297-linked carbohydrates (International Publication No. WO 03/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogs to culture medium during antibody production to inhibit incorporation of fucose into the carbohydrate on the antibody.

Other methods of producing antibodies with little or no fucose on the Fc glycosylation site (N297) are well known in the art. For example, the GlymaX® technology (ProBioGen AG) (see von Horsten et al., 2010, Glycobiology, 20(12):1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants can have reduced fucosylation and/or improved ADCC function. See, for example, International Publication No. WO 2003/011878, U.S. Pat. No. 6,602,684 and U.S. Pat. Application Publication No. US 2005/0123546. Useful glycosylation variants also include those having at least one galactose residue in the oligosaccharide attached to the Fc region, which can have improved CDC function (see, for example, International Publication Nos. WO 1997/030087, WO 1998/58964 and WO 1999/22764).

### Polypeptide Scaffolds

In certain embodiments, the biologically functional protein of the fusion proteins described herein is a polypeptide scaffold, which can function, e.g., to stabilize or extend the in vivo half-life of the ligand receptor pair.

In certain embodiments, the biologically functional protein consists of a dimeric Fc region. In certain embodiments, the first and second polypeptide of the biologically functional protein consists of a dimeric Fc, wherein the first polypeptide consists of a first Fc polypeptide and the second polypeptide consists of a second Fc polypeptide, the first and second Fc polypeptides forming a dimeric Fc region. In certain embodiments, the dimeric Fc region is a heterodimeric Fc. Heterodimeric Fc regions are described in more detail herein.

In certain embodiments, the polypeptide scaffolds are comprised of a first and second polypeptide. In certain embodiments, the ligand of the ligand receptor pair is fused via a peptidic linker to the first polypeptide and the receptor is fused via a peptidic linker to the same respective terminus of the second polypeptide. Thus, in certain embodiments, the ligand is fused to the N-terminus of a first polypeptide via a peptidic linker, and the receptor is fused to the N-terminus of a second polypeptide via a second peptidic linker. Conversely, in certain embodiments, the ligand is fused to the C-terminus of a first polypeptide via a peptidic linker, and the receptor is fused to a second polypeptide via a second peptidic linker.

In certain more specific embodiments, the biologically functional protein comprises a polypeptide scaffold that consists of a dimeric Fc region and a ligand-receptor pair that is PDL-1 and PD-1. In certain embodiments, the fusion protein comprises a biologically functional protein that consists of a dimeric Fc region and a ligand-receptor pair that is CD80 and CTLA4. In certain embodiments, an Fc domain of the polypeptide scaffold comprises an amino acid sequence corresponding to SEQ ID NOs: 4 and 5, and optionally SEQ ID NO: 6. In certain embodiments, the polypeptide scaffold consists of a heterodimeric Fc comprising SEQ ID NO: 4 and SEQ ID NO: 5; wherein a first Fc polypeptide comprises SEQ ID NO: 4 and a second Fc polypeptide comprises SEQ ID NO: 5. In some embodiments, the polypeptide scaffold consisting of a heterodimeric Fc comprises a modified CH3 and/or CH2 domain of Table 2 and Table 3, respectively.

### Fc Domains

In certain embodiments, the fusion proteins described herein include biologically functional proteins, e.g., antibodies or polypeptide scaffolds, comprising a dimeric immunoglobulin Fc region. The term “Fc region” includes native sequence Fc regions and variant Fc regions. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD (1991). An “Fc polypeptide” of a dimeric Fc refers to one of the two polypeptides forming the dimeric Fc region, that is a polypeptide comprising C-terminal constant regions of an immunoglobulin heavy chain that is capable of stable selfassociation.

An Fc region can comprise either a CH3 domain or a CH3 and a CH2 domain. The CH3 domain comprises two CH3 sequences, each comprised by one of the two Fc polypeptides of the dimeric Fc. Similarly, the CH2 domain comprises two CH2 sequences, each comprised by one of the two Fc polypeptides of the dimeric Fc.

In certain embodiments, the fusion protein comprises an Fc based on a human IgG Fc. In some embodiments, the fusion protein comprises an Fc based on a human IgG1 Fc. In some embodiments, the fusion protein comprises an Fc based on a heterodimeric Fc comprising two different Fc polypeptides.

In certain embodiments, the fusion protein comprises an Fc based on a modified IgG Fc in which the CH3 domain comprises one or more amino acid modifications. In some embodiments, the fusion protein comprises an Fc based on a modified IgG Fc in which the CH2 domain comprises one or more amino acid modifications. In some embodiments, the fusion protein comprises an Fc based on a modified IgG Fc in which the CH3 domain comprises one or more amino acid modifications and the CH2 domain comprises one or more amino acid modifications.

### Modified Fc CH3 Domains

In certain embodiments, the fusion protein comprises a heterodimeric immunoglobulin Fc comprising a modified CH3 domain, wherein the modified CH3 domain comprises one or more asymmetric amino acid modifications. As used herein, an “asymmetric amino acid modification” refers to a modification in which an amino acid at a specific position on the first Fc polypeptide is different to the amino acid at the corresponding position on the second Fc polypeptide. These asymmetric amino acid modifications can comprise modification of only one of the two amino acids at the corresponding position on each Fc polypeptide, or they can comprise modifications of both amino acids at the corresponding positions on each of the first and second Fc polypeptides.

In certain embodiments, the fusion protein comprises a heterodimeric Fc comprising a modified CH3 domain, wherein the modified CH3 domain comprises one or more asymmetric amino acid modifications that promote formation of the heterodimeric Fc over formation of a homodimeric Fc. Amino acid modifications that can be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Publication Nos. WO 2012/058768 and WO 2013/063702.

In certain embodiments, the fusion protein comprises a heterodimeric Fc having a modified CH3 domain as described in International Publication No. WO 2012/058768 or International Patent Publication No. WO 2013/063702.

In some embodiments, the fusion protein comprises a heterodimeric human IgG1 Fc having a modified CH3 domain. Table 2 below provides the amino acid sequence of the human IgG1 Fc sequence, corresponding to amino acids 231 to 447 of the full-length human IgG1 heavy chain. The CH2 domain is typically defined as comprising amino acids 231-340 of the full-length human IgG1 heavy chain and the CH3 domain is typically defined as comprising amino acids 341-447 of the full-length human IgG1 heavy chain.

In certain embodiments, the fusion protein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including amino acid modifications at positions F405 and Y407, and a second Fc polypeptide including amino acid modifications at positions T366 and T394. In some embodiments, the amino acid modification at position F405 of the first Fc polypeptide of the modified CH3 domain is F405A, F405I, F405M, F405S, F405T or F405V. In some embodiments, the amino acid modification at position Y407 of the first Fc polypeptide of the modified CH3 domain is Y407I or Y407V. In some embodiments, the amino acid modification at position T366 of the second Fc polypeptide of the modified CH3 domain is T366I, T366L or T366M. In some embodiments, the amino acid modification at position T394 of the second Fc polypeptide of the modified CH3 domain is T394W. In some embodiments, the first Fc polypeptide of the modified CH3 domain further includes an amino acid modification at position L351. In some embodiments, the amino acid modification at position L351 in the first Fc polypeptide of the modified CH3 domain is L351Y. In some embodiments, the second Fc polypeptide of the modified CH3 domain further includes an amino acid modification at position K392. In some embodiments, the amino acid modification at position K392 in the second Fc polypeptide of the modified CH3 domain is K392F, K392L or K392M. In some embodiments, one or both of the first and second Fc polypeptides of the modified CH3 domain further comprises the amino acid modification T350V.

In certain embodiments, the fusion protein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including the amino acid modification F405A, F405I, F405M, F405S, F405T or F405V together with the amino acid modification Y407I or Y407V, and a second Fc polypeptide including the amino acid modification T366I, T366L or T366M, together with the amino acid modification T394W. In some embodiments, the first Fc polypeptide of the modified CH3 domain further includes the amino acid modification L351Y. In some embodiments, the second Fc polypeptide of the modified CH3 domain further includes the amino acid modification K392F, K392L or K392M. In some embodiments, one or both of the first and second Fc polypeptides of the modified CH3 domain further comprises the amino acid modification T350V.

In certain embodiments, the fusion protein comprises a heterodimeric Fc comprising a modified CH3 domain having a first Fc polypeptide that comprises amino acid modifications at positions F405 and Y407, and optionally further comprises an amino acid modification at position L351, and a second Fc polypeptide that comprises amino acid modifications at positions T366 and T394, and optionally further comprises an amino acid modification at position K392, as described above, and the first Fc polypeptide further comprises an amino acid modification at one or both of positions S400 or Q347 and/or the second Fc polypeptide further comprises an amino acid modification at one or both of positions K360 or N390, where the amino acid modification at position S400 is S400E, S400D, S400R or S400K; the amino acid modification at position Q347 is Q347R, Q347E or Q347K; the amino acid modification at position K360 is K360D or K360E, and the amino acid modification at position N390 is N390R, N390K or N390D.

In certain embodiments, the fusion protein comprises a heterodimeric Fc comprising a modified CH3 domain comprising the modifications of any one of Variant 1, Variant 2, Variant 3, Variant 4 or Variant 5, as shown in Table 2. In certain embodiments, the CH3 domain has an amino acid sequence corresponding to SEQ ID NO: 4 or SEQ ID NO: 5. In certain embodiments, the CH3 has an amino acid sequence that is substantially identical to SEQ ID NO: 4 or SEQ ID NO: 5. In certain embodiments, the CH3 domain has an amino acid sequence that is about 80%, about 85%, about 90%, or about 95% identical to SEQ ID NO: 4 or SEQ ID NO: 5.

TABLE 2

Human IgG1 Fc Sequences and Variants

| Human IgG1 Fc sequence 231-447 (EU-numbering) | APELLGGPSVFLFPPKPKDTLMISRTPEVTCVVVDVSH EDPEVKFNWYVDGVEVHNAKTKPREEQYNSTYRVVS VLTVLHQDWLNGKEYKCKVSNKALPAPIEKTISKAKG QPREPQVYTLPPSRDELTKNQVSLTCLVKGFYPSDIAV EWESNGQPENNYKTTPPVLDSDGSFFLYSKLTVDKSR WQQGNVFSCSVMHEALHNHYTQKSLSLSPGK (SEQ ID NO: NO: 29) | |
|---|---|---|
| Variant # | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

### Modified Fc CH2 Domains

In certain embodiments, the fusion protein comprises an Fc based on an IgG Fc having a modified CH2 domain. In some embodiments, the fusion protein comprises an Fc based on an IgG Fc having a modified CH2 domain, wherein the modification of the CH2 domain results in altered binding to one or more Fc receptors (FcRs) such as receptors of the FcγRI, FcγRII and FcγRIII subclasses.

A number of amino acid modifications to the CH2 domain that selectively alter the affinity of the Fc for different Fcγ receptors are known in the art. Amino acid modifications that result in increased binding and amino acid modifications that result in decreased binding can both be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise can be beneficial in some circumstances. In certain indications, a decrease in, or elimination of, ADCC and complement-mediated cytotoxicity (CDC) can be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) can be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom JL, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, the fusion protein comprises an Fc based on an IgG Fc having a modified CH2 domain, in which the modified CH2 domain comprises one or more amino acid modifications that result in decreased or eliminated binding of the Fc region to all of the Fcγ receptors (i.e., a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation (described in more detail below), use of IgG2/IgG4 scaffolds, or the introduction of mutations in the hinge or CH2 domain of the Fc (see also, U.S. Pat. Publication No. 2011/0212087, International Publication No. WO 2006/105338, U.S. Pat. Publication No. 2012/0225058, U.S. Pat. Publication No. 2012/0251531 and Strop et al., 2012, J. Mol. Biol., 420: 204-219).

Specific, non-limiting examples of known amino acid modifications to reduce FcγR and/or complement binding to the Fc include those identified in Table 3.

TABLE 3

Modifications to Reduce Fcγ Receptor or Complement Binding to the Fc

| | |
|---|---|
| Company | Mutations |
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples include Fc regions engineered to include the amino acid modifications L235A/L236A/D265S. In addition, asymmetric amino acid modifications in the CH2 domain that decrease binding of the Fc to all Fcγ receptors are described in International Publication No. WO 2014/190441.

In certain embodiments, the CH2 domain has an amino acid sequence corresponding to SEQ ID NO: 6. In certain embodiments, the CH2 has an amino acid sequence that is substantially identical to SEQ ID NO: 6. In certain embodiments, the CH2 domain has an amino acid sequence that is about 80%, about 85%, about 90%, or about 95% identical to SEQ ID NO: 6.

### Antibody Drug Conjugates

Certain embodiments of the fusion proteins described herein comprise biologically functional proteins that are an antibody conjugated to a drug, i.e., an antibody drug conjugate (ADC). The drug of an ADC can be any therapeutic molecule, e.g., a toxin, a chemotherapeutic agent, a small molecule inhibitor. The ADC can be conjugated to the drug via a linker, which may be a cleavable linker or a non-cleavable linker. A cleavable linker can be susceptible to cleavage under intracellular conditions, for example, through lysosomal processes. Examples of cleavable linkers include linkers that are protease-sensitive, acid-sensitive, reduction-sensitive or photolabile. Conjugation of the drug can be performed by any method known in the art including, but not limited to, lysine or cysteine conjugation, bis-thiol linkers, conjugation using glycosylation sites of antibodies, ultraviolet light conjugation, and use of unnatural amino acids.

### Peptidic Linkers, Proteases and Protease Cleavage Sites

The fusion proteins described herein comprise at least a first and a second peptidic linker. A peptidic linker is a peptide that joins or links other peptides or polypeptides. In certain embodiments, the peptidic linker fuses a polypeptide of the biologically functional protein, e.g., the antibody or dimeric Fc scaffold, to the ligand and/or receptor of the ligand-receptor pair.

In certain embodiments, where the biologically functional protein comprises an Fc region, an Fc polypeptide is fused to a ligand or receptor of the ligand-receptor pair, or a linker can join an Fc polypeptide to a ligand or receptor of the ligand-receptor pair. In certain embodiments, the ligand is fused to a terminus of the first polypeptide via the first peptidic linker; the receptor is fused to the same respective terminus of the second polypeptide via the second peptidic linker. In certain embodiments of the fusion proteins described herein, the receptor and the ligand are both fused to the respective N-termini of the first and second polypeptides via the peptidic linkers. In certain embodiments of the fusion proteins described herein, the receptor and the ligand are both fused to the respective C-termini of the first and second polypeptides via the peptidic linkers.

The peptidic linker is of sufficient length to allow pairing of ligand and receptor. In addition to providing a spacing function, a peptidic linker can provide flexibility or rigidity suitable for properly orienting the one or more domains of the fusion proteins herein, both within the fusion protein and between or among the fusion proteins and their target(s). Further, a peptidic linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a peptidic linker can comprises part or all of a human immunoglobulin hinge, a stalk region of C-type lectins, a family of type II membrane proteins, or combinations thereof.

In certain embodiments, the peptidic linker is of sufficient length to allow pairing of ligand and receptor and is of about 2 to about 150 amino acids. In certain embodiments, peptidic linkers range in length from about 3 to about 50 amino acids, or about 5 to about 20 amino acids, or about 10 to about 50 amino acids, or about 2 to about 40 amino acids, or about 8 to about 20 amino acids, about 10 to about 60 amino acids, about 10 to about 30 amino acids, or about 15 to about 25 amino acids. In some embodiments, the peptidic linker is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids.

At least one of the peptidic linkers of the fusion proteins described herein comprise a protease cleavage site, also referred to as cleavage sequences. In certain embodiments, the fusion protein comprises at least one peptidic linker comprising a protease cleavage site and at least one peptidic linker that does not comprise a protease cleavage site. Where used, the protease cleavage sites are positioned within the peptidic linkers so as to maximize recognition and cleavage by the desired protease or proteases and minimize recognition and non-specific cleavage by other proteases. The peptidic linker can comprise one or more cleavage sites. In these regards, a fusion protein can be cleaved by 1, 2, 3, 4, 5 or more proteases. Additionally, the protease cleavage site or sites can be positioned within the peptidic linkers (or said differently, can be surrounded by linkers) and are positioned within the fusion protein as a whole so as to achieve the best desired cleavage and release of fusion protein fragments (e.g., the ligand of the receptor ligand pair, the receptor of the ligand receptor pair or both the ligand and the receptor) post-cleavage. The polypeptide moiety that is fused to the fusion protein by the peptidic linker and that is released from the fusion protein following cleavage of the peptidic linker can be referred to herein as the cleavable moiety (CM). In certain embodiments where a fusion protein comprises more than one CM, they can be fused to the fusion protein by the same or different peptidic linkers, that is having the same cleavage site or different cleavage sites.

The protease cleavage site or cleavage sequence can be selected based on a protease that is co-localized in tissue where the activity of the fusion protein or biologically functional protein is desired. A cleavage site can serve as a substrate for multiple proteases, e.g., a substrate for a serine protease and a second different protease, e.g., a matrix metalloproteinase (an MMP). In some embodiments, a cleavage site can serve as a substrate for more than one serine protease, e.g., a matriptase and a urokinase-type plasminogen activator (uPA). In some embodiments, a peptidic linker can serve as a substrate for more than one MMP, e.g, an MMP9 and an MMP 14.

In certain embodiments, the peptidic linker is specifically cleaved by a protease at a rate of about 0.001-1500 × 10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500 × 10⁴ M⁻¹S⁻¹.

For specific cleavage by an enzyme, contact between the enzyme and the peptidic linker is made. In certain embodiments, when the fusion protein comprises at least a first peptidic linker and is in the presence of sufficient enzyme activity, the peptidic linker is cleaved. Sufficient enzyme activity can refer to the ability of the enzyme to make contact with the peptidic linker and effect cleavage. It can readily be envisioned that an enzyme can be in the vicinity of the peptidic linker but is unable to cleave because of other cellular factors or protein modification of the enzyme.

In certain embodiments, the peptidic linker comprises a protease cleavage site of 5-10 amino acids, or 7-10 amino acids, or 8-10 amino acids in length. In another embodiment, the peptidic linker consists of a protease cleavage site of 5-10 amino acids, or 7-10 amino acids, or 8-10 amino acids in length. In an embodiment, the protease cleavage site is preceded on the N-terminus by a linker sequence of about 1-20 amino acids, 2-5 amino acids, 5-10 amino acids, 10-15 amino acids, 10-20 amino acids, 12-16 amino acids, or about 5 or about 10 amino acids in length. In another embodiment, the protease cleavage site is followed on the C-terminus by a linker sequence of about 1-20 amino acids, 2 -5 amino acids, 5-10 amino acids, 10- 15 amino acids, 10-20 amino acids, 12-16 amino acids amino acids, or in some cases, about 5 or about 10 amino acids in length. In yet another embodiment, the protease cleavage site is preceded by a linker sequence on the N-terminus and followed by a linker sequence on the C-terminus. Thus, in certain embodiments, the protease cleavage site is situated between two linkers. The linkers on either the N or C-terminal end of the protease cleavage site can be of varying lengths, for example, between about 2-20, 6-20, 8-15, 8-10, 10-18, or 12-16 amino acids in length. In certain embodiments, the N- or C-terminal linker sequence is about 3 or about 5 amino acids in length.

Exemplary peptidic linkers of the disclosure comprise one or more protease cleavage sites recognized by any of a variety of proteases, such as, but not limited to, serine proteases, MMPs (MMP1, MMP2, MMP3, MMP7, MMP8, MMP9, MMP10, MMP11, MMP12, MMP13, MMP14, MMP15, MMP16, MMP17, MMP18 (collagenase 4), MMP19, MMP20, MMP21, etc), adamalysins, serralysins, astacins, caspases (e.g., caspase 1, caspase 2, caspase 3, caspase 4, caspase 5, caspase 6, caspase 7, caspase 8, caspase 9, caspase 10, caspase 11, caspase 12, caspase 13, caspase 14), cathepsins, (e.g., cathepsin A, cathepsin B, cathepsin D, cathepsin E, cathepsin K, cathepsin S), granyme B, guanidinobenzoatase (GB), hepsin, elastase, legumain, matriptase, matriptase 2, meprin, neurosin, MT-SP1, neprilysin, plasmin, PSA, PSMA, TACE, TMPRSS¾, uPA, and calpain, FAP and KLK. In some embodiments the protease is uPA or matriptase.

In certain embodiments, a peptidic linker comprises a cleavage site that is cleaved by more than one protease. In this regard, an individual cleavage site can be cleaved by 1, 2, 3, 4, 5 or more proteases. In another embodiment, a peptidic linker can comprise a cleavage site that is substantially cleaved by one enzyme but not by others. Thus, in some embodiments, a peptidic linker comprises a cleavage site that has high specificity. By “high specificity” is meant >90% cleavage observed by a particular protease and less than 50% cleavage observed by other proteases. In certain embodiments, a peptidic linker comprises a cleavage site that demonstrates >80% cleavage by one protease but less than 50% cleavage by other proteases. In certain embodiments, a peptidic linker comprises a cleavage site that demonstrates >70%, 75%, 76%, 77%, 78%, or 79%, cleavage by one protease but less than 65%, 60%, 55%, 54%, 53%, 52%, 51%, 50%, 49%, 48%, 47%, 46%, or 45% cleavage by other proteases. By way of example, in an embodiment, the cleavage site can be >90% cleaved by matriptase and ~75% cleaved by uPa and plasmin. In another embodiment, the cleavage site can be cleaved by uPa and matriptase but no specific cleavage by plasmin is observed. In yet another embodiment, the cleavage site can be cleaved by uPa and not by matriptase or plasmin. In an embodiment, a cleavage site can demonstrate some level of resistance to non-specific protease cleavage, e.g., cleavage by plasmin or other non-specific proteases. In this regard, a protease cleavage site can have “high non-specific protease resistance” (<25% cleavage by plasmin or an equivalent non-specific protease), “moderate non-specific protease resistance” (<75% cleavage by plasmin or an equivalent non-specific protease), or “low non-specific protease resistance” (up to about 90% cleavage by plasmin or an equivalent non-specific protease). Such cleavage activity can be measured using assays known in the art, such as by incubation with the appropriate protease followed by SDS-PAGE or other analysis. In certain embodiments, a protease cleavage site may display up to complete resistance to protease cleavage to 24 hours contact with protease. In other embodiments, a protease cleavage sequence may display up to complete resistance to non-specific protease cleavage after 0.5 hour to 36 hours contact with protease. In another embodiment, a protease cleavage sequence displays up to complete resistance to non-specific protease cleavage after 0.5, 1, 2, 3, 4, 5, 6, 7, 8, 9, 10, 12, 20, 24, 36, 48, or 72 hours contact with an appropriate protease.

Thus, in certain embodiments, the cleavage sites are selected based on preferences for various desired proteases. In this way, a desired cleavage profile for a particular peptidic linker comprising a cleavage site can be selected for a desired purpose (e.g., high specific cleavage in particular tumor microenvironments or specific organs) where a particular protease or set of proteases can demonstrate high, specific, elevated, efficient, moderate, low or no cleavage of a particular cleavage site within a peptidic linker. Methods for determining cleavage are known in the art.

In certain embodiments, a peptidic linker can comprise one or more cleavage sites arranged in tandem, with or without additional linkers in between each cleavage site. In certain embodiments, a peptidic linker comprises a first cleavage site and a second cleavage site where the first cleavage site is cleaved by a first protease and the second cleavage site is cleaved by a second protease. As a non-limiting example, a peptidic linker can comprise a first cleavage site cleaved by matriptase and uPa and a second cleavage site cleaved by an MMP. In certain embodiments, a peptidic linker comprises a first cleavage site, a second cleavage site and a third cleavage site where the first cleavage site is cleaved by a first protease, the second cleavage site is cleaved by a second protease and the third cleavage site is cleaved by a third protease.

Illustrative proteolytic enzymes and their recognition sequences useful in the fusion proteins herein can be identified by one of skill and are known in the art, such as those described in MEROPS database (see e.g., Rawlings, et al. Nucleic Acids Research, Volume 46, Issue D1, 4 Jan. 2018, Pages D624-D632), and elsewhere (Hoadley et al, Cell, 2018; GTEX Consortium, Nature, 2017; Robinson et al, Nature, 2017).

Other methods can also be used for identifying cleavage sites for use herein, such as described in U.S. Pat. Nos. 9,453,078, 10,138,272, 9,562,073 and published international application numbers WO 2015048329; WO2015116933; WO2016118629.

Accordingly, an embodiment of the present disclosure provides a fusion protein that comprises at least two peptidic linkers wherein at least one of the peptidic linkers comprises one or more of the cleavage sites set forth herein. In an embodiment, the present disclosure provides a fusion protein that comprises a peptidic linker wherein the peptidic linker comprises a protease cleavage site and is cleavable by uPA. In an embodiment, the present disclosure provides a fusion protein that comprises a peptidic linker wherein the peptidic linker comprises the amino acid sequence MSGRSANA (SEQ ID NO: NO:28). In certain embodiments the peptidic linker sequence comprises at least one protease cleavage site selected from TSGRSANP, LSGRSDNH, GSGRSAQV, GSSRNADV, GTARSDNV, GTARSDNV. GGGRVNNV, MSARILQV or GKGRSANA (SEQ ID NOS: 30-37 respectively).

In certain embodiments, the fusion protein comprising the peptidic linker described herein comprises two heterologous polypeptides, a first polypeptide located amino (N) terminally to the peptidic linker and a second polypeptide located carboxyl (C) terminally to the peptidic linker, the two heterologous polypeptides thus separated by the peptidic linker.

In certain embodiments, the fusion protein comprises at least one peptidic linker that does not comprise a protease cleavage site. In certain embodiments, the peptidic linker comprises an amino acid sequence (EAAAK)n where n is an integer of 1 to 5. In some embodiments, the peptidic linker is EAAAK (SEQ ID NO:39). In some embodiments the peptidic linker EAAAKEAAAK (SEQ ID NO:38). In some embodiments, the peptidic linker comprises a polyproline linker, optionally having an amino acid sequence of PPP (SEQ ID NO: 41) or PPPP (SEQ ID NO: 40). In certain embodiments the linker is glycine (G)-proline (P) polypeptide linker, optionally GPPPG, GGPPPGG, GPPPPG or GGPPPGG.In certain embodiments, the peptidic linker is a GlyₙSer linker. In certain embodiments, the peptidic linker comprises an amino acid sequence of (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. In certain embodiments, the peptidic linkers are suitable for connecting the different domains include sequences comprising glycine-serine linkers, for example, but not limited to, (GₘS)ₙ-GG, (SGn)m, (SEGn)m, wherein m and n are between 0-20.

In certain embodiments, a peptidic linker is an amino acid sequence obtained, derived, or designed from an antibody hinge region sequence, a sequence linking a binding domain to a receptor, or a sequence linking a binding domain to a cell surface transmembrane region or membrane anchor. In some embodiments, a peptidic linker has at least one cysteine capable of participating in at least one disulfide bond under physiological conditions or other standard peptide conditions (e.g., peptide purification conditions, conditions for peptide storage). In certain embodiments, a peptidic linker corresponding or similar to an immunoglobulin hinge peptide retains a cysteine that corresponds to the hinge cysteine disposed toward the amino-terminus of that hinge. In further embodiments, a peptidic linker is from an IgG1 hinge and has been modified to remove any cysteine residues or is an IgG1 hinge that has one cysteine or two cysteines corresponding to hinge cysteines.

In certain embodiments, a peptidic linker for use herein can comprise an “altered wild type immunoglobulin hinge region” or “altered immunoglobulin hinge region”. Such altered hinge regions refers to (a) a wild type immunoglobulin hinge region with up to 30 percent amino acid changes (e.g., up to 25 percent, 20 percent, 15 percent, 10 percent, or 5 percent amino acid substitutions or deletions), (b) a portion of a wild type immunoglobulin hinge region that is at least 10 amino acids (e.g., at least 12, 13, 14 or 15 amino acids) in length with up to 30 percent amino acid changes (e.g., up to 25 percent, 20 percent, 15 percent, 10 percent, or 5 percent amino acid substitutions or deletions), or (c) a portion of a wild type immunoglobulin hinge region that comprises the core hinge region (which portion can be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild type immunoglobulin hinge region, such as an IgG1 hinge comprising the upper and core regions, can be substituted by one or more other amino acid residues (e.g., one or more serine residues). An altered immunoglobulin hinge region can alternatively or additionally have a proline residue of a wild type immunoglobulin hinge region, such as an IgG1 hinge comprising the upper and core regions, substituted by another amino acid residue (e.g., a serine residue).

Alternative hinge and linker sequences that can be used as connecting regions can be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from 5 to 60 amino acids long, and can be primarily flexible, but can also provide more rigid characteristics, can contain primarily a helical structure with minimal beta sheet structure.

In certain embodiments, proteases described herein are expressed at higher amounts near a particular target cell of interest, e.g., the tumor microenvironment of target tumor cell, in vivo. A variety of different conditions or diseases are known in which a target of interest (such as a particular tumor type, a particular tumor that expresses a particular tumor associated antigen) is co-localized with a protease, where the substrate of the protease is known in the art. In the example of cancer, the target tissue can be a cancerous tissue, particularly cancerous tissue of a solid tumor. There are reports in the literature of increased levels of proteases in a number of cancers, e.g., liquid tumors or solid tumors. See, e.g., La Rocca et al, (2004) British J. of Cancer 90(7): 1414-1421.

In certain embodiments, the fusion protein comprises, from N terminus to C terminus, Ligand-Linker-VL, Receptor-Linker-VL, Ligand-Linker-VH, or Receptor-Linker-VH.

In certain embodiments, the fusion protein comprises from N terminus to C terminus, Ligand-cleavable Linker-VL, Receptor- cleavable Linker-VL, Ligand- cleavable Linker-VH, or Receptor- cleavable Linker-VH.

In certain embodiments, the fusion protein comprises from N terminus to C terminus, Ligand-linker (SEQ ID NO:114)-VL, Receptor-linker (SEQ ID NO:114)-VL, Ligand-linker (SEQ ID NO: 14)-VH, or Receptor-linker (SEQ ID NO: 14)-VH.

In certain embodiments, the fusion protein comprises from N terminus to C terminus, Ligand-linker (SEQ ID NO:145)-VL, Receptor-linker (SEQ ID NO:145)-VL, Ligand-linker (SEQ ID NO:145)-VH, or Receptor-linker (SEQ ID NO:145)-VH.

In certain embodiments, the fusion protein comprises from N terminus to C terminus, Ligand-linker (SEQ ID NO:147)-VL, Receptor-linker (SEQ ID NO:147)-VL, Ligand-linker (SEQ ID NO:147)-VH, or Receptor-linker (SEQ ID NO:147)-VH.

In certain embodiments, the fusion protein comprises from N terminus to C terminus, Ligand-linker (SEQ ID NO:154)-VL, Receptor-linker (SEQ ID NO:154)-VL, Ligand-linker (SEQ ID NO:154)-VH, or Receptor-linker (SEQ ID NO:154)-VH.

In certain embodiments, the fusion protein comprises from N terminus to C terminus, Ligand-linker (SEQ ID NO:203)-VL, Receptor-linker (SEQ ID NO:203)-VL, Ligand-linker (SEQ ID NO:203)-VH, or Receptor-linker (SEQ ID NO:203)-VH.

In certain embodiments, the fusion protein comprises, from N terminus to C terminus, Ligand-linker-Fc or Receptor-linker-Fc.

In certain embodiments, the fusion protein comprises, from N terminus to C terminus, Ligand-cleavable linker-Fc or Receptor- cleavable linker-Fc.

In certain embodiments, the fusion protein comprises, from N terminus to C terminus, Ligand-cleavable linker (SEQ ID NO:28)-Fc or Receptor- cleavable linker (SEQ ID NO:28)-Fc.

In certain embodiments, the fusion protein comprises, from N terminus to C terminus, Ligand-linker-Fc1 or Receptor-linker-Fc1.

In certain embodiments, the fusion protein comprises, from N terminus to C terminus, Ligand-cleavable linker-Fc2 or Receptor-cleavable linker-Fc2.

In certain embodiments, Fc1 and Fc2 can form heterodimers. In certain embodiments, Fc1 is linked to a Ligand and Fc2 is linked to a Receptor. In certain embodiments, the linker connecting the Ligand with Fc1 is cleavable and the linker connecting the Receptor with Fc2 is non- cleavable. In certain embodiments, the linker connecting the Ligand with Fc1 is non-cleavable and the linker connecting the Receptor with Fc2 is cleavable. In certain embodiments, the linker connecting the Ligand with Fc1 is cleavable and the linker connecting the Receptor with Fc2 is cleavable. In certain embodiments, the linker connecting the Ligand with Fc1 is non-cleavable and the linker connecting the Receptor with Fc2 is non-cleavable.

### Targets

In some embodiments, an antigen-binding domain of the fusion protein described herein specifically binds to a cell surface molecule. In certain embodiments, an antigen-binding domain of the fusion protein specifically binds to a tumor-associated antigen (TAA). The TAA is any antigenic substance expressed on a tumor cell surface. In some embodiments, an antigen-binding domain specifically and binds to a TAA selected from Fibroblast activation protein alpha (FAPa), Trophoblast glycoprotein (5T4), Tumor-associated calcium signal transducer 2 (Trop2), Fibronectin EDB (EDB-FN), fibronectin F.IIIB domain, CGS-2, EpCAM, EGER, HER-2, HER-3, cMet, CEA, and FOLR1, EpCAM, EGFR, HER-2, HER-3, cMet, CEA, and FOLR1, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, DR5, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin (MSLN), Mucl, Mucl6, NaPi2b, Nectin-4, P-cadherin, NY-ESO-1, PRLR, PSCA, PTK7, ROR1, SLC44A4, SLTRK5, SLTRK6, STEAP1, TIM1, tissue factor (TF), Trop2, WT1.

In some embodiments, an antigen-binding domain specifically binds to an immune checkpoint protein. Examples of immune checkpoint proteins include but are not limited to CD27, CD137, 2B4, TIGIT, CD155, ICOS, HVEM, CD40L, LIGHT, TIM-1, 0X40, DNAM-1, PD-L1, PD1, PD-L2, CTLA-4, CD80, CD40, CEACAM1, CD48, CD70, A2AR, CD39, CD73, B7-H3, B7-H4, BTLA, IDO1, ID02, TDO, KIR, LAG-3, TIM-3, VISTA, CD47, or SIRPα.

In some embodiments, an antigen-binding domain specifically binds to an antigen expressed on a virally infected cell, bacterially infected cell, damaged red blood cell, arterial plaque cell, inflamed or fibrotic tissue cell.

In certain embodiments, an antigen-binding domain specifically binds a cytokine receptor. Examples of cytokine receptors include, but are not limited to, Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGR1, IFNGR2), Type II IF receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSFlA/TNFR1/CD120a, TNFRSF1B / TNFR2 / CD120b; TGF-beta receptors, such as TGF-beta receptor 1, TGF-beta receptor 2; Ig super family receptors, such as IF-1 receptors, CSF-1R, PDGFR (PDGFRA, PDGFRB), SCFR.

In certain embodiments, the antigen-binding domains of the fusion proteins described herein specifically bind to at least one molecule or target of interest in vivo. In certain embodiments, the target of interest is: Cluster of Differentiation 3 (CD3), Human Epidermal Growth Factor Receptor 2 (HER2), Epidermal Growth Factor Receptor (EGFR), Mesothelin (MSLN), Tissue Factor (TF), Cluster of Differentiation 19 (CD19), tyrosine-protein kinase Met (c-Met), Cluster of Differentiation 40 (CD40), Cadherin 3 (CDH3), or combinations thereof. In certain embodiments, the fusion protein comprises an antibody and at least one antigen binding domain of the antibody binds to an epitope on CD3, HER2, EGFR, MSLN, TF, CD19, c-Met, CD40, CDH3, or combinations thereof.

In some embodiments, the target of interest is HER2, and the anti-HER2 paratope of the fusion protein has a VH having an amino acid sequence corresponding to SEQ ID NO: 120 and a VL having an amino acid sequence corresponding to SEQ ID NO: 124. In certain embodiments, the anti-HER2 paratope has a VH amino acid sequence that is substantially identical to SEQ ID NO: 120 and a VL amino acid sequence that is substantially identical to SEQ ID NO: 124. In certain embodiments, the anti-HER2 paratope has a VH amino acid sequence that is about 80%, about 85%, about 90%, or about 95% identical to SEQ ID NO: 120 and a VL amino acid sequence that is about 80%, about 85%, about 90%, or about 95% identical to SEQ ID NO: 124. In certain embodiments, the anti-HER2 paratope has a VH amino acid sequence that is about 96%, about 97%, about 98%, or about 99% identical to SEQ ID NO: 120 and a VL amino acid sequence that is about 96%, about 97%, about 98%, or about 99% identical to SEQ ID NO: 124. In some embodiments, the anti-HER2 paratope comprises an scFv having an amino acid sequence corresponding to SEQ ID NO: 3. In some embodiments, the anti- HER2 has a VH having 3 CDRS, HCDR1, HDR2 and HCDR3 having amino acid sequences corresponding to SEQ ID NOS: 121, 122 and 123 respectively, and a VL having 3 CDRs LCDR1, LCDR2 and LCDR3 having amino acid sequences corresponding to SEQ ID NOS: 125, 126 and 127 respectively.

In some embodiments, the target of interest is EGFR, and the anti-EGFR paratope of the fusion protein has a VH having an amino acid sequence corresponding to SEQ ID NO: 14 and a VL having an amino acid sequence corresponding to SEQ ID NO: 13. In certain embodiments, the anti- EGFR paratope has a VH amino acid sequence that is substantially identical to SEQ ID NO: 14 and a VL amino acid sequence that is substantially identical to SEQ ID NO: 13. In certain embodiments, the anti-EGFR paratope has a VH amino acid sequence that is about 80%, about 85%, about 90%, or about 95% identical to SEQ ID NO: 14 and a VL amino acid sequence that is about 80%, about 85%, about 90%, or about 95% identical to SEQ ID NO: 13. In certain embodiments, the anti-EGFR paratope has a VH amino acid sequence that is about 96%, about 97%, about 98%, or about 99% identical to SEQ ID NO: 14 and a VL amino acid sequence that is about 96%, about 97%, about 98%, or about 99% identical to SEQ ID NO: 13. In some embodiments, the anti-EGFR has a VH having 3 CDRS, HCDR1, HDR2 and HCDR3 having amino acid sequences corresponding to SEQ ID NOS: 84, 85 and 86 respectively, and a VL having 3 CDRs LCDR1, LCDR2 and LCDR3 having amino acid sequences corresponding to SEQ ID NOS: 59, 60 and 61 respectively.

In some embodiments, the target of interest is MSLN, and the anti- MSLN paratope of the fusion protein has a VH having an amino acid sequence corresponding to SEQ ID NO: 16 and a VL having an amino acid sequence corresponding to SEQ ID NO: 15. In certain embodiments, the anti- MSLN paratope has a VH amino acid sequence that is substantially identical to SEQ ID NO: 16 and a VL amino acid sequence that is substantially identical to SEQ ID NO: 15. In certain embodiments, the anti-MSLN paratope has a VH amino acid sequence that is about 80%, about 85%, about 90%, or about 95% identical to SEQ ID NO: 16 and a VL amino acid sequence that is about 80%, about 85%, about 90%, or about 95% identical to SEQ ID NO: 15. In certain embodiments, the anti- MSLN paratope has a VH amino acid sequence that is about 96%, about 97%, about 98%, or about 99% identical to SEQ ID NO: 16 and a VL amino acid sequence that is about 96%, about 97%, about 98%, or about 99% identical to SEQ ID NO: 15. In some embodiments, the anti-MSLN has a VH having 3 CDRS, HCDR1, HDR2 and HCDR3 having amino acid sequences corresponding to SEQ ID NOS: 69, 70 and 71 respectively, and a VL having 3 CDRs LCDR1, LCDR2 and LCDR3 having amino acid sequences corresponding to SEQ ID NOS: 74, 75 and 76 respectively.

In some embodiments, the target of interest is TF (Tissue Factor), and the anti- TF paratope of the fusion protein has a VH having an amino acid sequence corresponding to SEQ ID NO: 18 and a VL having an amino acid sequence corresponding to SEQ ID NO: 17. In certain embodiments, the anti- TF paratope has a VH amino acid sequence that is substantially identical to SEQ ID NO: 18 and a VL amino acid sequence that is substantially identical to SEQ ID NO: 17. In certain embodiments, the anti-TF paratope has a VH amino acid sequence that is about 80%, about 85%, about 90%, or about 95% identical to SEQ ID NO: 18 and a VL amino acid sequence that is about 80%, about 85%, about 90%, or about 95% identical to SEQ ID NO: 17. In certain embodiments, the anti- TF paratope has a VH amino acid sequence that is about 96%, about 97%, about 98%, or about 99% identical to SEQ ID NO: 18 and a VL amino acid sequence that is about 96%, about 97%, about 98%, or about 99% identical to SEQ ID NO: 17. In some embodiments, the anti-TF has a VH having 3 CDRS, HCDR1, HDR2 and HCDR3 having amino acid sequences corresponding to SEQ ID NOS: 54, 55 and 56 respectively, and a VL having 3 CDRs LCDR1, LCDR2 and LCDR3 having amino acid sequences corresponding to SEQ ID NOS: 48, 49 and 50 respectively.

In some embodiments, the target of interest is CD19 and the anti-CD19 paratope of the fusion protein has a VH having an amino acid sequence corresponding to SEQ ID NO: 20 and a VL having an amino acid sequence corresponding to SEQ ID NO: 19 In certain embodiments, the anti- CD19 paratope has a VH amino acid sequence that is substantially identical to SEQ ID NO: 20 and a VL amino acid sequence that is substantially identical to SEQ ID NO: 19. In certain embodiments, the anti-CD19 paratope has a VH amino acid sequence that is about 80%, about 85%, about 90%, or about 95% identical to SEQ ID NO: 20 and a VL amino acid sequence that is about 80%, about 85%, about 90%, or about 95% identical to SEQ ID NO: 19. In certain embodiments, the anti-CD19 paratope has a VH amino acid sequence that is about 96%, about 97%, about 98%, or about 99% identical to SEQ ID NO: 20 and a VL amino acid sequence that is about 96%, about 97%, about 98%, or about 99% identical to SEQ ID NO: 19. In some embodiments, the anti-CD19 has a VH having 3 CDRS, HCDR1, HDR2 and HCDR3 having amino acid sequences corresponding to SEQ ID NOS: 64, 65 and 66 respectively, and a VL having 3 CDRs LCDR1, LCDR2 and LCDR3 having amino acid sequences corresponding to SEQ ID NOS: 74, 75 and 165 respectively.

In some embodiments, the target of interest is c-Met and the anti-c-Met paratope of the fusion protein has a VH having an amino acid sequence corresponding to SEQ ID NO: 22 and a VL having an amino acid sequence corresponding to SEQ ID NO: 21. In certain embodiments, the anti- c-Met paratope has a VH amino acid sequence that is substantially identical to SEQ ID NO: 22 and a VL amino acid sequence that is substantially identical to SEQ ID NO: 21. In certain embodiments, the anti-c-Met paratope has a VH amino acid sequence that is about 80%, about 85%, about 90%, or about 95% identical to SEQ ID NO: 22 and a VL amino acid sequence that is about 80%, about 85%, about 90%, or about 95% identical to SEQ ID NO: 21. In certain embodiments, the anti-c-Met paratope has a VH amino acid sequence that is about 96%, about 97%, about 98%, or about 99% identical to SEQ ID NO: 22 and a VL amino acid sequence that is about 96%, about 97%, about 98%, or about 99% identical to SEQ ID NO: 21. In some embodiments, the anti- c-Met has a VH having 3 CDRS, HCDR1, HDR2 and HCDR3 having amino acid sequences corresponding to SEQ ID NOS: 99, 100 and 101 respectively, and a VL having 3 CDRs LCDR1, LCDR2 and LCDR3 having amino acid sequences corresponding to SEQ ID NOS: 94, 95 and 96 respectively.

In some embodiments, the target of interest is CDH3 and the anti-CDH3 paratope of the fusion protein has a VH having an amino acid sequence corresponding to SEQ ID NO: 24 and a VL having an amino acid sequence corresponding to SEQ ID NO: 23. In certain embodiments, the anti- CDH3 paratope has a VH amino acid sequence that is substantially identical to SEQ ID NO: 24 and a VL amino acid sequence that is substantially identical to SEQ ID NO: 23. In certain embodiments, the anti- CDH3 paratope has a VH amino acid sequence that is about 80%, about 85%, about 90%, or about 95% identical to SEQ ID NO: 24 and a VL amino acid sequence that is about 80%, about 85%, about 90%, or about 95% identical to SEQ ID NO: 23. In certain embodiments, the anti- CDH3 paratope has a VH amino acid sequence that is about 96%, about 97%, about 98%, or about 99% identical to SEQ ID NO: 24 and a VL amino acid sequence that is about 96%, about 97%, about 98%, or about 99% identical to SEQ ID NO: 23. In some embodiments, the anti-CDH3 has a VH having 3 CDRS, HCDR1, HDR2 and HCDR3 having amino acid sequences corresponding to SEQ ID NOS: 89, 90 and 91 respectively, and a VL having 3 CDRs LCDR1, LCDR2 and LCDR3 having amino acid sequences corresponding to SEQ ID NOS: 94, 95 and 96 respectively.

In some embodiments, the target of interest is CD40 and the anti-CD40 paratope of the fusion protein has a VH having an amino acid sequence corresponding to SEQ ID NO: 172 and a VL having an amino acid sequence corresponding to SEQ ID NO: 177. In certain embodiments, the anti-CD40 paratope has a VH amino acid sequence that is substantially identical to SEQ ID NO: 172 and a VL amino acid sequence that is substantially identical to SEQ ID NO: 177. In certain embodiments, the anti-CD40 paratope has a VH amino acid sequence that is about 80%, about 85%, about 90%, or about 95% identical to SEQ ID NO: 172 and a VL amino acid sequence that is about 80%, about 85%, about 90%, or about 95% identical to SEQ ID NO: 177. In certain embodiments, the anti-CD40 paratope has a VH amino acid sequence that is about 96%, about 97%, about 98%, or about 99% identical to SEQ ID NO: 172 and a VL amino acid sequence that is about 96%, about 97%, about 98%, or about 99% identical to SEQ ID NO: 177. In some embodiments, the anti-CD40 has a VH having 3 CDRS, HCDR1, HDR2 and HCDR3 having amino acid sequences corresponding to SEQ ID NOS: 173, 174 and 175 respectively, and a VL having 3 CDRs LCDR1, LCDR2 and LCDR3 having amino acid sequences corresponding to SEQ ID NOS: 178, 179 and 180 respectively.

In certain embodiments, an antigen-binding domain of the fusion protein binds specifically to a molecule, e.g., a polypeptide, on an immune cell. In certain embodiments, the fusion protein comprises an antigen-binding domain that binds specifically to both a TAA and an antigen-binding domain that specifically binds to a molecule, e.g., a polypeptide, on an immune cell. Thus, in certain embodiments, the fusion protein binds to both a tumor cell and an immune cell. In certain embodiments, the immune cell is a T cell. In certain embodiments the immune cell is a macrophage, a dendritic cell, a neutrophil, a B-cell or an NK cell.

In certain embodiments the fusion protein binds to a CD3 antigen on a T cell and one or more TAAs on a tumor cell.

### Masked T Cell Engagers

A T cell engager (TCE) is a polypeptide construct, often a bispecific antibody, that simultaneously binds a TAA on a tumor cell and CD3 epitope on a T-cell to form a TCR-independent artificial immune synapse. This causes the T cell to become activated and to exert a cytotoxic effect on the tumor cell. Bi-specific antibodies capable of targeting T cells to tumor cells have been identified and tested for their efficacy in the treatment of cancers. Blinatumomab is an example of a bi-specific anti-CD3-CD19 antibody in a format called BiTE™ (Bi-specific T-cell Engager) that has been identified for the treatment of B-cell diseases such as relapsed B-cell non-Hodgkin lymphoma and chronic lymphocytic leukemia (Baeuerle et al (2009) Cancer Research12:4941-4944) and is FDA approved. T cell engagers directed against other tumor-associated target antigens have also been made, and several have entered clinical trials: AMG110/MT110 EpCAM for lung cancer, gastric cancer and colorectal cancer; AMG211/MEDI565 CEA for gastrointestinal adenocarcinoma; and AMG 212 / BAY2010112 PSMA for prostate cancer (see Suruadevara, C. M. et al, Oncoimmunology. 2015 Jun; 4(6): e1008339). While these studies showed promising clinical efficacy, they were also hampered by severe dose-limiting toxicities primarily due to cytokine release syndrome (CRS). This resulted in narrow therapeutic windows. The use of masked T cell-binding paratopes which are activated primarily in a tumor microenvironment might reduce the toxicity of TCEs.

In certain embodiments the fusion protein binds a CD3 antigen on a T cell and a TAA on a tumor cell. In certain embodiments the fusion protein binds a CD3 antigen on a T cell, a TAA on a tumor cell and an IgSF extracellular domain on a tumor cell. In certain embodiments, the fusion protein binds a CD3 antigen on a T cell, a TAA on a tumor cell and an IgSF extracellular domain on the T cell.

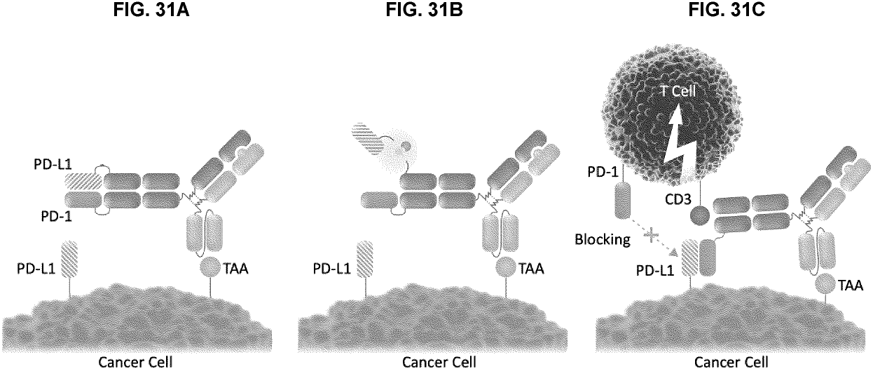

In certain embodiments, a fusion protein becomes unmasked by a protease in a tumor microenvironment, and binds to a TAA on a tumor cell and a CD3 antigen on a T cell, causing bridging of the T cell and the tumor cell, as is demonstrated in Example 20. In certain embodiments, an unmasked fusion protein binds a CD3 antigen on a T cell, and both a TAA and a IgSF ligand on a tumor cell, as is illustrated in FIG. 31. In certain embodiments, the binding of the IgSF ligand (e.g. PD-L1) on the tumor cell prevents the binding of its IgSF receptor (e.g. PD-1) on the T cell, thus blocking checkpoint inhibition (FIG. 31 C).

In certain embodiments the fusion proteins comprise an anti-CD3 paratope VH and a VL substantially identical to those of the paratopes shown in Table BB. In certain embodiments, the CD3 paratope comprises VH and VL amino acid sequences of:
(a) a VH comprising an amino acid sequence corresponding to SEQ ID NO: 2 and a VL comprising an amino acid sequence according to SEQ ID NO: 1;
(b) a VH comprising an amino acid sequence corresponding to SEQ ID NO: 206 and a VL comprising an amino acid sequence according to SEQ ID NO: 210;
(c) a VH comprising an amino acid sequence corresponding to SEQ ID NO: 215 and a VL comprising an amino acid sequence according to SEQ ID NO: 219;
(d) a VH comprising an amino acid sequence corresponding to SEQ ID NO: 223 and a VL comprising an amino acid sequence according to SEQ ID NO: 227;
(d) a VH comprising an amino acid sequence corresponding to SEQ ID NO: 231 and a VL comprising an amino acid sequence according to SEQ ID NO: 235; or
(e) a VH comprising an amino acid sequence corresponding to SEQ ID NO: 239 and a VL comprising an amino acid sequence according to SEQ ID NO: 243.

In certain embodiments, the CD3 paratope comprises VH and VL that are about 90%, about 91%, about 92%, about 93%, about 94%, about 95%, about 96%, about 97%, about 98% or about 99% identical to:
(a) a VH comprising an amino acid sequence corresponding to SEQ ID NO: 2 and a VL comprising an amino acid sequence according to SEQ ID NO: 1;
(b) a VH comprising an amino acid sequence corresponding to SEQ ID NO: 206 and a VL comprising an amino acid sequence according to SEQ ID NO: 210;
(c) a VH comprising an amino acid sequence corresponding to SEQ ID NO: 215 and a VL comprising an amino acid sequence according to SEQ ID NO: 219;
a VH comprising an amino acid sequence corresponding to SEQ ID NO: 223 and a VL comprising an amino acid sequence according to SEQ ID NO: 227;
a VH comprising an amino acid sequence corresponding to SEQ ID NO: 231 and a VL comprising an amino acid sequence according to SEQ ID NO: 235; or
a VH comprising an amino acid sequence corresponding to SEQ ID NO: 239 and a VL comprising an amino acid sequence according to SEQ ID NO: 243.

In certain embodiments, the anti-CD3 paratope comprises a VH comprising 3 heavy chain CDRs HCDR1, HCDR2 and HCDR3 comprising amino acid sequences corresponding to SEQ ID NOS: 207, 208 and 209, and a VL comprising 3 light chain CDRs LCDR1, LCDR2 and LCDR3 comprising amino acid sequences corresponding to SEQ ID NOS: 211, 212 and 214. In certain embodiments, the anti-CD3 paratope comprises a VH comprising 3 heavy chain CDRs HCDR1, HCDR2 and HCDR3 comprising amino acid sequences corresponding to SEQ ID NOS: 224, 225 and 226, and a VL comprising 3 light chain CDRS LCDR1, LCDR2 and LCDR3 comprising amino acid sequences corresponding to SEQ ID NOS: 228, 229 and 230. In certain embodiments, the anti-CD3 paratope comprises a VH comprising 3 heavy chain CDRs HCDR1, HCDR2 and HCDR3 comprising amino acid sequences corresponding to SEQ ID NOS: 232, 233 and 234, and a VL comprising 3 light chain CDRS LCDR1, LCDR2 and LCDR3 comprising amino acid sequences corresponding to SEQ ID NOS: 236, 237 and 238. In certain embodiments, the anti-CD3 paratope comprises a VH comprising 3 heavy chain CDRs HCDR1, HCDR2 and HCDR3 comprising amino acid sequences corresponding to SEQ ID NOS: 240, 241 and 242, and a VL comprising 3 light chain CDRS LCDR1, LCDR2 and LCDR3 comprising amino acid sequences corresponding to SEQ ID NOS: 244, 245 and 246.

### CAR Constructs

In certain embodiments, the fusion protein can be included in a chimeric antigen receptor (CAR) or a CAR fragment. A CAR can comprise one or more extracellular ligand binding domains, optionally a hinge region, a transmembrane region, and an intracellular signaling region. The one or more extracellular ligand binding domains can include one or more fusion proteins. The extracellular ligand binding domain can typically comprises a single-chain immunoglobulin variable fragment (scFv) or other ligand binding domain, such as a Fab or a natural protein ligand. The hinge region can generally comprise a polypeptide hinge of variable length such as one or more amino acids, a CD8 alpha hinge region or an IgG4 region (or others), and combinations thereof. The transmembrane domain can typically include a transmembrane region derived from CD8 alpha, CD28, or other transmembrane proteins such as DAP10, DAP12, or NKG2D, and combinations thereof. The intracellular signaling region can include one or more intracellular signaling domains such as CD28, 4-1BB, CD3 zeta, OX40, 2B4, or other intracellular signaling domains, and combinations thereof. For example the one or more intracellular signaling domains can include CD28 and CD3 zeta, 4-1BB and CD3 zeta, or CD3 zeta. Lymphocytes such as T cells and NK cells can be modified to produce chimeric antigen receptor cells (e.g., CAR-Ts). CAR-T cells can recognize specific soluble antigens or antigens on a target cell surface, such as a tumor cell surface, or on cells in the tumor microenvironment. When the extracellular ligand binding domain binds to a cognate ligand, the intracellular signaling domain of the CAR can activate the lymphocyte. See, e.g., Brudno et al., Nature Rev. Clin. Oncol. (2018) 15:31 -46; Maude et al., N. Engl. J. Med. (2014) 371 : 1507-1517; Sadelain et al, Cancer Disc. (2013) 3:388-398 (2018); U.S. Pat. Nos. 7,446, 190 and 8,399,645.

In certain embodiments, a CAR construct is provided that comprises a ligand receptor pair construct as described herein. In certain embodiments, the CAR construct comprises an scFv that can be fused to the ligand receptor pair construct. In certain embodiments, the ligand receptor pair construct is a single chain ligand receptor pair construct that can be fused to the N-terminus of the scFv with or without a linker. In certain embodiments, the single chain ligand receptor pair construct comprises a protease cleavable linker. In certain embodiments, the receptor is fused with or without a first linker to the N-terminus of the scFv and the ligand is internally fused to a second linker connecting the heavy chain and the light chain of the scFv. In certain embodiments the linkers comprise a protease cleavage site cleavable by a protease. In certain embodiments, the ligand is fused with or without a first linker to the N-terminus of the scFv and the receptor is internally fused to a second linker connecting the heavy chain and the light chain of the scFv. In certain embodiments the first linker is cleavable and the second linker is uncleavable by a protease. In certain embodiments a T-cell can be modified to express a ligand receptor pair CAR.

### Sequence Homology

Certain embodiments of the present disclosure relate to an isolated polynucleotide or a set of polynucleotides encoding a fusion protein described herein. A polynucleotide in this context can encode all or part of a fusion protein.

The terms “nucleic acid,” “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence can be located 3′ to the coding sequence.

In certain embodiments, the present disclosure relates to polynucleotide and polypeptide sequences that are identical or substantially identical to a polypeptide encoding at least a portion of a fusion protein described herein, e.g., a first or second polypeptide of a biologically functional protein. The term “identical” in the context of two or more polynucleotide or polypeptide sequences, refers to two or more sequences or subsequences that are the same. Sequences are “substantially identical” if they have a percentage of amino acid residues or nucleotides that are the same (for example, about 80%, about 85%, about 90%, or about 95% identity over a specified region) when compared and aligned for maximum correspondence over a comparison window or over a designated region as measured using one of the commonly used sequence comparison algorithms as known to persons of ordinary skill in the art or by manual alignment and visual inspection. This definition also refers to the complement of a test polynucleotide sequence. The identity can exist over a region that is at least about 50 amino acids or nucleotides in length, or over a region that is 75-100 amino acids or nucleotides in length, or, where not specified, across the entire sequence of a polypeptide or polynucleotide. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

“Comparison window,” as used herein refers to a segment of a sequence comprising contiguous amino acid or nucleotide positions which can be from 20 to 1000 contiguous amino acid or nucleotide positions, for example from about 50 to about 600 or from about 100 to about 300 or from about 150 to about 200 contiguous amino acid or nucleotide positions over which a test sequence can be compared to a reference sequence of the same number of contiguous positions after the two sequences are optimally aligned. Longer segments up to and including the full-length sequence may also be used as a comparison window in certain embodiments. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2:482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

Certain embodiments described herein relate to variant sequences that comprise one or more amino acid substitutions. In some embodiments, the amino acid substitutions are conservative substitutions. In general, a “conservative substitution” is considered to be a substitution of one amino acid with another amino acid having similar physical, chemical and/or structural properties. Common conservative substitutions are listed under Column 1 of Table 4. One skilled in the art will appreciate that the main factors in determining what constitutes a conservative substitution are usually the size of the amino acid side chain and its physical/chemical properties, but that certain environments allow for substitution of a given amino acid with a broader range of amino acids than those listed in Column 1. These additional amino acids tend to either have similar properties to the amino acid being substituted but to vary more widely in size, or be of similar size but vary more widely in physical/chemical properties. This broader range of conservative substitutions is listed under Column 2 of Table 4. The skilled person could readily ascertain the most appropriate group of substituents to select from in view of the particular protein environment in which the amino acid substitution is being made.

TABLE 4

Conservative Amino Acid Substitutions

| | | |
|---|---|---|
| Original Amino Acid | Column 1 | Column 2 |
| Ala (A) | Gly, Ile, Leu, Met, Norleucine, Val | Cys, Gly, Ile, Leu, Met, Norleucine, Phe, Trp, Tyr, Val |
| Arg (R) | His, Lys | His, Lys |
| Asn (N) | Cys, Gln, Ser, Thr | Asp, Cys, Gln, Glu, Ser, Thr |
| Asp (D) | Glu | Asn, Cys, Gln, Glu, Ser, Thr |
| Cys (C) | Asn, Gln, Ser, Thr | Asn, Asp, Gln, Glu, Ser, Thr |
| Gln (Q) | Asn, Cys, Ser, Thr | Asn, Asp, Cys, Glu, Ser, Thr |
| Glu (E) | Asp | Asp, Asn, Cys, Gln, Ser, Thr |
| Gly (G) | Pro | Ala, Ile, Leu, Met, Norleucine, Pro, Val |
| His (H) | Arg, Lys | Arg, Lys, Phe, Trp, Tyr |
| Ile (I) | Ala, Gly, Leu, Met, Norleucine, Val | Ala, Cys, Gly, Leu, Met, Norleucine, Phe, Trp, Tyr, Val |
| Leu (L) | Ala, Gly, Ile, Met, Norleucine, Val | Ala, Cys, Gly, Ile, Met, Norleucine, Phe, Trp, Tyr, Val |
| Lys (K) | Arg, His | Arg, His |
| Met (M) | Ala, Gly, Ile, Leu, Norleucine, Val | Ala, Cys, Gly, Ile, Leu, Norleucine, Phe, Trp, Tyr, Val |
| Phe (F) | Tyr, Trp | Ala, Cys, Gly, His, Ile, Leu, Met, Norleucine, Trp, Tyr, Val |
| Pro (P) | Gly | Gly |
| Ser (S) | Asn, Cys, Gln, Thr | Asp, Asn, Cys, Gln, Glu, Thr |
| Thr (T) | Asn, Cys, Gln, Ser | Asp, Asn, Cys, Gln, Glu, Ser |
| Trp (W) | Phe, Tyr | Ala, Cys, Gly, His, Ile, Leu, Met, Norleucine, Phe, Tyr, Val |
| Tyr (Y) | Phe, Trp | Ala, Cys, Gly, His, Ile, Leu, Met, Norleucine, Phe, Trp, Val |
| Val (V) | Ala, Gly, Ile, Leu, Met, Norleucine | Ala, Cys, Gly, Ile, Leu, Met, Norleucine, Phe, Trp, Tyr |

### Preparation of Fusion Proteins

The fusion proteins described herein can be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

### Vectors Encoding Fusion Proteins

For recombinant production of a fusion protein described herein, a polynucleotide or set of polynucleotides encoding the fusion protein is generated and inserted into one or more vectors for further cloning and/or expression in a host cell. Polynucleotide(s) encoding the fusion protein can be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the fusion protein will be dependent on the format of the fusion protein, including whether or not the fusion protein comprises an antibody and the number of polypeptides within the fusion protein. For example, when a fusion protein comprises two polypeptide chains, two polynucleotides each encoding one polypeptide chain will be required. Similarly, in certain embodiments, when the fusion protein comprises a biologically functional protein in a mAb format, two polynucleotides each encoding one polypeptide chain are required. When multiple polynucleotides are required, they can be incorporated into one vector or into more than one vector.

Generally, for expression, the polynucleotide or set of polynucleotides is incorporated into an expression vector together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the polypeptides of the fusion protein and that such regulatory elements can be derived from a variety of sources, including bacterial, fungal, viral, mammalian or insect genes. The expression vector can optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector can be an extrachromosomal vector or an integrating vector.

Certain embodiments of the present disclosure relate to vectors (such as expression vectors) comprising one or more polynucleotides encoding at least a portion of a fusion protein described herein. The polynucleotide(s) can be comprised by a single vector or by more than one vector. In some embodiments, the polynucleotides are comprised by a multicistronic vector.

Expression vectors to be used to express polynucleotides include, but are not limited to, pTT5 and pUC15, Cells comprising vectors encoding fusion proteins.

Suitable host cells for cloning or expression of the fusion protein polypeptides include various prokaryotic or eukaryotic cells as known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells.

In certain embodiments, the fusion proteins are produced in bacteria, in particular when glycosylation and Fc effector function are not needed, as described for example in U.S. Pat. Nos. 5,648,237, 5,789,199, and 5,840,523, and in Charlton, Methods in Molecular Biology, Vol. 248, pp. 245-254, B.K.C. Lo, ed., Humana Press, Totowa, N.J., 2003.

Eukaryotic microbes such as filamentous fungi or yeast are suitable expression host cells in certain embodiments, in particular fungi and yeast strains whose glycosylation pathways have been “humanized” resulting in the production of an antibody with a partially or fully human glycosylation pattern (see, for example, Gerngross, 2004, Nat. Biotech. 22:1409-1414, and Li et al., 2006, Nat. Biotech. 24:210-215).

Suitable host cells for the expression of glycosylated fusion proteins are usually eukaryotic cells. For example, U.S. Pat. Nos. 5,959,177, 6,040,498, 6,420,548, 7,125,978 and 6,417,429 describe PLANTIBODIES™ technology for producing antibodies in transgenic plants. Mammalian cell lines adapted to grow in suspension are particularly useful for expression of fusion proteins. Examples include, but are not limited to, monkey kidney CV1 line transformed by SV40 (COS-7), human embryonic kidney (HEK) line 293 or 293 cells (see, for example, Graham et al., 1977, J. Gen Virol., 36:59), baby hamster kidney cells (BHK), mouse sertoli TM4 cells (see, for example, Mather, 1980, Biol Reprod, 23:243-251); monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR⁻ CHO cells, see Urlaub et al., 1980, Proc Natl Acad Sci USA, 77:4216), and myeloma cell lines (such as Y0, NS0 and Sp2/0). Exemplary mammalian host cell lines suitable for production of antibodies are reviewed in Yazaki & Wu, Methods in Molecular Biology, Vol. 248, pp. 255-268 (B.K.C. Lo, ed., Humana Press, Totowa, N.J., 2003).

In certain embodiments, the host cell is a transient or stable higher eukaryotic cell line, such as a mammalian cell line. In some embodiments, the host cell is a mammalian HEK293T, CHO, HeLa, NS0 or COS cell. In some embodiments, the host cell is a stable cell line that allows for mature glycosylation of the fusion protein.

The host cells comprising the expression vector(s) encoding the fusion protein can be cultured using routine methods to produce the fusion protein. Alternatively, in some embodiments, host cells comprising the expression vector(s) encoding the fusion protein can be used therapeutically or prophylactically to deliver the fusion protein to a subject, or polynucleotides or expression vectors can be administered to a cell from a subject ex vivo and the cell then returned to the body of the subject.

In some embodiments, a host cell comprises (for example, has been transformed with) a vector comprising a polynucleotide that encodes the VL of a binding domain described herein and the VH of the binding domain. In some embodiments, a host cell comprises a first vector comprising a polynucleotide that encodes the VL of a binding domain described herein and a second vector comprising a polynucleotide that encodes the corresponding VH of the binding domain. In some embodiments, the host cell is eukaryotic, for example, a Chinese Hamster Ovary (CHO) cell, a human embryonic kidney (HEK) cell or a lymphoid cell (e.g. Y0, NS0, Sp20 cell).

In certain embodiments, the host cell is Expi293™ (Thermo Fisher, Waltham, MA). In certain embodiments, the host cell is CHO-S cells (National Research Council Canada) or HEK293 cells.

Certain embodiments of the present disclosure relate to a method of making a fusion protein comprising culturing a host cell into which one or more polynucleotides encoding the fusion protein, or one or more expression vectors encoding the fusion protein, have been introduced, under conditions suitable for expression of the fusion protein, and optionally recovering the fusion protein from the host cell (or from host cell culture medium).

Cell culture media that can be used include, but are not limited to, DMEM (Thermo Fisher, Waltham, MA), Opti-MEM™ (Thermo Fisher, Waltham, MA), Opti-MEM™ I Reduced Serum Medium (Thermo Fisher, Waltham, MA), RPMI-1640 medium, Expi293™ Expression Medium (Thermo Fisher, Waltham, MA), and FreeStyle CHO expression medium (Thermo Fisher Scientific, Waltham, MA).

The cell culture medium can be supplemented with serum, e.g., fetal bovine serum (FBS), amino acids, e.g., L-glutamine, antibiotics, e.g., penicillin, and streptomycin, and/or antimycotics, e.g., amphotericin, or any other supplements routinely used in the to support cell culture.

### Purification of Fusion Proteins

Typically, the fusion proteins are purified after expression. Proteins can be isolated or purified in a variety of ways known to those skilled in the art (see, for example, Protein Purification: Principles and Practice, 3^{rd}Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, are also useful. As is well known in the art, a variety of natural proteins bind Fc and antibodies, and these proteins are used for purification of certain antibodies. For example, the bacterial proteins A and G bind to the Fc region. Likewise, the bacterial protein L binds to the Fab region of some antibodies. Purification can also be enabled by a particular fusion partner. For example, antibodies can be purified using glutathione resin if a GST fusion is employed, Ni⁺² affinity chromatography if a His-tag is employed, or immobilized anti-flag antibody if a flag-tag is used. The degree of purification necessary will vary depending on the use of the antibodies. In some instances, no purification can be necessary.

In certain embodiments, fusion proteins are substantially pure. The term “substantially pure” (or “substantially purified”) when used in reference to a fusion protein described herein, means that the fusion protein is substantially or essentially free of components that normally accompany or interact with the protein as found in its naturally occurring environment, such as a native cell, or a host cell in the case of recombinantly produced fusion protein. In certain embodiments, a fusion protein that is substantially pure is a protein preparation having less than about 30%, less than about 25%, less than about 20%, less than about 15%, less than about 10%, or less than about 5% (by dry weight) of contaminating protein.

Assessment of protein purification and/or homogeneity can be performed by any method known in the art, including, but not limited to, non-reducing/reducing CE-SDS, non-reducing/reducing SDS-PAGE, Ultra-high performance liquid chromatography-size exclusion chromatography (UPLC-SEC), High Performance Liquid Chromoatography (HPLC), mass spectrometry, multi angle light scattering (MALS), dynamic light scattering (DLS).

### Post-Translational Modifications

In certain embodiments, the fusion proteins described herein comprise one or more post-translational modifications. Such post-translational modifications can occur in vivo, or they be conducted in vitro after isolation of the fusion protein from the host cell.

Post-translational modifications include various modifications as are known in the art (see, for example, Proteins - Structure and Molecular Properties, 2nd Ed., T. E. Creighton, W. H. Freeman and Company, New York, 1993; Post-Translational Covalent Modification of Proteins, B. C. Johnson, Ed., Academic Press, New York, pgs. 1-12, 1983; Seifter et al., 1990, Meth. Enzymol., 182:626-646, and Rattan et al., 1992, Ann. N.Y. Acad. Sci., 663:48-62). In those embodiments in which the fusion proteins comprise one or more post-translational modifications, the fusion proteins can comprise the same type of modification at one or several sites, or it can comprise different modifications at different sites.

Examples of post-translational modifications include glycosylation, acetylation, phosphorylation, amidation, derivatization by known protecting/blocking groups, formylation, oxidation, reduction, proteolytic cleavage or specific chemical cleavage by cyanogen bromide, trypsin, chymotrypsin, papain, V8 protease or NaBH₄.

Other examples of post-translational modifications include, for example, addition or removal of N-linked or O-linked carbohydrate chains, chemical modifications of N-linked or O-linked carbohydrate chains, processing of N-terminal or C-terminal ends, attachment of chemical moieties to the amino acid backbone, and addition or deletion of an N-terminal methionine residue resulting from prokaryotic host cell expression. Post-translational modifications can also include modification with a detectable label, such as an enzymatic, fluorescent, isotopic or affinity label to allow for detection and isolation of the protein. Examples of suitable enzyme labels include, but are not limited to, horseradish peroxidase, alkaline phosphatase, beta-galactosidase and acetylcholinesterase. Examples of suitable prosthetic group complexes include, but are not limited to, streptavidin/biotin and avidin/biotin. Examples of suitable fluorescent materials include, but are not limited to, umbelliferone, fluorescein, fluorescein isothiocyanate, rhodamine, dichlorotriazinylamine fluorescein, dansyl chloride and phycoerythrin. An example of a luminescent material is luminol, examples of bioluminescent materials include luciferase, luciferin and aequorin, and examples of suitable radioactive materials include iodine, carbon, sulfur, tritium, indium, technetium, thallium, gallium, palladium, molybdenum, xenon and fluorine.

Additional examples of post-translational modifications include acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, gamma-carboxylation, GPI anchor formation, hydroxylation, iodination, methylation, myristylation, pegylation, prenylation, racemization, selenoylation, sulfation, transfer-RNA mediated addition of amino acids to proteins such as arginylation, and ubiquitination.

### Masking and Programmed Activation of the Fusion Proteins

In accordance with the present disclosure, the fusion proteins are masked from engaging their intended target(s). The extent to which binding of the fusion proteins to their target(s) is decreased may be measured by standard techniques such as enzyme-linked immunosorbent assay (ELISA,), bio-layer interferometery (BLI), surface plasmon resonance (SPR), fluorescence-activated cell sorting (FACS), flow cytometry, kinetic exclusion assay (KinExA), meso scale discovey (MSD)), microfluidics, or isothermal titration calorimetry (ITC). In certain embodiments, the fusion protein comprises an antigen-binding domain that is masked by the ligand-receptor pair and binding of the antigen-binding domain to its cognate antigen is decreased by at least 3-fold as compared to a corresponding unmasked antigen-binding domain, for example, binding of the antigen-binding domain to its cognate antigen is decreased by at least 5-fold, at least 10-fold, at least 20-fold, at least 25-fold or at least 30-fold, or at least 40-fold, or at least 50-fold, or at least 70-fold or at least 80-fold, or at least 90-fold or at least 100-fold, or at least 200-fold or at least 400-fold, or at least 600-fold or at least 800-fold or at least 1000-fold or at least 2000-fold or at least 5000-fold or at least 10,000-fold.

In accordance with the present disclosure, protease cleavage of at least one of the peptidic linkers between the ligand or receptor of the ligand-receptor pair and the biologically functional protein unmasks (activates) the fusion protein such that it can bind its intended target(s). The susceptibility of the peptidic linker to cleavage may be tested in vitro by standard techniques including those described in the Examples herein. The extent to which binding of the fusion protein to its target(s) is recovered after protease cleavage may also be tested by standard techniques such as enzyme-linked immunosorbent assay (ELISA), bio-layer interferometery (BLI), surface plasmon resonance (SPR), fluorescence-activated cell sorting (FACS), flow cytometry, kinetic exclusion assay (KinExA), meso scale discovey (MSD), microfluidics, or isothermal titration calorimetry (ITC). Recovery of binding of the fusion protein to its target(s) may be partial or complete. Partial recovery of binding is defined as measurable binding of the relevant domain of the fusion protein (e.g., ligand, receptor or antigen-binding domain) to its intended target and may be, for example, between 100-fold and 2-fold less than binding of the parental domain. Partial recovery may be about 100-fold, 75-fold, 50-fold, 25-fold, 10-fold, 5-fold- or 2-fold less than the binding of the parental domain.

### Methods of Treatment

In certain aspects, the present disclosure includes methods for the treatment of a disease or condition comprising administration of a fusion protein described herein to a subject in need thereof. In certain embodiments, the subject is a mammal. In certain embodiments, the subject is human.

In certain embodiments, the methods disclosed herein are for the treatment of cancer. Cancers can include, but are not limited to, hematologic neoplasms (including leukemias, myelomas and lymphomas), carcinomas (including adenocarcinomas and squamous cell carcinomas), melanomas and sarcomas. Carcinomas and sarcomas are also frequently referred to as “solid tumors”. In certain embodiments, the cancer is a solid tumor. In certain embodiments, the cancer is leukemia. In certain embodiments, the cancer is lymphoma.

The fusion protein can exert either a cytotoxic or cytostatic effect and can result in one or more of a reduction in the size of a tumor, the slowing or prevention of an increase in the size of a tumor, an increase in the disease-free survival time between the disappearance or removal of a tumor and its reappearance, prevention of an initial or subsequent occurrence of a tumor (for example, metastasis), an increase in the time to progression, reduction of one or more adverse symptom associated with a tumor, or an increase in the overall survival time of a subject having a tumor.

In certain embodiments, the methods disclosed herein are for the treatment of an immunodeficiency disorder or disease.

In certain embodiments, the methods disclosed herein are for the treatment of autoimmune diseases or conditions.

The methods described herein comprise administering a fusion protein described herein to a subject in need thereof. The fusion protein can be administered to a subject by an appropriate route of administration. As will be appreciated by the person of skill in the art, the route and/or mode of administration will vary depending upon the desired results. Typically, immunotherapeutic antibodies are administered by systemic administration or local administration. Local administration can be at the site of a tumor or into a tumor draining lymph node. Generally, the fusion proteins will be administered by parenteral administration, for example, by intravenous, intramuscular, intradermal, intraperitoneal, subcutaneous or spinal administration, such as by injection or infusion.

Treatment is achieved by administration of a “therapeutically effective amount” of the fusion protein. A “therapeutically effective amount” refers to an amount that is effective, at dosages and for periods of time necessary, to achieve a desired therapeutic result. A therapeutically effective amount can vary according to factors such as the disease state, age, sex, and weight of the subject. A therapeutically effective amount is also one in which any toxic or detrimental effects of the fusion protein are outweighed by the therapeutically beneficial effects. “Sufficient amount” means an amount sufficient to produce a desired effect, e.g., an amount sufficient to modulate an immune response to a target cell or tissue, e.g., by immunomodulatory ligand-receptor binding to an immune cell.

A suitable dosage of the fusion protein can be determined by a skilled medical practitioner. The selected dosage level will depend upon a variety of pharmacokinetic factors including the activity of the particular fusion protein employed, the route of administration, the time of administration, the rate of excretion of the polypeptide, the duration of the treatment, other drugs, compounds and/or materials used in combination with the fusion protein, e.g., anti-cancer agents, the age, sex, weight, condition, general health and prior medical history of the subject being treated, and like factors well known in the medical arts.

### Methods of Modulating an Immune Cell or an Immune Response

In certain embodiments, the fusion proteins described herein are administered to a subject in need thereof, for example a subject having cancer, in order to modulate the immune system of the subject. Thus, in certain embodiments, the fusion proteins described herein downregulate an immune response or upregulate an immune response.

In accordance with this embodiment, administration of a sufficient amount of fusion protein to the subject can effect one or more of the following to activate or upregulate an immune response: modulation of an immune checkpoint, modulation of T-cell receptor signaling, modulation of T-cell activation, modulation of pro-inflammatory cytokines, modulation of interferon-γ production by T cells, modulation of T-cell suppression, modulation of M2-type tumor associated macrophages (TAM) or myeloid-derived suppressor cell (MDSC) survival and/or differentiation, and/or modulation of cytotoxic or cytostatic effects on cells.

In certain embodiments, provided herein are methods of modulating an immune response, comprising inhibition of an immune checkpoint, stimulation of an immune checkpoint, immune cell activation, stimulation of T-cell receptor signaling, and stimulation of antibody-dependent cellular cytotoxicity (ADCC), T cell-dependent cytotoxicity (TDCC)), Cell-dependent cytotoxicity (CDC), or antibody-dependent cellular phagocytosis (ADCP).

In certain embodiments, the fusion protein, when activated by a protease, is capable of agonizing a target leukocyte costimulatory receptor. Functional effects of leukocyte costimulatory receptor agonism include activation of T effector cells, differentiation and activation of inflammatory myeloid cells and/or recruitment of B cells and/or NKT cells. Activation of T effector cells can result in increased production of one or more cytokines by the T cells, such as interferon gamma (IFN-y), interleukin-2 (IL-2), interleukin-12 (IL-12), interleukin-17 (IL-17), interleukin-21 (IL-21), granulocyte-macrophage colony-stimulating factor (GM-CSF), tumor necrosis factor-α (TNF-α), macrophage inflammatory protein 1β (MIP-1β) and/or C-X-C motif ligand 13 (CXCL13). Increased production of IL-21 and CXCL13 by T effector cells may, for example, support the differentiation and activation of inflammatory myeloid cells in the TME, recruit anti-tumor lymphoid cells such as B and NKT cells and/or support the formation of tertiary lymphoid structures.

In certain embodiments, the fusion protein activates T effector cells. In some embodiments, the fusion protein increases production of GM-CSF, TNF-α, MIP-1β, IL-17, IL-12, IL-21 and/or C-X-C motif ligand 13 (CXCL13) by T effector cells.

In certain embodiments, the fusion protein decreases CSF1-dependent viability of monocytes and activate T effector cells.

Certain embodiments of the present disclosure relate to methods of using the fusion proteins to modulate leukocyte costimulatory receptor agonism in vivo, for example, in order to treat cancer.

In certain embodiments, the methods relate to inhibition or downregulation of an immune cell or immune response, e.g., for treating an autoimmune disease or disorder. Thus in certain embodiments, the fusion protein is administered in a sufficient amount to modulate an immune cell. In certain embodiments, the downregulation of an immune response is by modulation of an immune checkpoint, modulation of T-cell receptor signaling, modulation of T cell activation, modulation of pro-inflammatory cytokines, modulation of interferon-γ production by T cells, modulation of T cell suppression, modulation of M2-type tumor associated macrophages (TAM) or myeloid-derived suppressor cell (MDSC) survival and/or differentiation, and/or modulation of cytotoxic or cytostatic effects on cells.

### Methods to Modify ADCC of a Target Cell

In certain embodiments, the fusion proteins described herein induce antibody dependent cell-mediated cytotoxicity (ADCC), which in turn results in increased lysis of the target cell. In certain embodiments, the fusion protein comprises an Fc region with increased binding affinity of the Fc for FcγRIIIa (an activating receptor) resulting in increased antibody dependent cell-mediated cytotoxicity (ADCC) and increased lysis of the target cell. In certain embodiments, the Fc region is with modified CH2 domains comprising amino acid modifications that result in increased binding affinity of the Fc for FcγRIIIa (an activating receptor) resulting in increased antibody dependent cell-mediated cytotoxicity (ADCC).

In certain embodiments, fusion proteins described herein reduce antibody dependent cell-mediated cytotoxicity (ADCC). In certain indications, a decrease in, or elimination of, ADCC and complement-mediated cytotoxicity (CDC) is desirable. In certain embodiments, fusion proteins comprise and Fc region with modified CH2 domains comprising amino acid modifications that result in increased binding to FcγRIIb or amino acid modifications that decrease or eliminate binding of the Fc region to all of the Fcγ receptors (“knock-out” variants) can be useful. In certain embodiments, the fusion protein comprises an Fc region with decreased binding to FcγRIIb (an inhibitory receptor).

### Pharmaceutical Compositions

The fusion proteins according to the present disclosure can be formulated in pharmaceutical compositions. These compositions can comprise, in addition to one or more of the fusion proteins, a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer’s Injection, Lactated Ringer’s Injection. Preservatives, stabilisers, buffers, antioxidants and/or other additives can be included, as required.

For fusion proteins according to the present disclosure that are to be given to an individual, administration is preferably in a “therapeutically effective amount” that is sufficient to show benefit to the individual. A “prophylactically effective amount” can also be administered, when sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington’s Pharmaceutical Sciences, 16th edition, Osol, A. (ed), 1980.

A composition can be administered alone or in combination with other treatments, either simultaneously or sequentially dependent upon the condition to be treated.

### Kits

The present disclosure also provides for kits comprising one or more of the compositions described herein and instructions for use. Thus, in certain embodiments, described herein are kits comprising vectors for expressing a fusion protein described herein and instructions for use. In certain embodiments, described herein are kits comprising host cells comprising a vector for expressing a fusion protein and instructions for use. In certain embodiments, are kits comprising a purified fusion protein and instructions for use. The purified fusion protein can be lyophilized or provided in a dry form, such as a powder or granules, and the kit can additionally contain a suitable solvent for reconstitution of the lyophilized or dried component(s).

The kit typically will comprise a container and a label and/or package insert on or associated with the container. The label or package insert contains instructions customarily included in commercial packages of therapeutic products, providing information or instructions about the indications, usage, dosage, administration, contraindications and/or warnings concerning the use of such therapeutic products. The label or package insert can further include a notice in the form prescribed by a governmental agency regulating the manufacture, use or sale of pharmaceuticals or biological products, which notice reflects approval by the agency of manufacture, for use or sale for human or animal administration. The container holds a composition comprising the fusion protein. In some embodiments, the container can have a sterile access port. For example, the container can be an intravenous solution bag or a vial having a stopper that can be pierced by a hypodermic injection needle.

In addition to the container containing the composition comprising the fusion protein, the kit can comprise one or more additional containers comprising other components of the kit. For example, a pharmaceutically-acceptable buffer (such as bacteriostatic water for injection) (BWFI), phosphate-buffered saline, Ringer’s solution or dextrose solution), other buffers or diluents.

Suitable containers include, for example, bottles, vials, syringes, intravenous solution bags, and the like. The containers can be formed from a variety of materials such as glass or plastic. If appropriate, one or more components of the kit can be lyophilized or provided in a dry form, such as a powder or granules, and the kit can additionally contain a suitable solvent for reconstitution of the lyophilized or dried component(s).

The kit can further include other materials desirable from a commercial or user standpoint, such as filters, needles, and syringes.

### EXAMPLES

The following examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); A.L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington’s Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1 Design of Masked Anti-CD3 × Anti-Her2 T Cell-Engager Fusion Proteins

An anti-CD3 Fab × anti-Her2 scFv Fc was appended with a mask on the anti-CD3 Fab by linking one of the ligand-receptor pair PD-1-PDL-1 to the N-terminus of the light chain of the Fab and the other to the N-terminus of the heavy chain. The fusion protein constructs were designed as follows.

### Methods

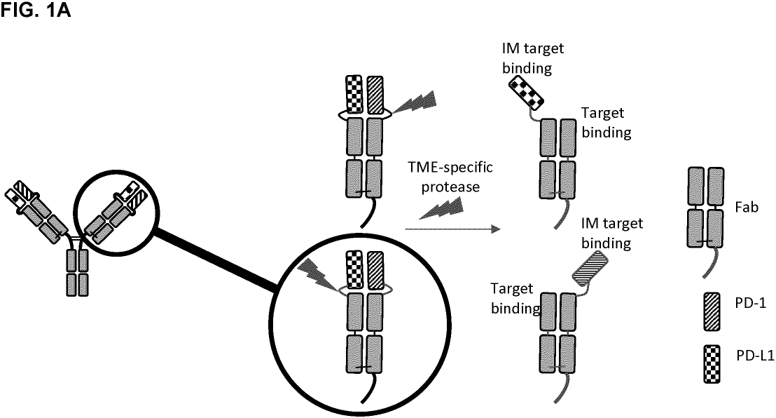

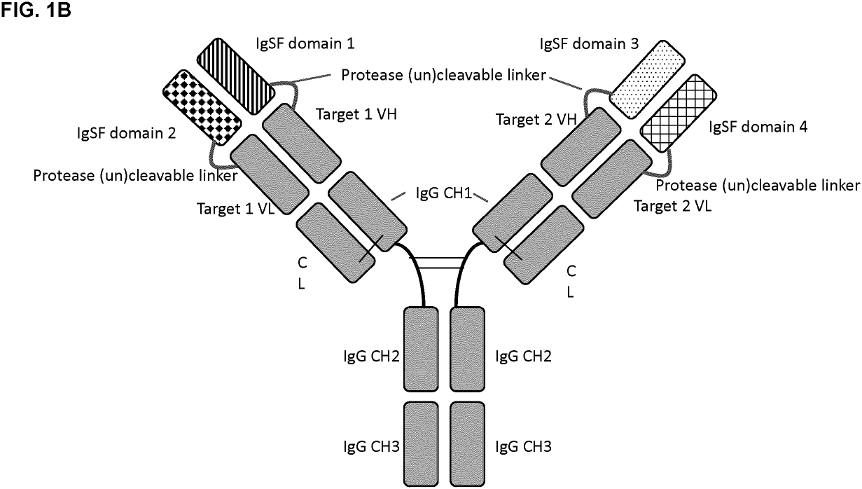

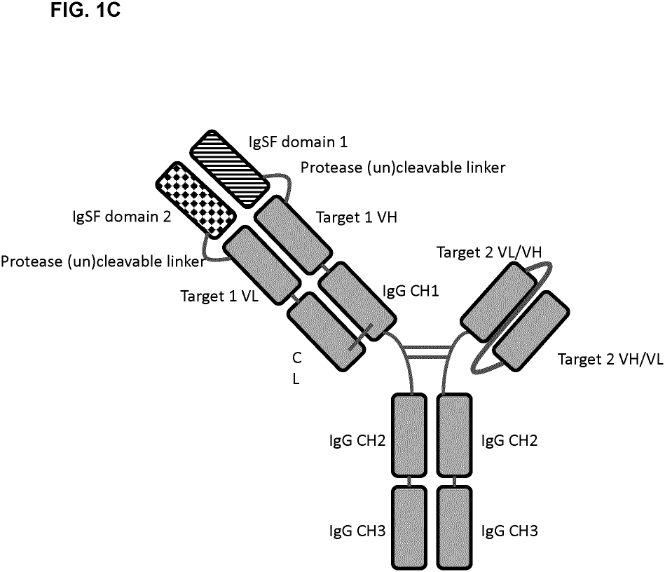

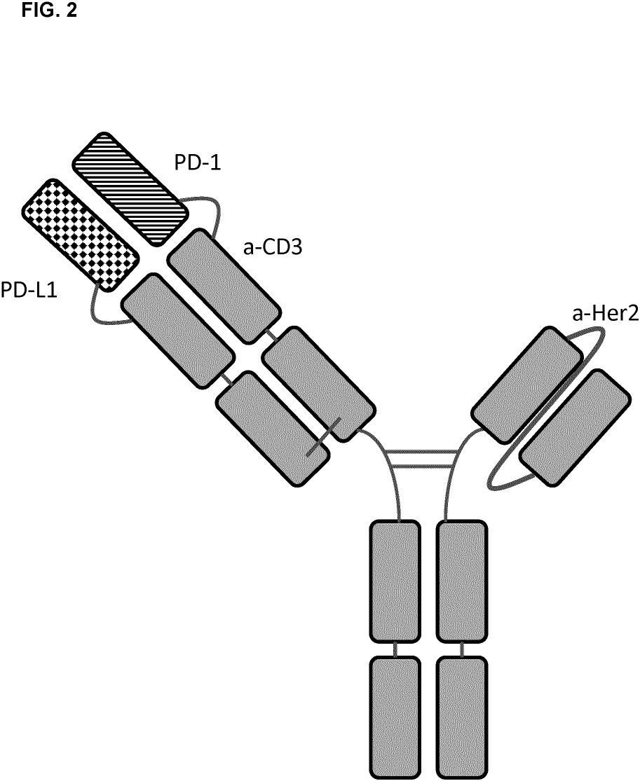

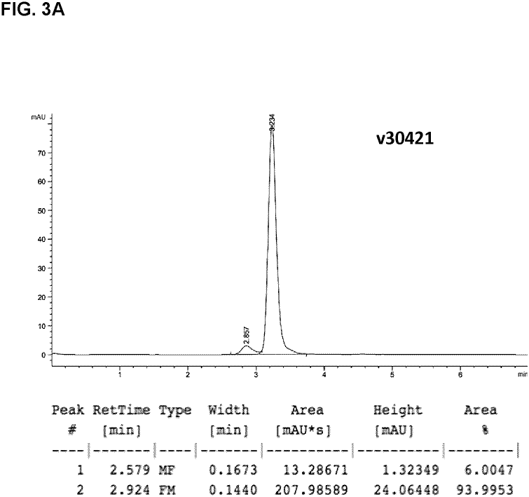

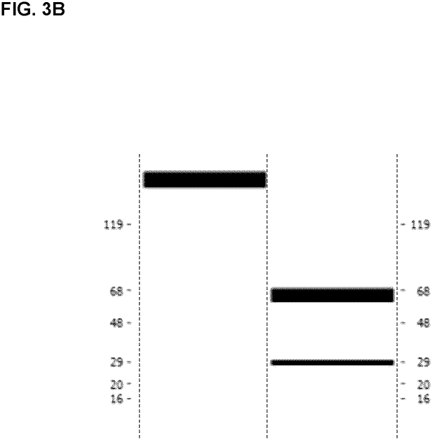

The fusion proteins were in a modified bispecific Fab × scFv Fc format with a half-antibody comprising the anti-CD3 heavy and light chain that forms a heterodimer with an anti-Her2 scFv fused to an Fc. The anti-CD3 paratope was described in US20150232557A1 (VL SEQ ID NO: 1, VH SEQ ID NO: 2). The anti-Her2 paratope was in an scFv format that is based on trastuzumab VL and VH (Carter, P. et al. Humanization of an anti-p185HER2 antibody for human cancer therapy. Proc Natl Acad Sci U S A 89, 4285-4289, doi:10.1073/pnas.89.10.4285 (1992)) connected by a glycine serine linker as described in US10000576B1 (SEQ ID NO: 3). To allow for selective heterodimeric pairing, mutations were introduced in the anti-CD3 CH3 as well as the anti-Her2 scFv-Fc CH3 chain as described previously (Von Kreudenstein, T. S. et al. Improving biophysical properties of a bispecific antibody scaffold to aid developability: quality by molecular design. MAbs 5, 646-654, doi:10.4161/mabs.25632 (2013); (A chain CH3 domain, SEQ ID NO: 4, B chain CH3 domain SEQ ID NO: 5). Mutations (L234A L235A_D265S as compared to a wild type human IgG1 CH2) were also introduced in both CH2 domains to reduce binding to the Fc gamma receptors (SEQ ID NO: 6). Furthermore, polypeptides based on the modified protein sequences of the IgV domains of human PD-1 (SEQ ID NO: 7) and/or PD-L1 (SEQ ID NO: 8) (West, S. M. & Deng, X. A. Considering B7-CD28 as a family through sequence and structure. Exp Biol Med (Maywood), 1535370219855970, doi:10.1177/1535370219855970 (2019) were fused to the N-termini of heavy chain (VH-CH1-hinge-CH2-CH3) and kappa light chain (VL-CL) of the anti-CD3 variable domains, respectively, using linkers that were comprised of a variable number of repeats of sequences predicted to form helical turns ((EAAAK)ₙ, Chen, X., Zaro, J. L. & Shen, W. C. Fusion protein linkers: property, design and functionality. Adv Drug Deliv Rev 65, 1357-1369, doi:10.1016/j.addr.2012.09.039 (2013)). These PD-1 and PD-L1 moieties were predicted to dimerize and sterically block epitope binding. In all variants, either the PD-1 or the PD-L1 sequence used as one half of the mask contained mutations to increase the affinity of the PD-1:PD-L1 complex as described before (Maute, R. L. et al. Engineering high-affinity PD-1 variants for optimized immunotherapy and immuno-PET imaging. Proc Natl Acad Sci USA 112, E6506-6514, doi:10.1073/pnas. 1519623112 (2015); SEQ ID NO: 9; Liang, Z. et al. High-affinity human PD-L1 variants attenuate the suppression of T cell activation. Oncotarget 8, 88360-88375, doi:10.18632/oncotarget.21729 (2017); SEQ ID NO: 10). Additionally, in all WT PD-1 moieties, an unpaired cysteine was mutated to serine to remove the liability of an exposed reducing group (SEQ ID NO: 11). Some variants also contained a cleavage sequence for the tumor microenvironment (TME)-associated protease uPa (MSGRSANA SEQ ID NO: 28), to allow for the removal of part or all of the mask by exposure of the fusion protein to protease. A schematic of the construct design for a masked Fab as well as the intended mechanism of action is shown in FIG. 1. The final designs were bispecific Fab x scFv Fc molecules that contain a masked anti-CD3 Fab as well as an anti-Her2 scFv. A schematic is shown in FIG. 2 and the sequences used are listed in Table A.

TABLE A

Sequence composition of tested Variants*

| Variant No | Schematic | Description | Clone H1 | Clone L1 | Clone H2 |
|---|---|---|---|---|---|
| 30421 | 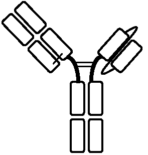 | CD3 × Her2 Fab × scfv Fc without mask | 12989 | 12985 | 21490 |
| 30423 | 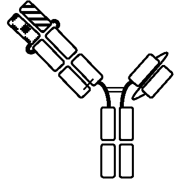 | HA PD-1:WT PD-L1 masked CD3 × Her2 Fab × scfv Fc, with an uncleavable linker | 22080 | 22091 | 21490 |
| 30426 | 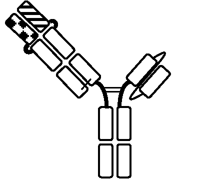 | WT PD-1:HA PD-L1 masked CD3 × Her2 Fab × scfv Fc, with an uncleavable linker | 22082 | 22092 | 21490 |
| 30430 | 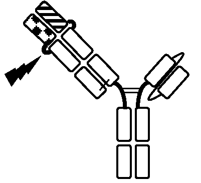 | HA PD-1:WT PD-L1 masked CD3 × Her2 Fab × scFv Fc, PD-L1 with a cleavable linker | 22080 | 22096 | 21490 |
| 30436 | 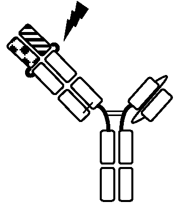 | WT PD-1:HA PD-L1 masked CD3 × Her2 Fab × scfv Fc, PD-1 cleavable | 22086 | 22092 | 21490 |
| 31934 | 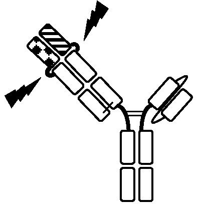 | WT PD-1:WT PD-L1 masked CD3 × Her2 Fab × scfv Fc, PD-1 and PD-L1 cleavable | 22083 | 22094 | 21490 |
| 31929 | 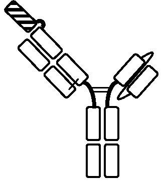 | Half-masked CD3 × Her2 Fab × scfv Fc, HA PD-1 attached to HC | 22080 | 12985 | 21490 |
| 31931 | 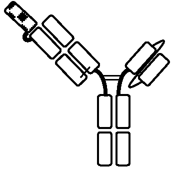 | Half-masked CD3 × Her2 Fab × scfv Fc, HA PD-L1 attached to LC | 12989 | 22092 | 21490 |
| 32497 | 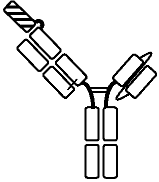 | Half-masked CD3 × Her2 Fab × scfv Fc, PD-1 KO attached to HC | 23734 | 12985 | 21490 |
| 33551 | 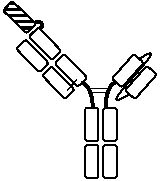 | Half-masked CD3 × hemagglutinin Fab × scfv Fc, HA PD-1 attached to HC | 22080 | 12985 | 11018 |

^{∗} The PD-1 IgV domain attached to the heavy chain is indicated with a striped pattern in the cartoons and the PD-L1 IgV domain attached to the light chain is shown as a checkered pattern.

### Example 2 Production of Masked Anti-CD3 Variants

Sequences of modified CD3 × Her2 Fab × scFv variants designed in Example 1 were ported into expression vectors and expressed and purified as follows.

### Methods

Heavy chain vector inserts comprising a signal peptide (Barash et al., 2002, Biochem and Biophys Res. Comm., 294:835-842, SEQ ID 27) and the heavy chain clone terminating at G446 (EU numbering) of CH3 were ligated into a pTT5 vector to produce heavy chain expression vectors. Light chain vector inserts comprising the same signal peptide and the light chain clone were ligated into a pTT5 vector to produce light chain expression vectors. The resulting heavy and light chain expression vectors were sequenced to confirm correct reading frame and sequence of the coding DNA.

Heavy and light chains of the modified CD3 × Her2 Fab × scFv Fc variants were co-expressed in 25 mL cultures of Expi293F™ cells (Thermo Fisher, Waltham, MA). Expi293™ cells were cultured at 37° C. in Expi293™ Expression Medium (Thermo Fisher, Waltham, MA) on an orbital shaker rotating at 125 rpm in a humidified atmosphere of 8% CO₂. A volume of 25 mL with a total cell count of 7.5 × 10⁷ cells was transfected with a total of 25 µg DNA at a transfection ratio of 40:40:20 for H1:L1:H2. Prior to transfection the DNA was diluted in 1.5 mL Opti-MEM™ IReduced Serum Medium (Thermo Fisher, Waltham, MA). In a volume of 1.42 mL Opti-MEM™ I Reduced Serum Medium, 80 µL of ExpiFectamine™ 293 reagent (Thermo Fisher, Waltham, MA) were diluted and, after incubation for five minutes, combined with the DNA transfection mix to a total volume of 3 mL. After 10 to 20 minutes the DNA-ExpiFectamine™293 reagent mixture was added to the cell culture. After incubation at 37° C. for 18-22 hours, 150 µL of ExpiFectamine™ 293 Enhancer 1 and 1.5 mL of ExpiFectamine™ 293 Enhancer 2 (Thermo Fisher, Waltham, MA) were added to each culture. Cells were incubated for five to seven days and supernatants were harvested for protein purification.

Clarified supernatant samples were applied to 1 mL of slurry containing 50% mAb Select SuRe resin (GE Healthcare, Chicago, IL) in batch mode. Columns were equilibrated in PBS. After loading, columns were washed with PBS and protein eluted with 100 mM sodium citrate buffer pH 3.5. The eluted samples were pH adjusted by adding 10% (v/v) 1 M Tris pH 9 to yield a final pH of 6-7. After concentration, all of the material was injected into an AKTA Pure FPLC System (GE Life Sciences) and run on a Superdex 200 Increase 10/300 GL (GE Life Sciences) column pre-equilibrated with PBS pH 7.4. The protein was eluted from the column at a rate of 0.75 mL/min and collected in 0.5 mL fractions. Peak fractions were pooled and concentrated using Vivaspin 20, 30 kDa MWCO polyethersulfone concentrators (MilliporeSigma Burlington MA, USA). After sterile filtering through 0.2 µm PALL Acrodisc™ Syringe Filters with Supor™ Membrane, proteins were quantitated based on A280 nm (Nanodrop), frozen and stored at -80° C. until further use.

### Results

Samples contained significant amounts of higher molecular weight species as determined by UPLC-SEC after protein A purification (not shown) and preparative SEC was used in order to obtain samples of high purity. Yields after preparative SEC ranged from 1.5 - 5 mg per variant. Sample purity and stability was assessed in Example 3 and Example 4.

### Example 3 Purity and Homogeneity Assessment of Masked Anti-CD3 Variants

Purified variants were assessed for purity and sample homogeneity by non-reducing/reducing CE-SDS UPLC-SEC as described below.

### Methods

Following purification, purity of samples was assessed by non-reducing and reducing High Throughput Protein Express assay using CE-SDS LabChip® GXII (Perkin Elmer, Waltham, MA). Procedures were carried out according to HT Protein Express LabChip® User Guide version 2 with the following modifications. mAb samples, at either 2 uL or 5 uL(concentration range 5-2000 ng/ul), were added to separate wells in 96 well plates (BioRad, Hercules, CA) along with 7 uL of HT Protein Express Sample Buffer (Perkin Elmer # 760328). The reducing buffer is prepared by adding 3.5 µl of DTT(1 M) to 100 µl of HT Protein Express Sample Buffer. mAb samples were then denatured at 90° C. for 5 mins and 35 µl of water is added to each sample well. The LabChip® instrument was operated using the HT Protein Express Chip (Perkin Elmer #760499) and the HT Protein Express 200 assay setting (14 kDa-200 kDa).

UPLC-SEC was performed on an Agilent Technologies 1260 Infinity LC system using an Agilent Technologies AdvanceBio SEC 300A column at 25° C. Before injection, samples were centrifuged at 10000 g for 5 minutes, and 5 µl was injected into the column. Samples were run for 7 min at a flow rate of 1 mL/min in PBS, pH 7.4 and elution was monitored by UV absorbance at 190-400 nm. Chromatograms were extracted at 280 nm. Peak integration was performed using the OpenLAB CDS ChemStation software.

### Results

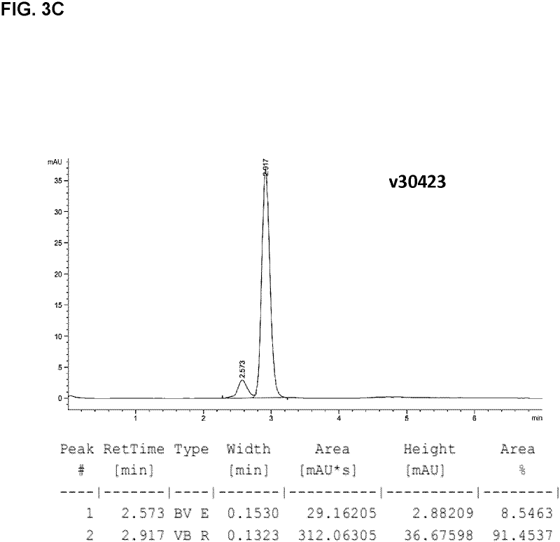

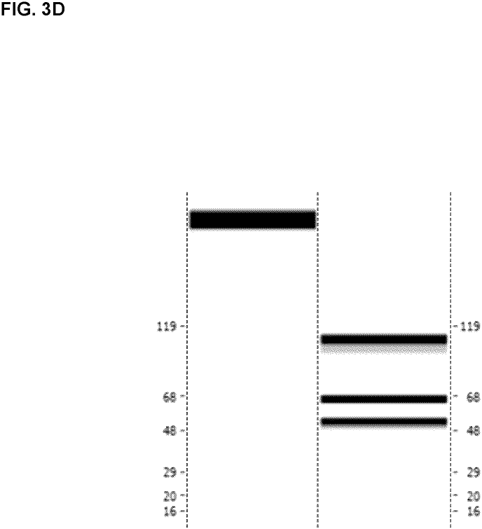

Representative UPLC-SEC traces of samples after preparative SEC purification of the variants in FIGS. 3A, 3C, 3E, and 3G showed highly homogeneous samples that contained 89% - 94% of correct species. The presence of a small peak at a low retention time compared to the main species indicates the presence of small amounts of high molecular weight species such as oligomers and aggregates in all samples.

Analysis of non-reducing CE-SDS (FIGS. 3B, 3D, 3E and 3F) showed a single predominant species and only bands corresponding to the intact chains of all variants are found in the reducing CE-SDS run. Notably, the masked heavy and light chains showed a significantly higher apparent molecular weight than what would be expected (110 kDa vs 63 kDa for the HC, 54 kDa vs 37 kDa for the LC). This was also reflected in the high apparent molecular weight of the non-reduced, disulfide bonded species (215 kDa vs 152 kDa). Glycosylation of both the PD1 and PD-L1 moieties in the designs is likely causing the increase in apparent molecular weight (Tan, S. et al. An unexpected N-terminal loop in PD-1 dominates binding by nivolumab. Nat Commun 8, 14369, doi:10.1038/ncomms14369 (2017), Li, C. W. et al. Glycosylation and stabilization of programmed death ligand-1 suppresses T-cell activity. Nat Commun 7, 12632, doi:10.1038/ncomms12632 (2016)).

### Example 4 Stability Assessment of Masked Anti-CD3 Variants

Purified variants were assessed for thermal stability by differential scanning calorimetry (DSC) as described below.

### Methods

Samples of a representative set of modified CD3 × Her2 Fab × scFv Fc variants were diluted in PBS to 0.5-1 mg/ml. For DSC analysis using NanoDSC (TA Instruments, New Castle, DE, USA), 950 µl of sample and matching buffer (PBS) were added to sample and reference 96 well plates, respectively. At the start of DSC run, a buffer (PBS) blank injection was performed to stabilize the baseline. Each sample was then injected and scanned from 25 to 95° C. at 1° C./min with 60 psi nitrogen pressure. Thermograms were analyzed using the NanoAnalyze software. The matching buffer thermogram was subtracted from sample thermogram and baseline fit using a sigmoidal curve. Data was then fit with a two-state scaled DSC model.

### Results

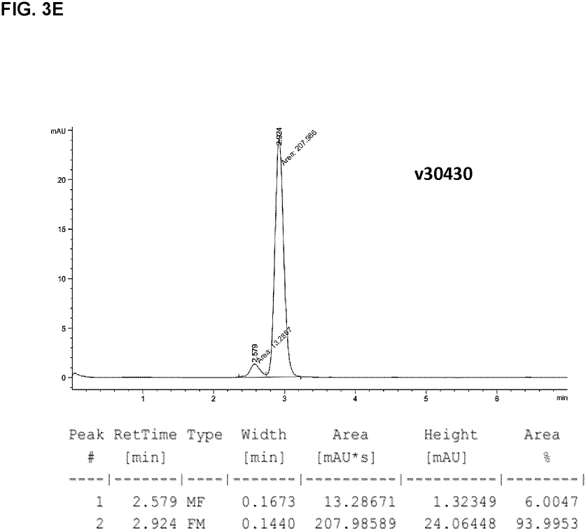

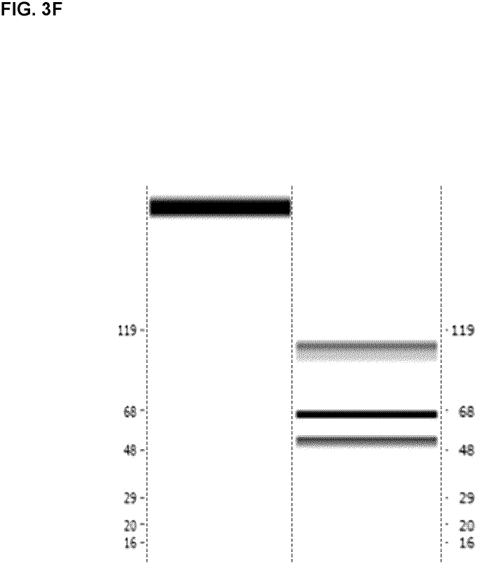

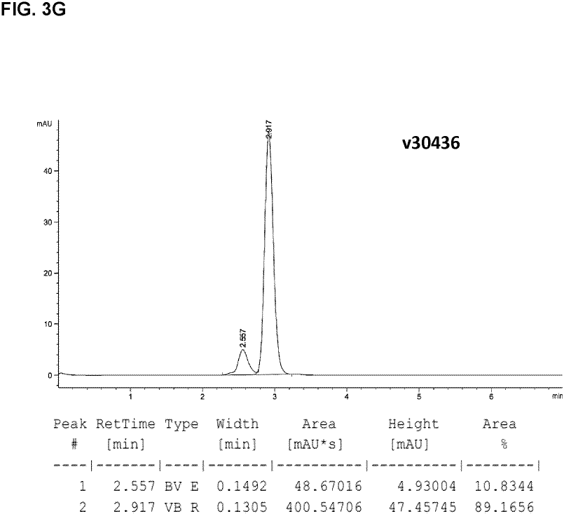

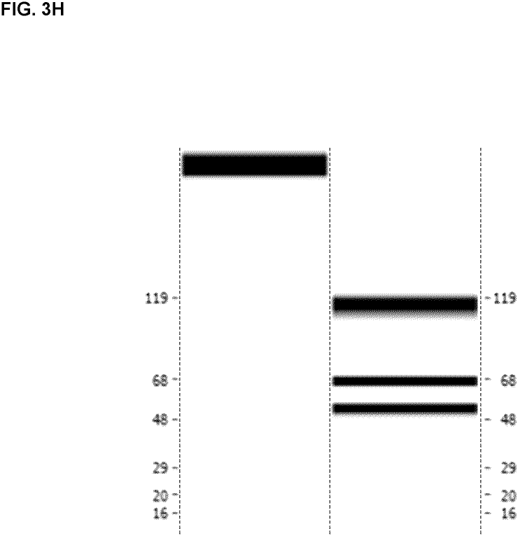

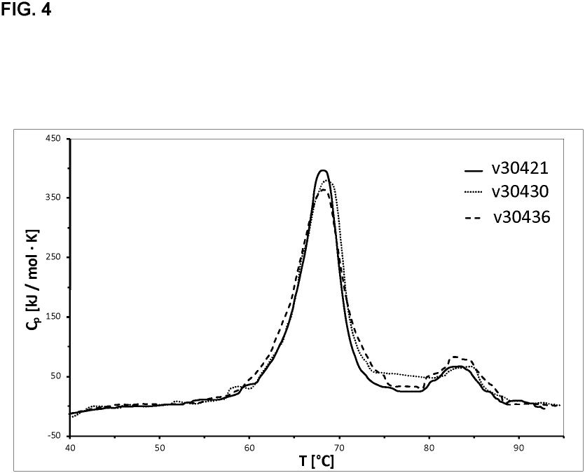

The DSC thermogram of the unmodified CD3 × Her2 Fab × scFv Fc variant (30421, FIG. 4) showed transitions at 68 and 83° C. While the transition with a Tₘ of 68° C. likely corresponds to unresolved individual transitions for unfolding of the anti-CD3 Fab, anti-Her2 scFv and CH2 domain, the transition at Tₘ = 83° C. likely corresponds to unfolding of the CH3 domain in the heavy chain. Thermograms of variants bearing a PD-1:PD-L1 mask (30430, 30436; FIG. 4) also showed two transitions at similar temperatures and with similar thermogram traces to the unmasked variant. This indicates that the fused masking domains do not affect the Tₘ of the anti-CD3 Fab, and either unfold cooperatively with the Fab or uncooperatively but with a similar Tₘ to Fab, scFv and CH2.

### Example 5 uPa Cleavage of Anti-CD3 Variants

In order to assess release of part of or all of the mask from the anti-CD3 Fab of the fusion proteins by cleavage of the introduced protease cleavage sites in the linkers, samples were treated with uPa in vitro. Reactions were monitored by reducing CE-SDS as follows.

### Methods

For a preparative cleavage of the variants, 25-100 ug of purified sample was diluted to a final variant concentration of 0.2 mg/mL in PBS + 0.05 % Tween20 and Recombinant Human u-Plasminogen Activator (uPa)/Urokinase (R&D Systems #P00749) was added at a 1:50 protease:substrate molar:molar ratio. After incubation at 37° C. for 24 h, sample fragments were analyzed in reducing CE-SDS as described in Example 2 and then frozen and stored at -80° C. until further use.

### Results

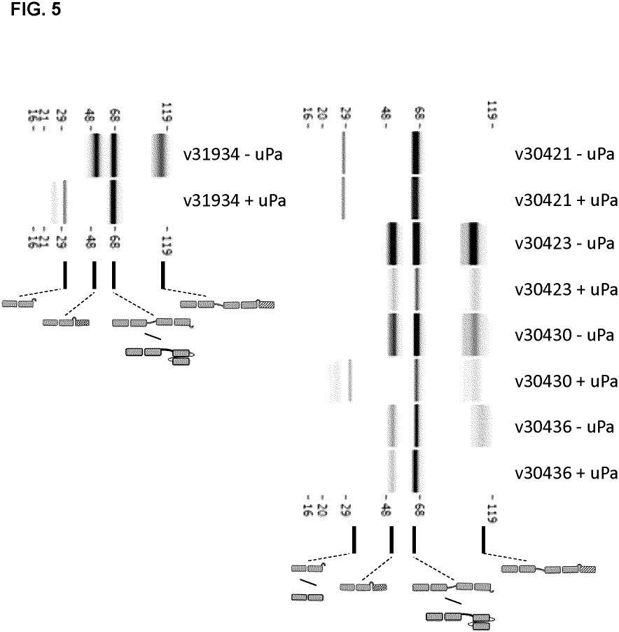

Analysis of reducing CE-SDS profiles of the masked variants with and without uPa treatment revealed that under the investigated conditions, part or all of the mask was removed from the Fab effectively by cleavage at the introduced cleavage sites (FIG. 5). For successfully cleaved variants (30430, 30436, 31934), bands representing fragments of masked heavy and/or light chain disappeared completely upon cleavage while fragments of un-masked heavy and/or light chain appear. While a broad band of low intensity corresponding to a fragment of free PD-1 can be observed for variant 30430, this was not the case for the released PD-L1 in variant 30436. Small size and size heterogeneity due to glycosylation (Tan, S. et al. An unexpected N-terminal loop in PD-1 dominates binding by nivolumab. Nat Commun 8, 14369, doi:10.1038/ncomms14369 (2017), Li, C. W. et al. Glycosylation and stabilization of programmed death ligand-1 suppresses T-cell activity. Nat Commun 7, 12632, doi:10.1038/ncomms12632 (2016)) likely rendered the free PD-1 and PD-L1 fragments barely detectable and undetectable, respectively. In variants that do not contain the cleavage sequence (30421, 30423), no cleavage was observed.

### Example 6 Masking/Unmasking of CD3-Binding

Uncleaved and cleaved samples of anti-CD3 variants from Example 5 were tested for binding to CD3 expressing Jurkat cells by ELISA and to Pan T-cells by flow cytometry as follows.

### Methods

### Elisa

Human Jurkat cells (Fujisaki Cell Center, Japan) were maintained in RPMI-1640 medium supplemented with 2 mM L-glutamine and 10% of heat-inactivated fetal bovine serum (FBS) with 1X Penicillin/Streptomycin, in a humidified + 5% CO2 incubator at 37° C. Samples of modified CD3 × Her2 variants from Example 5 were diluted 2X in blocking buffer, containing saturating amounts of irrelevant human Ig, followed by seven three-fold serial dilutions in blocking buffer for a total of eight concentration points. Blocking buffer alone was added to control wells to measure background signal on cells (negative/blank control).

All incubations were performed at 4° C. On the day of the assay, exponentially growing cells were centrifuged and seeded in a 96-well filter plate (MilliporeSigma, Burlington, MA, USA) in a 1:1 mixture of complete culture medium and blocking buffer. Equal volumes of 2X variants or controls were added to cells and incubated for 1 hour. The plate was then washed 4 times using vacuum filtration. An HRP-conjugated anti-human IgG Fc gamma specific secondary antibody (Jackson ImmunoResearch, West Grove, PA, USA) was added to the wells and further incubated for 1 h. Plates were washed 7 times by vacuum filtration followed by the addition of TMB substrate (Thermo Scientific, Waltham MA, USA) at room temperature. The reaction was stopped by adding 0.5 volume of 1 M sulfuric acid and the supernatant was transferred by filtration into a clear 96-well plate (Corning, Corning, NY, USA). Absorbance at 450 nm was read on a Spectramax 340PC plate reader with path-check correction.

Binding curves of blank-subtracted OD450 versus linear or log antibody concentration were fitted with GraphPad Prism 8 (GraphPad Software, La Jolla, CA, USA). A one-site specific, four-parameter nonlinear regression curve fitting model with Hill slope was employed in order to determine Bmax and apparent Kd values for each test article.

### Flow Cytometry

Antibodies were titrated in a v-bottom 96-well plate (Sarstedt AG, Nümbrecht, Germany) from 300 nM to 1.7 pM at a 1:3 dilution in a total of 20 uL/well in FACS buffer - PBS containing 2% FBS (Thermo Fisher Scientific, Waltham, MA). Healthy donor peripheral blood pan T cells (BioIVT, Westbury, NY) were thawed and washed in medium that consisted of RPMI 1640 medium (A1049101, ATCC modification) (Thermo Fisher Scientific, Waltham, MA) supplemented with 10% Fetal Bovine Serum (Thermo Fisher Scientific, Waltham, MA). The cells were counted, resuspended in FACS buffer, and added to the 96-well plate at 50,000 cells per well. The cells were incubated with the variants at 4° C. for 1 hr and then washed 2x with FACS buffer and 1 mg/mL of secondary antibody AF647 Goat anti-human IgG Fc (Jackson ImmunoResearch, West Grove, PA). A 1000-fold diluted viability dye (Biolegend, San Diego, CA) was also added to the wells. The plate was incubated at room temperature for 30 min while shaking (200 rpm). Cells were then washed 2x in FACS buffer and resuspended in 100 uL of FACS buffer. For assay read-out, Geometric mean of APC fluorescence was measured by flow cytometry on a BD LSRFortessa (BD Biosciences, San Jose, CA). Raw data was analyzed on FlowJo, LLC Software (Becton, Dickinson & Company, Ashland, OR). Graphs were generated using GraphPad Prism version 8.1.2 for Mac OS X (GraphPad Software, La Jolla, CA).

### Results

### Elisa

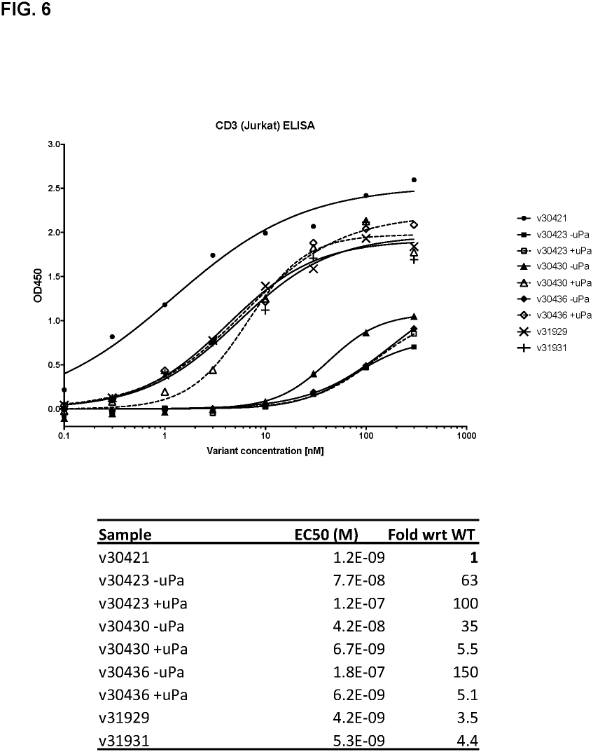

As can be seen in FIG. 6, variants containing a full PD1:PD-L1 based mask appended to the CD3 Fab (30423, 30430, 30436) showed 40-180 fold reduced binding compared to the unmasked control (30421). Upon treatment with uPa, CD3 binding of the cleavable variants 30430 and 30436 was partially restored (within 6-7 fold of the unmasked control). This partial recovery might be caused by a steric hinderance of epitope binding by the portion of the mask that is left on the mask after cleavage. Concomitantly, controls that only had PD-1 or PD-L1 appended to either heavy or light chain, respectively (31929, 31931), showed a similar reduction (4-5 fold) in binding compared to the unmasked control as the uPa-cleaved samples of the fully masked variants.

### Flow Cytometry

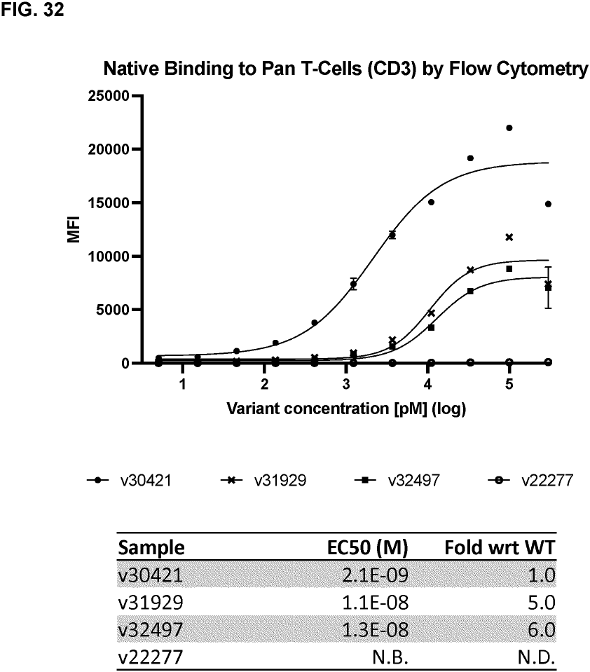

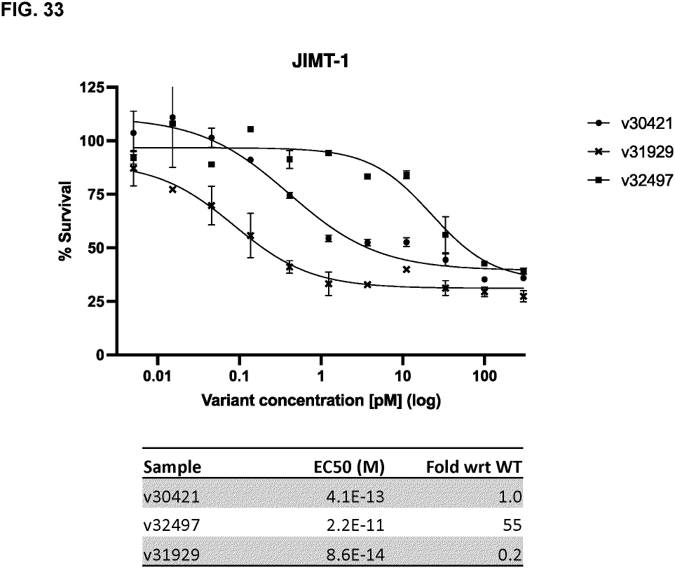

As can be seen in FIG. 22, variants containing a full PD1:PD-L1 based mask appended to the CD3 Fab (30423, 30430) showed > 43 fold reduced binding compared to the unmasked control (30421). Upon treatment with uPa, CD3 binding of the cleavable variant 30430 was partially restored (within 29 fold of the unmasked control). This partial recovery might be caused by a steric hinderance of epitope binding by the portion of the mask that is left on the mask after cleavage. Concomitantly, a control that only had PD-1 appended to the heavy chain (31929), showed a similar reduction (14 fold) in binding compared to the unmasked control as the uPa-cleaved samples of the fully masked variants. In a separate experiment, a variant with a non-functional PD-1 domain appended to the heavy chain (32497), showed a similar reduction (6-fold) in binding compared to the unmasked control as seen for the equivalent variant with a functional PD-1 (31929, 5-fold) (FIG. 32).

### Example 7 T-Cell Dependent Cellular Cytotoxicity of Masked and Unmasked Variants

The functional impact of the PD-1:PD-L1 based mask on the ability of the CD3 × Her2 Fab × scFv Fc variants to engage and activate T-cells for the killing of Her2-bearing cells was assessed in a T-cell dependent cellular cytotoxicity (TDCC) assay as follows.

### Methods

### Coculture Assay

JIMT-1 (Leibniz Institute, Braunschweig, Germany) cultured in growth medium consisting of DMEM medium (Thermo Fisher Scientific, Waltham, MA) supplemented with 10% Fetal Bovine Serum (Thermo Fisher Scientific, Waltham, MA), HCC1954 (ATCC, Manassas, VA) and HCC827 (ATCC, Manassas, VA) cultured in growth medium consisting of RPMI-1640 ATCC modification (Thermo Fisher Scientific, Waltham, MA) supplemented with 10% Fetal Bovine Serum, and MCF-7 (ATCC, Manassas, VA) cultured in growth medium consisting of MEM medium (Thermo Fisher Scientific, Waltham, MA) supplemented with 10% Fetal Bovine Serum and 0.01 mg/mL of human recombinant Insulin (Thermo Fisher Scientific, Waltham, MA) were maintained horizontally in T-175 flasks (Corning, Corning, NY) in an incubator at 37° C. with 5% carbon dioxide. On the day of setting up the assay, the variants were titrated in triplicate at 1:3 dilution directly in 384-well cell culture treated optical bottom plates (ThermoFisher Scientific, Waltham, MA) from 5 nM to 0.08 pM. Tumor cells were rinsed with PBS (Thermo Fisher Scientific, Waltham, MA), harvested with TrypLE Express (Thermo Fisher Scientific, Waltham, MA), diluted in media, and counted using Vi-Cell (Beckman Coulter, Indianapolis, IN). A vial of primary human pan-T cells (BioIVT, Westbury, NY), was thawed in a 37° C. water bath, washed in media, and counted using Vi-Cell. Pan T cell suspension was mixed with tumor cells at 5:1 effector to target ratio, washed and resuspended at 0.55 E6 cell/ml. 20 uL of the mixed cell suspension was added to the plate containing the titrated variants. The plates were incubated for 48 h in an incubator at 37° C. with 5% carbon dioxide. The samples were then subjected to a high-content cytotoxicity assessment and the supernatants were collected for IFNγ analysis.

### High Content Cytotoxicity Analysis

For visualization of nuclei and assessment of viability, cells were stained with Hoechst33342. 10 µL of Hoechst33342 (Thermo Fisher Scientific, Waltham, MA). was diluted 1:1000 in media, added to the cells after the 48 h period and incubated for a further 1 h at 37° C. Then, the plate was subjected to high content image analysis on CellInsight CX-5 (ThermoFisher Scientific, Waltham, MA) in order to distinguish and quantify viable and dead tumor cells as well as effector cells. The plate was scanned on the CellInsight CX5 high content instrument using the SpotAnalysis.V4 Bioapplication with the following settings: Objective: 10x, Channel 1 -386 nm: Hoechst (Fixed exposure time 0.008 ms with a Gain of 2).

### IFNγ Quantification

For IFNγ quantification in a MSD U-PLEX 384-well single-spot assay, streptavidin coated multi-array plates (MA6000 384 SA plates, Meso Scale Diagnostics, Rockville, MD) were blocked with 50 uL of Diluent 100, sealed, and incubated at room temperature with shaking (800 rpm) for 30 mins. At the end of incubation, all wells were aspirated. Biotinylated capture IFNγ antibody was added to Diluent 100 at 1:16.5 ratio, and 10 uL of capture antibody solution was added to each well of the blocked plates. The plates were sealed and incubated at 4° C. overnight. The next day, frozen supernatants from co-culture assay were thawed on wet ice. The plates were washed and 5 µl of Diluent 43 was added to each well followed by 5 µl of thawed supernatant sample or standard. The plates were sealed and incubated at room temperature for 1 hr while shaking (800 rpm). Following incubation, plates were washed and 10 uL of SULFO-TAG detection antibody diluted 1:1000 in Diluent 3 was added to each well. The plate was sealed and incubated at room temperature for 1 hr while shaking (800 rpm). After incubation, the plate was washed and 40 µl of MSD GOLD Read Buffer was added to each well. The plate was read on MESO SECTOR R600 instrument (Meso Scale Diagnostics, Rockville, MD).

### PD-L1 and Her2 Receptor Quantification

Her2 and PD-L1 receptor quantification were performed via flow cytometry using Quantum Simply Cellular anti-human and anti-mouse IgG kits respectively (Bangs Laboratories, Fishers, Indiana). Tumor cells were rinsed with PBS (Thermo Fisher Scientific, Waltham, MA), and harvested with TrypLE Express (Thermo Fisher Scientific, Waltham, MA). Cells were counted using Vi-Cell (Beckman Coulter, Indianapolis, IN), washed, and resuspended in FACS buffer -PBS containing 2% FBS (Thermo Fisher Scientific, Waltham, MA) at 4×10^6 c/mL. 25 uL of tumor cell suspension was added in triplicate to a 96-well V-bottom plate (Sarstedt AG, Nümbrecht, Germany). Anti-Her2-AF647 (Trastuzumab, monovalent antibody, Zymeworks, Vancouver, BC), anti-PDL1-APC (Clone MIH1, BD Biosciences, San Jose, CA) or irrelevant negative control IgG-AF657 (Zymeworks, Vancouver, BC) antibody at 15 ug/mL was added to the wells and Eppendorf tubes (Thermo Fisher Scientific, Waltham, MA) containing Quantum Simply Cellular IgG beads (anti-human or anti-mouse) and blank beads. Cells and beads were incubated with the antibodies for 1 hr at 4° C. in the dark. Cells and beads were washed, resuspended, and analyzed by flow cytometry. For analysis, a standard curve was generated using the spreadsheet provided by Bangs Laboratories (Fishers, Indiana) for the specific lot of beads, and the surface antigen binding capacity (ABC) was generated by entering the geometric means of the cell populations using the same spreadsheet. ABC values represent the number of molecules of receptor expressed on the cell surface assuming a monovalent binding model. The standard curve, determining the range for confident determination of the receptor number ranged from 3500 receptors/cell to 330000 receptors/cell for Her2 and from 4400 receptors/cell to 630000 receptors/cell for PD-L1.

### Results

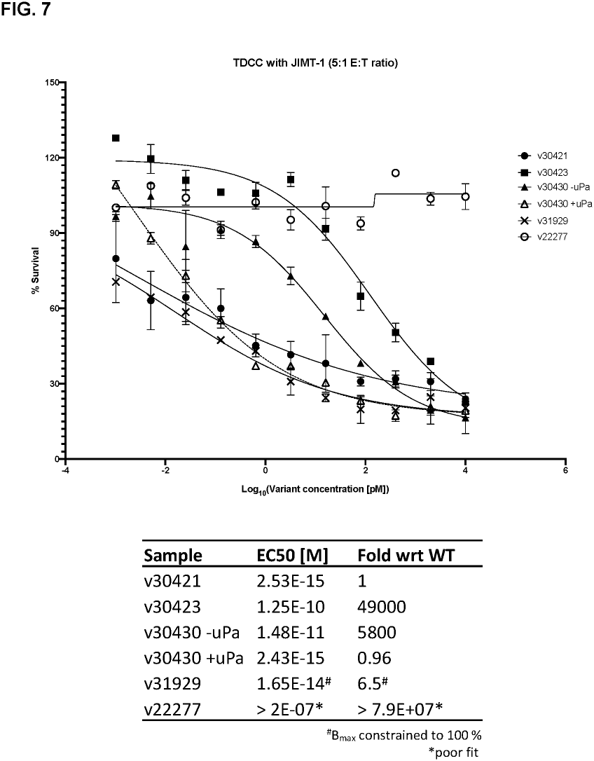

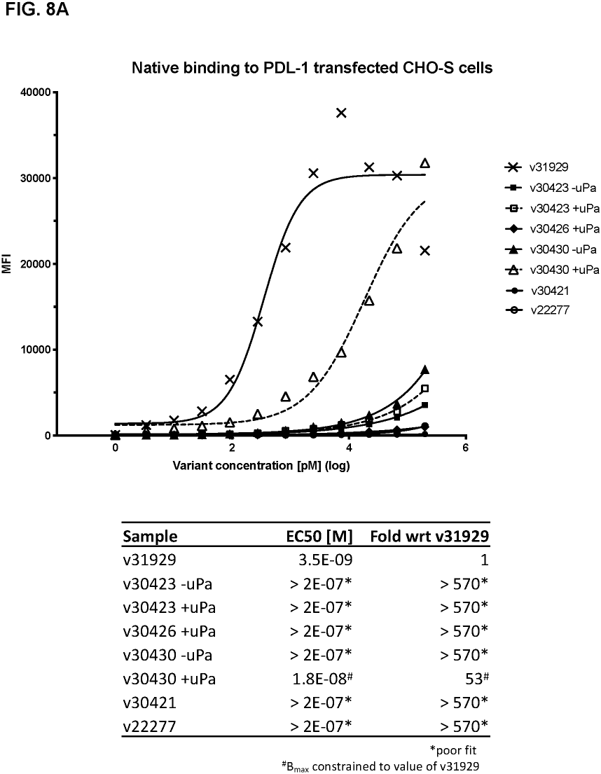

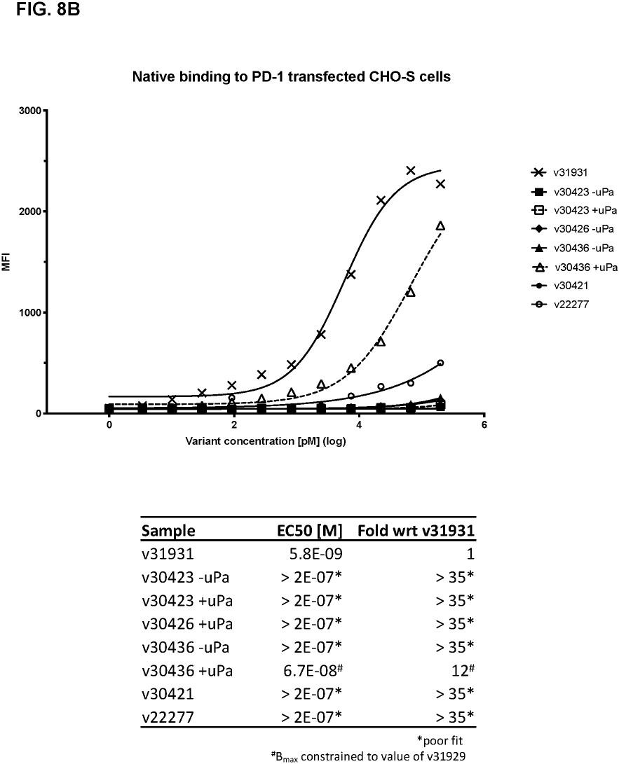

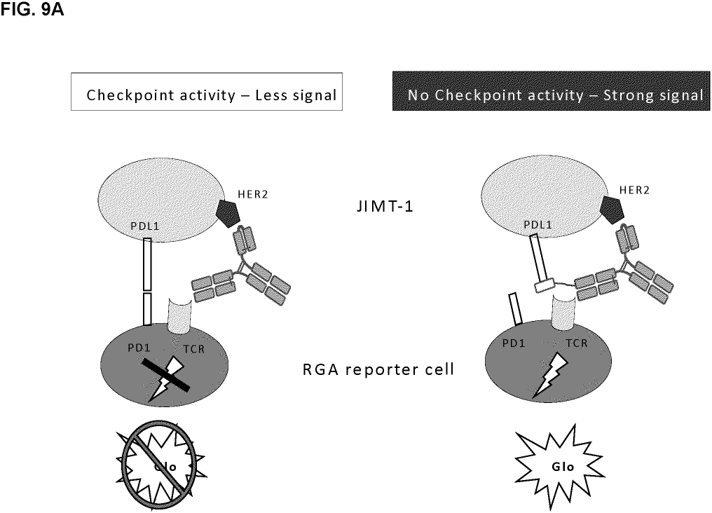

The masking effects seen for the CD3 x Her2 Fab x scFv Fc variants in binding to CD3 in Example 6 were recapitulated when the same samples were interrogated for function in a TDCC assay with Her2-expressing JIMT-1 cells (FIG. 7). While the unmasked variant (30421) showed robust tumor cell killing at low variant concentrations, the potency of a masked, uncleavable variant (30423) was decreased by 49000 X. A fully masked variant with a cleavable PD-L1 moiety on the light chain (30430) was also reduced in potency without uPa treatment, by 5800 X. This discrepancy in masking between uncleavable and cleavable variants was seen for CD3 binding as well (Example 6). When the mask was cleaved by uPa, the potency of 30430 returned to that of an unmasked (30421) variant. A control variant with only the PD-1 moiety of the mask attached (31929) showed similar potency to 30421 and uPa-treated 30430. An irrelevant anti Respiratory Syncytial Virus (RSV) antibody (22277) showed no activation of T cells for tumor cell killing.

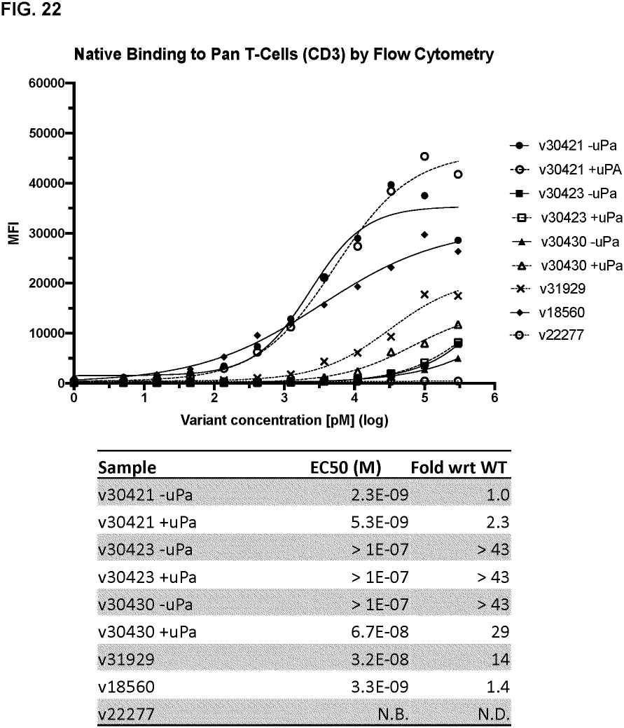

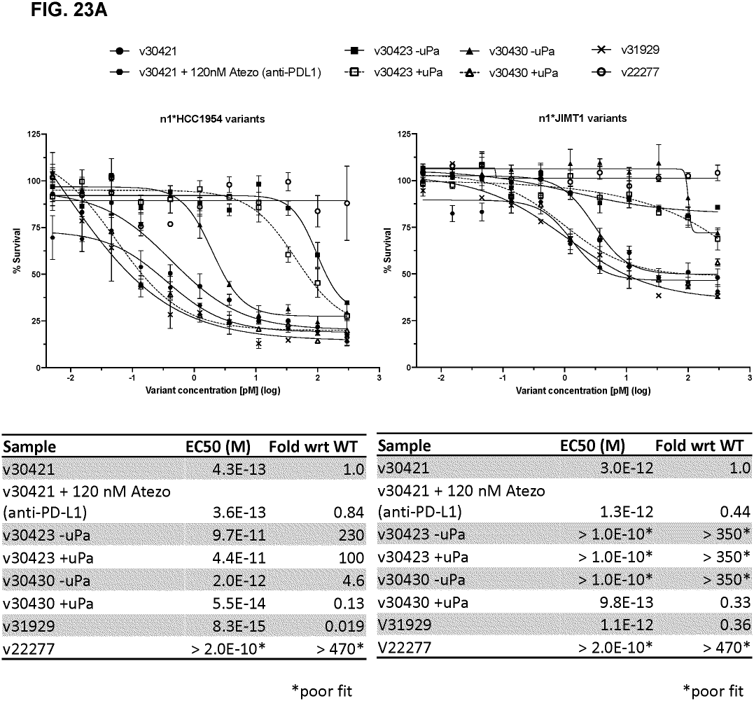

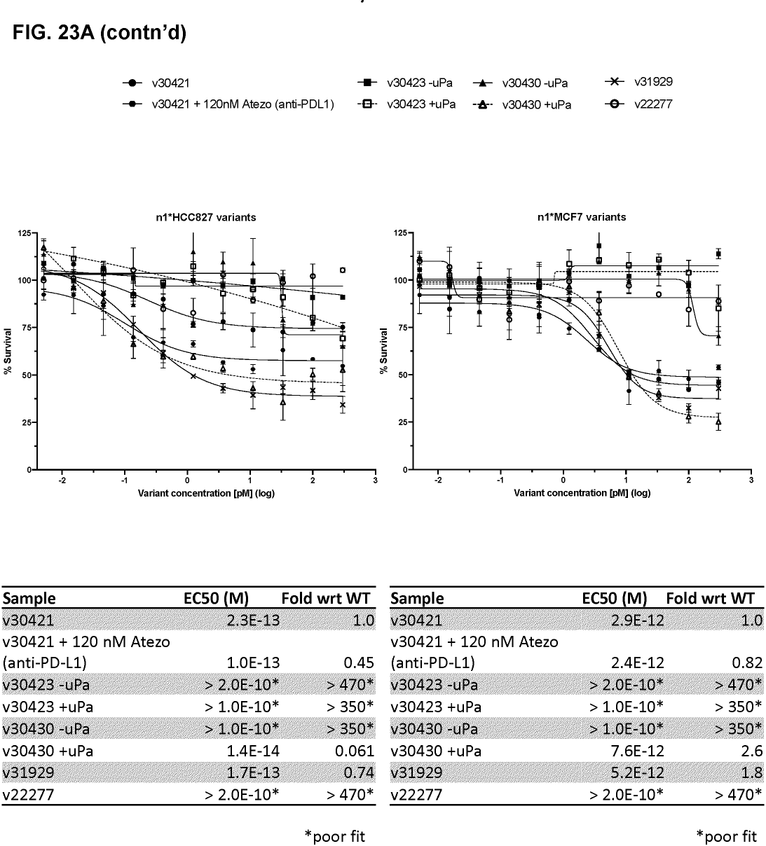

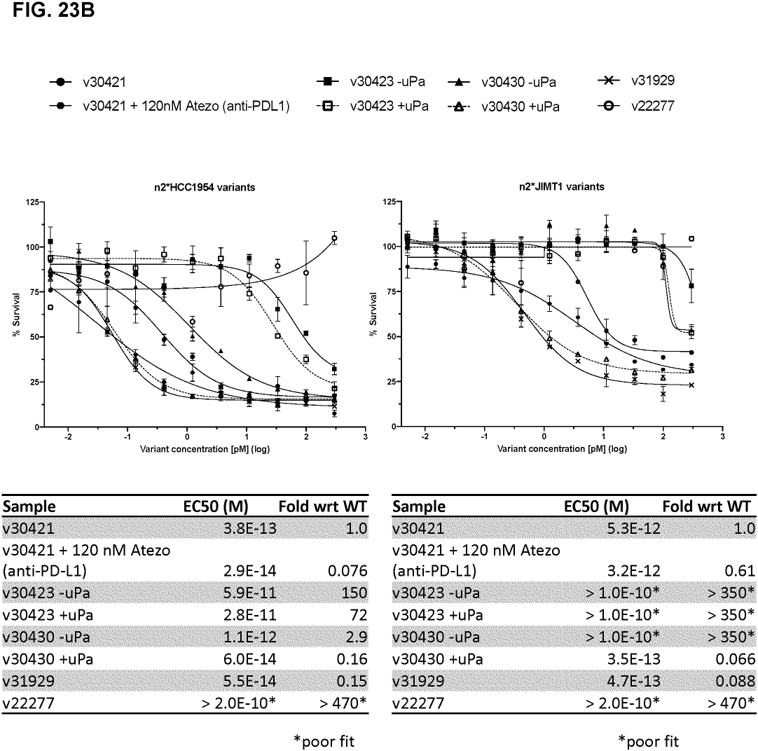

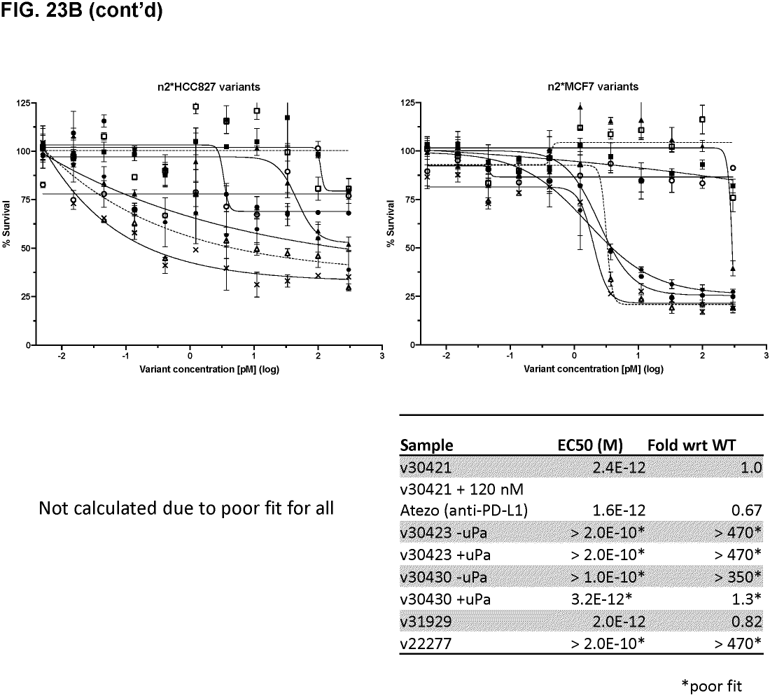

The TDCC using JIMT-1 as the Her2 and PD-L1 positive cell line was repeated and expanded to 3 other cell lines with differing levels of those receptors and using a different T-cell donor than in the previous experiment. Cytotoxicity data for two repeats is shown in FIG. 23. Levels of the cytokine IFNγ were also monitored as a proxy of immune activation of the T-cells for repeat n=1 (FIG. 24). The receptor numbers were determined for all cell lines used and are shown in FIG. 25. The potency of an unmasked control (30421) was determined to be between 0.03 pM (HCC1954: high Her2, high PD-L1) and 3 pM (MCF-7: medium Her2, low PD-L1) for cytotoxicity of the different cell lines. The potency of this unmasked control as determined by IFNγ release was between 8.4 pM (HCC1954: high Her2, high PD-L1) and 50 pM (HCC829: low Her2, medium PD-L1). Masking as measured by an increase in EC50 for uncleavable (30423) and cleavable (30430) masked variants was confirmed in all cell lines and ranged from 72 to >450 fold in the cytotoxicity readout and 8.2 to >350 fold in the IFNγ readout. A variant with only the PD-1 moiety attached to the heavy chain (31929), a cleavable masked variant after uPa treatment (30430 +uPa) as well as a combination of unmasked control and saturating amounts of an anti-PD-L1 antibody (30421 + 120 nM atezolizumab) showed higher potency (0.019 to 0.84 fold lower EC50 in cytotoxicity) compared to the unmasked control (30421) in cell lines with significant PD-L1 expression (HCC1954, JIMT-1, HCC827) due to their ability to also engage PD-L1. A cell line with very low PD-L1 expression (MCF-7) showed no significant differentiation in the cytotoxicity readout between unmasked control (30421) and those variants capable of engaging PD-L1 (31929, 30421 + 120 nM atezolizumab, 30430 +uPa). However, these variants with an anti-PD-L1 moiety did show a higher potency in IFNγ release for all tested cell lines compared to the unmasked control (30421). An irrelevant anti-RSV antibody (22277) showed no activity in the TDCC for either of the cell lines.

### Example 8 Pd1 and Pd-L1 Binding Analysis of Masked Anti-CD3 Variants

As a proxy for the biological activity of the PD-1 and PD-L1 moieties used as a masking domain, binding of the modified variants to CHO cells expressing PD-L1 and PD-1 was determined as follows.

### Methods

### Transfection of CHO Cells

CHO-S cells (National Research Council Canada) were cultured in FreeStyle CHO expression medium (Thermo Fisher Scientific, Waltham, MA) with 1% Fetal Bovine Serum (Thermo Fisher Scientific, Waltham, MA). Neon Transfection system (Thermo Fisher Scientific, Waltham, MA) was used to perform transfection. CHO-S cells were counted and washed 2x with PBS and once in Resuspension buffer R (Thermo Fisher Scientific, Waltham, MA) before being resuspended at 100 E6 cells/mL. PD-1, PDL-1 or GFP plasmid DNA (GenScript, Piscataway, NJ) was added at 1 ug/1 E6 cells. Neon tube was filled with 3 mL Electrolytic buffer E2 (Thermo Fisher Scientific, Waltham, MA). Using a 100 µL Neon tip (Thermo Fisher Scientific, Waltham, MA), transfection for each plasmid was carried out at the following settings: Voltage - 1620, Width -10, Pulse - 3. Transfected cells were transferred to a pre-warmed flask at a concentration of 1 E6 cells/mL for each condition.

### PD1/PDL1 Binding by Flow Cytometry

Variants purified in Example 2 and uPa treated in Example 5 were titrated directly in a v-bottom 96-well plate (VWR, Radnor, PA, USA) from 200 nM at a 1:3 dilution. CHO-PD1, CHO-PDL-1, and CHO-GFP cells were thawed and washed in RPMI 1640 medium (A1049101, ATCC modification) (Thermo Fisher Scientific, Waltham, MA, USA) supplemented with 10% Fetal Bovine Serum (Thermo Fisher Scientific, Waltham, MA, USA) and resuspended in FACS buffer (PBS + 2% FBS). Each of the CHO-PD1 and CHO-PDL-1 cells were combined 2:1 with CHO-GFP cells and 20 uL of cell suspension was added to the plate with the titrated variants. The cells were incubated with the variants at 4° C. for 1 h. Following incubation, the cells were washed 2x with FACS buffer and 1 ug/mL of secondary antibody AF647 Goat anti-human IgG Fc (Jackson ImmunoResearch, West Grove, PA, USA) along with 1000-fold diluted viability dye (Biolegend, San Diego, CA, USA) was added to the wells. Plate was incubated at room temperature for 30 min. Cells were washed 2x in FACS buffer and resuspended in 50 uL of FACS buffer.

For the assay read-out, Geometric mean of APC fluorescence was measured by flow cytometry on a BD LSRFortessa (BD Life Sciences, Gurugram, India). Non-specific binding was determined by measuring APC fluorescence Geometric mean of GFP positive cells. Graphs were generated using GraphPad Prism version 8.1.2 for Mac OS X (GraphPad Software, La Jolla, CA, USA).

### Results

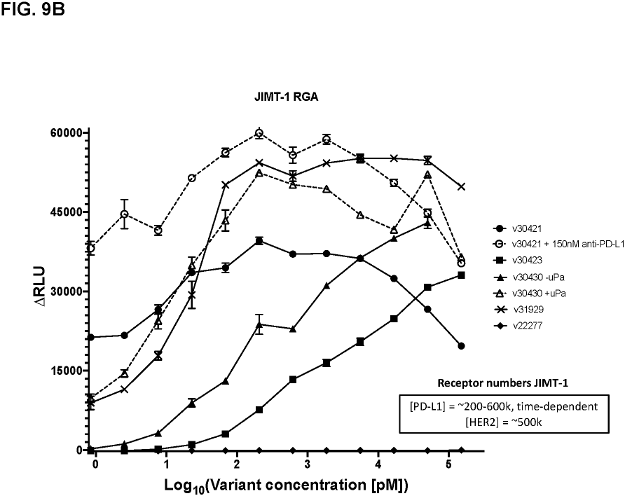

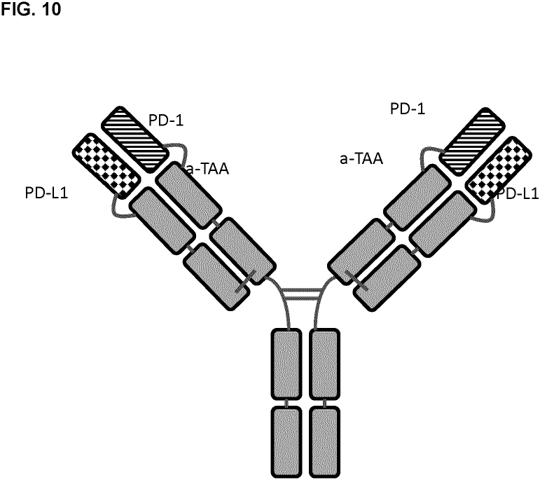

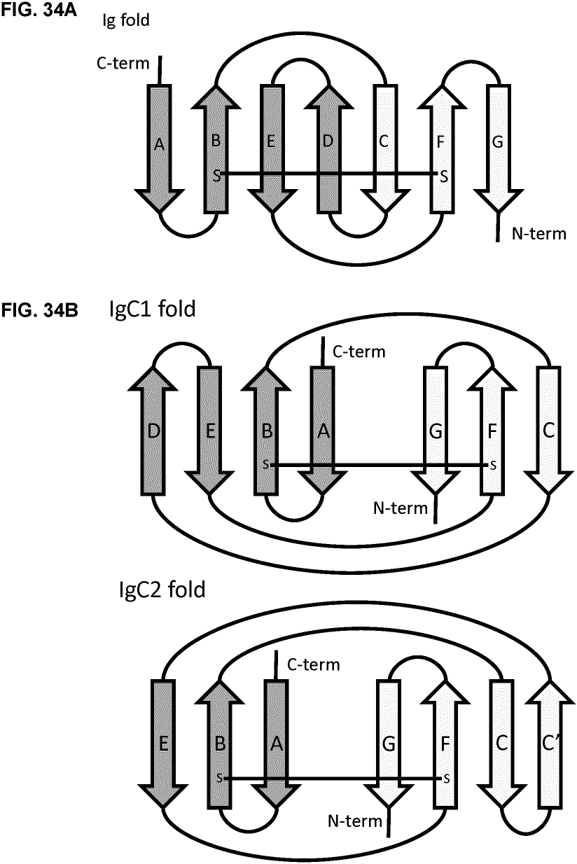

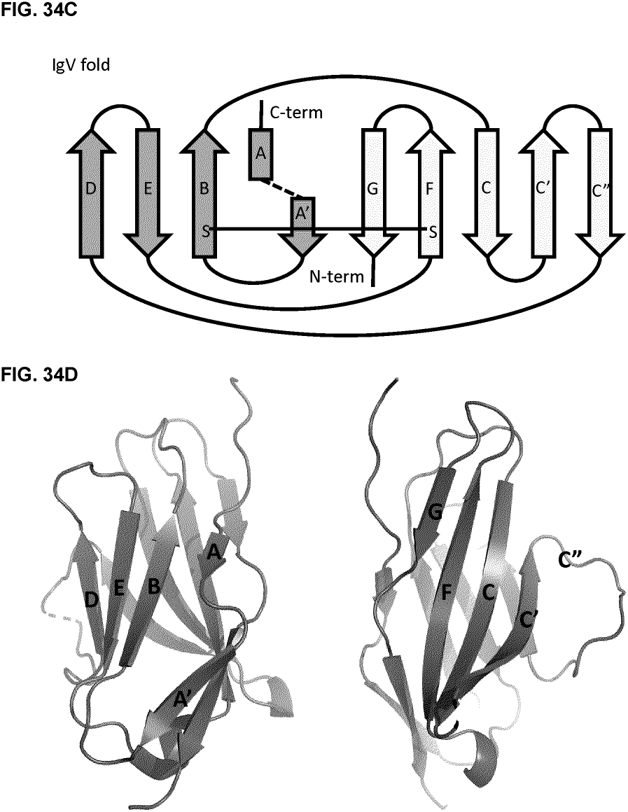

As shown in FIG. 8, binding to PD-L1 (A) or PD-1 (B) was not observed for masked variants (30423, 30426, 30430, 30436) without uPa treatment (-uPa). Variants with only an affinity matured PD-1 or PD-L1 moiety attached to either heavy or light chain showed binding with an IC50 of 0.3 nM and 6 nM, respectively. The uncleavable variants (30423, 30426), did not bind to PD-L1 or PD-1 when treated with protease (+uPa) whereas partial binding was recovered for the uPa-treated samples that contain a uPa cleavage sequence between the Fab and PD-1:PD-L1 mask. Specifically, binding to PD-L1 was partially recovered for 30430, within 53 fold of the relevant one-sided mask control 31929 (A). Binding to PD-1 was partially recovered for 30436, within 12 fold of the one sided mask control 31931 (B). This is consistent with the identity of the immune modulator that was designed to be left on these variants when cleaved by the protease (PD-1 on 30430, PD-L1 on 30436). A variant without a PD-1:PD-L1 based mask (30421) and an irrelevant control (22277) showed no binding to PD-L1 or PD-1 as expected. In a separate experiment using JIMT-1 as target cells, a variant with a non-functional PD-1 domain appended to the heavy chain (32497) showed a reduction in TDCC potency (55-fold EC₅₀, FIG. 33) compared to an unmasked control (v30421), while the equivalent variant with a functional PD-1 (31929) showed increased TDCC potency (0.2 fold EC₅₀) compared to the unmasked control (v30421) that was seen before.

### Example 9 Investigation of Added Functionality of Pd-1 Mask in Hybrid PD-1/PD-L1 Reporter Gene Assay

To investigate blocking of the PD-1:PD-L1 checkpoint engagement by the PD-1 moiety of the mask in addition to the T-cell engagement function of the variants, a custom hybrid PD-1/PD-L1 Reporter Gene Assay (RGA) was performed as follows.

### Methods

JIMT-1 (Leibniz Institute, Braunschweig, Germany) cultured in growth medium consisting of DMEM medium (Thermo Fisher Scientific, Waltham, MA) supplemented with 10% Fetal Bovine Serum (Thermo Fisher Scientific, Waltham, MA), HCC1954 (ATCC, Manassas, VA) and HCC827 (ATCC, Manassas, VA) cultured in growth medium consisting of RPMI-1640 ATCC modification (Thermo Fisher Scientific, Waltham, MA) supplemented with 10% Fetal Bovine Serum, MCF-7 (ATCC, Manassas, VA) cultured in growth medium consisting of MEM medium (Thermo Fisher Scientific, Waltham, MA) supplemented with 10% Fetal Bovine Serum and 0.01 mg/mL of human recombinant Insulin (Thermo Fisher Scientific, Waltham, MA) and Jurkat T cells stably expressing human PD-1 and NFAT-induced luciferase (PD-1/PD-L1 Blockade Bioassay Promega Cat# J1250, Madison, WI) cultured in RPMI-1640 medium ATCC modification supplemented with 10% Fetal Bovine serum were maintained in T-75 or T-175 flasks (Corning, Corning, NY) in an incubator at 37° C. with 5% carbon dioxide prior to assay set-up. On the day of the experiment, the variants were titrated in triplicate at 1:3 dilution directly into 384-well Low Flange White Flat Bottom Polystyrene TC-treated Microplates, (Corning Cat# 3570, Corning, NY) from 150 nM to 0.85 pM in 20 uL total volume per well. Tumor cells were dissociated using cell dissociation buffer and mixed with Jurkat cells at a 1:1 ratio in RPMI 1640 supplemented with 1% Fetal Bovine serum. 20 uL of the mixed cell suspension was added to the plate containing the titrated variants. The plates were incubated for 16 h at 37° C. with 5% carbon dioxide. Post incubation, 40 uLof Bio-Glo™ Luciferase Assay reagent (Promega Cat# G7940, Madison, WI) was added to all wells ensuring no bubbles were formed and the plate was read after 10 min in Luminesence mode on the microplate reader (Biotek Synergy H1, Winooski, VT) with a gain of 150. A schematic of the setup of the assay is shown in FIG. 9A.

### Results

The analysis of the custom RGA to interrogate added functionality by the mask is shown FIG. 9B. When cells were treated with an unmasked variant capable of crosslinking T-cells and tumor cells (30421) in combination with a saturating amount (150 nM) of an anti-PD-L1 antibody, a high RGA response was seen. While the unmasked bispecific CD3 × Her2 antibody could productively cross-link T-cells and tumor cells, high concentrations of the anti-PD-L1 antibody robustly blocked the PD-1:PD-L1 checkpoint engagement, leading to a high signal across all tested variant concentrations. Conversely, when treated with only an unmasked variant (30421), the signal was significantly reduced due to the engagement of PD-1 and PD-L1 between modified T-cell and JIMT-1 cells. Uncleavable (30423) and cleavable (30430) masked variants not treated with uPa (-uPa) show significantly reduced activity below 10 nM variant concentration when compared to the unmasked 30421, pointing to a productive inhibition of the T-cell engager functionality by steric blocking of the CD3 paratope. A uPa-untreated sample of 30430 was more potent in eliciting an RGA response than 30423. When treated with uPa (+uPa), a cleavable masked variant (30430) showed activity in the RGA that was higher than that of the unmasked control (30421) at variant concentrations above 100 pM, pointing to an unmasking of the CD3 paratope as well as a blocking of the PD-1:PD-L1 checkpoint engagement by the functional PD-1 moiety of the mask left on the variant after cleavage. In line with this finding, a control with just the PD-1 domain attached to the heavy chain of the CD3 Fab showed a similar profile and increased activity in the RGA at variant concentrations higher than 100 pM when compared to the unmasked control (30421). An irrelevant anti-RSV antibody (22277) showed no activity in the RGA.

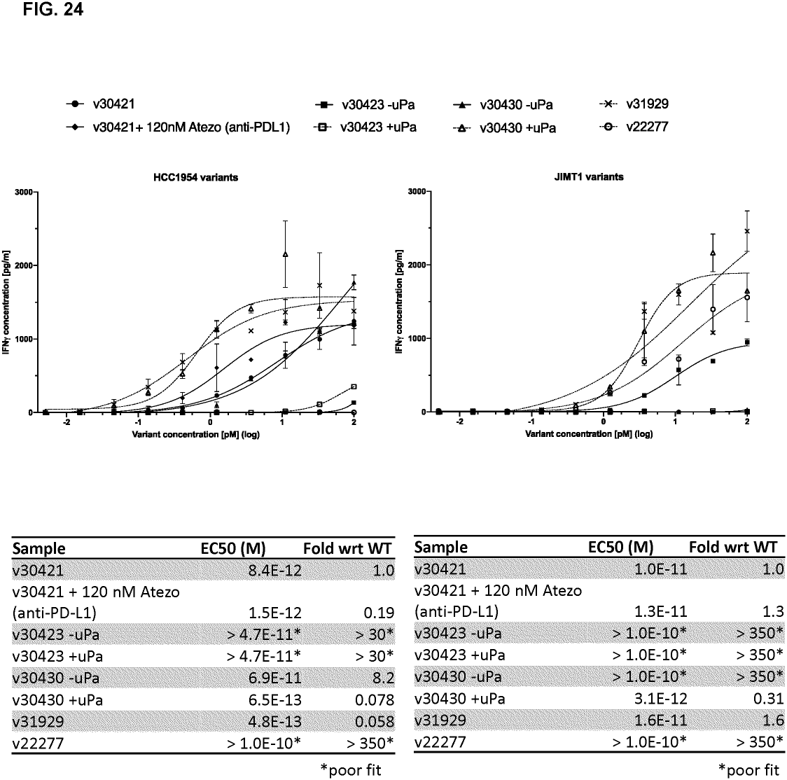

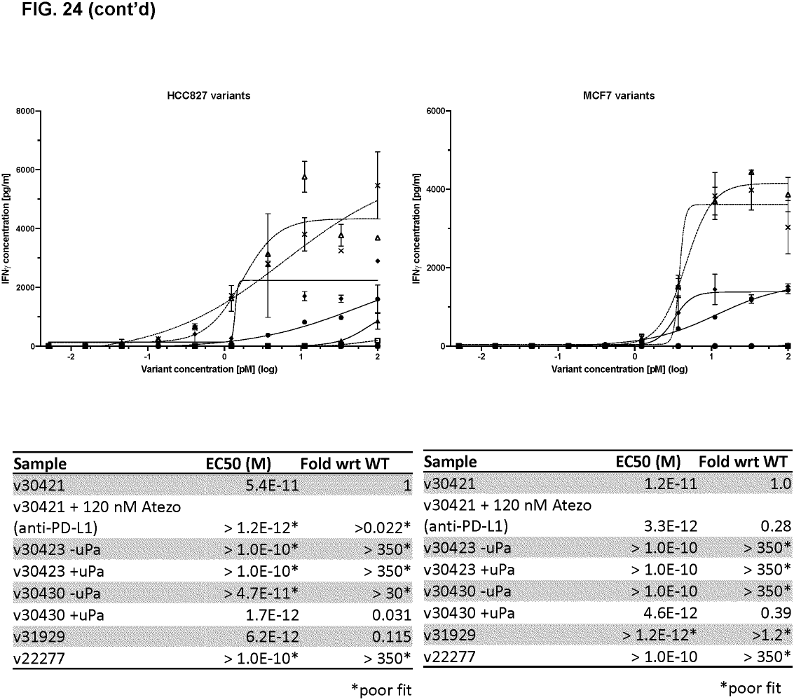

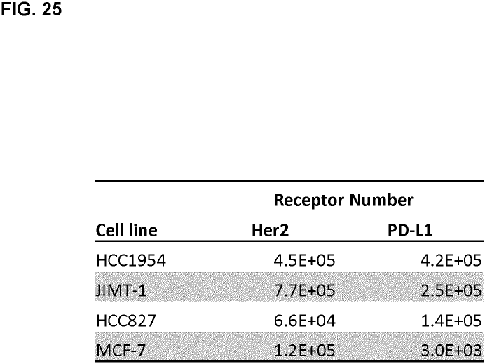

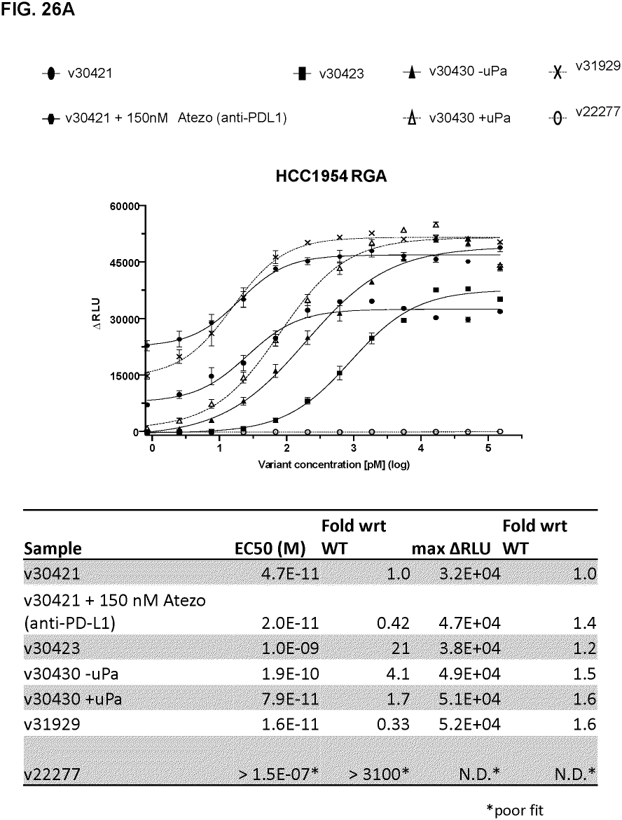

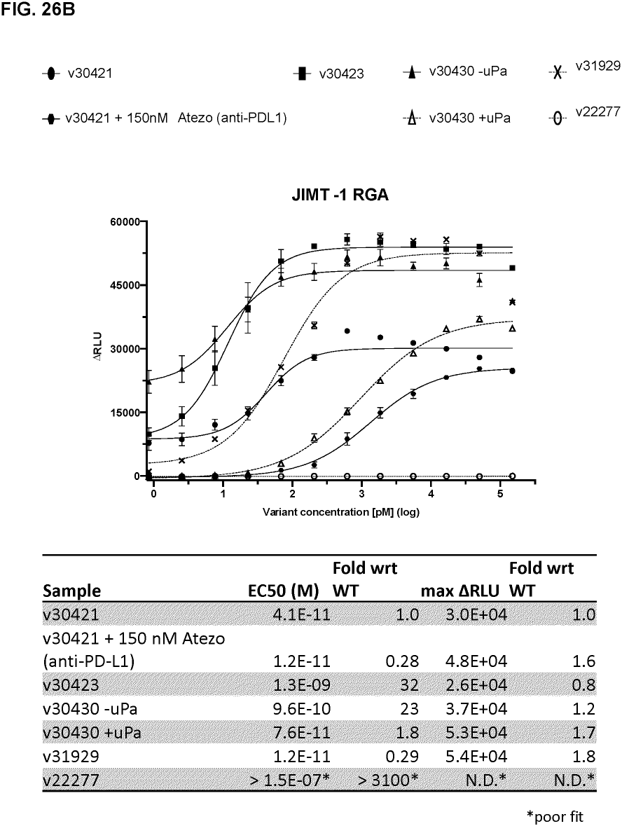

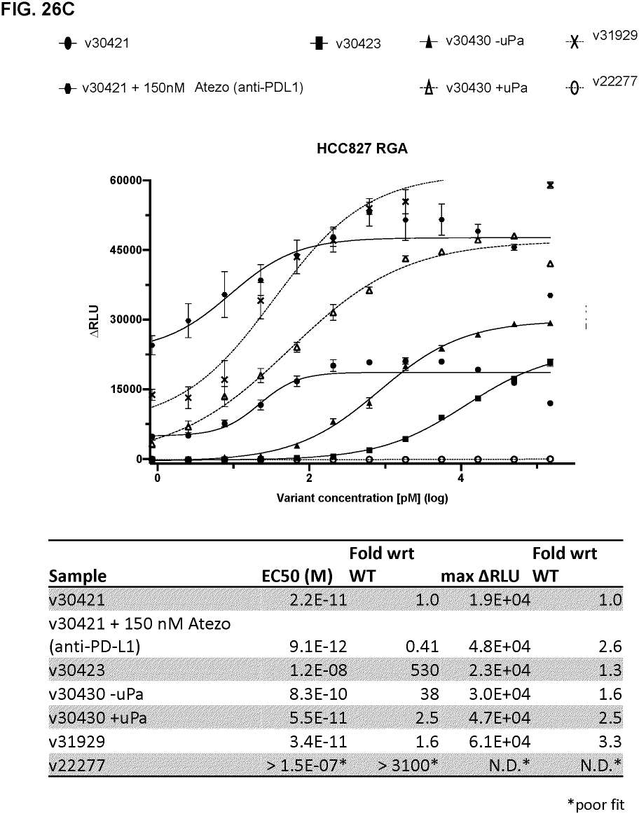

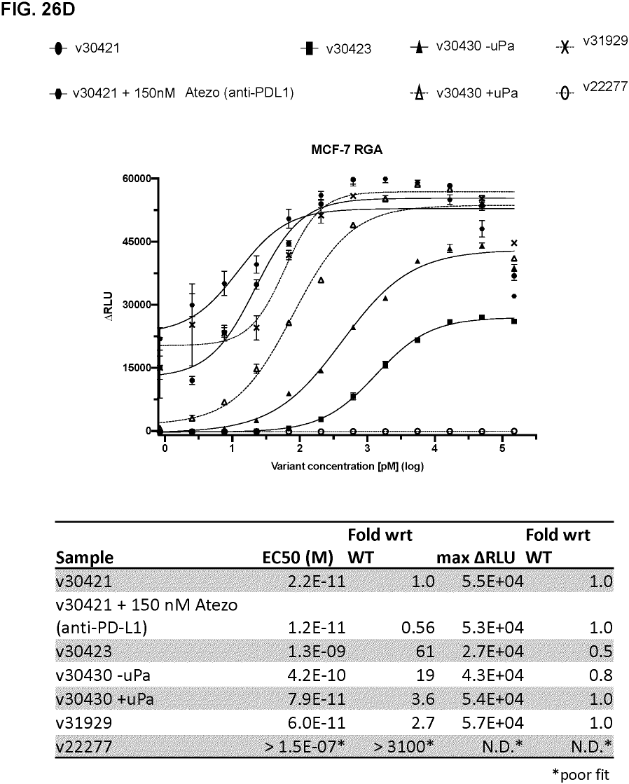

The RGA using JIMT-1 as the Her2 and PD-L1 positive cell line was repeated and expanded to 3 other cell lines with differing levels of those receptors, as determined in Example 7. Data for the RGA performed here is shown in FIG. 26. Masking as measured by an increase in EC50 for uncleavable (30423) and cleavable (30430) masked variants was confirmed in all cell lines and ranged from 4 to 530-fold compared to an unmasked control (30421). While the potency of that unmasked control (30421) was comparable between for cell lines (EC50 = 20-50 pM), variants tested on cell lines with lower Her2 and/or PD-L1 receptor numbers (HCC827 and MCF-7) showed stronger masking than on those with higher receptor expression (HCC1954 and JIMT-1). Upon treatment with uPa (+uPa) a cleavable masked variant (30430) recovered potency within 1.7 to 3.6-fold of the unmasked control. A variant with only the PD-1 moiety attached to the heavy chain (31929), a cleavable masked variant after uPa treatment (30430 +uPa) as well as a combination of unmasked control and saturating amounts of an anti-PD-L1 antibody (30421 + 120 nM atezolizumab) showed higher efficacy (1.6 to 3.3-fold higher maximum RLU) in cell lines with significant PD-L1 expression (HCC1954, JIMT-1, HCC827) due to their ability to also engage PD-L1. In cell lines with high TAA and PD-L1 expression (HCC1954, JIMT-1) a higher potency was also seen for these variants (0.2 to 0.4-fold in EC50). A cell line with very low PD-L1 expression (MCF-7) showed no differentiation of unmasked control (30421) and those capable of engaging PD-L1 (31929, 30421 + 120 nM atezolizumab, 30430 +uPa). An irrelevant anti-RSV antibody (22277) showed no activity in the RGA for either of the cell lines.

### Example 10 Preparation of Masked Anti-EGFR, Anti-Mesothelin, Anti-TF, Anti-CD19, Anti-CMET and Anti-CDH3 Variants

To investigate applicability of the masking technology to antibodies targeting different antigens, variable domains of mAbs targeted against several different epitopes were appended with a masking domain comprising a PD-1:PD-L1 complex. The fusion protein constructs were designed as follows.

### Methods

Protein sequences of WT and modified IgV domains of human PD-1 and PD-L1 were fused via non-cleavable and uPa-cleavable linkers to the N-termini of the IgG1 heavy chain and kappa light chain (VL-CL), respectively, of antibodies targeted against several different epitopes (EGFR, Mesothelin, TF, CD19, cMet, CDH3) as described in Example 1. Sequences of VL and VH and their sources are described in Table 2. The notable difference to the constructs in Example 1 is the use of a wild type (WT) CH3 (SEQ ID 12), allowing for the assembly of homodimeric, full sized antibodies. A schematic of the construct design for the masked Fab as well as the intended mechanism of action (MoA) is shown in FIG. 1. A schematic of the final design, a bivalent, fully masked mAb with two identical heavy and light chains, is shown in FIG. 10. The used sequences of the final variants are listed in Table B.

TABLE B

Sequences of paratopes investigated for compatibility with mask

| Epitope targeted | Reference | Ref. SEQ ID VL | Ref. SEQ ID VH |
|---|---|---|---|
| EGFR | US6217866B1 | 13 | 14 |
| Mesothelin | Bauss, F. et al. Characterization of a re-engineered, mesothelin-targeted Pseudomonas exotoxin fusion protein for lung cancer therapy.Mol Oncol 10, 1317-1329, doi:10.1016/j.molonc.2016.07.003 (2016). | 15 | 16 |
| TF | Presta, L. et al. Generation of a humanized, high affinity anti-tissue factor antibody for use as a novel antithrombotic therapeutic. Thromb Haemost 85, 379-389 (2001). | 17 | 18 |
| CD19 | Gerber, H. P. et al. Potent antitumor activity of the anti-CD19 auristatin antibody drug conjugate hBU12-vcMMAE against rituximab-sensitive and -resistant lymphomas. Blood 113, 4352-4361, doi: 10.1182/blood-2008-09-179143 (2009). | 19 | 20 |
| cMet | US8741290 | 21 | 22 |
| CDH3 | Zhang, C. C. et al. PF-03732010: a fully human monoclonal antibody against P-cadherin with antitumor and antimetastatic activity. Clin Cancer Res16, 5177-5188, doi:10.1158/1078-0432.CCR-10-1343 (2010). | 23 | 24 |

TABLE C

Sequence composition of tested variants

| Variant No | Cartoon | Description | Clone H1 | Clone L1 | Clone H2 |
|---|---|---|---|---|---|
| EGFR | | | | | |
| 32474 | 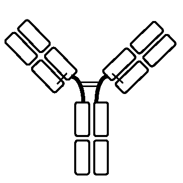 | Unmasked aEGFR mAb | 23567 | 3232 | |
| 16427 | 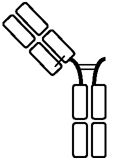 | Unmasked aEGFR OAA | 10606 | 3357 | 1380 |
| 31722 | 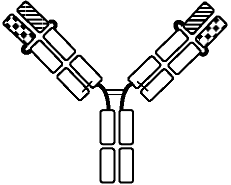 | HA PD-1:WT PD-L1 masked aEGFR Mab, uncleavable | 23246 | 23247 | |
| 31723 | 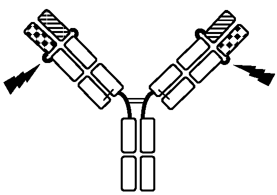 | HA PD-1:WT PD-L1 masked aEGFR Mab, PD-L1 cleavable | 23246 | 23248 | |

| | | | | | |
|---|---|---|---|---|---|
| MSLN | | | | | |
| 16417 | 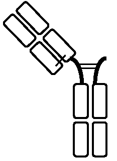 | Unmasked aMSLN OAA | 10564 | 10565 | 1380 |
| 31728 | 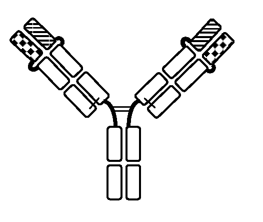 | HA PD-1:WT PD-L1 masked aMSLN Mab, uncleavable | 23253 | 23254 | |
| 31729 | 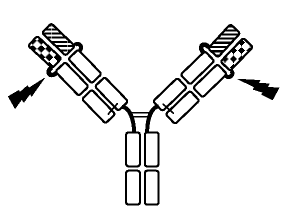 | HA PD-1:WT PD-L1 masked aMSLN Mab, PD-L1 cleavable | 23253 | 23256 | |

TF

| | | | | |
|---|---|---|---|---|
| 6323 | 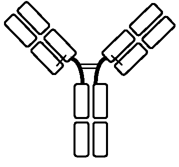 | Unmasked aTF mAb | 2932 | 787 |
| 31736 | 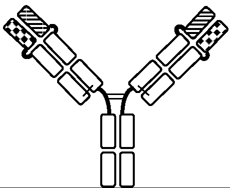 | HA PD-1:WT PD-L1 masked aTF Mab, uncleavable | 23261 | 23262 |
| 31737 | 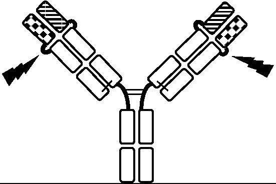 | HA PD-1:WT PD-L1 masked aTF Mab, PD-L1 cleavable | 23261 | 23264 |

| | | | | | |
|---|---|---|---|---|---|
| CD19 | | | | | |
| 4372 | 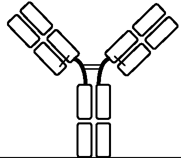 | Unmasked aCD19 mAb | 3344 | 3346 | 3345 |

| | | | | |
|---|---|---|---|---|
| 31732 | 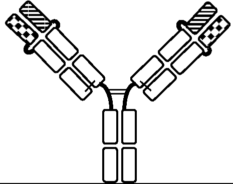 | HA PD-1:WT PD-L1 masked aCD19 Mab, uncleavable | 23257 | 23258 |
| 31733 | 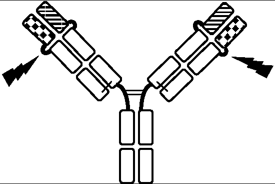 | HA PD-1:WT PD-L1 masked aCD19 Mab, PD-L1 cleavable | 23257 | 23260 |

cMET

| | | | | |
|---|---|---|---|---|
| 17606 | 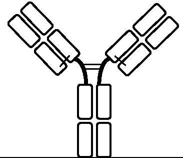 | Unmasked acMet mAb | 11509 | 11462 |
| 28647 | 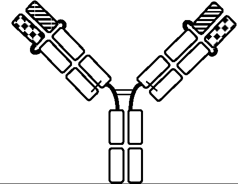 | HA PD-1:WT PD-L1 masked acMet Mab, uncleavable | 20859 | 20855 |

CDH3

| | | | | |
|---|---|---|---|---|
| 17214 | 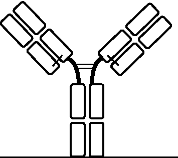 | Unmasked aCDH3 mAb | 11274 | 10567 |
| 28662 | 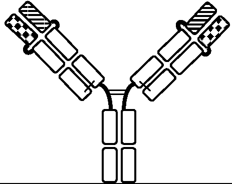 | HA PD-1:WT PD-L1 masked aCDH3 Mab, uncleavable | 20875 | 20871 |

### Example 11 Production of Masked Anti-EGFR, Anti-Mesothelin, Anti-TF, Anti-CD19, Anti-CMET and Anti-CDH3 Variants

Sequences of modified variants designed in Example 10 were cloned into expression vectors and expressed and purified as follows.

### Methods

Heavy and light chain sequences of the modified variants targeted against several different epitopes (EGFR, MSLN, TF, CD19, cMet, CDH3) from Example 10 were transfected into Expi293F™ cells in an equal molar ratio and otherwise expressed and purified as described in Example 2.

### Results

Preparative SEC as described in Example 2 was used in order to obtain samples of high purity. Yields after preparative SEC ranged from 1.5 - 6 mg per variant. Sample purity was assessed as described in Example 12.

### Example 12 Quality Assessment of Masked Anti-EGFR, Anti-Mesothelin, Anti-TF, Anti-CD19, Anti-CMET and Anti-CDH3 Variants

Purified samples from Example 10 were assessed for purity and sample homogeneity by UPLC-SEC and non-reducing SDS-PAGE as described below.

### Methods

For the non-reducing SDS-PAGE, 2 µL of sample was diluted with 10 µL PBS and then mixed with 4 µL of 4X Laemmli buffer (BioRad, Hercules, CA). Samples were then heated for 5 min at 95° C. and run on mini-PROTEAN 4-20% Precast Gels (BioRad, Hercules, CA) in the provided Tris/Glycine/SDS buffer before being stained with Coomassie G-250, destained and imaged. UPLC-SEC and non-reducing and reducing CE-SDS were performed as described in Example 3.

### Results

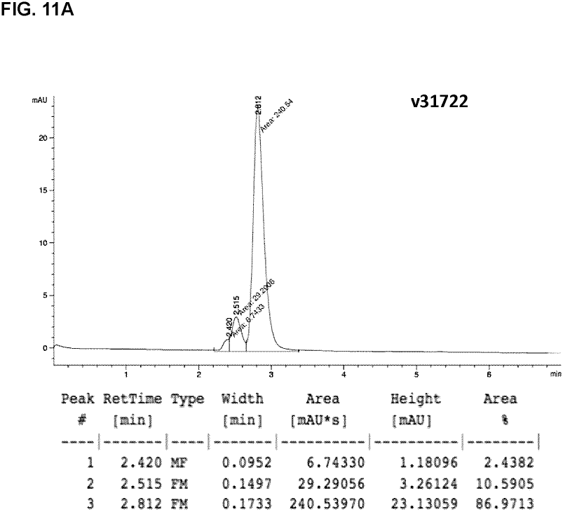

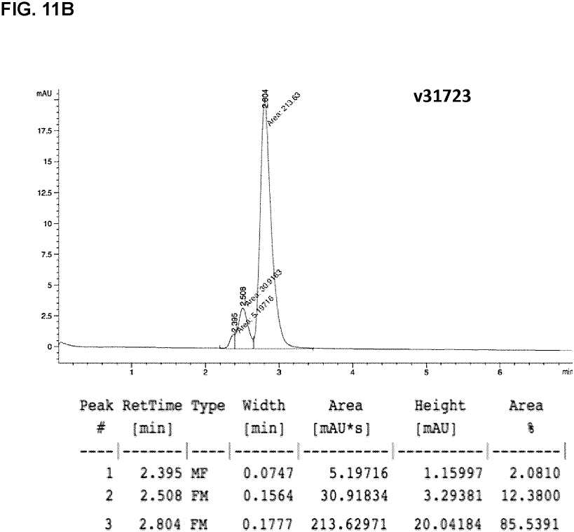

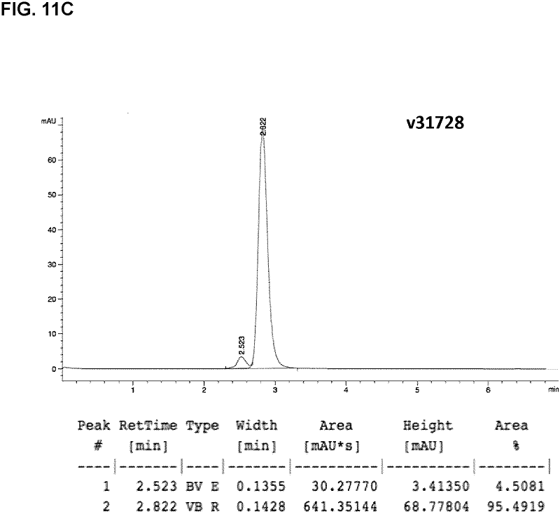

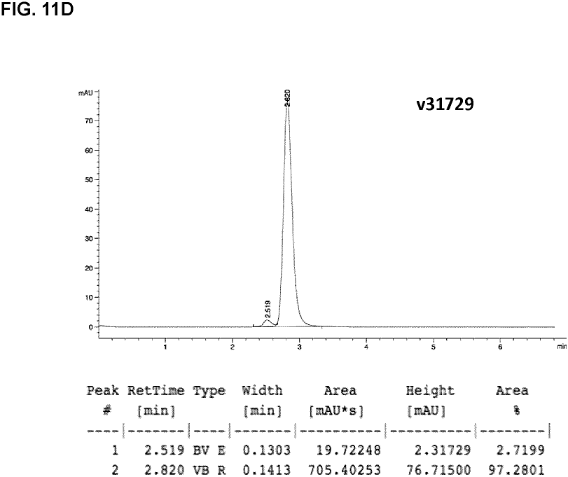

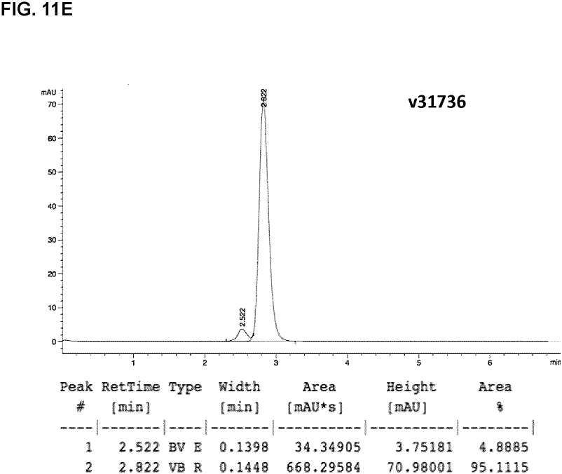

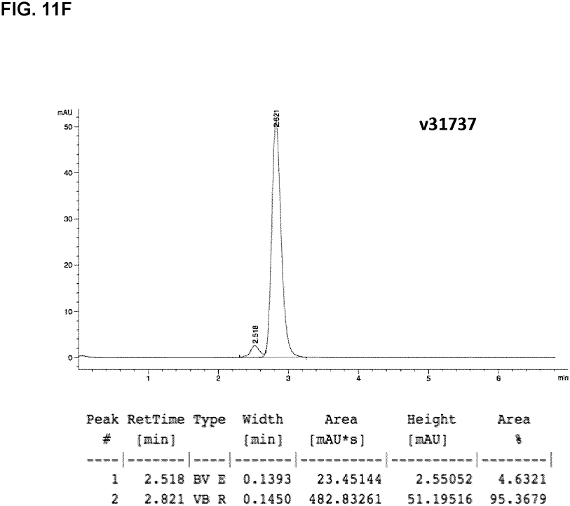

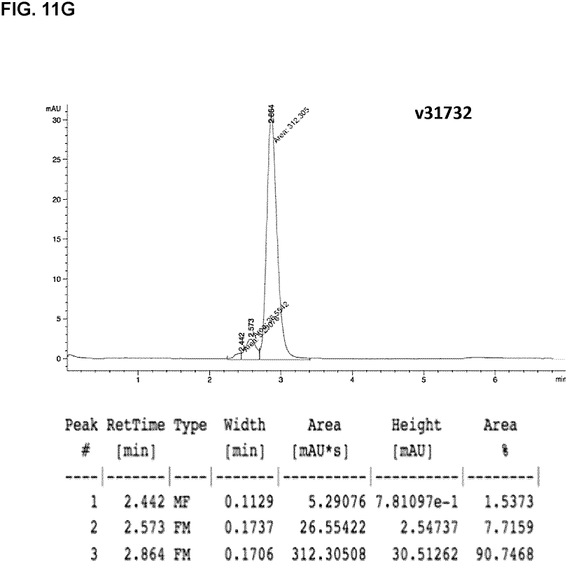

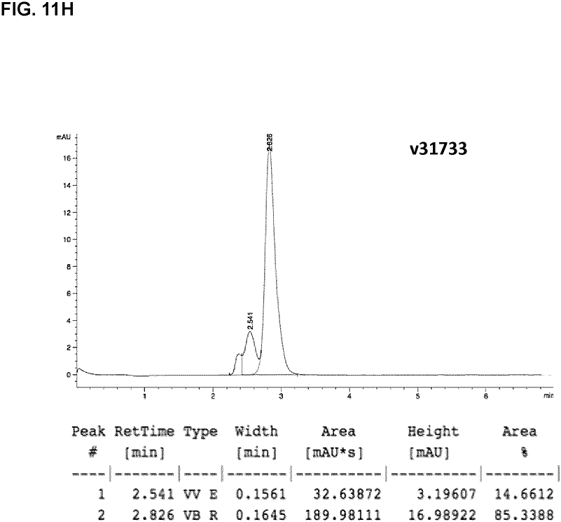

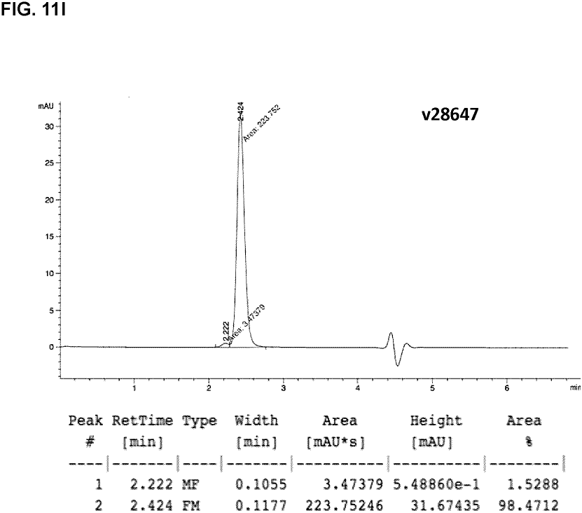

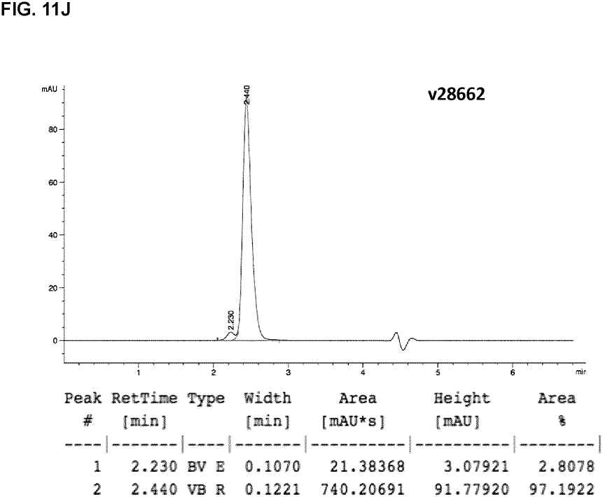

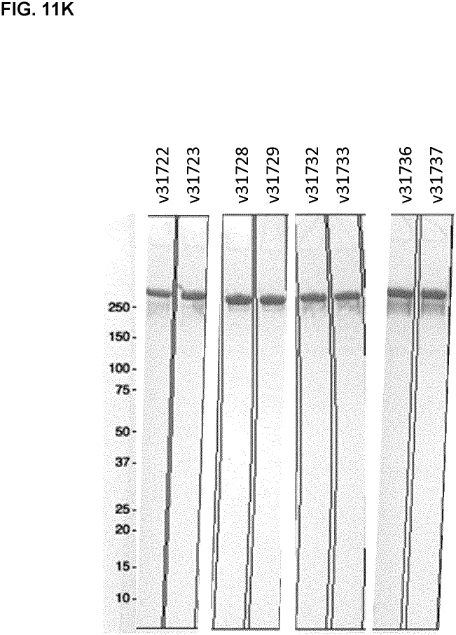

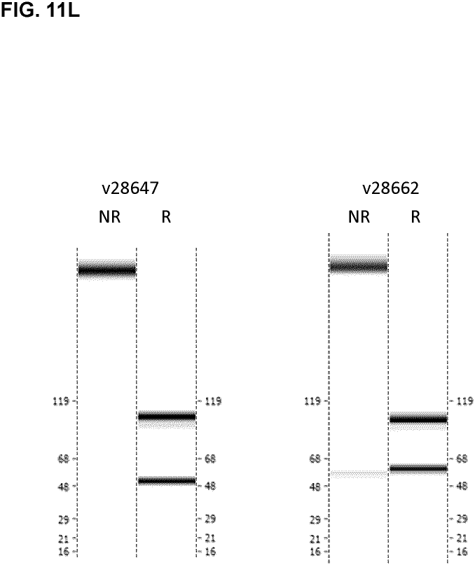

UPLC-SEC traces of samples after preparative SEC purification in FIGS. 11A-J showed homogeneous samples that contained 85% - 98% of correct species. The presence of a small peak at a low retention time compared to the main species indicates the presence of small amounts of high molecular weight species such as oligomers and aggregates in all samples. These high molecular weight species were more prevalent for the CD19 and EGFR-targeted samples as compared to the ones targeted to MSLN, TF, c-Met and CDH3.

Analysis of non-reducing SDS-PAGE and CE-SDS (FIGS. 11K,L) showed a single predominant species for all variants. Notably, the apparent molecular weight of this species is significantly higher than what would be expected (> 250 kDa vs 200 kDa). Reducing CE-SDS of representative variants targeted against c-Met and CDH3 showed only bands corresponding to the intact heavy and light chains. These show the same high apparent molecular weight as described in Example 3. Glycosylation of both the PD1 and PD-L1 moieties in the designs is likely causing the increase in apparent molecular weight (Tan, S. et al. An unexpected N-terminal loop in PD-1 dominates binding by nivolumab. Nat Commun 8, 14369, doi:10.1038/ncomms14369 (2017), Li, C. W. et al. Glycosylation and stabilization of programmed death ligand-1 suppresses T-cell activity. Nat Commun 7, 12632, doi:10.1038/ncomms12632 (2016)).

### Example 13uPa Cleavage of Masked Anti-EGFR, Anti-Mesothelin, Anti-TF, Anti-CD19, Anti-CMET and Anti-CDH3 Variants

In order to assess release of part of or all of the mask from Fabs of several different paratopes by cleavage of the intended protease sites in the linkers, select samples produced in Example 11 were treated with uPa in vitro. Reactions were monitored by reducing SDS-PAGE as follows.

### Methods

Preparative cleavage assays of the modified variants targeting different epitopes were set up as described in Example 5 and analyzed by non-reducing SDS-PAGE. The SDS-PAGE was set up as described in Example 12 with the exception of the usage of reducing Laemmli buffer for the denaturation of the sample. The reducing buffer was obtained by supplementing 4X Laemmli buffer with 10% β-ME.

### Results

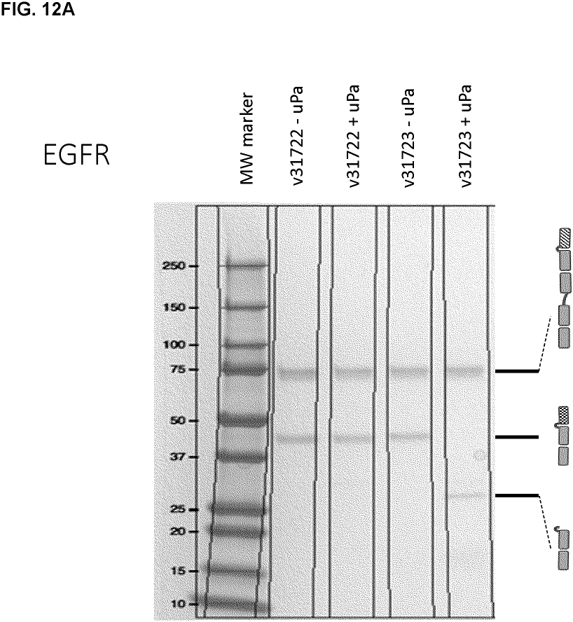

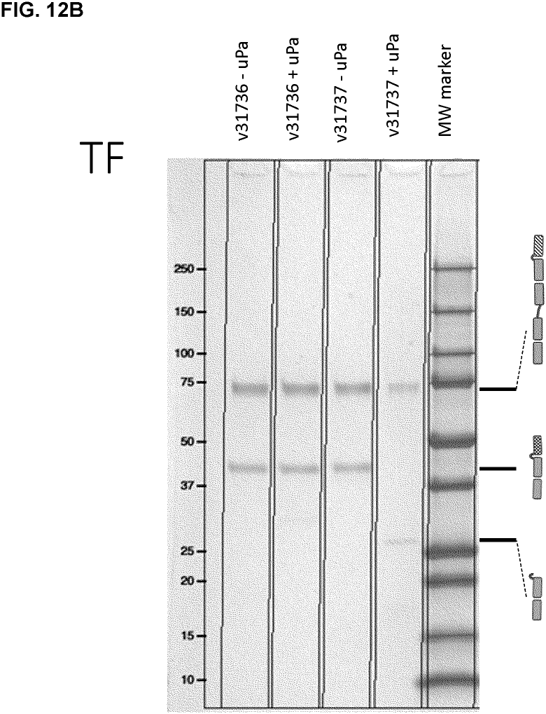

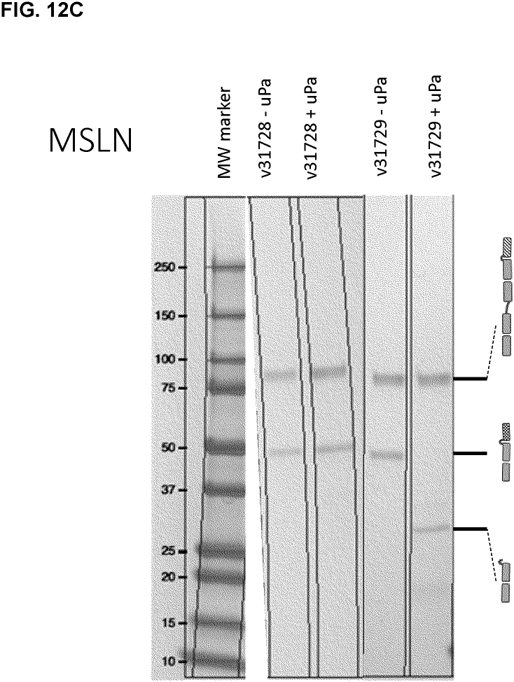

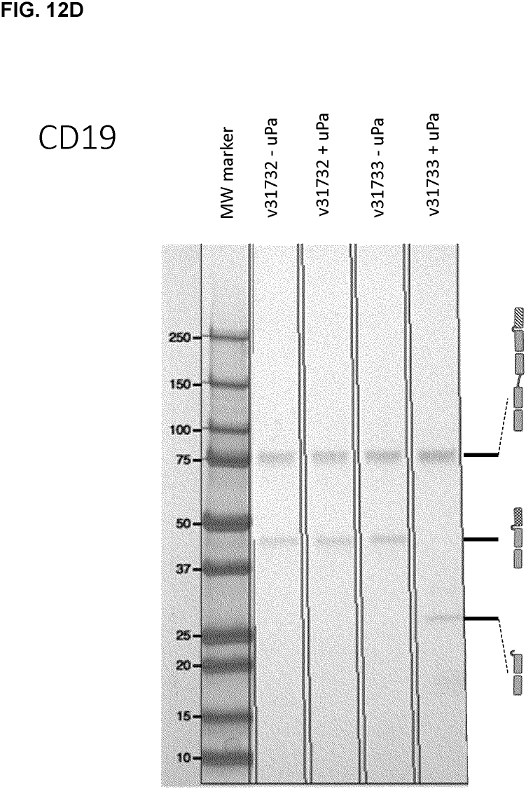

While variants not containing a uPa cleavage sequence did not show any processing under the conditions tested, all variants that did include a uPa-specific sequence between PD-L1 moiety and the VL of the Fab showed complete cleavage (FIG. 12) and a release of the PD-L1 domain from the light chain. This could be seen by a decrease in the apparent MW of the LC to ~ 25 kDa after uPa treatment as expected for an unprotected kappa light chain. Likely due to heterogenous glycosylation (Li, C. W. et al. Glycosylation and stabilization of programmed death ligand-1 suppresses T-cell activity. Nat Commun 7, 12632, doi:10.1038/ncomms12632 (2016)) and the small molecular weight (~ 13 kDa), the free PD-L1 moiety was not detected for variants targeted for EGFR and TF. A faint band indicating a species of an apparent molecular weight of 15 - 20 kDa was detected for MSLN and CD19.

### Example 14 Masking/Unmasking of Anti-EGFR, Anti-Mesothelin, Anti-TF, Anti-CD19, Anti-CMET and Anti-CDH3 Variants

Target binding of the different paratope/epitope pairs was assessed by SPR and flow cytometry on the samples produced in Example 11 and treated by uPa in Example 13 as follows.

### Methods

### Native Binding by Flow Cytometry

Various cancer cell lines expressing the surface proteins containing the of interest (MDA-MB231, OVCAR3, MDA-MB468, Raji) were maintained in their recommended culture medium, supplemented with L-glutamine and the appropriate concentration of serum (complete medium) in a humidified + 5% CO2 incubator at 37° C.

Modified variants targeted against the different epitopes were diluted 2X in complete medium, followed by three-fold serial dilutions in cold complete medium for a total of eight to ten concentration points starting at 300 nM or 150 nM.

All media were kept are 4° C. and all incubations were performed on wet ice. On the day of the assay, exponentially growing cells were harvested using warm non-enzymatic cell dissociation solution, centrifuged and resuspended in complete medium at a cell density of 2E+06 cells/mL. 50 µL/well of cells were distributed in a polypropylene v-bottom 96 well plate (Corning, Corning, NY, USA). Equal volumes of 2X test antibodies or controls were added to cells and incubated for 2 hours. Cells were then washed twice by centrifugation and the supernatants removed. Detection of bound variants was achieved by an additional incubation with a fluorescently labeled, Fc-specific secondary antibody (Jackson ImmunoResearch, West Grove, PA, USA) for an hour. Cells were washed twice by centrifugation and cell pellets were resuspended in complete medium with Propidium Iodide (Invitrogen, Carlsbad, CA, USA), filtered using a 0.60 µm size-pore 96 well filter plate (MilliporeSigma, Burlington, MA, USA) and analyzed by flow cytometry using the HTS automated sampler unit (installed on BD-LSRII or BD-LSRFortessa). Two thousands alive/single-cell events were acquired per sample.

Specific MFI was calculated for each sample point by subtracting the MFI value of a negative control (background). Binding curves (specific MFI versus linear or log antibody concentration) were fitted with GraphPad Prism 8 (GraphPad Software, La Jolla, CA, USA) using a One-site specific with Hill slope four-parameter nonlinear regression curve fit model to determine Bmax and apparent Kd values for each test article.

### SPR

SPR (surface plasmon resonance) binding assays for determining kinetics and affinities of a subset of the different antigens (EGFR, TF, Mesothelin) to the modified mAb variants were carried out on Biacore™ T200 instrument (GE Healthcare, Mississauga, ON, Canada) with PBS-T (PBS + 0.05% (v/v) Tween 20, pH 7.4) running buffer at a temperature of 25° C. CM5 Series S sensor chip, Biacore amine coupling kit (NHS, EDC and 1 M ethanolamine) and 10 mM sodium acetate buffers were all purchased from GE Healthcare. PBS running buffer with 0.05% (v/v) Tween20 (PBS-T) was purchased from Teknova Inc. (Hollister, CA). Goat polyclonal anti-human Fc antibody was purchased from Jackson Immuno Research Laboratories Inc. (West Grove, PA). Recombinant protein of the extracellular domain of human EGFR (Genscript, Cat# Z03194-50) and mature human mesothelin (R&D systems, Cat# 3265-MS-050) were purchased and purified by SEC prior to SPR analysis to ensure purity and homogeneity of the analytes. Recombinant protein of human TF was expressed in HEK293 cells and purified by Anion Exchange (Q Sepharose HP, GE Healthcare) followed by SEC purification prior to usage in SPR.

The screening of mAb variants for binding to the different antigens occurred in two steps: an indirect capture of mAb variants onto the anti-human Fc-specific polyclonal antibody surface, followed by injection of five concentrations of SEC-purified antigen. The anti-human Fc surface was prepared on a CM5 Series S sensor chip by standard amine coupling methods as described by the manufacturer (GE Healthcare). Briefly, immediately after EDC/NHS activation, a 25 µg/mL solution of anti-human Fc in 10 mM NaOAc, pH 4.5 was injected at a flow rate of 10 µL/min for 7 min until approximately 4500 resonance units (RUs) were immobilized on all four flow cells. The remaining active groups were quenched by an injection of 1 M ethanolamine at 10 uL/min for 7 min. MAbs for analysis were indirectly captured onto the anti-Fc surfaces (flow cells 2 - 4) by injecting 2-20 µg/mL solutions at a flow rate of 10 µL/min for 60 s, resulting in mAb capture levels ranging from 130 - 470 RUs depending on the mAb variant. Using single-cycle kinetics, five concentrations of a two-fold dilution series of the antigens were sequentially injected at 40 µL/min over all flow cells, including reference flow cell 1, and a buffer blank injection over all flow cells served as control. For details on concentration ranges and contact and dissociation times of analytes, see Table 53. The anti-human Fc surfaces were regenerated to prepare for the next injection cycle by one pulse of 10 mM glycine/HCl, pH 1.5, for 120 s at 30 µL/min. Double reference-subtracted sensograms were analyzed using Biacore™ T200 Evaluation Software v3.0 and then fit to the 1:1 Langmuir binding model.

TABLE D

SPR analyte parameters

| Analyte | Concentration range [nM] | Contact time [s] | Dissociation time [s] |
|---|---|---|---|
| EGFR | 2.5 - 40 | 180 | 300 |
| TF | 0.125 - 2 | 300 | 1800 |
| Mesothelin | 0.125 -2 / 1.25 - 20 | 300 / 180 | 1800 |

### Results

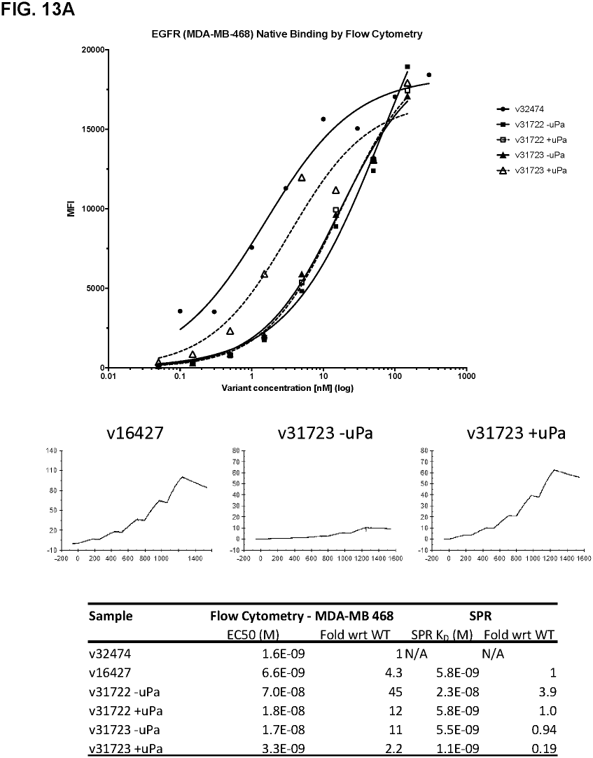

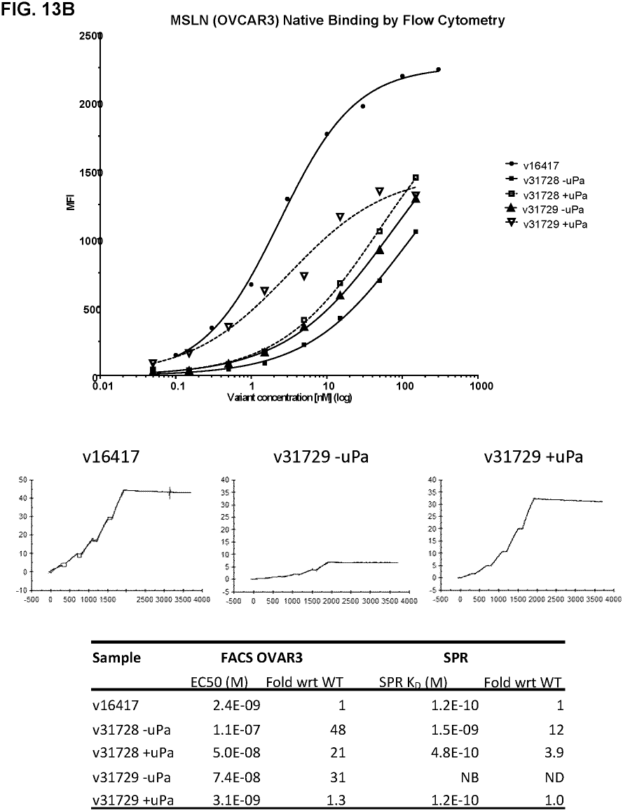

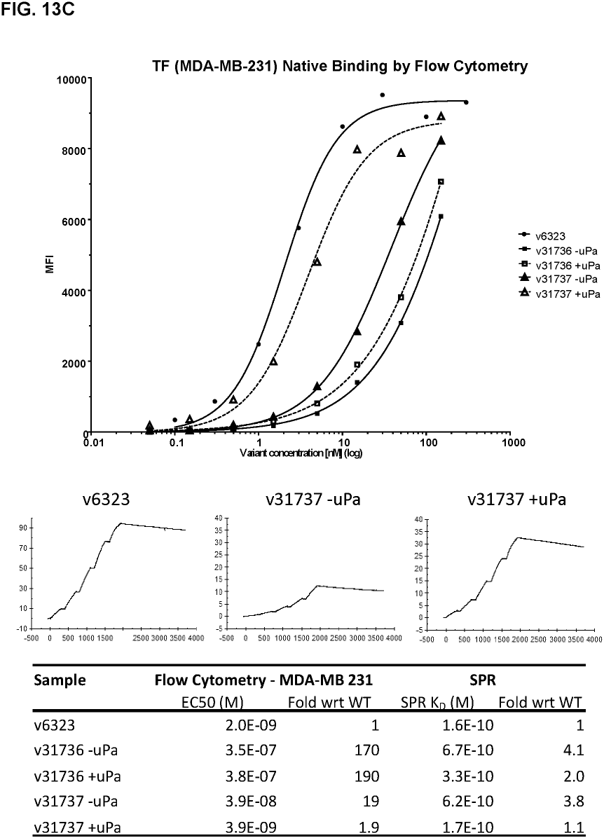

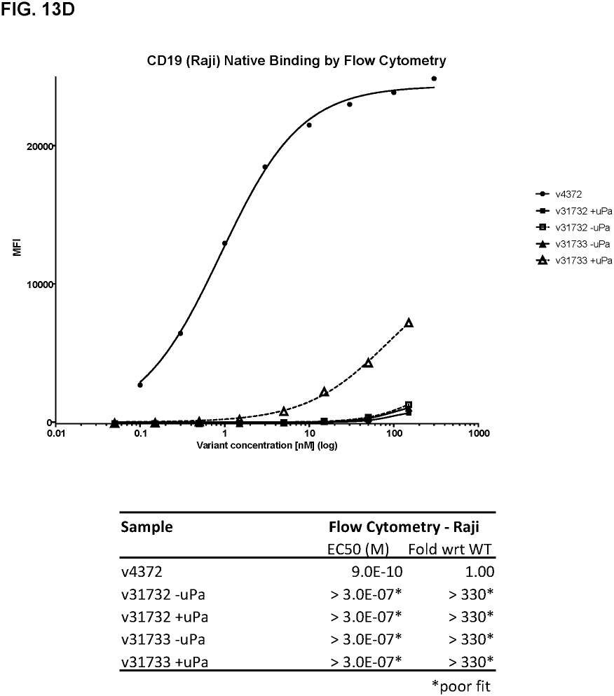

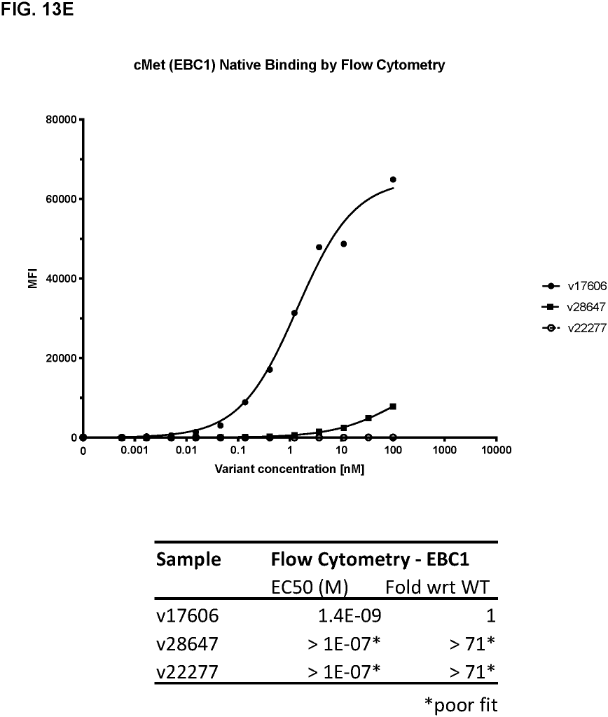

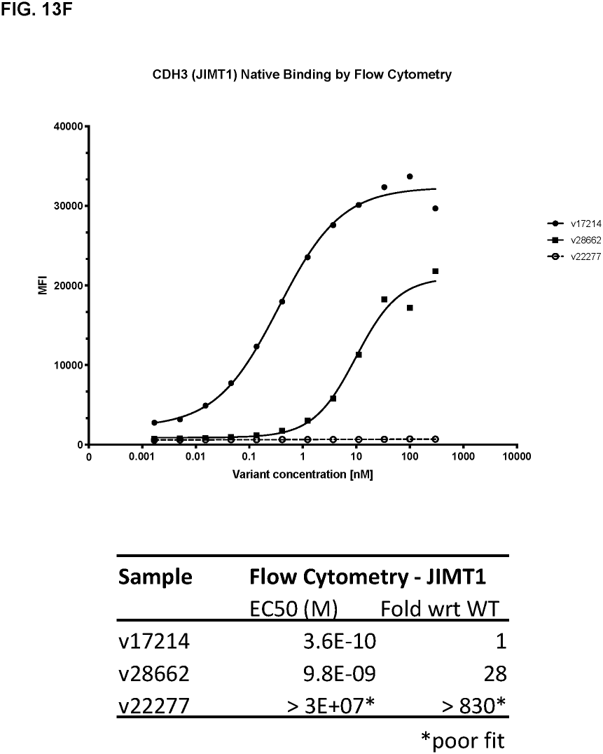

FIG. 13 shows that antigen binding for all uncleavable variants (variants 31722, 31728, 31736, 31732, 28647, 28664 for EGFR, MSLN, TF, CD19, cMet, CDH3, respectively) was reduced 30-190 fold when compared to the respective unmasked controls (variants 32474, 16417, 6323, 4372, 17606, 17214 for EGFR, MSLN, TF, CD19, cMet, CDH3, respectively) as determined by on-cell binding studies. Where cleavable variants were included, samples were tested without (-uPa) and with uPa treatment (+uPa). While uncleavable variants (31722, 31728, 31736, 31732 for EGFR, MSLN, TF, CD19, respectively) showed only minor differences between uncleaved and uPa-treated samples, cleavable samples (31723, 31729, 31737, 31733 for EGFR, MSLN, TF, CD19, respectively) recovered binding significantly upon uPa treatment. Specifically, binding levels were similar to uncleavable variants before being subjected to the protease while upon uPa cleavage, binding was recovered within 1.3-85 fold of the unmasked control. Where available (EGFR, TF, Mesothelin), SPR binding results show the same trends of masking and recovery of binding after cleavage.

### Example 15 Functional Analysis of Masked Anti-EGFR Variants

To investigate in a cell-based assay the impact of the mask on the function of EGFR-targeted variants produced in Example 11, treated by uPa in Example 13 and tested for target-binding in Example 14, a growth inhibition study on NCI-H292 cells was conducted as follows.

### Methods

For this assay, NCI-H292 cells were routinely grown in 75 cm² (T75) flasks at 37° C. +5% CO₂ and passaged twice a week in FBS culture medium without the addition of antibiotic. Cells were seeded the day before addition of antibodies at 300, 1000 and 125 cells / 25 µL / well in 384-well plates (Corning 3570) in their culture media with the addition of 1000 units of penicillin, 1000 µg of streptomycin, and 2.5 µg of Amphotericin B per mL. On the day of the assay, antibodies and controls were serially diluted in 11-points dose-response curves at 6X the desired final concentrations, then added to the plated cells for final incubation concentrations described in Table 5: Variant concentration ranges. Their effects on cell proliferation were measured after 5 days of incubation at 37° C., 5% CO₂. Incubation with an irrelevant antibody (22277) was used to assess non-target-directed cytotoxicity. Cell viability was determined using CellTiterGlo™ (Promega, Madison), based on quantitation of the ATP present in each well, which signals the presence of metabolically active cells. Signal output was measured on a luminescence plate reader (Envision, Perkin Elmer) set at an integration time of 0.1 sec. Integration time is adjusted to minimize signal saturation at high ATP concentration.

Data expressed as Relative Luminescence Unit (RLU) is normalized to non-treated control wells and expressed as % survival, calculated according to Formula: % survival = RLU Ab / RLU non treated X 100.

Using GraphPad Prism software, dose-response curves were generated to measure efficacy (the maximum saturable growth inhibition response observed at high concentrations) and potency (relative IC50, the concentration needed to reach half-maximal efficacy).

TABLE E

Variant concentration ranges

| | |
|---|---|
| Samples | Variant concentration |
| Test samples (31722, 31722 +uPa, 31723, 31723 +uPa) | 825 nM to 0.0008 nM |
| Controls (32474, 32474 +uPa, 22277, 22277 +uPa) | 1000 nM to 0.001 nM |

### Results

As shown in FIG. 14, an anti-EGFR antibody based on Cetuximab (32474) inhibited growth of NCI-H292 cells with an IC50 of 0.11 nM. Treatment with uPa only minimally affected this function. PD-1:PD-L1 masked variants (31722, 31723) were less potent (40-80 fold increased IC50) without treatment with uPa. When treated with uPa, while the uncleavable variant 31722 was still significantly inhibited for function (100 fold), the cleavable 31723 showed recovery of function within 2.5 fold of the unmasked v32474. An irrelevant antibody (22277) showed no function in the growth inhibition assay

### Example 16B7:CD28 Family Ligand Receptor Pairs as Masks -CTLA4:CD80

To determine whether other members of the B7:CD28 family can be utilized to mask a Fab efficiently, a CTLA4:CD80-masked version of the CD3 x Her2 Fab x scFv Fc antibody from Example 1 was produced and assessed for CD3 binding as follows.

### Methods

A masked CTLA4:CD80 CD3 Fab was designed to be equivalent to the PD1:PD-L1 masked variants in Example 1. Briefly, sequences of the IgV domains of human CD80 and CTLA4 (West, S. M. & Deng, X. A. Considering B7-CD28 as a family through sequence and structure. Exp Biol Med (Maywood), 1535370219855970, doi:10.1177/1535370219855970 (2019); SEQ ID 25, 26) were appended to the N-termini of heavy and light chains of the CD3 Fab, respectively, using one of the linker combinations described in Example 1 and Example 10. Specifically, the CTLA4 IgV domain was fused to the LC with a uPa-cleavable sequence while the CD80 moiety could not be removed by the protease. A schematic of the architecture of the investigated variant is shown in FIG. 15. Additionally, to reduce homo-dimerization via CD80 that was described previously (C. C. Stamper et al., Crystal structure of the B7-1/CTLA-4 complex that inhibits human immune responses. Nature 410, 608-611 (2001)), mutations were introduced in the CD80 moiety in some variants. Sequences of the individual chains of the variant are listed in Table F. Antibodies were produced, their sample purity and cleavage by uPa assessed and binding to CD3-bearing Jurkat cells assessed as in Example 2, Example 3, Example 5 and Example 6, respectively.

TABLE F

Sequence composition of tested Variants*

| Variant No | Schematic | Description | Clone H1 | Clone L1 | Clone H2 |
|---|---|---|---|---|---|
| 30444 | 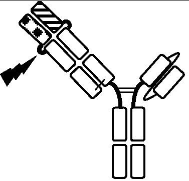 | WT CTLA4:WT CD80 masked CD3 × Her2 Fab × scFv Fc, CTLA4 cleavable | 22088 | 22105 | 21490 |
| ‘33525 | 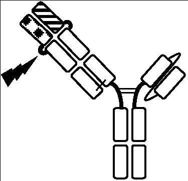 | WT CTLA4:mut1 CD80 masked CD3 × Her2 Fab × scFv Fc, CTLA4 cleavable | 24659 | 22105 | 21490 |
| 33526 | 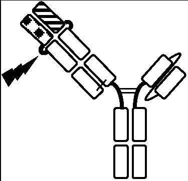 | WT CTLA4:mut2 CD80 masked CD3 × Her2 Fab × scFv Fc, CTLA4 cleavable | 24660 | 22105 | 21490 |
| 33527 | 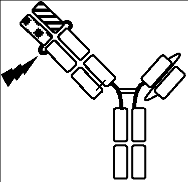 | WT CTLA4:mut3 CD80 masked CD3 × Her2 Fab × scFv Fc, CTLA4 cleavable | 24661 | 22105 | 21490 |

^{∗} The CD80 IgV domain attached to the heavy chain is indicated with a striped pattern in the cartoons and the CTLA-4 IgV domain attached to the light chain is shown as a checkered pattern.

### Results

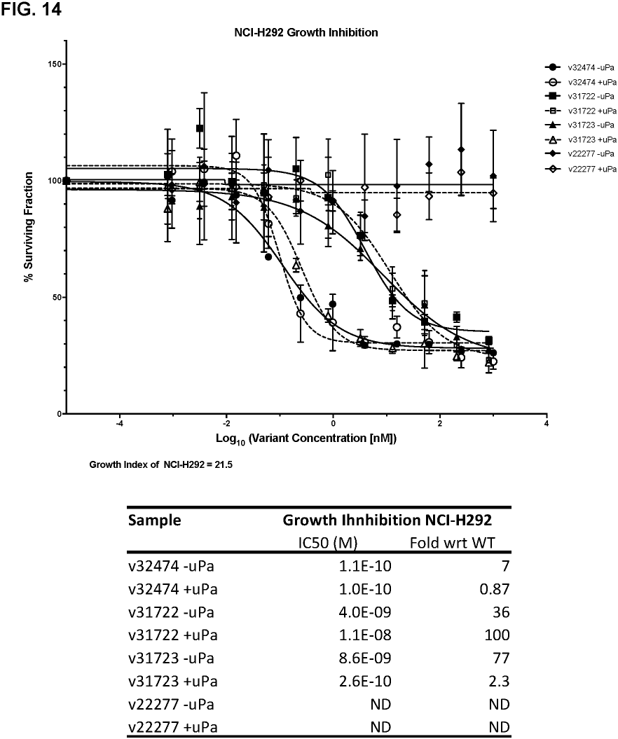

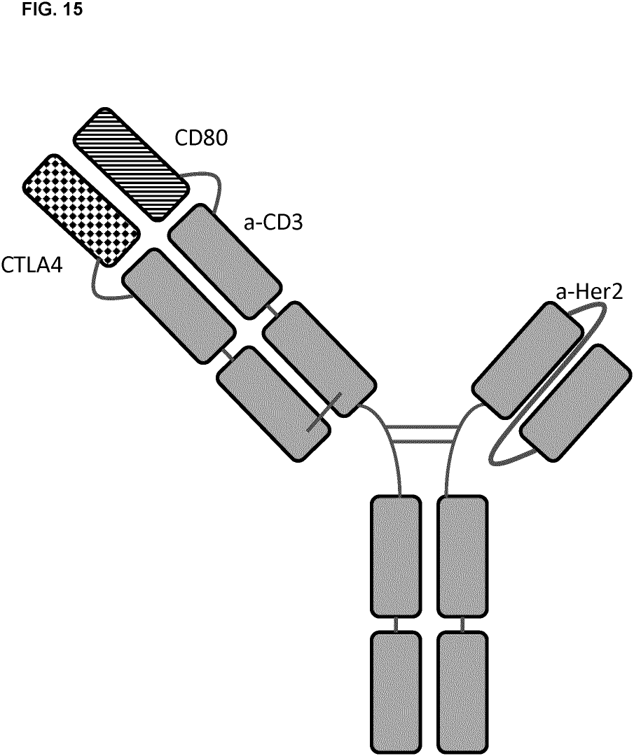

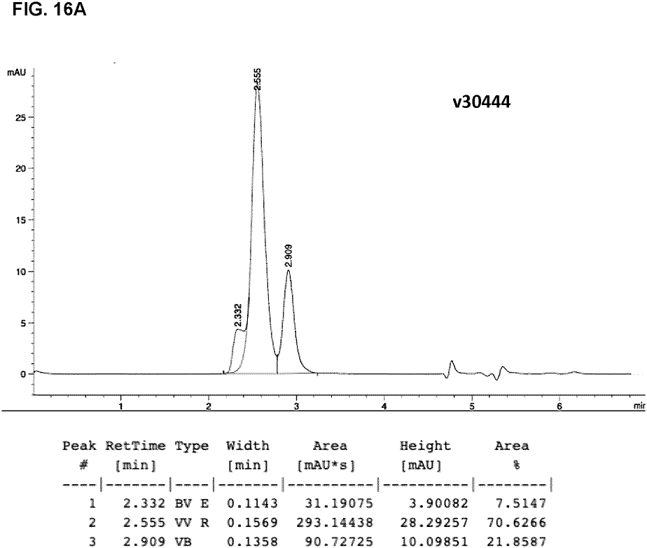

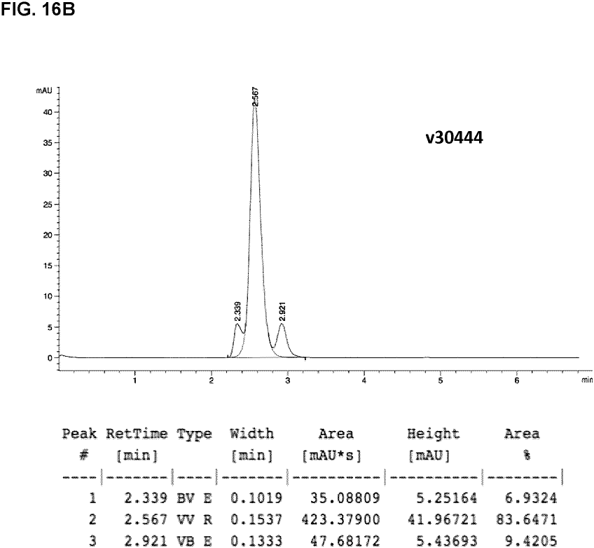

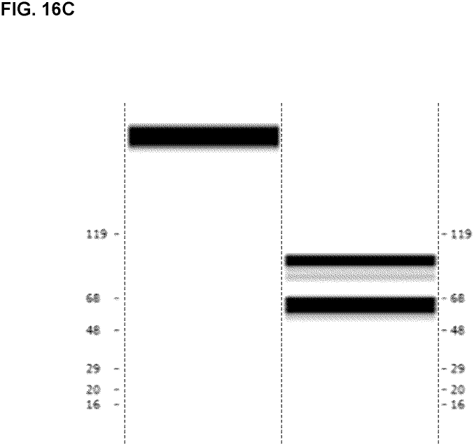

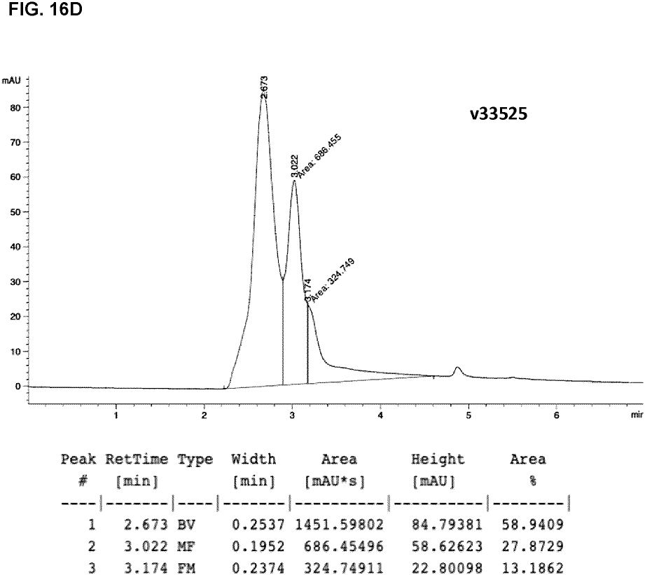

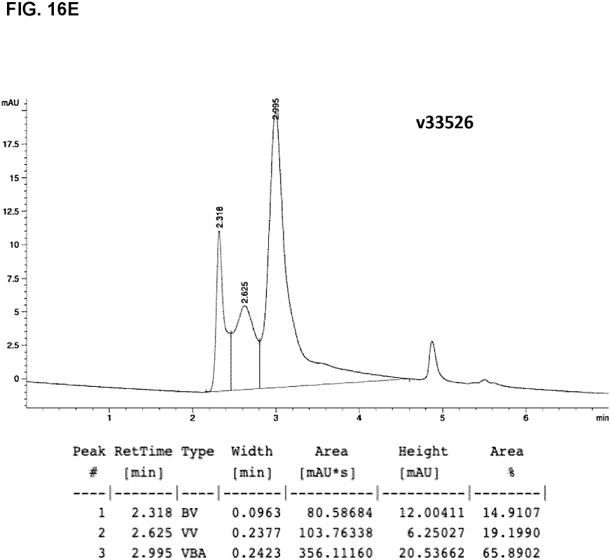

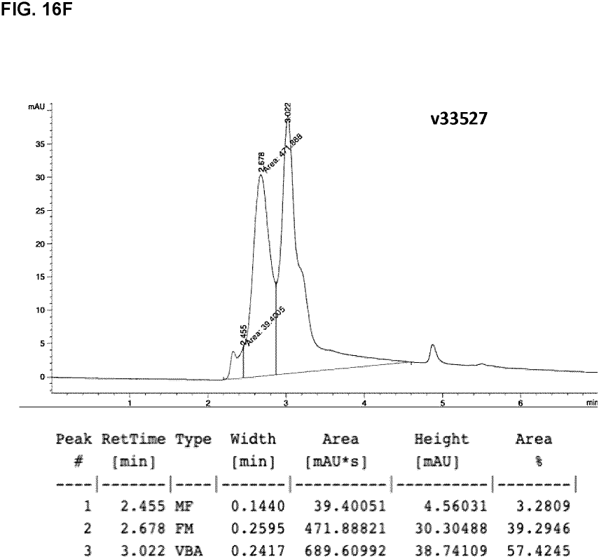

Production of the modified CD3 × Her2 Fab × scFv Fc variant bearing a CTLA4:CD80 based mask (30444) yielded 6.7 mg after preparative SEC, a similar amount to the equivalent PD-1:PD-L1 masked variants in Example 2. UPLC-SEC analysis after protein A purification (FIG. 16A) showed a dimer as the main species, which is consistent with homodimerization interfaces on CD80 and CTLA4 that are distant from the heterodimer interface (Trang, V. H. et al. A coiled-coil masking domain for selective activation of therapeutic antibodies. Nat Biotechnol 37, 761-765, doi:10.1038/s41587-019-0135-x (2019)). A significant amount of high molecular weight species such as aggregates and oligomers were also observed and preparative SEC was performed to remove these undesired particles. UPLC-SEC of the final, SEC purified sample (FIG. 16B) showed 84% of dimeric and 9% of monomeric species. Additionally, 7% of high molecular weight species were still present. Non-reducing CE-SDS (FIG. 16C) showed a profile corresponding to a single predominant species at significantly higher molecular weight than what would be expected for the intact molecule. Bands for modified heavy and light chains show a significantly higher than expected apparent molecular weight in the reducing CE-SDS profile. Similar to the PD-1:PD-L1 based modifications in Example 3, this is likely caused by extensive glycosylation of CD80 and CTLA4 (Stamper, C. C. et al. Crystal structure of the B7-1/CTLA-4 complex that inhibits human immune responses. Nature 410, 608-611, doi:10.1038/35069118 (2001)). When mutations were introduced in the homo-dimerization interface of the CD80 moiety, the amount of dimeric species found in UPLC-SEC after protein A purification was decreased to 19 - 59% while the amount of monomeric species was increased to 28 - 66% (FIGS. 16D-F).

When treated with uPa, the CTLA4 moiety was effectively removed from the light chain as seen in FIG. 17. Here, the band corresponding to the modified light chain disappeared upon cleavage and a band corresponding to the molecular weight of the unmasked light chain appeared. The released CTLA4 component was not detected after cleavage, likely due to the small size and heterogeneity caused by glycosylation.

The assessment of binding to CD3 on Jurkat cells was assessed by ELISA as described in Example 6 (FIG. 18) showed that the CD80:CTLA4 based modification (v30444) decreased target binding ~ 80 fold. This is similar to what is seen for the an equivalent variant with a PD-1:PD-L1 based mask (Example 6, v30430 included here for reference). Upon uPa cleavage of the CTLA4 moiety, CD3 binding is partially restored (within ~ 4 fold of WT).

### Example 17 Conditionally Active Immunomodulators Based On Masked-Immunomodulator-FC-Fusions

The immunomodulatory pairs (e.g. PD-1:PD-L1 (Table G), CD80:CTLA-4) are used as untargeted, conditionally activated molecules in this example. Here, the immunomodulatory pairs do not serve a masking function with regard to a particular paratope but are directly fused to an Fc as follows.

### Methods

The constructs investigated here are based on IgV domains of immunomodulator pairs such as PD-1:PD-L1 that are N-terminally fused to the hinge of a heterodimeric IgG Fc. The Fc portion of these constructs contains mutations in the CH3 domain that drive heterodimeric pairing of the two chains as described previously (for example: Von Kreudenstein, T. S. et al. Improving biophysical properties of a bispecific antibody scaffold to aid developability: quality by molecular design. MAbs 5, 646-654, doi:10.4161/mabs.25632 (2013); SEQ ID 4,5; other heterodimeric Fc forming mutations are also available in the literature). In an embodiment, mutations are also introduced in both CH2 domains to abrogate binding to the Fc gamma receptors (SEQ ID 6,). While one immunomodulator IgV domain (e.g. a high-affinity version of PD-1, Maute, R. L. et al. Engineering high-affinity PD-1 variants for optimized immunotherapy and immuno-PET imaging. Proc Natl Acad Sci U S A 112, E6506-6514, doi:10.1073/pnas.1519623112 (2015), SEQ ID 9) is fused directly to the N terminus of the IgG hinge, an amino acid sequence that is recognized and cleaved by uPa (MSGRSANA) is introduced between hinge and the other immunomodulatory IgV domain (e.g. WT PD-L1, SEQ ID 8) on the other chain.

This design results in a conditionally active, monovalent, PD-L1 targeting molecule that is directly fused to an IgG Fc via a protease cleavable peptidic linker(FIG. 19). In the absence of uPa, the high affinity PD-1:PD-L1 dimer is formed intramolecularly and undesired systemic binding to PD-L1 is prevented. When exposed to uPa, for example, in a tumor microenvironment (TME), PD-L1 is released and the PD-1 moiety can bind to PD-L1 expressed on tumor cells. In the TME, checkpoint activity is thereby selectively blocked and the susceptibility of tumor cells to cytotoxic T-cells is enhanced. Other immunoregulatory ligand receptor pairs such as CD80:CTLA-4 or SIRPa:CD47 are also used as masks. For CD80:CTLA-4, only in the presence of the right TME-associated protease, CTLA-4 is released and the remaining CD80 can bind to CD28 or CTLA-4 on T-cells and in turn exert its immunomodulatory function. For a SIRPa:CD47 mask, the CD47 moiety is released by proteolytic cleavage in the TME, leaving SIRPα free to bind to CD47 on macrophages, thereby inhibiting the checkpoint activity and increasing phagocytosis and tumor cell killing.

Variants with or without treatment with uPa are tested for binding to PD-L1 by flow cytometry as described in Example 8. The same samples are tested in a reporter gene assay (RGA) sensitive to PD-1:PD-L1 checkpoint inhibition (Promega, Madison, WI, USA). The RGA is performed similar to the RGA in Example 9, with the exception that PD-L1 expressing and TCR directed CHO cells are used together with the modified Jurkat T-cells as per the manufacturers protocol.

TABLE G

Sequence composition of tested Variants*

| Variant No | Schematic | Description | SEQ ID H1 | SEQ ID H2 |
|---|---|---|---|---|
| ZW Fc1 | 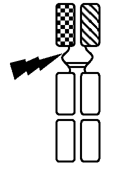 | HAC PD1:PD-L1 (MSGRSANA) IgG1 Fc | 28 | 29 |

^{∗}The PD-1 IgV is indicated with a striped pattern in the cartoons and the PD-L1 IgV domain is shown as a checkered pattern.

### Results

Without treatment with uPa, ZW Fc1 does not bind to PD-L1 in a flow cytometry assay. This is due to the tight intramolecular interaction of the high affinity version of the PD-1 IgV domain with PD-L1 in the Fc assembly. When treated with uPa, ZW Fc1 binds PD-L1 tightly in SPR and flow cytometry assays. This is expected as after cleavage of the uPa-specific sequence in the linker, the PD-L1 moiety is released and the PD-1 domain remaining on the Fc is free to bind PD-L1 in the assays. Similarly, ZW Fc1 without cleavage by uPa has no activity in the PD-1:PD-L1 RGA while it shows robust activity when treated with uPa.

### Example 18 Assessment of the Masking Technology in an Anti-CD40 System

The PD-1:PD-L1 based mask described in examples 1-15 was applied to a CD40-targeted paratope and the sample quality of resulting variants, target binding and impact of the mask on function were assessed as follows.

### Methods

### Variant Design and Production

PD-1:PD-L1 masked versions of full sized antibodies containing a previously described anti-CD40 paratope (R. H. Vonderheide et al., Clinical activity and immune modulation in cancer patients treated with CP-870,893, a novel CD40 agonist monoclonal antibody. J Clin Oncol 25, 876-883 (2007)) were constructed as described in Example 10. The resulting constructs and their sequences are summarized in Table H.

TABLE H

Sequences of anti-CD40 variants

| Variant No | Cartoon | Description | SEQ ID H1 | SEQ ID L1 |
|---|---|---|---|---|
| CD40 | | | | |
| 32477 | 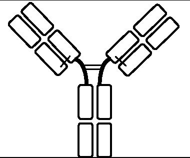 | Unmasked aCD40 mAb | 23712 | 23713 |
| 32478 | 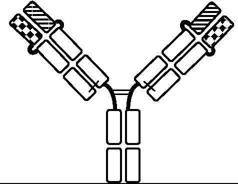 | HA PD-1:WT PD-L1 masked aCD40 Mab, uncleavable | 23714 | 23715 |
| 32479 | 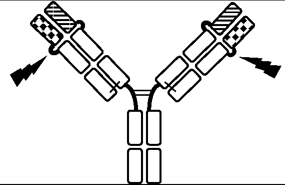 | HA PD-1:WT PD-L1 masked aCD40 Mab, PD-L1 cleavable | 23714 | 23716 |

Heavy and light chain sequences of the described variants were ported into expression vectors, expressed in Expi293F™ cells and purified using the 2-step purification process described in Example 11. Purified samples were then assessed for purity and sample homogeneity by UPLC-SEC and non-reducing gel electrophoresis as described in Example 3. After purification, samples were treated with uPa and their processing assessed by non-reducing CE-SDS as described in Example 5. Both uPa-untreated and uPa-treated samples were then assessed by flow cytometry for target binding to Raji cells as described in Example 14.

### CD40 RGA

To assess uPa-untreated (-uPA) and uPa-treated (+uPA) variants functionally, a CD40 Reporter Gene Assay (RGA) was performed. HEK Blue CD40L cells (Invivogen, San Diego, CA, USA, hkb-cd40 Lot 38-01-hkbcd40) cells were detached with PBS then resuspended at 2.78 × 10⁵ cells/mL in pre-warmed test media (Gibco™ DMEM (Thermo Fisher Scientific, Waltham, MS, USA, 1195-040) plus 10 % heat inactivated Gibco™ FBS (Thermo Fisher Scientific, Waltham, MS, USA, 12483-020 Lot 1996160) (56° C., 30 min) and 100 U/mL Gibco™ Pen-Strep (Thermo Fisher Scientific, Waltham, MS, USA, 15070-063 Lot 1989510)). WT-CHOK1 (ATCC, Manassas, VA, USA, ATCC CCL-61, Lot 70014310) and FcgR2B-CHOK1 cells (BPS Bioscience, San Diego, CA, USA, 79511, Lot 191104-41) were detached with trypsin and resuspended at 5.56 × 10⁵ cells/mL with test media. 25,000 HEK Blue CD40 cells (90 µL) were then added to 20 µL of variants serially diluted in test media (10 µg/mL - 0.000001 µg/mL), followed by addition of 50,000 WT-CHOK1, FcYR2B-CHOK1 cells (90 µL) or 90 µL of test media. After incubation for 20-24 h at 37° C., 5 % CO₂, 20 µL of supernatant were mixed with 180 µL of Quanti-Blue™ solution (Invivogen, San Diego, CA, USA), incubated for 3 h at 37° C., 5 % CO₂ and the OD_{620 nm} measured. Test articles included uPa-untreated and uPa-treated CD40-targeted variants as well as an irrelevant control antibody targeted against RSV and CD40L (Invivogen, San Diego, CA, USA) as negative and positive controls, respectively.

### Results

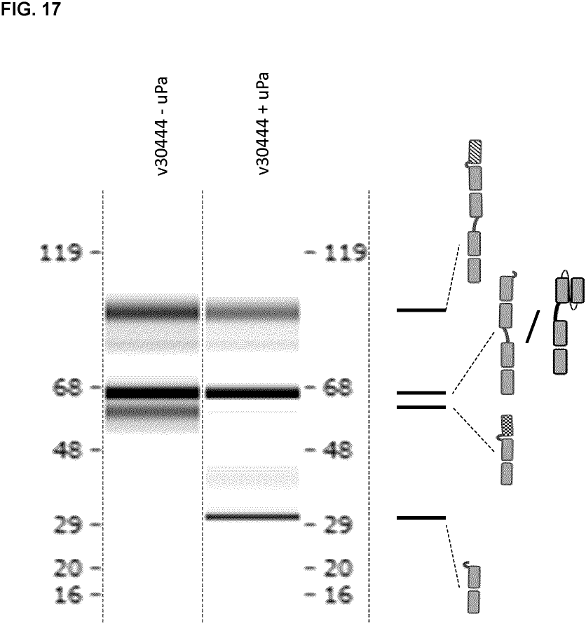

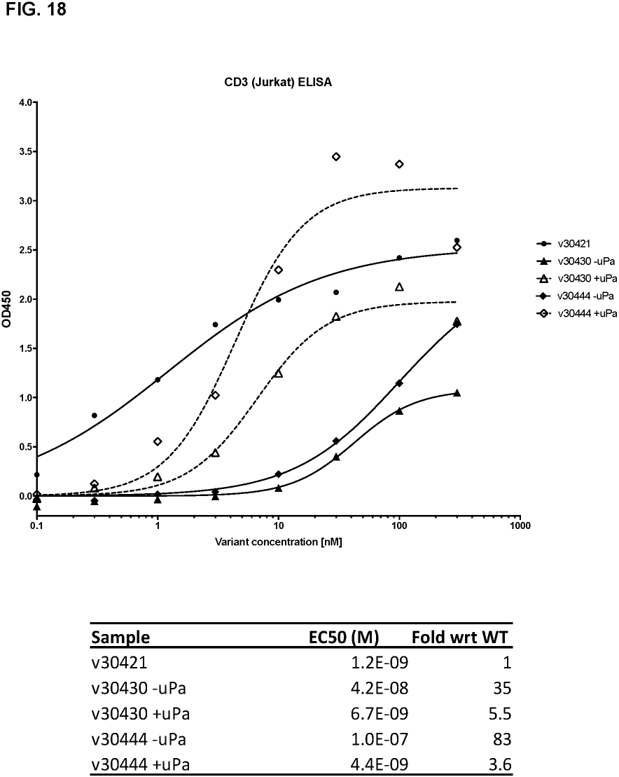

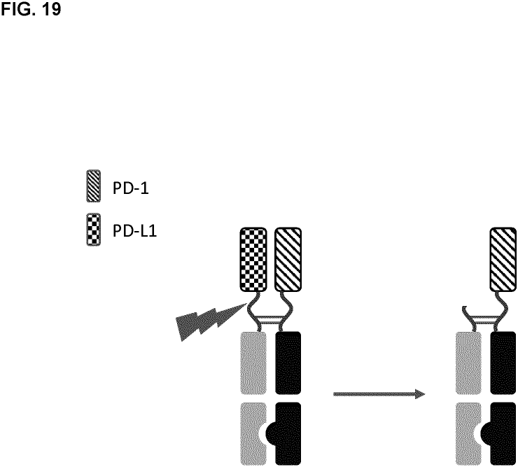

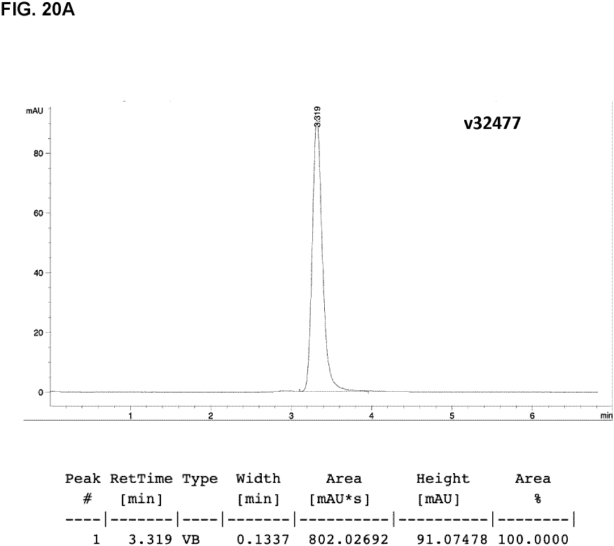

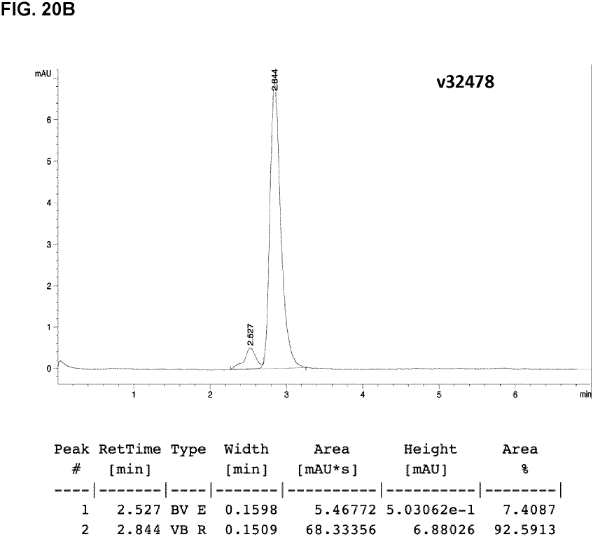

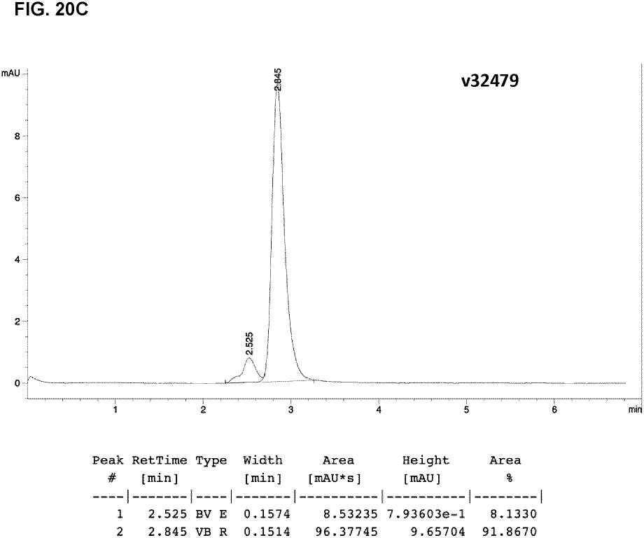

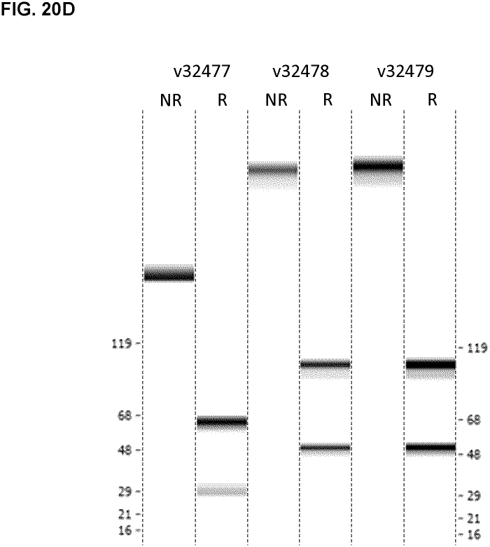

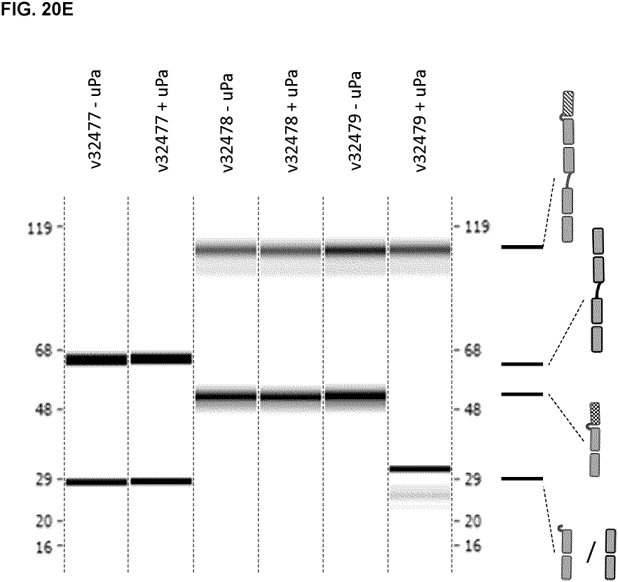

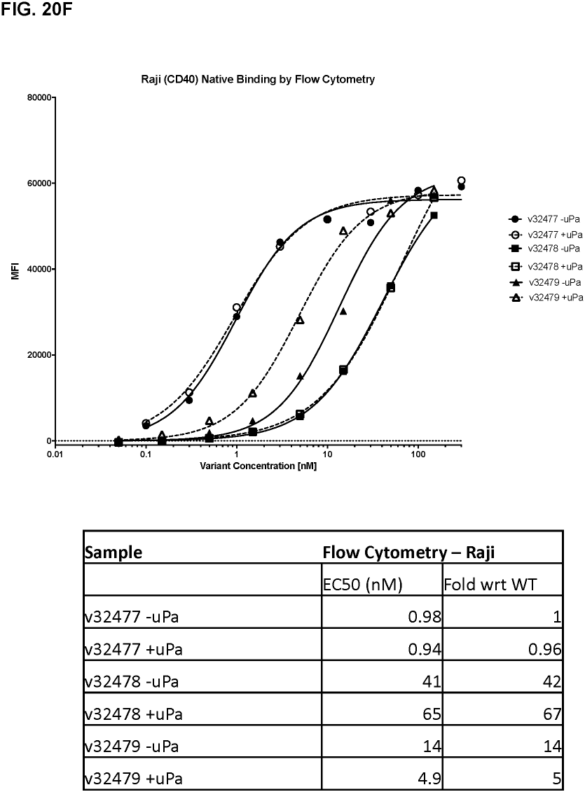

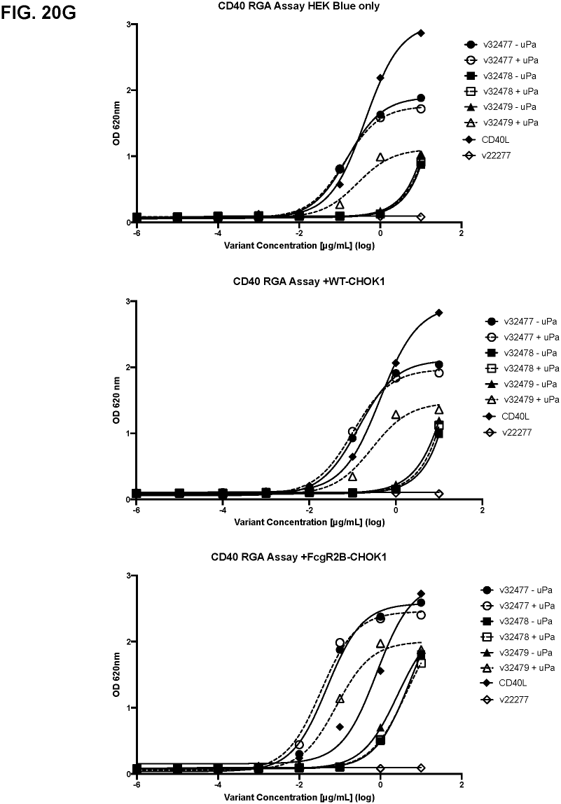

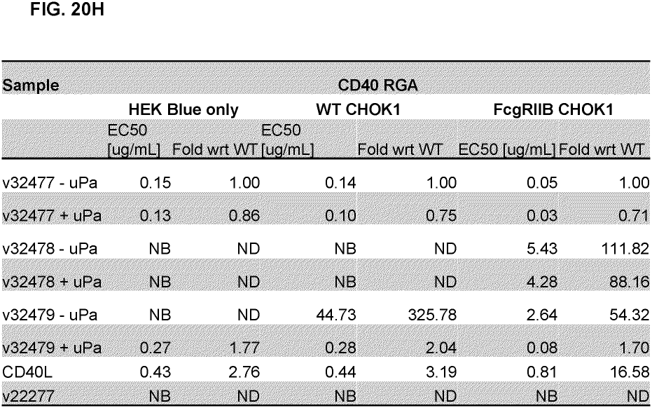

As shown in FIGS. 20 A-C, after SEC purification, the antiCD40 variants showed one predominant species in UPLC-SEC at a purity of 92 % - 100 % with low amounts of higher molecular weight species (7-8 %) present for the masked variants v32478 and v32479. Non-reducing CE-SDS analysis (FIG. 20 D) also showed a single predominant species for all variants. While the apparent molecular weight of the main species of the unmasked v32477 was ~ 150 kDa, as expected, the PD-1:PD-L1 masked variants v32478 and v32479 showed a significantly higher apparent molecular weight (> 250 kDa), likely due to glycosylation, as seen for constructs using the same masking domains in Example 3 and Example 12. Reducing CE-SDS (FIG. 20 D) showed two species of distinct molecular weight corresponding to heavy and light chains for all variants. For the masked variants v32478 and v32479, the apparent molecular weight of both heavy and light was also higher than expected (~ 100 kDa vs 63 kDa for the HC, ~ 50 kDa vs 37 kDa for the LC), likely due to glycosylation of both PD-1 and PD-L1 and as seen in Example 3 and Example 12.

The three anti-CD40 variants investigated here were treated with uPa after production and the cleavage monitored by reducing CE-SDS (FIG. 20 E). While v32477 and v32478 did not show any change upon incubation with uPa due to the lack of a specific cleavage site, processing was seen for the light chain of v32479. Here, the PD-L1 moiety was removed by cleavage of the uPa specific sequence in the linker between the C-terminus of PD-L1 and the N-terminus of VL domain. This resulted in the three fragments detected in reducing CE-SDS after cleavage: the unchanged PD-1 masked heavy chain lacking a uPa-site, a chain corresponding to VL-CL of the kappa light chain and one corresponding to the released PD-L1 moiety.

Samples with and without treatment with uPa were tested for binding to CD40 on Raji cells by flow cytometry. As shown in FIG. 20 F, the unmasked v32477 showed binding curves with EC50 values of 1 nM while binding was decreased 40-70-fold for the masked v32478. Both variants were lacking a uPa cleavage site and hence, binding was not affected by uPa treatment. Binding was reduced 14-fold for untreated v32479, but was recovered within 5-fold when treated with uPa.

These trends were recapitulated when the same samples were interrogated for their functionality in a CD40 specific RGA (FIG. 20 G). While v32477 showed robust independent activity that could be further enhanced by FcγR2B-CHOK1, reduced function by 90-110-fold could be seen for v32478. As both variants lack a uPa cleavage site, they showed the same activity in the RGA experiment with or without uPa treatment. Masking of activity of similar levels to v32478 was seen for v32479 untreated by uPa (55-fold). Activity within 2-fold of v32477 could be detected v32487 treated with uPa. The positive control CD40L induced CD40 activity independently of the presence of FcγR2B and the negative control (v22277) could not activate CD40 in the assay. The maximum levels of activity seen for tested variants in the assay (Bₘₐₓ) were larger in the presence of a FcgR2B positive cell line as opposed to if FcgR2B on a secondary cell line was not present. Treatment with CD40L caused the same increase in Bₘₐₓ, even in the absence of a FcgR2B positive cell line.

### Example 19: SIRPα:CD47 Immunomodulatory Pairs as Masks

To determine whether immunomodulatory pairs outside the B7:CD28 family can be utilized to mask a Fab efficiently, a CD47:SIRPα-masked version of the anti-EGFR antibodies described in Example 10 was produced and assessed for EGFR binding as follows.

### Methods

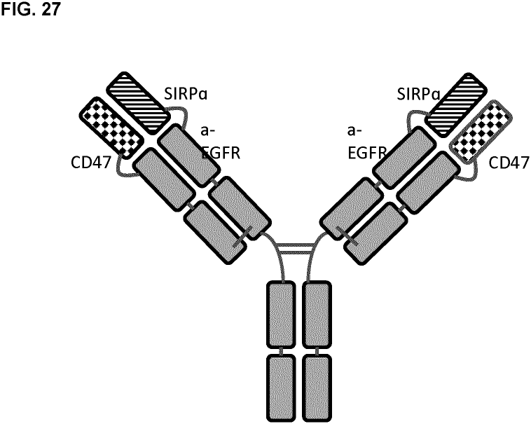

A CD47:SIRPα-masked anti-EGFR antibody was designed to be equivalent to the PD1:PD-L1 masked variants described in Example 10. Briefly, sequences of the IgV domains of human CD47 and a modified, affinity increased variant of human SIRPα (K. Weiskopf et al., Engineered SIRPalpha variants as immunotherapeutic adjuvants to anticancer antibodies. Science 341, 88-91 (2013)) were appended to the N-termini of heavy and light chains of the anti-EGFR Fab, respectively, using uPa cleavable linkers described in Example 1 and Example 10. A schematic of the architecture of the investigated variant is shown in FIG. 27. Sequences of the individual chains of the variant are listed in Table I. Antibodies were produced, their sample purity and cleavage by uPa assessed as in Example 2, Example 3 and Example 5, respectively. Binding to EGFR-bearing H292 cells was then assessed by quantitative fluorescence microscopy

TABLE I

Sequence composition of tested Variants*

| Variant No | Schematic | Description | Clone H1 | Clone L1 |
|---|---|---|---|---|
| 34164 | 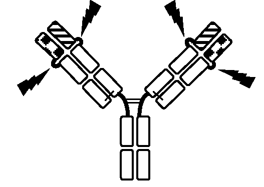 | CD47:SIRPα CV1 masked aEGFR Mab, PD-L1 cleavable | 25321 | 25325 |

^{∗}The SIRPα IgV domain attached to the heavy chain is indicated with a striped pattern in the cartoons and the CD47 IgV domain attached to the light chain is shown as a checkered pattern.

### Native Binding to H292 Cells by Fluorescence Microscopy

The NCI-H292 cell line expressing EGFR was maintained in RPMI-1640, supplemented with L-glutamine and 10% FBS (complete medium) in a humidified + 5% CO2 incubator at 37° C. On the day before the assay, exponentially growing cells were harvested using 0.05% trypsin (Gibco®), resuspended in complete medium at a cell density of 1.2×10⁵ cells/mL. 50 µL of cells were distributed per well in Corning® 96 Half Area Well Flat Clear Bottom Black Polystyrene TC-treated Microplates (Code 3882, Corning, Corning, NY, USA) for a final concentration of 6000 cells/well and incubated overnight in a humidified + 5% CO₂ incubator at 37° C. On the day of the experiment, the plates with cells were allowed to cool down to 4° C. for 30 minutes before performing the assay. Modified variants were diluted to 2X their final concentration in cold DPBS containing Ca²⁺ and Mg²⁺ (Wisent Bioproduct, St-Bruno, Quebec, Canada), followed by three-fold serial dilutions for a total of eleven concentration points starting at 100 nM. All solutions were kept are 4° C. and all incubations were performed at 4° C. Equal volumes of 2X test variants or controls were added to cells and incubated for 2 hours. Cells were then washed with cold DPBS containing Ca²⁺ and Mg²⁺ in the BioTek EL405 select plate washer (BioTek, Winooski, VT, USA) for a 3-cycles wash with 150 µL per well per cycle, with a residual final volume of 25 uL. Detection of bound variants was achieved by an additional incubation with a fluorescent labeling mix containing AF488-labeled, human Fc-specific secondary antibody (Jackson ImmunoResearch, West Grove, PA, USA), Deep Red CellMask (Molecular Probes, Eugene, Oregon, USA) and Hoechst33342 (Molecular probes, Eugene, Oregon, USA) in the presence of FBS (Wisent Bioproduct, St-Bruno, Quebec, Canada) for an hour. Cells were washed twice (3-cycle, 150 µL/well washes each time) in the BioTek EL405select (BioTek, Winooski, VT, USA) plate washer. Images were captured in the ImageXpress Micro XLS (Molecular Devices, San Jose, CA, USA) using the Transmitted Light, DAPI (blue channel), Cy5 (Far red channel) and FITC (green channel). Image analysis was done with the MetaXpress analysis software Custom Module Editor (CME) (Molecular Devices, San Jose, CA, USA). For each well, total green fluorescence intensity was measured in the well area that was covered by cells, then normalized to cell area. This normalized value “Total Intensity per cell area” was used for curve fitting analysis in GraphPad Prism 8 (GraphPad Software, La Jolla, CA, USA). Baseline values were calculated with the average normalized green background fluorescence signal of the control wells (these are wells that were incubated only with the fluorescent labeling mix of secondary antibody). Baseline values in each plate were subtracted from all data before applying the non-linear fit model. Specific Total Intensity per cell area (baseline corrected) versus log antibody concentration were fitted with a “One-Site-Specific binding with Hill slope” nonlinear regression curve fit model for each test article.

### Results

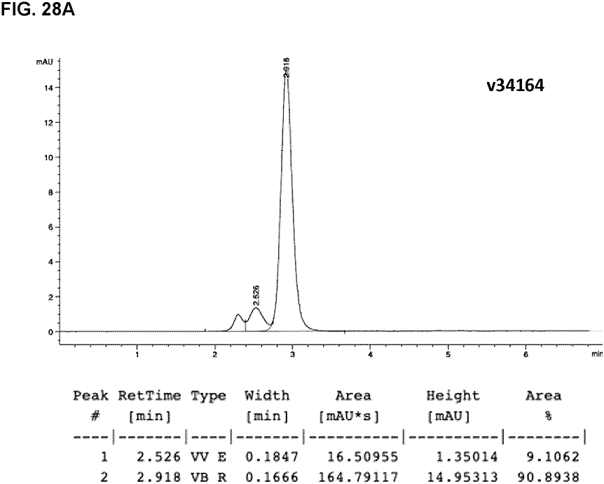

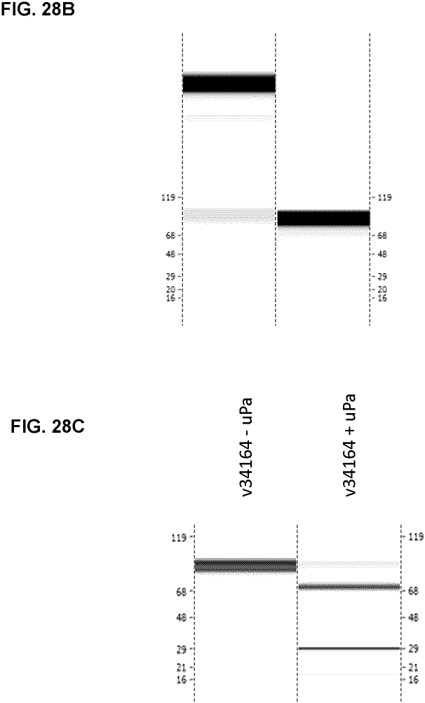

Production of the modified anti-EGFR variant bearing a CD47:SIRPα-based mask (34164) yielded 0.33 mg after preparative SEC. UPLC-SEC analysis after protein A purification showed significant amount of high molecular weight species such as aggregates and oligomers in addition to the main species and preparative SEC was performed to remove these undesired particles. UPLC-SEC of the final, SEC purified sample (FIG. 28A) showed 91 % of the desired species. Non-reducing CE-SDS (FIG. 28B) showed a profile corresponding to a single predominant species at significantly higher molecular weight than what would be expected for the intact molecule. The band for the CD47-modified light chain shows a significantly higher than expected apparent molecular weight in the reducing CE-SDS profile, overlapping with the modified heavy chain. Similar to the PD-1:PD-L1 based modifications in Example 3, this is likely caused by extensive glycosylation of CD47 (W. J. Mawby, C. H. Holmes, D. J. Anstee, F. A. Spring, M. J. Tanner, Isolation and characterization of CD47 glycoprotein: a multispanning membrane protein which is the same as integrin-associated protein (IAP) and the ovarian tumour marker OA3. Biochem J 304 ( Pt 2), 525-530 (1994)).

When treated with uPa, both the CD47 as well as the SIRPα moiety were effectively removed from the light chain as seen in FIG. 29. Here, the bands corresponding to the modified heavy and light chains disappeared upon cleavage and bands corresponding to the molecular weight of the unmasked heavy and light chain appeared. The released CD47 and SIRPα components could not unambiguously identified after cleavage, likely due to their small size and heterogeneity caused by glycosylation.

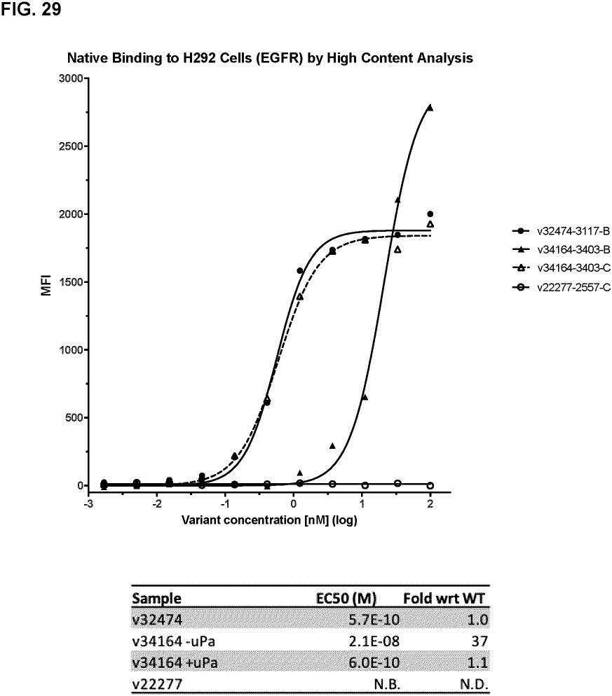

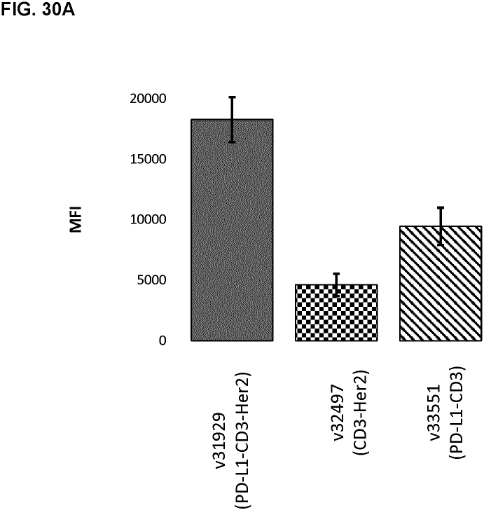

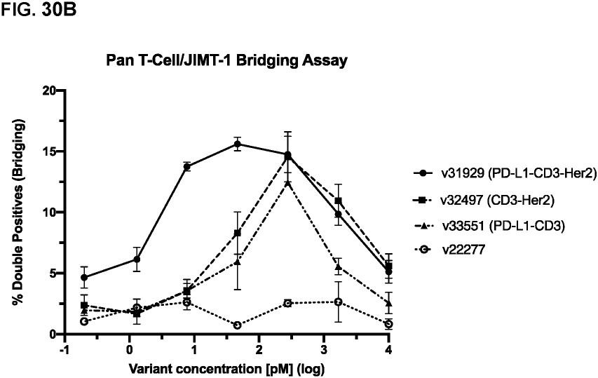

Binding to EGFR on H292 cells as assessed by high-content analysis (FIG. 30) showed that the CD47:SIRPα-based mask in v34164 decreased target binding 37-fold. This is similar to what was seen in Example 14 for an equivalent variant with a PD-1:PD-L1 based mask. Upon uPa cleavage of both mask components, EGFR binding was restored within 1.1-fold of WT.

### Example 20: Co-Engagement and Bridging of Targets by Anti-CD3 Trispecific Variants

To determine whether PD-L1, Her2 and CD3 can be engaged simultaneously by the anti-CD3 variants described in Examples 1-9, Her2-PD-L1 co-engagement as well as T-cell bridging studies were performed as follows.

### Methods

### Assessment of Simultaneous Her2 and PD-L1 Binding by Flow Cytometry

JIMT-1 (Leibniz Institute, Braunschweig, Germany) cultured in growth medium consisting of DMEM medium (Thermo Fisher Scientific, Waltham, MA) supplemented with 10% Fetal Bovine Serum (Thermo Fisher Scientific, Waltham, MA) were maintained horizontally in T-175 flasks (Corning, Corning, NY) in an incubator at 37° C. with 5% carbon dioxide. Antibodies were titrated in a 96-well v-bottom plate (Thermo Fisher Scientific, Waltham, MA) from 100 nM to 1.7 pM at a 1:3 dilution in a total of 20 uL/well in FACS buffer - PBS containing 2% FBS (Thermo Fisher Scientific, Waltham, MA). Tumor cells were rinsed with PBS (Thermo Fisher Scientific, Waltham, MA), harvested with TrypLE Express (Thermo Fisher Scientific, Waltham, MA), diluted in media, and counted using Countess automated cell counter (Thermo Fisher Scientific, Waltham, MA). The tumor cells were washed and resuspended in FACS buffer, and added to the 96-well plate at 50,000 cells per well. The cells were incubated with the variants at 4° C. for 1 hr. Following incubation, the cells were washed 2x with FACS buffer and 1 mg/mL of secondary antibody AF647 Goat anti-human IgG Fc (Jackson ImmunoResearch, West Grove, PA) along with 1000-fold diluted viability dye (Thermo Fisher Scientific, Waltham, MA) was added to the wells. Plate was incubated at room temperature for 30 min. Cells were washed 2x in FACS buffer and resuspended in 100 uL of FACS buffer. For assay read-out, Geometric mean of APC fluorescence was measured by flow cytometry on a BD Celesta (BD Biosciences, San Jose, CA). Raw data was analyzed on FlowJo, LLC Software (Becton, Dickinson & Company, Ashland, OR). Graphs were generated using GraphPad Prism version 8.1.2 for Mac OS X (GraphPad Software, La Jolla, CA).

### CD3/Her2/PD-L1 Bridging Assay

JIMT-1 (Leibniz Institute, Braunschweig, Germany) cultured in growth medium consisting of DMEM medium (Thermo Fisher Scientific, Waltham, MA) supplemented with 10% Fetal Bovine Serum (Thermo Fisher Scientific, Waltham, MA) were maintained horizontally in T-175 flasks (Corning, Corning, NY) in an incubator at 37° C. with 5% carbon dioxide. Tumor cells were rinsed with PBS (Thermo Fisher Scientific, Waltham, MA), harvested with TrypLE Express (Thermo Fisher Scientific, Waltham, MA), diluted in PBS, and washed twice in PBS. A vial of primary human Pan-T cells (BioIVT, Westbury, NY), was thawed in a 37° C. water bath, washed in growth medium consisting of RPMI-1640 ATCC modification (Thermo Fisher Scientific, Waltham, MA) supplemented with 10% Fetal Bovine Serum, subsequently washed in PBS, and resuspended in PBS. T cells and tumor cells were counted using Countess automated cell counter (Thermo Fisher Scientific, Waltham, MA) and resuspended at 5 M/mL in PBS. Cell Proliferation dye-eF670 (Thermo Fisher Scientific, Waltham, MA) was added to tumor cells at 1.25 uM. Cell Tracker Green (Thermo Fisher Scientific, Waltham, MA) was added to T cells at 2 uM. T cells and Tumor cells were incubated at 37° C. for 20 min in the dark and washed twice in FACS buffer - PBS containing 2% FBS (Thermo Fisher Scientific, Waltham, MA). Antibodies were titrated down a v-bottom 96-well plate (Thermo Fisher Scientific, Waltham, MA) from 10 nM to 0.2 pM at a 1:6 dilution in a total of 50 uL/well in FACS buffer. Pan T cells were mixed with tumor cells at 5:1 effector to target ratio at 1.44 E6 cell/mL. 50 uL of the mixed cell suspension was added to the plate containing the titrated variants. The cells were incubated with the variants at 4° C. for 1 hr. For assay read-out, double positive population of cells was measured by flow cytometry on a BD Celesta (BD Biosciences, San Jose, CA). Raw data was analyzed on FlowJo, LLC Software (Becton, Dickinson & Company, Ashland, OR). Graphs were generated using GraphPad Prism version 8.1.2 for Mac OS X (GraphPad Software, La Jolla, CA).

### Results

Binding to an endogenous Her2+/PD-L1+ cancer cell line (JIMT-1, see Example 7 for Her2 and PD-L1 receptor quantification) was measured by flow cytometry (FIG. 30A). A trispecific (PD-1-CD3-Her2) variant that only has PD-1 appended to the heavy chain (31929) and represents a fully unmasked version of the masked variant 30430, shows a higher MFI compared to bispecific controls (v32497 (CD3-Her2), v33551 (PD-L1-CD3)). To achieve bispecific controls in the same format, mutations in the PD-1 moiety that abrogate PD-L1 binding were introduced in v32497, while an irrelevant scFv targeted to hemagglutinin replaced the Her2 targeted scFv in v33551. This is evidence that both PD-L1 and Her2 are simultaneously engaged on the cancer cell by the trispecific variant.

Furthermore, antibody-dependent bridging of a Her2+/PDL1+ cancer cell line and Pan T-cells was assessed by the presence of a double positive signal (fluorescent signal for both T-cell and target cell at the same time) in flow cytometry (FIG. 30B). A higher percentage of double positive signal for the trispecific (PD-1-CD3-Her2) variant (v31929) compared to bispecific controls (v32497 (CD3-Her2), v33551 (PD-L1-CD3)) shows that the variants described here are capable of bridging T-cells and cancer cells and that the simultaneous engagement of all three targets by v31929 increases this T-cell bridging.

### Example 21: In Vivo Functional Evaluation of Anti-CD3 × Anti-Her2 T Cell-Engager Fusion Proteins

The functional impact of the PD-1:PD-L1 based mask on the ability of the CD3 x Her2 Fab x scFv Fc variants described in Examples 1-9 to engage and activate T-cells for the killing of Her2-bearing tumor cells is assessed in an in vivo study in a humanized mouse model as follows.

### Methods

Mice (NSG [NOD-scid-gamma]) are implanted subcutaneously with 5×106 cells from a human Her2+ tumor line (JIMT-1) and simultaneously engrafted intravenously with 1×107 PBMCs from healthy human donors. After establishment and initial growth of the tumor to approximately 150-200 mm3, the mice are dosed intravenously with antibody variants described and produced in examples 1-9. Mice are monitored for both body weight and tumor growth (measured by caliper) twice per week for duration of the study.

### Results

Trends seen for masked and unmasked CD3 × Her2 Fab × scFv Fc variants in binding to CD3 and functional studies in examples 6-9 are recapitulated when the same samples are interrogated for anti-tumor activity in an in vivo study using a humanized mouse model with PBMCs from healthy donors as well as a Her2 positive human cancer cell line engrafted. While the tumor grows rapidly in the animals treated with no drug or an irrelevant control antibody (22277), a variant with just a non-functional PD-L1 domain attached to the heavy chain (32497) shows robust tumor growth inhibition due to its ability to recruit T-cells for killing. When the same variant is paired in a combination with an anti PD-L1 antibody (32497 + 33449), additional inhibition can be seen due to the additional checkpoint activity. A variant with a functional PD-1 domain (31929) also shows additional tumor growth inhibition when compared to the equivalent construct with a non-functional PD-1 domain (32497). When variants with complete PD-1:PD-L1 based masks are evaluated, a construct with uncleavable linkers on both appended domains (30423) shows rapid tumor growth. Conversely, a construct with a cleavable linker between Fab and PD-L1 (30430) shows high anti-tumor activity, similar to an unmasked, trispecific control (31929) when a tumor cell line with high expression of the relevant protease is used in the model. When a tumor cell line with low protease expression is used, the same cleavable variant (30430) shows rapid tumor growth, similar to an uncleavable construct (30430).

### Example 22: CD80-CTLA-4, CD80-CD28, and CD80-PD-L1 Ligand-Receptor Pairs as Masks

CD80 affinities for CTLA-4, CD28, and PD-L1 are 0.2 uM, 4 uM, and 1.7 uM, respectively (Butte, M. J. et al, Programmed death-1 ligand 1 interacts specifically with the B7-1 costimulatory molecule to inhibit T cell responses. Immunity, 27, 111-122, doi:10.1016/j.immuni.2007.05.016 (2007)). To drive preferential binding of CD80 to CD28, mutations are introduced into CD80 IgV domain that are known to selectively increase affinity for CD28 (patent: US20210155668A1). In a “one-sided” CD80 mask format, multiple constructs were designed to evaluate what geometry optimally potentiates T cell activation. Briefly, the IgV domain of human CD80 with mutations to prevent CD80 homodimerization (as described above) and/or CD80 with mutations predicted to increase affinity for CD28 are appended to the N-termini of heavy or light chain of anti-CD3 Fab using an (EAAAK)2 linker and paired in a heterodimeric Fc format with anti-TAA scFv × Fc. Alternatively, the CD80 IgV domain is appended to the N-termini of heavy or light chain of anti-TAA Fab using an (EAAAK)2 linker and paired in a heterodimeric Fc format with an anti-CD3 scFv × Fc. The formats described above are illustrated in Table J.

As CD80 can bind CTLA-4, CD28, and PD-L1, all three are used as dimeric mask partners (CD80:CTLA-4, CD80:CD28, CD80:PD-L1). The resulting masked constructs were designed using a CD80 IgV domain with mutations to prevent CD80 homodimerization and known to increase affinity for CD28. In all instances, the CTLA-4, CD28, or PD-L1 IgV domains are fused to the heavy or light chain with a protease-cleavable sequence while the CD80 moiety is fused to the light or heavy chain with an alpha helical peptide linker sequence designed to not be removed by an endogenous protease. For the CD80:CTLA-4 mask design, a high affinity version of the CD80 IgV domain and a wildtype, human CTLA-4 IgV domain are appended to the N-termini of heavy and light chains of the anti-CD3 Fab, respectively, using peptide linkers and paired with an anti-TAA scFv Fc. For a CD80:CD28 mask, a high affinity version of the CD80 IgV domain and a wildtype, human CD28 IgV domain are appended to the N-termini of heavy and light chains of the anti-CD3 Fab, respectively, using peptide linkers and paired with an anti-TAA scFv Fc. Lastly, for a CD80:PD-L1 mask, a CD80 IgV domain with mutations to prevent CD80 homodimerization and predicted to increase affinity for CD28 and PD-L1 (patent: US20210155668A1) and wildtype, human PD-L1 IgV domain are appended to the N-termini of heavy and light chains of the anti-CD3 Fab, respectively, using peptide linkers and paired with an anti-TAA scFv Fc. Furthermore, molecules are designed wherein the CD80-containing mask (CD80:CTLA-4, CD80:CD28 or CD80:PD-L1) is used to block an anti-TAA Fab paratope and the chain is paired with an anti-CD3 scFv. The masked variants described above are illustrated in Table J.

In the above examples, constructs are described with one sided or dimeric, CD80-based masks (CD80:CTLA-4, CD80:CD28, CD80:PD-L1) used in a molecule that includes an anti-CD3 arm (Fab or scFv) and an anti-TAA arm (Fab or scFv). These designs could serve as a platform with a range of anti-CD3 paratopes and any TAA paratope.

TABLE J

Schematics of CD80 one-sided mask variants and fully masked CD80 variants

| Schematic | Description |
|---|---|
| 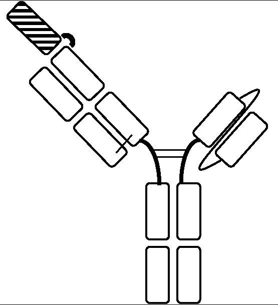 | CD80 (HC) a-CD3 Fab × a-TAA scFv |
| 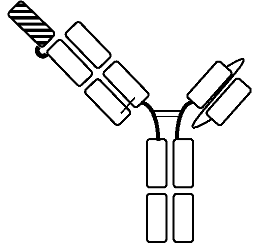 | CD80 (LC) a-CD3 Fab × a-TAA scFv |
| 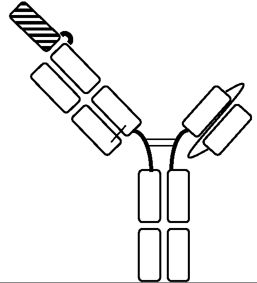 | CD80 (HC) a-TAA Fab × a-CD3 scFv |
| 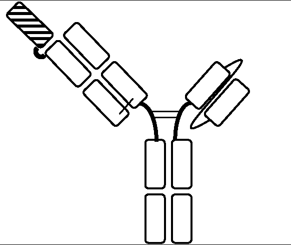 | CD80 (LC) a-TAA Fab × a-CD3 scFv |
| 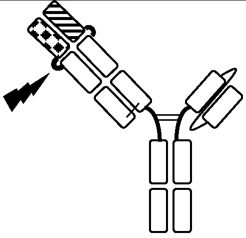 | CD80 (HC) and CTLA-4, CD28, or PD-L1 (LC) a-CD3 Fab × a-TAA scFv |
| 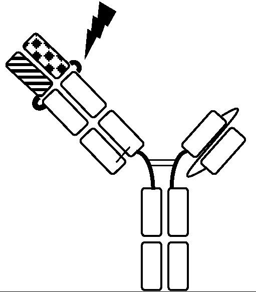 | CTLA-4, CD28, or PD-L1 (HC) and CD80 (LC) a-CD3 Fab × a-TAA scFv |
| 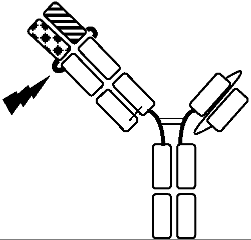 | CD80 (HC) and CTLA-4, CD28, or PD-L1 (LC) a-TAA Fab × a-CD3 scFv |
| 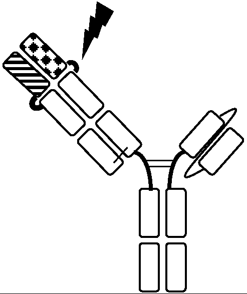 | CTLA-4, CD28, or PD-L1 (HC) and CD80 (LC) a-TAA Fab × a-CD3 scFv |

^{∗} The a-CD3 arms are shaded in dark grey and the a-TAA arms are shaded light grey. The CD80 IgV domain is indicated with a striped pattern in the cartoons and the CTLA-4, CD28, or PD-L1 IgV domains are shown with a checkered pattern. The thunderbolt indicates a protease-cleavable linker sequence.

Modifications of the specific embodiments described herein that would be apparent to those skilled in the art are intended to be included within the scope of the following claims.

All references issued patents and patent applications cited within the body of the instant specification are hereby incorporated by reference in their entirety, for all purposes.

### Sequences

TABLE AA

Part 1

| | | |
|---|---|---|
| SEQ ID | DESCRIPTION | SEQUENCE |
| SEQ ID NO:1 | CRIS7 CD3 VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT |
| SEQ ID NO:2 | CRIS7 CD3 VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS |
| SEQ ID NO:3 | Trastuzumab scFv | DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS |
| SEQ ID NO:4 | Chain A CH3 region | GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG |
| SEQ ID NO:5 | ChainB CH3 region | GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG |
| SEQ ID NO:6 | CH2 region with L234A-L235A_D265S mutations | APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK |
| SEQ ID NO:7 | Wild type PD-1 | NPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTE |
| SEQ ID NO:8 | Wild type PD-L1 | AFTVTVPKDLYVVEYGSNMTIECKFPVEKQLDLA ALIVYWEMEDKNIIQFVHGEEDLKVQHSSYRQRARLLKDQLSLGNAALQITDVKLQDAGVYRCMISYGGADYKRITVKVNA |
| SEQ ID NO:9 | High affinity PD-1 | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE |
| SEQ ID NO:10 | High affinity PD-L1 | AFTVTVPKDLYVVEYGSNMTIECKFPVEKQLDLA ALQVFWMMEDKNIIQFVHGEEDLKVQHSSYRQRARLLKDQLSLGNAALQITDVKLQDAGVYTCLIAYKGADYKRITVKVNA |
| SEQ ID NO:11 | WT CþS PD-1 | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE |
| SEQ ID NO:12 | Wild type CH3 region | GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG |
| SEQ ID NO:13 | EGFR VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK |
| SEQ ID NO:14 | EGFR VH | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA |
| SEQ ID NO:15 | MSLN VL | DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK |
| SEQ ID NO:16 | MSLN VH | QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS |
| SEQ ID NO:17 | TF VL | DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIK |
| SEQ ID NO:18 | TF VH | EVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSS |
| SEQ ID NO:19 | CD19A VL | EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIK |
| SEQ ID NO:20 | CD19A VH | QVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSS |
| SEQ ID NO:21 | cMET VL | DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK |
| SEQ ID NO:22 | cMET VH | QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS |
| SEQ ID NO:23 | CDH3 VL | QSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVL |
| SEQ ID NO:24 | CDH3 VH | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSS |
| SEQ ID NO:25 | WT CD80 | VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKA |
| SEQ ID NO:26 | WT CTLA4 | MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE |
| SEQ ID NO:27 | Signal peptide | EFATMRPTWAWWLFLVLLLALWAPARG |
| SEQ ID NO:28 | Protease cleavage site | MSGRSANA |
| SEQ ID NO:29 | Human IgG1 Fc sequence 231-447 (EU-numbering) | APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK |
| SEQ ID NO:30 | Protease cleavage site | TSGRSANP |
| SEQ ID NO:31 | Protease cleavage site | LSGRSDNH |
| SEQ ID NO:32 | Protease cleavage site | GSGRSAQV |
| SEQ ID NO:33 | Protease cleavage site | GSSRNADV |
| SEQ ID NO:34 | Protease cleavage site | GTARSDNV |
| SEQ ID NO:35 | Protease cleavage sequence | GGGRVNNV |
| SEQ ID NO:36 | Protease cleavage site | MSARILQV |
| SEQ ID NO:37 | Protease cleavage site | GKGRSANA |
| SEQ ID NO:38 | Linker | EAAAKEAAAK |
| SEQ ID NO:39 | Linker | EAAAK |
| SEQ ID NO:40 | Linker | PPPP |
| SEQ ID NO:41 | Linker | PPP |
| SEQ ID NO:42 | Linker | GGGGS |
| SEQ ID NO:43 | Linker with C-terminal protease cleavage site | EAAAKEAAAKMSGRSANA |
| SEQ ID NO:44 | Linker with N-terminal protease cleavage site | MSGRSANAEAAAKEAAAK |
| SEQ ID NO:45 | Linker with N-terminal protease cleavage sequence | MSGRSANAEAAAK |

TABLE AA

Part 2 Clone sequences

| Clone ID | Sequence Type | Sequence | Name | Target | Seq ID No. |
|---|---|---|---|---|---|
| 787 | Full | GDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 46 |
| | VL | DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIK | D3H44 | TF | 47 |
| | LCDR1 | RASRDIKSYLN | | | 48 |
| | LCDR2 | YATSLAE | | | 49 |
| | LCDR3 | LQHGESPWT | | | 50 |
| 1380 | Full | GEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK | | | 51 |
| 2932 | Full | GEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 52 |
| | VH | EVQLVESGGGLVQPGGSLRLSCAASGFNIKEYYMHWVRQAPGKGLEWVGLIDPEQGNTIYDPKQDRATISADNSKNTAYLQMNSLRAEDTAVYYCARDTAAYFDYWGQGTLVTVSS | D3H44 | TF | 53 |
| | HCDR1 | EYYMH | | | 54 |
| | HCDR2 | LIDPEQGNTIYDPKFQD | | | 55 |
| | HCDR3 | DTAAYFDY | | | 56 |
| 3232 | Full | GDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 57 |
| | VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK | Cetuximab | EGFR | 58 |
| | HCDR1 | RASQSIGTNIH | | | 59 |
| | HCDR2 | YASESIS | | | 60 |
| | HCDR3 | QQNNNWPTT | | | 61 |
| 3345 | Full | GQVTLRESGPALVKPTQTLTLTCTCTFSGFSLSTSGMGVGWIRQPPGKALEWLAHIWWDDDKRYNPALKSRLTISKDTSKNQVVLTMTNMDPVTAAYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPPREEQYNSTYRVVSVLTVLHODWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTV | | | 62 |
| | VH | QVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSS | SGN-CD19a | CD19 | 63 |
| | HCDR1 | TSGMGVG | | | 64 |
| | HCDR2 | HIWWDDDKRYNPALKS | | | 65 |
| | HCDR3 | MELWSYYFDY | | | 66 |
| 3357 | Full | GDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 57 |
| | VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK | Cetuxi mab | EGFR | 58 |
| | LCDR1 | RASQSIGTNIH | | | 59 |
| | LCDR2 | YASESIS | | | 60 |
| | LCDR3 | QQNNNWPTT | | | 61 |
| 10564 | Full | GQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKF NWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG | | | 67 |
| | VH | QVQLVQSGAEVKKPGASVKVSCKASGY SFTGYTMNWVRQAPGQGLEWMGLITPY NGASSYNQKFRGKATMTVDTSTSTVYM ELSSLRSEDTAVYYCARGGYDGRGFDY WGQGTLVTVSS | huRG7 787 | Mesoth elin | 68 |
| | HCDR1 | GYTMN | | | 69 |
| | HCDR2 | LITPYNGASSYNQKFRG | | | 70 |
| | HCDR3 | GGYDGRGFDY | | | 71 |
| 10565 | Full | GDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 72 |
| | VL | DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK | huRG7 787 | Mesothelin | 73 |
| | LCDR1 | SASSSVSYMH | | | 74 |
| | LCDR2 | DTSKLAS | | | 75 |
| | LCDR3 | QQWSKHPLT | | | 76 |
| 10567 | Full | GQSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS | | | 77 |
| | VL | QSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVL | PF03732010 | CDH3 | 78 |
| | LCDR1 | TGTSNDVGAYNYVS | | | 79 |
| | LCDR2 | EVNKRPS | | | 80 |
| | LCDR3 | SSFTSGLPWVV | | | 81 |
| 10606 | Full | GQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG | | | 82 |
| | VH | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA | Cetuximab | EGFR | 83 |
| | HCDR1 | NYGVH | | | 84 |
| | HCDR2 | VIWSGGNTDYNTPFTS | | | 85 |
| | HCDR3 | ALTYYDYEFAY | | | 86 |
| 11274 | Full | GEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 87 |
| | VH | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSS | PF03732010 | CDH3 | 88 |
| | HCDR1 | SYAMS | | | 89 |
| | HCDR2 | AISGSGGSTYYADSVKG | | | 90 |
| | HCDR3 | WGDGTLNP | | | 91 |
| 11462 | Full | GDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 92 |
| | VL | DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK | Telisotuzumab | c-Met | 93 |
| | LCDR1 | KSSESVDSYANSFLH | | | 94 |
| | LCDR2 | RASTRES | | | 95 |
| | LCDR3 | QQSKEDPLT | | | 96 |
| 11509 | Full | GQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG | | | 97 |
| | VH | QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS | Telisotuzumab | c-Met | 98 |
| | HCDR1 | AYTMH | | | 99 |
| | HCDR2 | WIKPNNGLANYAQKFQG | | | 100 |
| | HCDR3 | SEITTEFDY | | | 101 |
| 12985 | Full | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 102 |
| | VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT | hCris7 | CD3 | 103 |
| | LCDR1 | SASSSVSYMN | | | 104 |
| | LCDR2 | DSSKLAS | | | 105 |
| | LCDR3 | QQWSRNPPT | | | 106 |
| 12989 | Full | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 107 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 20855 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKV DNALQSGNSQESVTEQDSKDSTYSLSSTLTLSKADYEKHKVYACEVTHQGLSSPVTKSFNRGEC | | | 112 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAK | | | 114 |
| | VL | DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK | Telisotuzumab | c-Met | 93 |
| | LCDR1 | KSSESVDSYANSFLH | | | 94 |
| | LCDR2 | RASTRES | | | 95 |
| | LCDR3 | QQSKEDPLT | | | 96 |
| 20859 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG | | | 115 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS | Telisotuzumab | c-Met | 98 |
| | HCDR1 | AYTMH | | | 99 |
| | HCDR2 | WIKPNNGLANYAQKFQG | | | 100 |
| | HCDR3 | SEITTEFDY | | | 101 |
| 20871 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKQSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVLGQPKAAPSVTLFPPSSEELQANKATLVCLISDFYPGAVTVAWKADSSPVKAGVETTTPSKQSNNKYAASSY LSLTPEQWKSHRSYSCQVTHEGSTVEKTVAPTECS | | | 117 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAK | | | 114 |
| | VL | QSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVL | PF03732010 | CDH3 | 78 |
| | LCDR1 | TGTSNDVGAYNYVS | | | 79 |
| | LCDR2 | EVNKRPS | | | 80 |
| | LCDR3 | SSFTSGLPWVV | | | 81 |
| 20875 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSSASTKGPSVFPLAPSSKSTSGGTAALGCLVKDYFPEPV TVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 118 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSS | PF03732010 | CDH3 | 88 |
| | HCDR1 | SYAMS | | | 89 |
| | HCDR2 | AISGSGGSTYYADSVKG | | | 90 |
| | HCDR3 | WGDGTLNP | | | 91 |
| 21490 | Full | DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG | | | 119 |
| | VH | EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS | Trastuzumab | HER2 | 120 |
| | HCDR1 | DTYIH | | | 121 |
| | HCDR2 | RIYPTNGYTRYADSVKG | | | 122 |
| | HCDR3 | WGGDGFYAMDY | | | 123 |
| | VL | DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK | Trastuz umab | HER2 | 124 |
| | LCDR1 | RASQDVNTAVA | | | 125 |
| | LCDR2 | SASFLYS | | | 126 |
| | LCDR3 | QQHYTTPPT | | | 127 |
| 21496 | Full | EVQLVESGGGLVQPGRSLKLSCGASGFTFSDYYMAWVRQAPKKGLEWVASISYEGRSTYYGDSVKGRFTISRDNAKSTLYLQMNSLRSEDTATYYCARRAEGMDFDYWGQGVMVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCPPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYVIPPSKEQMAKDKVSLLCMITDFFPEDITVEWQWNGQPAENYLTWPPIMDTDGSYFVYSKLNVQKSNWEAGNTFTCSVLHEGLHNHHTEKSLSHSPGGGSGGGSGGGSGGGSGGGSDIVMTQTPASVEAAVGGTVTIKCQASQSIYSSLAWYQQKPGQSPKLLIYDASHLASGVPSRFSGSRYGTEFTLTISGVQCDDAATYYCQGGWYSSAATYVPNTFGGGTEVVVKGGGGSGGGGSGGGGSQEQLVESGGGLVQPEGSLTLTCKASGFTISNNYYMCWVRQAPGKGLEWIACIYGGISGRTYYASWAKGRFTISKTSSTTVTLQMTSLTAADTATYFCVRGYVGTSNLWGPGTLVTVSS | | | 128 |
| | VH | EVQLVESGGGLVQPGRSLKLSCGASGFTFSDYYMAWVRQAPKKGLEWVASISYEGRSTYYGDSVKGRFTISRDNAKSTLYLQMNSLRSEDTATYYCARRAEGMDFDYWGQGVMVTVSS | 158321 | 4-1BB | 129 |
| | HCDR1 | DYYMA | | | 130 |
| | HCDR2 | SISYEGRSTYYGDSVKG | | | 131 |
| | HCDR3 | RAEGMDFDY | | | 132 |
| | VH | QEQLVESGGGLVQPEGSLTLTCKASGFTISNNYYMCWVRQAPGKGLEWIACIYGGISGRTYYASWAKGRFTISKTSSTTVTLQMTSLTAADTATYFCVRGYVGTSNLWGPGTLVTVSS | 8K22 | FRa | 133 |
| | HCDR1 | NNYYMC | | | 134 |
| | HCDR2 | CIYGGISGRTYYASWAKG | | | 135 |
| | HCDR3 | GYVGTSNL | | | 136 |
| | VL | DIVMTQTPASVEAAVGGTVTIKCQASQSIYSSLAWYQQKPGQSPKLLIYDASHLASGVPSRFSGSRYGTEFTLTISGVQCDDAATYYCQGGWYSSAATYVPNTFGGGTEVVVK | 8K22 | FRa | 137 |
| | LCDR1 | QASQSIYSSLA | | | 138 |
| | LCDR2 | DASHLAS | | | 139 |
| | LCDR3 | QGGWYSSAATYVPNT | | | 140 |
| 22080 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALH NHYTQKSLSLSPG | | | 141 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 22082 | Full | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 142 |
| | Mask | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE | PD-133-146 | PD-L1 | 143 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 22083 | Full | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEMSGRSANAEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 144 |
| | Mask | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE | PD-133-146 | PD-L1 | 143 |
| | Mask Linker | MSGRSANAEAAAK | | | 145 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 22086 | Full | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEEAAAKEAAAKMSGRSANAQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGN VFSCSVMHEALHNHYTQKSLSLSPG | | | 146 |
| | Mask | NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE | PD-133-146 | PD-L1 | 143 |
| | Mask Linker | EAAAKEAAAKMSGRSANA | | | 147 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 22088 | Full | VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 148 |
| | Mask | VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKA | CD-80 V-set | | 149 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 22091 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 150 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAK | | | 114 |
| | VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT | hCris7 | CD3 | 103 |
| | LCDR1 | SASSSVSYMN | | | 104 |
| | LCDR2 | DSSKLAS | | | 105 |
| | LCDR3 | QQWSRNPPT | | | 106 |
| 22092 | Full | AFTVTVPKDLYVVEYGSNMTIECKFPVEKQLDLAALQVFWMMEDKNIIQFVHGEEDLKVQHSSYRQRARLLKDQLSLGNAALQITDVKLQDAGVYTCLIAYKGADYKRITVKVNAEAAAKEAAAKDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 151 |
| | Mask | AFTVTVPKDLYVVEYGSNMTIECKFPVEKQLDLAALQVFWMMEDKNIIQFVHGEEDLKVQHSSYRQRARLLKDQLSLGNAALQITDVKLQDAGVYTCLIAYKGADYKRITVKVNA | PD-L118-132 | PD-1 | 152 |
| | Mask Linker | EAAAKEAAAK | | | 114 |
| | VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT | hCris7 | CD3 | 103 |
| | LCDR1 | SASSSVSYMN | | | 104 |
| | LCDR2 | DSSKLAS | | | 105 |
| | LCDR3 | QQWSRNPPT | | | 106 |
| 22094 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKMSGRSANADIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLT LSKADYEKHKVYACEVTHQGLSSPVTKSFNRGEC | | | 153 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L118-132 | PD-1 | 113 |
| | Mask Linker | EAAAKMSGRSANA | | | 154 |
| | VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT | hCris7 | CD3 | 103 |
| | LCDR1 | SASSSVSYMN | | | 104 |
| | LCDR2 | DSSKLAS | | | 105 |
| | LCDR3 | QQWSRNPPT | | | 106 |
| 22105 | Full | MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEEAAAKEAAAKMSGRSANADIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDST YSLSSTLTLSKADYEKHKVYACEVTHQGLSSPVTKSFNRGEC | | | 155 |
| | Mask | MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE | CTLA4 38-155 IgV | | 156 |
| | Mask Linker | EAAAKEAAAKMSGRSANA | | | 147 |
| | VL | DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT | hCris7 | CD3 | 103 |
| | LCDR1 | SASSSVSYMN | | | 104 |
| | LCDR2 | DSSKLAS | | | 105 |
| | LCDR3 | QQWSRNPPT | | | 106 |
| 23246 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSL SSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 157 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA | Cetuxi mab | EGFR | 83 |
| | HCDR1 | NYGVH | | | 84 |
| | HCDR2 | VIWSGGNTDYNTPFTS | | | 85 |
| | HCDR3 | ALTYYDYEFAY | | | 86 |
| 23247 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 158 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L118-132 | PD-1 | 113 |
| | Mask Linker | EAAAKEAAAK | | | 114 |
| | VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK | Cetuxi mab | EGFR | 58 |
| | LCDR1 | RASQSIGTNIH | | | 59 |
| | LCDR2 | YASESIS | | | 60 |
| | LCDR3 | QQNNNWPTT | | | 61 |
| 23248 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 159 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAKMSGRSANA | | | 147 |
| | VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK | Cetuxi mab | EGFR | 58 |
| | LCDR1 | RASQSIGTNIH | | | 59 |
| | LCDR2 | YASESIS | | | 60 |
| | LCDR3 | QQNNNWPTT | | | 61 |
| 23253 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPE PVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 160 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS | huRG7 787 | Mesoth elin | 68 |
| | HCDR1 | GYTMN | | | 69 |
| | HCDR2 | LITPYNGASSYNQKFRG | | | 70 |
| | HCDR3 | GGYDGRGFDY | | | 71 |
| 23256 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQW KVDNALQSGNSQESVTEQDSKDSTYSLSSTLTLSKADYEKHKVYACEVTHQGLSSPVTKSFNRGEC | | | 161 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAKMSGRSANA | | | 147 |
| | VL | DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK | huRG7 787 | Mesoth elin | 73 |
| | LCDR1 | SASSSVSYMH | | | 74 |
| | LCDR2 | DTSKLAS | | | 75 |
| | LCDR3 | QQWSKHPLT | | | 76 |
| 23257 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSSASTKGPSVFPLAPSSKSTSGGTAALGCLVKDYFP EPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 162 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-1 33-146 | PD-L1 | 116 |
| | Mask Linker | EAAAKEAAAK | | | 114 |
| | VH | QVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSS | SGN-CD19a | CD19 | 63 |
| | HCDR1 | TSGMGVG | | | 64 |
| | HCDR2 | HIWWDDDKRYNPALKS | | | 65 |
| | HCDR3 | MELWSYYFDY | | | 66 |
| 23258 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 163 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAK | | | 114 |
| | VL | EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIK | SGN-CD19a | CD19 | 164 |
| | LCDR1 | SASSSVSYMH | | | 74 |
| | LCDR2 | DTSKLAS | | | 75 |
| | LCDR3 | FQGSVYPFT | | | 165 |
| 23260 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANAEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 166 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAKMSGRSANA | | | 147 |
| | VL | EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIK | SGN-CD19a | CD19 | 164 |
| | LCDR1 | SASSSVSYMH | | | 74 |
| | LCDR2 | DTSKLAS | | | 75 |
| | LCDR3 | FQGSVYPFT | | | 165 |
| 23261 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYS KLTVDKSRWQQGNVFSCSVMHEALHNHYTQKSLSLSPG | | | 167 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | EVQLVESGGGLVQPGGSLRLSCAASGFNIKEYYMHWVRQAPGKGLEWVGLIDPEQGNTIYDPKFQDRATISADNSKNTAYLQM | D3H44 | TF | 53 |
| | | NSLRAEDTAVYYCARDTAAYFDYWGQGTLVTVSS | | | |
| | HCDR1 | EYYMH | | | 54 |
| | HCDR2 | LIDPEQGNTIYDPKFQD | | | 55 |
| | HCDR3 | DTAAYFDY | | | 56 |
| 23262 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 168 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L118-132 | PD-1 | 113 |
| | Mask Linker | EAAAKEAAAK | | | 114 |
| | VL | DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIK | D3H44 | TF | 47 |
| | LCDR1 | RASRDIKSYLN | | | 48 |
| | LCDR2 | YATSLAE | | | 49 |
| | LCDR3 | LQHGESPWT | | | 50 |
| 23264 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 169 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L118-132 | PD-1 | 113 |
| | Mask Linker | EAAAKEAAAKMSGRSANA | | | 147 |
| | VL | DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIK | D3H44 | TF | 47 |
| | LCDR1 | RASRDIKSYLN | | | 48 |
| | LCDR2 | YATSLAE | | | 49 |
| | LCDR3 | LQHGESPWT | | | 50 |
| 23567 | Full | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 170 |
| | VH | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA | Cetuxi mab | EGFR | 83 |
| | HCDR1 | NYGVH | | | 84 |
| | HCDR2 | VIWSGGNTDYNTPFTS | | | 85 |
| | HCDR3 | ALTYYDYEFAY | | | 86 |
| 23712 | Full | QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 171 |
| | VH | QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS | CP-870\|893 | CD40 | 172 |
| | HCDR1 | GYYMH | | | 173 |
| | HCDR2 | WINPDSGGTNYAQKFQG | | | 174 |
| | HCDR3 | DQPLGYCTNGVCSYFDY | | | 175 |
| 23713 | Full | DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 176 |
| | VL | DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK | CP-870\|89 3 | CD40 | 177 |
| | LCDR1 | RASQGIYSWLA | | | 178 |
| | LCDR2 | TASTLQS | | | 179 |
| | LCDR3 | QQANIFPLT | | | 180 |
| 23714 | Full | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 181 |
| | Mask | NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE | PD-133-146 | PD-L1 | 116 |
| | Mask Linker | EAAAKEAAAK | | | 114 |
| | VH | QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS | CP-870\|89 3 | CD40 | 172 |
| | HCDR1 | GYYMH | | | 173 |
| | HCDR2 | WINPDSGGTNYAQKFQG | | | 174 |
| | HCDR3 | DQPLGYCTNGVCSYFDY | | | 175 |
| 23715 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSG TASVVCLLNNFYPREAKVQWKVDNALQSGNSQESVTEQDSKDSTYSLSSTLTLSKADYEKHKVYACEVTHQGLSSPVTKSFNRGEC | | | 182 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAK | | | 114 |
| | VL | DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK | CP-870\|89 3 | CD40 | 177 |
| | LCDR1 | RASQGIYSWLA | | | 178 |
| | LCDR2 | TASTLQS | | | 179 |
| | LCDR3 | QQANIFPLT | | | 180 |
| 23716 | Full | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 183 |
| | Mask | AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNA | PD-L1 18-132 | PD-1 | 113 |
| | Mask Linker | EAAAKEAAAKMSGRSANA | | | 147 |
| | VL | DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK | CP-870\|89 3 | CD40 | 177 |
| | LCDR1 | RASQGIYSWLA | | | 178 |
| | LCDR2 | TASTLQS | | | 179 |
| | LCDR3 | QQANIFPLT | | | 180 |
| 24659 | Full | VIHVTKEVKEVATLSCGHNVSVEELAQTRIYWQKEKKMVLTMMSGDMNIWPEYKNRTSFDITNNLSISISALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDY NGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 184 |
| | Mask | VIHVTKEVKEVATLSCGHNVSVEELAQTRIYWQKEKKMVLTMMSGDMNIWPEYKNRTSFDITNNLSISISALRPSDEGTYECVVLKYEKDAFKREHLAEVTLSVKA | CD-80 V-set | | 185 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 24660 | Full | VIHVTKEVKEVATLSCGHNVSVEELAQTRIYWQKEKKMVLTMMSGDSNIWPEYKNRTIFDSTNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDY NGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 186 |
| | Mask | VIHVTKEVKEVATLSCGHNVSVEELAQTRIYWQKEKKMVLTMMSGDSNIWPEYKNRTIFDSTNNLSIVILALRPSDEGTYECVVLKYEKDAFKREHLAEVTLSVKA | CD-80 V-set | | 187 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 24661 | Full | VIHVTKEVKEVATLSCGHNVSSEELAQTRIYWQKEKKMVLTMMSGDMNIWPEYKNRTIFDITNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDY NGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 188 |
| | Mask | VIHVTKEVKEVATLSCGHNVSSEELAQTRIYWQKEKKMVLTMMSGDMNIWPEYKNRTIFDITNNLSIVILALRPSDEGTYECVVLKYEKDAFKREHLAEVTLSVKA | CD-80 V-set | | 189 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| 23734 | Full | NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQ VYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG | | | 190 |
| | Mask | NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTE | PD-1 33-146 | PD-L1 | 191 |
| | Mask Linker | EAAAKEAAAK | | | 114 |
| | VH | QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS | hCris7 | CD3 | 108 |
| | HCDR1 | RSTMH | | | 109 |
| | HCDR2 | YINPSSAYTNYNQKFKD | | | 110 |
| | HCDR3 | PQVHYDYNGFPY | | | 111 |
| | LCDR2 | RSYQRPS | | | 199 |
| | LCDR3 | ATWDDSLDGWV | | | 200 |
| 11018 | Full | GQVQLVQSGAEVKKPGASVRVSCRASGYIFTESGITWVRQAPGQGLEWMGWISGYSGDTKYAQKLQGRVTMTKDTSTTTAYMELRSLRYDDTAVYYCARDVQYSGSYLGAYYFDYWSPGTLVTVSSGGGGSGGGGSGGGGSGGGQSVLTQPPSASGTPGQRVTISCSGSSSNIGTNYVYWYQQFPGTAPKLLIYRSYQRPSGVPDRFSGSKSGSSASLAISGLQSEDEADYYCATWDDSLDGWVFGGGTKLTVLAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG | | | 192 |
| | VH | QVQLVQSGAEVKKPGASVRVSCRASGYIFTESGITWVRQAPGQGLEWMGWISGYSGDTKYAQKLQGRVTMTKDTSTTTAYMELRSLRYDDTAVYYCARDVQYSGSYLGAYYFDYWSPGTLVTVSS | CR807 1 | Hemag glutinin | 193 |
| | VL | QSVLTQPPSASGTPGQRVTISCSGSSSNIGTNYVYWYQQFPGTAPKLLIYRSYQRPSGVPDRFSGSKSGSSASLAISGLQSEDEADYYCATWDDSLDGWVFGGGTKLTVL | CR807 1 | Hemag glutinin | 194 |
| | HCDR1 | ESGIT | | | 195 |
| | HCDR2 | WISGYSGDTKYAQKLQG | | | 196 |
| | HCDR3 | DVQYSGSYLGAYYFDY | | | 197 |
| | LCDR1 | SGSSSNIGTNYVY | | | 198 |
| | LCDR2 | RSYQRPS | | | 199 |
| | LCDR3 | ATWDDSLDGWV | | | 200 |
| 25321 | Full | EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRAMSGRSANAQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG | | | 201 |
| | Mask | EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRA | SIRPa_ d1_v2 | CD47 | 202 |
| | Mask Linker | MSGRSANA | | | 203 |
| | VH | QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA | Cetuxi mab | EGFR | 83 |
| | HCDR1 | NYGVH | | | 84 |
| | HCDR2 | VIWSGGNTDYNTPFTS | | | 85 |
| | HCDR3 | ALTYYDYEFAY | | | 86 |
| 25325 | Full | QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVMSGRSANADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC | | | 204 |
| | Mask | QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRV | CD47 | | 205 |
| | Mask Linker | MSGRSANA | | | 203 |
| | VL | DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK | Cetuxi mab | EGFR | 58 |
| | LCDR1 | RASQSIGTNIH | | | 59 |
| | LCDR2 | YASESIS | | | 60 |
| | LCDR3 | QQNNNWPTT | | | 61 |

TABLE BB

anti-CD3 Paratope Sequences

| Anti-CD3 paratope | Sequence Type | Sequence | Seq ID No. |
|---|---|---|---|
| 1 | VH | EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS | 206 |
| | Kabat HCDR1 | YYGMS | 207 |
| | Kabat HCDR2 | SITSSGGRIYYPDSVKG | 208 |
| | Kabat HCDR3 | DGRDGWVAY | 209 |
| | VL | NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVL | 210 |
| | Kabat LCDR1 | KRNTGNIGSNYVN | 211 |
| | Kabat LCDR2 | RNDKRPD | 212 |
| | Kabat LCDR3 | QSYSSGFI | 214 |
| 2 | | | |
| | VH | EVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSS | 215 |
| | Kabat HCDR1 | YYGMS | 216 |
| | Kabat HCDR2 | SITRSGGRIYYPDSVKG | 217 |
| | Kabat HCDR3 | DGRDGWVAY | 218 |
| | VL | NFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVL | 219 |
| | Kabat LCDR1 | TGNTGNIGSNYVN | 220 |
| | Kabat LCDR2 | RDDKRPS | 221 |
| | Kabat LCDR3 | QSYSSGFI | 222 |
| 3 | | | |
| | VH | EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS | 223 |
| | Kabat HCDR1 | KYAMN | 224 |
| | Kabat HCDR2 | RIRSKYNNYATYYADSVKD | 225 |
| | Kabat HCDR3 | HGNFGNSYISYWAY | 226 |
| | VL | QTVVTQEPSLTVSPGGTVTLTCGSSTGA VTSGNYPNWVQQKPGQAPRGLIGGTKF LAPGTPARFSGSLLGGKAALTLSGVQPE DEAEYYCVLWYSNRWVFGGGTKLTVL | 227 |
| | Kabat LCDR1 | GSSTGAVTSGNYPN | 228 |
| | Kabat LCDR2 | GTKFLAP | 229 |
| | Kabat LCDR3 | VLWYSNRWV | 230 |
| 4 | | | |
| | VH | EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS | 231 |
| | Kabat HCDR1 | TYAMN | 232 |
| | Kabat HCDR2 | RIRSKYNNYATYYADSVKG | 233 |
| | Kabat HCDR3 | HGNFGNSYVSWFAY | 234 |
| | VL | QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL | 235 |
| | Kabat LCDR1 | GSSTGAVTTSNYAN | 236 |
| | Kabat LCDR2 | GTNKRAP | 237 |
| | Kabat LCDR3 | ALWYSNLWV | 238 |
| 5 | VH | QVQLVQSGAEVKKPGASVKVSCKASGYTFTRSTMHWVRQAPGQGLEWIGYINPSSAYTNYNQKFKDRVTITADKSTSTAYMELSSLRSEDTAVYYCASPQVHYDYNGFPYWGQGTLVTVSS | 239 |
| | Kabat HCDR1 | RSTMH | 240 |
| | Kabat HCDR2 | YINPSSAYTNYNQKFKD | 241 |
| | Kabat HCDR3 | PQVHYDYNGFPY | 242 |
| | VL | DIQMTQSPSSLSASVGDRVTITCSASSSVSYMNWYQQKPGKAPKRLIYDSSKLASGVPSRFSGSGSGTEFTLTISSLQPEDFATYYCQQWSRNPPTFGGGTKVEIK | 243 |
| | Kabat LCDR1 | SASSSVSYMN | 244 |
| | Kabat LCDR2 | DSSKLAS | 245 |
| | Kabat LCDR3 | QQWSRNPPT | 246 |

TABLE CC

IgSF IgV Domain Sequences

| IgSF Member Name | Uniprot ID | Residues | Amino Acid Sequence of IgV Domain | SEQ ID NO |
|---|---|---|---|---|
| CD80 | P33681 | 35-135 | VIHVTK EVKEVATLSC GHNVSVEELA QTRIYWQKEKKMVLTMMSGDMNIW PEYKNRTIFDITNNLSIVILALRPSD EGTYECVVLK YEKDAFKREH LAEVT | 247 |
| CD86 | P42081 | 33-131 | NETADLPC QFANSQNQSLSELVVFWQDQ ENLVLNEVYL GKEKFDSVHS KYMGRTSFDS DSWTLRLHNLQIKDKGLYQC IIHHKKPTGM IRIHQMNSEL S | 248 |
| PD-L1 | Q9NZQ7 | 19-127 | FT VTVPKDLYVV EYGSNMTIEC KFPVEKQLDLAALIVYWEME DKNIIQFVHG EEDLKVQHSSYRQRARLLKDQLSLG NAALQITDVKLQDAG VYRCMISYGG ADYKRIT | 249 |
| PD-L2 | Q9BQ51 | 21-118 | FTVTVPKELY IIEHGSNVTL ECNFDTGSHV NLGAITASLQ KVENDTSPHR ERATLLEEQL PLGKASFHIP QVQVRDEGQYQCIIIYGVAW DYKYLTLK | 250 |
| CTLA-4 | P16410 | 39-140 | HV AQPAVVLASSRGIASFVCEY ASPGKATEVR VTVLRQADSQ VTEVCAATYM MGNELTFLDDSICTGTSSGN QVNLTIQGLR AMDTGLYICK VELMYPPPYY | 251 |
| PD-1 | Q15116 | 35-145 | PTFSPA LLVVTEGDNATFTCSFSNTS ESFVLNWYRM SPSNQTDKLA AFPEDRSQPG QDCRFRVTQLPNGRDFHMSV VRARRNDSGT YLCGAISLAP KAQIKESLRA ELRVT | 252 |
| CD28 | P10747 | 28-137 | MLVAYDNAVNLSCKYSYNLFSREFR ASLHKGLDSAVEVCVVYGNYSQQLQ VYSKTGFNCDGKL GNESVTFYLQNLYVNQTDIY FCKIEVMYPP PYLDNEKSNG TIIHVKG | 253 |
| CD47 | Q08722 | 19-127 | QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETII | 254 |
| SIRPa | P78324 | 32-137 | EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSG | 255 |
| ICOSL | 075144 | 19-129 | DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVE | 256 |
| ICOS | Q9Y6W8 | 30-132 | MFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYE | 257 |
| CD276 | Q5ZPR3 | 29-139 | LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVA | 258 |
| VTCN1 35-146 | Q7Z7D3 | 35-146 | HSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYK | 259 |
| VISTA | Q9H7M9 | 33-168 | FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV | 260 |
| NCR3LG1 | Q68D85 | 27-138 | KVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLE | 261 |
| HHLA2 | Q9UM44 | 61-131 | IHWKYQDSYKVHSYYKGSDHLESQDPRYANRTSLFYNEIQNGNASLFFRRVSLLDEGIYTCYVGTAIQVIT | 262 |
| CD28H | Q96BF3 | 23-129 | LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNIT | 263 |
| NKp30 | 014931 | 19-126 | LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLV | 264 |
| Mask linker | | | GPPPG | 265 |
| Mask linker | | | GGPPPGG | 266 |
| Mask linker | | | GPPPPG | 267 |
| Mask linker | | | GGPPPGG | 268 |

We claim:

1. A fusion protein comprising:
a biologically functional protein, a ligand-receptor pair, a first peptidic linker and a second peptidic linker; wherein
the biologically functional protein comprises at least a first polypeptide and a second polypeptide; and
the ligand-receptor pair comprises an extracellular portion of an immunoglobulin superfamily receptor and its cognate ligand or a receptor-binding fragment thereof; wherein
the ligand is fused to a terminus of the first polypeptide via the first peptidic linker;
the receptor is fused to the same respective terminus of the second polypeptide via the second peptidic linker; and the first and second peptidic linkers are of sufficient length to allow pairing of the ligand and receptor, and at least one of the first and second peptidic linkers comprises a protease cleavage site.

2. The fusion protein according to claim 1, wherein the ligand and the receptor comprise an extracellular portion of an immunoglobulin superfamily (IgSF) polypeptide.

3. The fusion protein according to claim 1, wherein the ligand and the receptor comprise an extracellular portion of an immunoglobulin variable (IgV) polypeptide.

4. The fusion protein according to any one of claims 1-3, wherein the biologically functional protein comprises an antibody or antigen-binding antibody fragment.

5. The fusion protein according to claim 1, wherein the biologically functional protein consists of a polypeptide scaffold.

6. The fusion protein according to claim 5, wherein the polypeptide scaffold is a dimeric Fc region, wherein the first polypeptide consists of a first Fc polypeptide and the second polypeptide consists of a second Fc polypeptide, the first and second Fc polypeptides forming the dimeric Fc region.

7. The fusion protein according to claim 1, wherein the biologically functional protein comprises a polypeptide scaffold.

8. The fusion protein according to claim 7, wherein the polypeptide scaffold comprises a dimeric Fc region.

9. The fusion protein according to one of claims 6 or 8, wherein the dimeric Fc region is a heterodimeric Fc.

10. The fusion protein according to any one of the above claims, wherein at least one of the ligand or the receptor in the ligand-receptor pair is capable of binding to an immunomodulatory target.

11. The fusion protein according to any one of the above claims, wherein the ligand receptor pair is involved in a cellular response selected from the group consisting of: modulation of an immune checkpoint, modulation of immune cell activity, modulation of T-cell receptor signaling, modulation of T-cell dependent cytotoxicity (TDCC), modulation of antibody-dependent cellular phagocytosis (ADCP) and modulation of antibody-dependent cellular cytotoxicity (ADCC).

12. The fusion protein according to any one of the above claims, wherein the receptor comprises one or more mutations that increase or decrease binding affinity of the receptor for its cognate ligand as compared to a wild-type receptor.

13. The fusion protein according to any one of the above claims, wherein the ligand comprises one or more mutations that increase or decrease binding affinity of the ligand for its cognate receptor as compared to a wild-type ligand.

14. The fusion protein according to any one of the above claims, wherein the ligand-receptor pair is selected from the group consisting of: PD1-PDL1, PD1-PDL2, CTLA4-CD80, CD28-CD80, CD28-CD86, CTLA4-CD86, PDL1-CD80, ICOS-ICOSL, NCRSRLG1-NKp30 and CD47-SIRPa.

15. The fusion protein according to claim 14, wherein the ligand-receptor pair is PD1-PDL1.

16. The fusion protein according to claim 15, wherein the ligand PDL1 comprises an amino acid sequence according to SEQ ID NO: 8.

17. The fusion protein according to claim 15 or claim 16, wherein the receptor PD1 comprises an amino acid sequence according to SEQ ID NO: 9.

18. The fusion protein according to claim 14, wherein the ligand-receptor pair is CTLA4-CD80.

19. The fusion protein according to claim 18, wherein the ligand CD80 comprises an amino acid sequence according to SEQ ID NO: 25, SEQ ID NO: 185, SEQ ID NO: 187 or SEQ ID NO: 189.

20. The fusion protein according to claim 18 or 19, wherein the receptor CTLA4 comprises an amino acid sequence according to SEQ ID NO: 26.

21. The fusion protein according to claim 14, wherein the ligand-receptor pair is selected from the group consisting of: CTLA4-CD80, PDL1-CD80 and CD28-CD80 and wherein the ligand CD80 comprises an amino acid sequence according to SEQ ID NO:25 having mutations selected from the group consisting of:
(a) H18Y, A26E, E35D, M47S, I61S and D90G; (b) E35D, M47S, N48K, I61S, K89N; (c) E35D, D46V, M47S, I61S, D90G, K93E; or (d) H18Y, A26E, E35D, M47S, I61S, V68M, A71G, D90G; (e) I58S, V68S, L70S; (f) M47S, I61S or (g) V22S.

22. The fusion protein according to any one of the above claims, wherein the receptor and the ligand are fused to the respective N- termini of the first and second polypeptides.

23. The fusion protein according to any one of the above claims, wherein one of the first or second peptidic linkers comprises more than one protease cleavage site.

24. The fusion protein according to any one of the above claims, wherein one of the peptidic linkers fused to the ligand or the receptor is engineered to comprise one or more additional protease cleavage sites, and wherein the one or more protease cleavage sites in the ligand or the receptor and the protease cleavage site in the first or second peptidic linker are cleavable by the same protease or a different protease.

25. The fusion protein according to any one of the above claims, wherein the protease is selected from the group consisting of: a serine protease, MMP1, MMP2, MMP3, MMP7, MMP8, MMP9, MMP10, MMP11, MMP12, MMP13, MMP14, MMP15, MMP16, MMP17, MMP18 (collagenase 4), MMP19, MMP20, MMP21, an adamalysin, a serralysin, an astacin, caspase 1, caspase 2, caspase 3, caspase 4, caspase 5, caspase 6, caspase 7, caspase 8, caspase 9, caspase 10, caspase 11, caspase 12, caspase 13, caspase 14, cathepsin A, cathepsin B, cathepsin D, cathepsin E, cathepsin K, cathepsin S, granzyme B, guanidinobenzoatase (GB), hepsin, elastase, legumain, matriptase, matriptase 2, meprin, neurosin, MT-SP1, neprilysin, plasmin, PSA, PSMA, TACE, TMPRSS3, TMPRSS4, uPA, calpain, FAP and KLK.

26. The fusion protein according to claim 25, wherein the protease is uPA or matriptase.

27. The fusion protein according to any one of the above claims, wherein the peptidic linker is 3-50 or 5-20 amino acids in length.

28. The fusion protein according to any one of the above claims, wherein one of the first or second peptidic linkers does not have a protease cleavage site.

29. The fusion protein any one of the above claims, wherein the peptidic linker is a (GlyₙSer) linker, wherein the (GlyₙSer) linker comprises an amino acid sequence selected from the group consisting of (Gly₃Ser)ₙ(Gly₄Ser)₁, (Gly₃Ser)₁(Gly₄Ser)ₙ, (Gly₃Ser)ₙ(Gly₄Ser)ₙ, and (Gly₄Ser)ₙ, wherein n is an integer of 1 to 5.

30. The fusion protein any one of the above claims, wherein the peptidic linker is an (EAAAK)ₙ linker, wherein n is an integer between 1 and 5.

31. The fusion protein according to claim 30, wherein the peptidic linker comprises the amino acid sequence EAAAKEAAAK (SEQ ID. NO: 38).

32. The fusion protein any one of the above claims, wherein the peptidic linker is a polyproline linker, optionally PPP or PPPP, or a glycine-proline linker, optionally GPPPG. GGPPPGG, GPPPPG or GGPPPPGG.

33. The fusion protein any one of the above claims, wherein the peptidic linker comprises an immunoglobulin hinge region sequence comprising an amino acid sequence having up to a 30 percent difference in amino acid sequence identity compared to a wild type immunoglobulin hinge region amino acid sequence.

34. The fusion protein according to any one of the above claims, wherein the peptidic linker comprises a protease cleavage site comprising the amino acid sequence MSGRSANA (SEQ ID NO: 28).

35. The fusion protein according to any one of claims 1 to 4, wherein at least one of the first and second polypeptides comprise a first VH polypeptide and a first VL polypeptide, the first VH and VL polypeptides forming a first antigen-binding domain of the antibody, wherein the ligand is fused to one of the first VH or VL polypeptides via the first peptidic linker and the receptor is fused to the other of the first VH or VL polypeptides via the second peptidic linker, and wherein the ligand-receptor pair sterically hinders binding of the first antigen-binding domain to its cognate antigen.

36. The fusion protein according to claim 35, wherein the first and second polypeptides further comprise a dimeric Fc.

37. The fusion protein according to claim 36, wherein the dimeric Fc region is a heterodimeric Fc.

38. The fusion protein according to any one of claims 35-37, wherein at least one of the ligand or the receptor of the ligand-receptor pair is capable of binding to an immunomodulatory target.

39. The fusion protein according to any one of claims 35-38, wherein the ligand receptor pair is involved in a cellular response selected from the group consisting of: modulation of an immune checkpoint, modulation of immune cell activity, modulation of T-cell receptor signaling, modulation of T-cell dependent cytotoxicity (TDCC), modulation of antibody-dependent cellular phagocytosis (ADCP) and modulation of antibody-dependent cellular cytotoxicity (ADCC).

40. The fusion protein according to any one of claims 35-38, wherein the receptor comprises one or more mutations that increase or decrease binding affinity of the receptor for its cognate ligand as compared to a wild-type receptor.

41. The fusion protein according to any one of claims 35-40, wherein the ligand comprises one or more mutations that increase or decrease binding affinity of the ligand for its cognate receptor as compared to a wild-type ligand.

42. The fusion protein according to any one of claims 35-41, wherein the ligand-receptor pair is selected from the group consisting of: PD1-PDL1, PD1-PDL2, CTLA4-CD80, CD28-CD80, CD28-PDL1, CD28-CD86, CTLA4-CD86, PDL1-CD80, ICOS-ICOSL, NCRSRLG1-NKp30 and CD47-SIRPa.

43. The fusion protein according to claim 42, wherein the ligand-receptor pair is PD1-PDL1.

44. The fusion protein according to claim 43, wherein the ligand PD-L1 comprises an amino acid sequence according to SEQ ID NO: 8.

45. The fusion protein according to claim 43 or claim 44, wherein the receptor PD1 comprises an amino acid sequence according to SEQ ID NO: 9.

46. The fusion protein according to claim 42, wherein the ligand-receptor pair is CTLA4-CD80.

47. The fusion protein according to claim 46, wherein the ligand CD80 comprises an amino acid sequence according to SEQ ID NO: 25.

48. The fusion protein according to claim 42, wherein the ligand-receptor pair is selected from the group consisting of: CTLA4-CD80, PDL1-CD80 and CD28-CD80 wherein the ligand CD80 comprises an amino acid sequence according to SEQ ID NO: 25 having mutations selected from the group consisting of:
(a) H18Y, A26E, E35D, M47S, I61S and D90G; (b) E35D, M47S, N48K, I61S, K89N; (c) E35D, D46V, M47S, 161S, D90G, K93E; (d) H18Y, A26E, E35D, M47S, 161S, V68M, A71G, D90G; (e) I58S, V68S, L70S; (f) M47S, I61S or (g) V22S.

49. The fusion protein according to any one of claims 46 to 48, wherein the receptor CTLA4 comprises an amino acid sequence according to SEQ ID NO: 26.

50. The fusion protein according to claim 48, wherein the ligand-receptor pair is PDL1-CD80 and the PDL1 comprises an amino acid sequence according to SEQ ID NO: 8.

51. The fusion protein according to claim 48, wherein the ligand-receptor pair is CD28-CD80 and the CD28 comprises an amino acid sequence according to SEQ ID NO: 254.

52. The fusion protein according to claim 43, wherein the ligand-receptor pair is CD28-PDL1.

53. The fusion protein according to claim 52, wherein the CD28 comprises an amino acid sequence according to SEQ ID NO: 254.

54. The fusion protein according to claim 52 or 53, wherein the PDL1 comprises an amino acid sequence according to SEQ ID NO: 8.

55. The fusion protein according to claim 42, wherein the ligand-receptor pair is CD47-SIRPa.

56. The fusion protein according to claim 55, wherein the SIRPa comprises an amino acid sequence according to SEQ ID NO: 255.

57. The fusion protein according to claim 55 or 56 wherein the CD47 comprises an amino acid sequence according to SEQ ID NO: 254.

58. The fusion protein according to any one of claims 35-57, wherein the receptor and the ligand are fused to the respective N- termini of the first and second polypeptides.

59. The fusion protein according to any one of claims 35-58, wherein one of the first or second peptidic linkers comprises more than one protease cleavage site.

60. The fusion protein according to any one of claims 35-59, wherein one of the ligand or the receptor is engineered to comprise one or more additional protease cleavage sites, and wherein the one or more protease cleavage sites in the ligand or the receptor and the protease cleavage site in the first or second peptidic linker are cleavable by the same protease or by different proteases.

61. The fusion protein according to any one of claims 35-60, wherein the protease is selected from the group consisting of: a serine protease, MMP1, MMP2, MMP3, MMP7, MMP8, MMP9, MMP10, MMP11, MMP12, MMP13, MMP14, MMP15, MMP16, MMP17, MMP18 (collagenase 4), MMP19, MMP20, MMP21, an adamalysin, a serralysin, an astacin, caspase 1, caspase 2, caspase 3, caspase 4, caspase 5, caspase 6, caspase 7, caspase 8, caspase 9, caspase 10, caspase 11, caspase 12, caspase 13, caspase 14, cathepsin A, cathepsin B, cathepsin D, cathepsin E, cathepsin K, cathepsin S, granzyme B, guanidinobenzoatase (GB), hepsin, elastase, legumain, matriptase, matriptase 2, meprin, neurosin, MT-SP1, neprilysin, plasmin, PSA, PSMA, TACE, TMPRSS3, TMPRSS4, uPA, calpain, FAP and KLK.

62. The fusion protein according to claim 61, wherein the protease is uPA or matriptase.

63. The fusion protein according to any one of claims 35-62, wherein the peptidic linker is 3-50 or 5-20 amino acids in length.

64. The fusion protein according to any one of claims 35-63, wherein one of the first or second peptidic linkers does not have a protease cleavage site.

65. The fusion protein according to any one of claims 35-64, wherein the peptidic linker is a (GlyₙSer) linker, wherein the (GlyₙSer) linker comprises an amino acid sequence selected from the group consisting of (Gly₃Ser)ₙ(Gly₄Ser)₁, (Gly₃Ser)₁(Gly₄Ser)ₙ, (Gly₃Ser)ₙ(Gly₄Ser)ₙ, and (Gly₄Ser)ₙ, wherein n is an integer of 1 to 5.

66. The fusion protein according to any one of claims 35-64, wherein the peptidic linker is an (EAAAK)ₙ linker, wherein n is an integer between 1 and 5.

67. The fusion protein according to claim 64, wherein the peptidic linker that does not have a protease cleavage site comprises the amino acid sequence EAAAKEAAAK (SEQ ID. NO: 38).

68. The fusion protein according to claim 51 or 52 wherein the peptidic linker is a polyproline linker, optionally PPP or PPPP, or a glycine-proline linker, optionally GPPPG. GGPPPGG, GPPPPG or GGPPPPGG.

69. The fusion protein according to any one of claims 35-64, wherein the peptidic linker comprises an immunoglobulin hinge region sequence comprising an amino acid sequence having up to a 30 percent difference in amino acid sequence identity compared to a wild type immunoglobulin hinge region amino acid sequence.

70. The fusion protein according to any one of claims 35-69, wherein the peptidic linker comprising a protease cleavage site comprises the amino acid sequence MSGRSANA (SEQ ID NO: 28).

71. The fusion protein according any one of claims 35-70, wherein binding of the first antigen-binding domain to its cognate antigen is reduced by 10-fold or more as compared to a parental antigen-binding domain that is not fused to the ligand-receptor pair.

72. The fusion protein according to any one of claims 35-71, wherein cleavage of the protease cleavage site in a cellular environment releases one member of the ligand-receptor pair from the fusion protein, thereby allowing the antigen-binding domain to bind its cognate antigen.

73. The fusion protein according to any one of claims 35-72, wherein the first antigen-binding domain is a Fab.

74. The fusion protein according to any one of claims 35-73, wherein the first antigen-binding domain binds an antigen that is expressed on a cancer cell or an immune cell.

75. The fusion protein according to any one of claims 35-74, wherein the first antigen-binding domain binds an antigen that is expressed on a T-cell.

76. The fusion protein according to any one of claims 35-74, wherein the first antigen- binding domain binds to a tumor-associated antigen (TAA).

77. The fusion protein according to any one of claims 35-74, wherein the first antigen binding domain binds to a TAA and wherein at least one of the ligand or the receptor in the ligand-receptor pair is capable of binding to an immunomodulatory target.

78. The fusion protein according to any one of claims 35-77, wherein the first antigen-binding domain binds to an antigen selected from the group consisting of: Cluster of Differentiation 3 (CD3), Human Epidermal Growth Factor Receptor 2 (HER2), Epidermal Growth Factor Receptor (EGFR), Mesothelin (MSLN), Tissue Factor (TF), Cluster of Differentiation 19 (CD19), tyrosine-protein kinase Met (c-Met), and Cadherin 3 (CDH3).

79. The fusion protein of any one of claims 32-78, wherein the antibody or antibody fragment comprises a second antigen binding domain comprising a second VH polypeptide and a second VL polypeptide.

80. The fusion protein of claim 79, wherein the fusion protein comprises a second ligand-receptor pair, wherein the ligand of the second ligand-receptor pair is fused to one of the second VH or VL polypeptides via a third peptidic linker and the receptor of the second ligand-receptor pair is fused to the other of the second VH or VL polypeptides via a fourth peptidic linker, wherein at least one of the third and fourth peptidic linkers comprise a protease cleavage site, and wherein the ligand-receptor pair sterically hinders binding of the second antigen-binding domain to its cognate antigen.

81. The fusion protein according to claim 79 or claim 80, wherein the fusion protein binds to two distinct antigens.

82. The fusion protein according to claim 81, wherein one antigen is an antigen expressed by T cells and the other antigen is an antigen expressed by cancer cells.

83. The fusion protein according to claim 82, wherein the antigen expressed by T cells is CD3.

84. The fusion protein according to claim 83 comprising (a) an anti-CD3 paratope comprising a VH and a VL, wherein the VH comprises three CDRs HCDR1, HCDR2 and HCDR3 and the VL comprises three CDRs LCDR1, LCDR2 and LCDR3, wherein
(a) HCDR1, HCDR2 and HCDR3 are SEQ ID NOS: 207, 208 and 209 respectively, and LCDR1, LCDR2 and LCDR3 are 211, 212 and 214 respectively;
(b) HCDR1, HCDR2 and HCDR3 are SEQ ID NOS: 224, 225 and 226 respectively, and LCDR1, LCDR2 and LCDR3 are 228, 229 and 230 respectively;
(c) HCDR1, HCDR2 and HCDR3 are SEQ ID NOS: 232, 233 and 234 respectively, and LCDR1, LCDR2 and LCDR3 are 236, 237 and 238 respectively; or
(d) HCDR1, HCDR2 and HCDR3 are SEQ ID NOS: 240, 241 and 242 respectively, and LCDR1, LCDR2 and LCDR3 are 244, 245 and 246 respectively.

85. The fusion protein according to any one of claims 81-84, wherein the fusion protein binds to CD3 and HER2.

86. A fusion protein comprising:
an Fc region comprising a first Fc polypeptide and a second Fc polypeptide, and
a ligand-receptor pair comprising an extracellular portion of an immunoglobulin superfamily receptor and its cognate ligand or a receptor-binding fragment thereof; wherein
the ligand is fused to a terminus of the first Fc polypeptide via a first peptidic linker and the receptor is fused to the same respective terminus of the second Fc polypeptide via a second peptidic linker; wherein
the first and second peptidic linkers are of sufficient length to allow pairing of the ligand and receptor; and wherein
at least one of the first and second peptidic linkers comprises a protease cleavage site.

87. A fusion protein comprising:
a biologically functional protein, a ligand-receptor pair, a first peptidic linker and a second peptidic linker; wherein
the biologically functional protein comprises at least a first polypeptide and a second polypeptide; and
the ligand-receptor pair comprises an extracellular portion of an immunoglobulin superfamily receptor and its cognate ligand or a receptor-binding fragment thereof; wherein
the ligand is fused to a terminus of the first polypeptide via the first peptidic linker;
the receptor is fused to the same respective terminus of the second polypeptide via the second peptidic linker; and the first and second peptidic linkers are of sufficient length to allow pairing of the ligand and receptor.

88. The fusion protein according to claim 86, wherein the ligand and receptor are fused to the respective N-termini of the first and second Fc polypeptides.

89. A fusion protein comprising:
a Fab region and an Fc region; wherein
the Fab region comprises a VH polypeptide and a VL polypeptide that form an antigen-binding domain, and
a ligand-receptor pair comprising an extracellular portion of an immunoglobulin superfamily receptor and its cognate ligand or a receptor-binding fragment thereof; wherein the ligand is fused to the N-terminus of one of the VH or VL polypeptides via a first peptidic linker and the receptor is fused to the N-terminus of the other VH or VL polypeptide via a second peptidic linker; wherein
the first and second peptidic linkers are of sufficient length to allow pairing of the ligand and receptor; wherein
at least one of the first and second peptidic linkers comprises a protease cleavage site; and wherein
the ligand-receptor pair sterically hinders binding of the antigen-binding domain to its cognate antigen.

90. The fusion protein according to claim 89, further comprising an additional Fab region or an scFv.

91. A method of treating cancer, comprising administering to a patient in need thereof a sufficient amount of the fusion protein of any one of the above claims.

92. A method of modulating an immune response, comprising administering to a patient in need thereof a sufficient amount of the fusion protein of any one of the above claims.

93. The method according to claim 92, wherein the immune response is selected from the group consisting of: inhibition of an immune checkpoint, stimulation of an immune checkpoint, immune cell activation, stimulation of T-cell receptor signaling, T-cell dependent cytotoxicity (TDCC), antibody-dependent cellular phagocytosis (ADCP) and stimulation of antibody-dependent cellular cytotoxicity (ADCC).

94. The method according to any one of claims 91 to 93, wherein the fusion protein is administered intravenously.

95. A vector encoding an amino acid sequence comprising at least one polypeptide of the fusion protein of any one of claims 1-90.

96. A cell comprising a vector according to claim 95.

97. A kit comprising a vector according to claim 95 a cell according to claim 96, a purified fusion protein according to any one of claims 1 to 90, or combinations thereof, and instructions for use.

98. The fusion protein according to any of claims 1 to 90 wherein cleavage of the protease cleavage site in a cellular environment releases one member of the ligand-receptor pair from the fusion protein, thereby allowing the other member of the ligand-receptor pair to bind its cognate partner on a cell surface.


SEQUENCE LISTING (268 sequences)

SEQ ID NO: 1 - protein, 106 aa, Artificial Sequence
  CRIS7 CD3 VL
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQIT

SEQ ID NO: 2 - protein, 121 aa, Artificial Sequence
  CRIS7 CD3 VH
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSS

SEQ ID NO: 3 - protein, 247 aa, Artificial Sequence
  Trastuzumab scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 4 - protein, 106 aa, Artificial Sequence
  Chain A CH3 region
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 5 - protein, 106 aa, Artificial Sequence
  ChainB CH3 region
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 6 - protein, 110 aa, Artificial Sequence
  CH2 region with L234A-L235A_D265S mutations
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 7 - protein, 114 aa, Artificial Sequence
  Wild type PD-1
NPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTE

SEQ ID NO: 8 - protein, 115 aa, Artificial Sequence [sequence omitted]

SEQ ID NO: 9 - protein, 114 aa, Artificial Sequence
  High affinity PD-1
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTE

SEQ ID NO: 10 - protein, 115 aa, Artificial Sequence
  High affinity PD-L1
AFTVTVPKDLYVVEYGSNMTIECKFPVEKQLDLAALQVFWMMEDKNIIQFVHGEEDLKVQHSSYRQRARLLKDQLSLGNAALQITDVKLQDAGVYTCLIAYKGADYKRITVKVNA

SEQ ID NO: 11 - protein, 114 aa, Artificial Sequence
  WT CS PD-1
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTE

SEQ ID NO: 12 - protein, 106 aa, Artificial Sequence [sequence omitted]

SEQ ID NO: 13 - protein, 107 aa, Artificial Sequence
  EGFR VL
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 14 - protein, 119 aa, Artificial Sequence
  EGFR VH
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 15 - protein, 106 aa, Artificial Sequence
  MSLN VL
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 16 - protein, 119 aa, Artificial Sequence
  MSLN VH
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 17 - protein, 107 aa, Artificial Sequence
  TF VL
DIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIK

SEQ ID NO: 18 - protein, 117 aa, Artificial Sequence
  TF VH
EVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSS

SEQ ID NO: 19 - protein, 106 aa, Artificial Sequence
  CD19A VL
EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIK

SEQ ID NO: 20 - protein, 120 aa, Artificial Sequence
  CD19A VH
QVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSS

SEQ ID NO: 21 - protein, 111 aa, Artificial Sequence
  cMET VL
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 22 - protein, 118 aa, Artificial Sequence
  cMET VH
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 23 - protein, 111 aa, Artificial Sequence
  CDH3 VL
QSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVL

SEQ ID NO: 24 - protein, 117 aa, Artificial Sequence
  CDH3 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSS

SEQ ID NO: 25 - protein, 106 aa, Artificial Sequence
  WT CD80
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKA

SEQ ID NO: 26 - protein, 118 aa, Artificial Sequence
  WT CTLA4
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 27 - protein, 27 aa, Artificial Sequence
  Signal peptide
EFATMRPTWAWWLFLVLLLALWAPARG

SEQ ID NO: 28 - protein, 8 aa, Artificial Sequence
  Protease cleavage site
MSGRSANA

SEQ ID NO: 29 - protein, 217 aa, Artificial Sequence
  Human IgG1 Fc sequence 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 8 aa, Artificial Sequence
  Protease cleavage site
TSGRSANP

SEQ ID NO: 31 - protein, 8 aa, Artificial Sequence
  Protease cleavage site
LSGRSDNH

SEQ ID NO: 32 - protein, 8 aa, Artificial Sequence
  Protease cleavage site
GSGRSAQV

SEQ ID NO: 33 - protein, 8 aa, Artificial Sequence
  Protease cleavage site
GSSRNADV

SEQ ID NO: 34 - protein, 8 aa, Artificial Sequence
  Protease cleavage site
GTARSDNV

SEQ ID NO: 35 - protein, 8 aa, Artificial Sequence
  Protease cleavage sequence
GGGRVNNV

SEQ ID NO: 36 - protein, 8 aa, Artificial Sequence
  Protease cleavage site
MSARILQV

SEQ ID NO: 37 - protein, 8 aa, Artificial Sequence
  Protease cleavage site
GKGRSANA

SEQ ID NO: 38 - protein, 10 aa, Artificial Sequence
  Linker
EAAAKEAAAK

SEQ ID NO: 39 - protein, 5 aa, Artificial Sequence
  Linker
EAAAK

SEQ ID NO: 40 - protein, 4 aa, Artificial Sequence
  Linker
PPPP

SEQ ID NO: 41 - protein, 3 aa, Artificial Sequence
  Linker
PPP

SEQ ID NO: 42 - protein, 5 aa, Artificial Sequence
  Linker
GGGGS

SEQ ID NO: 43 - protein, 18 aa, Artificial Sequence
  Linker with C-terminal protease cleavage site
EAAAKEAAAKMSGRSANA

SEQ ID NO: 44 - protein, 18 aa, Artificial Sequence
  Linker with N-terminal protease cleavage site
MSGRSANAEAAAKEAAAK

SEQ ID NO: 45 - protein, 13 aa, Artificial Sequence
  Linker with N-terminal protease cleavage sequence
MSGRSANAEAAAK

SEQ ID NO: 46 - protein, 215 aa, Artificial Sequence
  Clone ID 787 Full
GDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 107 aa, Artificial Sequence; identical to SEQ ID NO: 17
  Clone ID 787, 23262, 23264 VL

SEQ ID NO: 48 - protein, 11 aa, Artificial Sequence
  Clone ID 787, 23262, 23264 LCDR1
RASRDIKSYLN

SEQ ID NO: 49 - protein, 7 aa, Artificial Sequence
  Clone ID 787, 23262, 23264 LCDR2
YATSLAE

SEQ ID NO: 50 - protein, 9 aa, Artificial Sequence
  Clone ID 787, 23262, 23264 LCDR3
LQHGESPWT

SEQ ID NO: 51 - protein, 233 aa, Artificial Sequence
  Clone ID 1380 Full
GEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 52 - protein, 447 aa, Artificial Sequence
  Clone ID 2932 Full
GEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 117 aa, Artificial Sequence; identical to SEQ ID NO: 18
  Clone ID 2932, 23261 VH

SEQ ID NO: 54 - protein, 5 aa, Artificial Sequence
  Clone ID 2932, 23261 HCDR1
EYYMH

SEQ ID NO: 55 - protein, 17 aa, Artificial Sequence
  Clone ID 2932, 23261 HCDR2
LIDPEQGNTIYDPKFQD

SEQ ID NO: 56 - protein, 8 aa, Artificial Sequence
  Clone ID 2932, 23261 HCDR3
DTAAYFDY

SEQ ID NO: 57 - protein, 215 aa, Artificial Sequence
  Clone ID 3232, 3357 Full
GDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 107 aa, Artificial Sequence; identical to SEQ ID NO: 13
  Clone ID 3232, 3357, 23247, 23248 VL

SEQ ID NO: 59 - protein, 11 aa, Artificial Sequence
  Clone ID 3232, 3357, 23247, 23248 LCDR1
RASQSIGTNIH

SEQ ID NO: 60 - protein, 7 aa, Artificial Sequence
  Clone ID 3232, 3357, 23247, 23248 LCDR2
YASESIS

SEQ ID NO: 61 - protein, 9 aa, Artificial Sequence
  Clone ID 3232,3357, 23247, 23248 LCDR3
QQNNNWPTT

SEQ ID NO: 62 - protein, 451 aa, Artificial Sequence
  Clone ID 3345 Full
GQVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 63 - protein, 120 aa, Artificial Sequence; identical to SEQ ID NO: 20
  Clone ID 3345, 23257 VH

SEQ ID NO: 64 - protein, 7 aa, Artificial Sequence
  Clone ID 3345, 23257 HCDR1
TSGMGVG

SEQ ID NO: 65 - protein, 16 aa, Artificial Sequence
  Clone ID 3345, 23257 HCDR2
HIWWDDDKRYNPALKS

SEQ ID NO: 66 - protein, 10 aa, Artificial Sequence
  Clone ID 3345, 23257 HCDR3
MELWSYYFDY

SEQ ID NO: 67 - protein, 449 aa, Artificial Sequence
  Clone ID 10564 Full
GQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 68 - protein, 119 aa, Artificial Sequence; identical to SEQ ID NO: 16
  Clone ID 10564, 23253 VH

SEQ ID NO: 69 - protein, 5 aa, Artificial Sequence
  Clone ID 10564, 23253 HCDR1
GYTMN

SEQ ID NO: 70 - protein, 17 aa, Artificial Sequence
  Clone ID 10564, 23253 HCDR2
LITPYNGASSYNQKFRG

SEQ ID NO: 71 - protein, 10 aa, Artificial Sequence
  Clone ID 10564, 23253 HCDR3
GGYDGRGFDY

SEQ ID NO: 72 - protein, 214 aa, Artificial Sequence
  Clone ID 10565 Full
GDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 106 aa, Artificial Sequence; identical to SEQ ID NO: 15
  Clone ID 10565, 23256 VL

SEQ ID NO: 74 - protein, 10 aa, Artificial Sequence
  Clone ID 10565, 23256, 23258, 23260 LCDR1
SASSSVSYMH

SEQ ID NO: 75 - protein, 7 aa, Artificial Sequence
  Clone ID 10565, 23256, 23258, 23260 LCDR2
DTSKLAS

SEQ ID NO: 76 - protein, 9 aa, Artificial Sequence
  Clone ID 10565, 23256 LCDR3
QQWSKHPLT

SEQ ID NO: 77 - protein, 218 aa, Artificial Sequence
  Clone ID 10567 Full
GQSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 111 aa, Artificial Sequence; identical to SEQ ID NO: 23
  Clone ID 10567, 20871 VL

SEQ ID NO: 79 - protein, 14 aa, Artificial Sequence
  Clone ID 10567, 20871 LCDR1
TGTSNDVGAYNYVS

SEQ ID NO: 80 - protein, 7 aa, Artificial Sequence
  Clone ID 10567, 20871 LCDR2
EVNKRPS

SEQ ID NO: 81 - protein, 11 aa, Artificial Sequence
  Clone ID 10567, 20871 LCDR3
SSFTSGLPWVV

SEQ ID NO: 82 - protein, 449 aa, Artificial Sequence
  Clone ID 10606 Full
GQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 83 - protein, 119 aa, Artificial Sequence; identical to SEQ ID NO: 14
  Clone ID 10606, 23246, 23567 VH

SEQ ID NO: 84 - protein, 5 aa, Artificial Sequence
  Clone ID 10606, 23246, 23567 HCDR1
NYGVH

SEQ ID NO: 85 - protein, 16 aa, Artificial Sequence
  Clone ID 10606, 23246, 23567 HCDR2
VIWSGGNTDYNTPFTS

SEQ ID NO: 86 - protein, 11 aa, Artificial Sequence
  Clone ID 10606, 23246, 23567 HCDR3
ALTYYDYEFAY

SEQ ID NO: 87 - protein, 447 aa, Artificial Sequence
  Clone ID 11274 Full
GEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 117 aa, Artificial Sequence; identical to SEQ ID NO: 24
  Clone ID 11274, 20875 VH

SEQ ID NO: 89 - protein, 5 aa, Artificial Sequence
  Clone ID 11274, 20875 HCDR1
SYAMS

SEQ ID NO: 90 - protein, 17 aa, Artificial Sequence
  Clone ID 11274, 20875 HCDR2
AISGSGGSTYYADSVKG

SEQ ID NO: 91 - protein, 8 aa, Artificial Sequence
  Clone ID 11274, 20875 HCDR3
WGDGTLNP

SEQ ID NO: 92 - protein, 219 aa, Artificial Sequence
  Clone ID 11462 Full
GDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 111 aa, Artificial Sequence; identical to SEQ ID NO: 21
  Clone ID 11462, 20855 VL

SEQ ID NO: 94 - protein, 15 aa, Artificial Sequence
  Clone ID 11462, 20855 LCDR1
KSSESVDSYANSFLH

SEQ ID NO: 95 - protein, 7 aa, Artificial Sequence
  Clone ID 11462, 20855 LCDR2
RASTRES

SEQ ID NO: 96 - protein, 9 aa, Artificial Sequence
  Clone ID 11462, 20855 LCDR3
QQSKEDPLT

SEQ ID NO: 97 - protein, 446 aa, Artificial Sequence
  Clone ID 11509 Full
GQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 118 aa, Artificial Sequence; identical to SEQ ID NO: 22
  Clone ID 11509, 20859 VH

SEQ ID NO: 99 - protein, 5 aa, Artificial Sequence
  Clone ID 11509, 20859 HCDR1
AYTMH

SEQ ID NO: 100 - protein, 17 aa, Artificial Sequence
  Clone ID 11509, 20859 HCDR2
WIKPNNGLANYAQKFQG

SEQ ID NO: 101 - protein, 9 aa, Artificial Sequence
  Clone ID 11509, 20859 HCDR3
SEITTEFDY

SEQ ID NO: 102 - protein, 213 aa, Artificial Sequence
  Clone ID 12985 Full
DIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 106 aa, Artificial Sequence; identical to SEQ ID NO: 1
  Clone ID 12985, 22091, 22092, 22094, 22105 VL

SEQ ID NO: 104 - protein, 10 aa, Artificial Sequence
  Clone ID 12985, 22091, 22092, 22094, 22105 LCDR1
SASSSVSYMN

SEQ ID NO: 105 - protein, 7 aa, Artificial Sequence
  Clone ID 12985, 22091, 22092, 22094, 22105 LCDR2
DSSKLAS

SEQ ID NO: 106 - protein, 9 aa, Artificial Sequence
  Clone ID 12985, 22091, 22092, 22094, 22105 LCDR3
QQWSRNPPT

SEQ ID NO: 107 - protein, 450 aa, Artificial Sequence
  Clone ID 12989 Full
QVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 108 - protein, 121 aa, Artificial Sequence; identical to SEQ ID NO: 2
  Clone ID 12989, 22080, 22082, 22083, 22086, 22088, 24659, 24660, 24661 VH

SEQ ID NO: 109 - protein, 5 aa, Artificial Sequence
  Clone ID 12989, 22080, 22082, 22083, 22086, 22088, 24659, 24660, 24661 HCDR1
RSTMH

SEQ ID NO: 110 - protein, 17 aa, Artificial Sequence
  Clone ID 12989, 22080, 22082, 22083, 22086, 22088, 24659, 24660, 24661 HCDR2
YINPSSAYTNYNQKFKD

SEQ ID NO: 111 - protein, 12 aa, Artificial Sequence
  Clone ID 12989, 22080, 22082, 22083, 22086, 22088, 24659, 24660, 24661 HCDR3
PQVHYDYNGFPY

SEQ ID NO: 112 - protein, 343 aa, Artificial Sequence
  Clone ID 20855 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 115 aa, Artificial Sequence [sequence omitted]

SEQ ID NO: 114 - protein, 10 aa, Artificial Sequence
  Clone ID 20855, 20859, 20871, 20875, 22080, 22082, 22088, 22091, 22092, 23246, 23247, 23253, 23257, 23258, 23261, 23262, 23714, 23715, 24659, 24660, 24661 Mask Linker
EAAAKEAAAK

SEQ ID NO: 115 - protein, 569 aa, Artificial Sequence
  Clone ID 20859 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDCHCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 114 aa, Artificial Sequence; identical to SEQ ID NO: 9
  Clone ID 20859, 20875, 22080, 23246, 23253, 23257, 23261, 23714 Mask

SEQ ID NO: 117 - protein, 342 aa, Artificial Sequence
  Clone ID 20871 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKQSALTQPASVSGSPGQSITISCTGTSNDVGAYNYVSWYQQHPGKAPKLMISEVNKRPSGVSNRFSGSKSGNTASLTISGLQAEDEADYYCSSFTSGLPWVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 118 - protein, 570 aa, Artificial Sequence
  Clone ID 20875 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGDGTLNPWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 478 aa, Artificial Sequence
  Clone ID 21490 Full
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGSGGGSGGGSGGGSGGGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 120 - protein, 120 aa, Artificial Sequence [sequence omitted]

SEQ ID NO: 121 - protein, 5 aa, Artificial Sequence
  Clone ID 21490 HCDR1
DTYIH

SEQ ID NO: 122 - protein, 17 aa, Artificial Sequence
  Clone ID 21490 HCDR2
RIYPTNGYTRYADSVKG

SEQ ID NO: 123 - protein, 11 aa, Artificial Sequence
  Clone ID 21490 HCDR3
WGGDGFYAMDY

SEQ ID NO: 124 - protein, 107 aa, Artificial Sequence [sequence omitted]

SEQ ID NO: 125 - protein, 11 aa, Artificial Sequence
  Clone ID 21490 LCDR1
RASQDVNTAVA

SEQ ID NO: 126 - protein, 7 aa, Artificial Sequence
  Clone ID 21490 LCDR2
SASFLYS

SEQ ID NO: 127 - protein, 9 aa, Artificial Sequence
  Clone ID 21490 LCDR3
QQHYTTPPT

SEQ ID NO: 128 - protein, 706 aa, Artificial Sequence
  Clone ID 21496 Full
EVQLVESGGGLVQPGRSLKLSCGASGFTFSDYYMAWVRQAPKKGLEWVASISYEGRSTYYGDSVKGRFTISRDNAKSTLYLQMNSLRSEDTATYYCARRAEGMDFDYWGQGVMVTVSSAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCPPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYVIPPSKEQMAKDKVSLLCMITDFFPEDITVEWQWNGQPAENYLTWPPIMDTDGSYFVYSKLNVQKSNWEAGNTFTCSVLHEGLHNHHTEKSLSHSPGGGSGGGSGGGSGGGSGGGSDIVMTQTPASVEAAVGGTVTIKCQASQSIYSSLAWYQQKPGQSPKLLIYDASHLASGVPSRFSGSRYGTEFTLTISGVQCDDAATYYCQGGWYSSAATYVPNTFGGGTEVVVKGGGGSGGGGSGGGGSQEQLVESGGGLVQPEGSLTLTCKASGFTISNNYYMCWVRQAPGKGLEWIACIYGGISGRTYYASWAKGRFTISKTSSTTVTLQMTSLTAADTATYFCVRGYVGTSNLWGPGTLVTVSS

SEQ ID NO: 129 - protein, 118 aa, Artificial Sequence
  Clone ID 21496 VH
EVQLVESGGGLVQPGRSLKLSCGASGFTFSDYYMAWVRQAPKKGLEWVASISYEGRSTYYGDSVKGRFTISRDNAKSTLYLQMNSLRSEDTATYYCARRAEGMDFDYWGQGVMVTVSS

SEQ ID NO: 130 - protein, 5 aa, Artificial Sequence
  Clone ID 21496 HCDR1
DYYMA

SEQ ID NO: 131 - protein, 17 aa, Artificial Sequence
  Clone ID 21496 HCDR2
SISYEGRSTYYGDSVKG

SEQ ID NO: 132 - protein, 9 aa, Artificial Sequence
  Clone ID 21496 HCDR3
RAEGMDFDY

SEQ ID NO: 133 - protein, 118 aa, Artificial Sequence
  Clone ID 21496 VH
QEQLVESGGGLVQPEGSLTLTCKASGFTISNNYYMCWVRQAPGKGLEWIACIYGGISGRTYYASWAKGRFTISKTSSTTVTLQMTSLTAADTATYFCVRGYVGTSNLWGPGTLVTVSS

SEQ ID NO: 134 - protein, 6 aa, Artificial Sequence
  Clone ID 21496 HCDR1
NNYYMC

SEQ ID NO: 135 - protein, 18 aa, Artificial Sequence
  Clone ID 21496 HCDR2
CIYGGISGRTYYASWAKG

SEQ ID NO: 136 - protein, 8 aa, Artificial Sequence
  Clone ID 21496 HCDR3
GYVGTSNL

SEQ ID NO: 137 - protein, 113 aa, Artificial Sequence
  Clone ID 21496 VL
DIVMTQTPASVEAAVGGTVTIKCQASQSIYSSLAWYQQKPGQSPKLLIYDASHLASGVPSRFSGSRYGTEFTLTISGVQCDDAATYYCQGGWYSSAATYVPNTFGGGTEVVVK

SEQ ID NO: 138 - protein, 11 aa, Artificial Sequence
  Clone ID 21496 LCDR1
QASQSIYSSLA

SEQ ID NO: 139 - protein, 7 aa, Artificial Sequence
  Clone ID 21496 LCDR2
DASHLAS

SEQ ID NO: 140 - protein, 15 aa, Artificial Sequence
  Clone ID 21496 LCDR3
QGGWYSSAATYVPNT

SEQ ID NO: 141 - protein, 574 aa, Artificial Sequence
  Clone ID 22080 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 142 - protein, 574 aa, Artificial Sequence
  Clone ID 22082 Full
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 143 - protein, 114 aa, Artificial Sequence; identical to SEQ ID NO: 11
  Clone ID 22082, 22083, 22086 Mask

SEQ ID NO: 144 - protein, 577 aa, Artificial Sequence
  Clone ID 22083 Full
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEMSGRSANAEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 145 - protein, 13 aa, Artificial Sequence
  Clone ID 22083 Mask Linker
MSGRSANAEAAAK

SEQ ID NO: 146 - protein, 582 aa, Artificial Sequence
  Clone ID 22086 Full
NPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTEEAAAKEAAAKMSGRSANAQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 147 - protein, 18 aa, Artificial Sequence
  Clone ID 22086, 22105, 23248, 23256, 23260, 23264, 23716 Mask Linker
EAAAKEAAAKMSGRSANA

SEQ ID NO: 148 - protein, 566 aa, Artificial Sequence
  Clone ID 22088 Full
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 149 - protein, 106 aa, Artificial Sequence; identical to SEQ ID NO: 25
  Clone ID 22088 Mask

SEQ ID NO: 150 - protein, 338 aa, Artificial Sequence
  Clone ID 22091 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 338 aa, Artificial Sequence
  Clone ID 22092 Full
AFTVTVPKDLYVVEYGSNMTIECKFPVEKQLDLAALQVFWMMEDKNIIQFVHGEEDLKVQHSSYRQRARLLKDQLSLGNAALQITDVKLQDAGVYTCLIAYKGADYKRITVKVNAEAAAKEAAAKDIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 115 aa, Artificial Sequence; identical to SEQ ID NO: 10
  Clone ID 22092 Mask

SEQ ID NO: 153 - protein, 341 aa, Artificial Sequence
  Clone ID 22094 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKMSGRSANADIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 13 aa, Artificial Sequence
  Clone ID 22094 Mask Linker
EAAAKMSGRSANA

SEQ ID NO: 155 - protein, 349 aa, Artificial Sequence
  Clone ID 22105 Full
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEEAAAKEAAAKMSGRSANADIQMTQSPSSLSASVGDRVTMTCSASSSVSYMNWYQQKPGKAPKRWIYDSSKLASGVPARFSGSGSGTDYTLTISSLQPEDFATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 118 aa, Artificial Sequence; identical to SEQ ID NO: 26
  Clone ID 22105 Mask

SEQ ID NO: 157 - protein, 572 aa, Artificial Sequence
  Clone ID 23246 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 339 aa, Artificial Sequence
  Clone ID 23247 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 347 aa, Artificial Sequence
  Clone ID 23248 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 572 aa, Artificial Sequence
  Clone ID 23253 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 346 aa, Artificial Sequence
  Clone ID 23256 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 573 aa, Artificial Sequence
  Clone ID 23257 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVTLRESGPALVKPTQTLTLTCTFSGFSLSTSGMGVGWIRQPPGKALEWLAHIWWDDDKRYNPALKSRLTISKDTSKNQVVLTMTNMDPVDTAAYYCARMELWSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 338 aa, Artificial Sequence
  Clone ID 23258 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 106 aa, Artificial Sequence; identical to SEQ ID NO: 19
  Clone ID 23258, 23260 VL

SEQ ID NO: 165 - protein, 9 aa, Artificial Sequence
  Clone ID 23258, 23260 LCDR3
FQGSVYPFT

SEQ ID NO: 166 - protein, 346 aa, Artificial Sequence
  Clone ID 23260 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANAEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 570 aa, Artificial Sequence
  Clone ID 23261 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKEVQLVESGGGLVQPGGSLRLSCAASGFNIKEYYMHWVRQAPGKGLEWVGLIDPEQGNTIYDPKFQDRATISADNSKNTAYLQMNSLRAEDTAVYYCARDTAAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 339 aa, Artificial Sequence
  Clone ID 23262 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 347 aa, Artificial Sequence
  Clone ID 23264 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSLSASVGDRVTITCRASRDIKSYLNWYQQKPGKAPKVLIYYATSLAEGVPSRFSGSGSGTDYTLTISSLQPEDFATYYCLQHGESPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 448 aa, Artificial Sequence
  Clone ID 23567 Full
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 171 - protein, 455 aa, Artificial Sequence
  Clone ID 23712 Full
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 126 aa, Artificial Sequence
  Clone ID 23712, 23714 VH
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 173 - protein, 5 aa, Artificial Sequence
  Clone ID 23712, 23714 HCDR1
GYYMH

SEQ ID NO: 174 - protein, 17 aa, Artificial Sequence
  Clone ID 23712, 23714 HCDR2
WINPDSGGTNYAQKFQG

SEQ ID NO: 175 - protein, 17 aa, Artificial Sequence
  Clone ID 23712, 23714 HCDR3
DQPLGYCTNGVCSYFDY

SEQ ID NO: 176 - protein, 214 aa, Artificial Sequence
  Clone ID 23713 Full
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 107 aa, Artificial Sequence
  Clone ID 23713, 23715, 23716 VL
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 178 - protein, 11 aa, Artificial Sequence
  Clone ID 23713, 23715, 23716 LCDR1
RASQGIYSWLA

SEQ ID NO: 179 - protein, 7 aa, Artificial Sequence
  Clone ID 23713, 23715, 23716 LCDR2
TASTLQS

SEQ ID NO: 180 - protein, 9 aa, Artificial Sequence
  Clone ID 23713, 23715, 23716 LCDR3
QQANIFPLT

SEQ ID NO: 181 - protein, 579 aa, Artificial Sequence
  Clone ID 23714 Full
NPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTEEAAAKEAAAKQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 339 aa, Artificial Sequence
  Clone ID 23715 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 347 aa, Artificial Sequence
  Clone ID 23716 Full
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAEAAAKEAAAKMSGRSANADIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 566 aa, Artificial Sequence
  Clone ID 24659 Full
VIHVTKEVKEVATLSCGHNVSVEELAQTRIYWQKEKKMVLTMMSGDMNIWPEYKNRTSFDITNNLSISISALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 185 - protein, 106 aa, Artificial Sequence
  Clone ID 24659 Mask
VIHVTKEVKEVATLSCGHNVSVEELAQTRIYWQKEKKMVLTMMSGDMNIWPEYKNRTSFDITNNLSISISALRPSDEGTYECVVLKYEKDAFKREHLAEVTLSVKA

SEQ ID NO: 186 - protein, 566 aa, Artificial Sequence
  Clone ID 24660 Full
VIHVTKEVKEVATLSCGHNVSVEELAQTRIYWQKEKKMVLTMMSGDSNIWPEYKNRTIFDSTNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 187 - protein, 106 aa, Artificial Sequence
  Clone ID 24660 Mask
VIHVTKEVKEVATLSCGHNVSVEELAQTRIYWQKEKKMVLTMMSGDSNIWPEYKNRTIFDSTNNLSIVILALRPSDEGTYECVVLKYEKDAFKREHLAEVTLSVKA

SEQ ID NO: 188 - protein, 566 aa, Artificial Sequence
  Clone ID 24661 Full
VIHVTKEVKEVATLSCGHNVSSEELAQTRIYWQKEKKMVLTMMSGDMNIWPEYKNRTIFDITNNLSIVILALRPSDEGTYECVVLKYEKDAFKREHLAEVTLSVKAEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 189 - protein, 106 aa, Artificial Sequence
  Clone ID 24661 Mask
VIHVTKEVKEVATLSCGHNVSSEELAQTRIYWQKEKKMVLTMMSGDMNIWPEYKNRTIFDITNNLSIVILALRPSDEGTYECVVLKYEKDAFKREHLAEVTLSVKA

SEQ ID NO: 190 - protein, 574 aa, Artificial Sequence
  Clone ID 23734
NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTEEAAAKEAAAKQVQLVESGGGVVQPGRSLRLSCKASGYTFTRSTMHWVRQAPGQGLEWIGYINPSSAYTNYNQKFKDRFTISADKSKSTAFLQMDSLRPEDTGVYFCARPQVHYDYNGFPYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 191 - protein, 114 aa, Artificial Sequence
  Clone ID 23734 Mask
NPPTFSPALLVVTEGDNATFTCSFSNTSESFVLNWYRMSPSNQTDALAAFPEDRSQPGQDSRFRVTQLPNGRDFHMSVVRARRNDSGTYLCGAASLAPKAQIKESLRAELRVTE

SEQ ID NO: 192 - protein, 487 aa, Artificial Sequence
  Clone ID 11018Full
GQVQLVQSGAEVKKPGASVRVSCRASGYIFTESGITWVRQAPGQGLEWMGWISGYSGDTKYAQKLQGRVTMTKDTSTTTAYMELRSLRYDDTAVYYCARDVQYSGSYLGAYYFDYWSPGTLVTVSSGGGGSGGGGSGGGGSGGGQSVLTQPPSASGTPGQRVTISCSGSSSNIGTNYVYWYQQFPGTAPKLLIYRSYQRPSGVPDRFSGSKSGSSASLAISGLQSEDEADYYCATWDDSLDGWVFGGGTKLTVLAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 193 - protein, 125 aa, Artificial Sequence
  Clone ID 11018 VH
QVQLVQSGAEVKKPGASVRVSCRASGYIFTESGITWVRQAPGQGLEWMGWISGYSGDTKYAQKLQGRVTMTKDTSTTTAYMELRSLRYDDTAVYYCARDVQYSGSYLGAYYFDYWSPGTLVTVSS

SEQ ID NO: 194 - protein, 110 aa, Artificial Sequence
  Clone ID 11018 VL
QSVLTQPPSASGTPGQRVTISCSGSSSNIGTNYVYWYQQFPGTAPKLLIYRSYQRPSGVPDRFSGSKSGSSASLAISGLQSEDEADYYCATWDDSLDGWVFGGGTKLTVL

SEQ ID NO: 195 - protein, 5 aa, Artificial Sequence
  Clone ID 11018 HCDR1
ESGIT

SEQ ID NO: 196 - protein, 17 aa, Artificial Sequence
  Clone ID 11018 HCDR2
WISGYSGDTKYAQKLQG

SEQ ID NO: 197 - protein, 16 aa, Artificial Sequence
  Clone ID 11018 HCDR3
DVQYSGSYLGAYYFDY

SEQ ID NO: 198 - protein, 13 aa, Artificial Sequence
  Clone ID 11018 LCDR1
SGSSSNIGTNYVY

SEQ ID NO: 199 - protein, 7 aa, Artificial Sequence
  Clone ID 11018 LCDR2
RSYQRPS

SEQ ID NO: 200 - protein, 11 aa, Artificial Sequence
  Clone ID 11018 LCDR3
ATWDDSLDGWV

SEQ ID NO: 201 - protein, 571 aa, Artificial Sequence
  Clone ID 25321 Full
EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRAMSGRSANAQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 115 aa, Artificial Sequence
  Clone ID 25321 Mask
EEELQIIQPDKSVSVAAGESAILHCTITSLFPVGPIQWFRGAGPARVLIYNQRQGPFPRVTTVSETTKRENMDFSISISNITPADAGTYYCIKFRKGSPDTEFKSGAGTELSVRA

SEQ ID NO: 203 - protein, 8 aa, Artificial Sequence
  Clone ID 25321 Mask Linker
MSGRSANA

SEQ ID NO: 204 - protein, 337 aa, Artificial Sequence
  Clone ID 25325 Full
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVMSGRSANADILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 115 aa, Artificial Sequence
  25325 Mask
QLLFNKTKSVEFTFGNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRV

SEQ ID NO: 206 - protein, 118 aa, Artificial Sequence
  Anti-CD3 paratope 1 VH
EVQLVESGGGLVQPGGSLRLSCAASGVTFNYYGMSWIRQAPGKGLEWVASITSSGGRIYYPDSVKGRFTISRENTQKTLYLQMNSLRAEDTAVYYCTLDGRDGWVAYWGQGTLVTVSS

SEQ ID NO: 207 - protein, 5 aa, Artificial Sequence
  Anti-CD3 paratope 1 Kabat HCDR1
YYGMS

SEQ ID NO: 208 - protein, 17 aa, Artificial Sequence
  Anti-CD3 paratope 1 Kabat HCDR2
SITSSGGRIYYPDSVKG

SEQ ID NO: 209 - protein, 9 aa, Artificial Sequence
  Anti-CD3 paratope 1 Kabat HCDR3
DGRDGWVAY

SEQ ID NO: 210 - protein, 109 aa, Artificial Sequence
  Anti-CD3 paratope 1 VL
NFMLTQPHSVSESPGKTVTISCKRNTGNIGSNYVNWYQQHEGSSPTTIIYRNDKRPDGVSDRFSGSIDRSSKSASLTISNLKTEDEADYFCQSYSSGFIFGGGTKLTVL

SEQ ID NO: 211 - protein, 13 aa, Artificial Sequence
  Anti-CD3 paratope 1 Kabat LCDR1
KRNTGNIGSNYVN

SEQ ID NO: 212 - protein, 7 aa, Artificial Sequence
  Anti-CD3 paratope 1 Kabat LCDR2
RNDKRPD

SEQ ID NO: 213 - skipped

SEQ ID NO: 214 - protein, 8 aa, Artificial Sequence
  Anti-CD3 paratope 1 Kabat LCDR3
QSYSSGFI

SEQ ID NO: 215 - protein, 118 aa, Artificial Sequence
  Anti-CD3 paratope 2 VH
EVQLVESGGGLVQPGGSLRLSCAASGVTFNYYGMSWIRQAPGKGLEWVASITRSGGRIYYPDSVKGRFTISRENTQKTLYLQMNSLRAEDTAVYYCTLDGRDGWVAYWGQGTLVTVSS

SEQ ID NO: 216 - protein, 5 aa, Artificial Sequence
  Anti-CD3 paratope 2 Kabat HCDR1
YYGMS

SEQ ID NO: 217 - protein, 17 aa, Artificial Sequence
  Anti-CD3 paratope 2 Kabat HCDR2
SITRSGGRIYYPDSVKG

SEQ ID NO: 218 - protein, 9 aa, Artificial Sequence
  Anti-CD3 paratope 2 Kabat HCDR3
DGRDGWVAY

SEQ ID NO: 219 - protein, 107 aa, Artificial Sequence
  Anti-CD3 paratope 2 VL
NFMLTQPSSVSGVPGQRVTISCTGNTGNIGSNYVNWYQQLPGTAPKLLIYRDDKRPSGVPDRFSGSKSGTSASLAITGFQAEDEADYYCQSYSSGFIFGGGTKLTVL

SEQ ID NO: 220 - protein, 13 aa, Artificial Sequence
  Anti-CD3 paratope 2 Kabat LCDR1
TGNTGNIGSNYVN

SEQ ID NO: 221 - protein, 7 aa, Artificial Sequence
  Anti-CD3 paratope 2 Kabat LCDR2
RDDKRPS

SEQ ID NO: 222 - protein, 8 aa, Artificial Sequence
  Anti-CD3 paratope 2 Kabat LCDR3
QSYSSGFI

SEQ ID NO: 223 - protein, 125 aa, Artificial Sequence
  Anti-CD3 paratope 3 VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 224 - protein, 5 aa, Artificial Sequence
  Anti-CD3 paratope 3 Kabat HCDR1
KYAMN

SEQ ID NO: 225 - protein, 19 aa, Artificial Sequence
  Anti-CD3 paratope 3 Kabat HCDR2
RIRSKYNNYATYYADSVKD

SEQ ID NO: 226 - protein, 14 aa, Artificial Sequence
  Anti-CD3 paratope 3 Kabat HCDR3
HGNFGNSYISYWAY

SEQ ID NO: 227 - protein, 109 aa, Artificial Sequence
  Anti-CD3 paratope 3 VL
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 228 - protein, 14 aa, Artificial Sequence
  Anti-CD3 paratope 3 Kabat LCDR1
GSSTGAVTSGNYPN

SEQ ID NO: 229 - protein, 7 aa, Artificial Sequence
  Anti-CD3 paratope 3 Kabat LCDR2
GTKFLAP

SEQ ID NO: 230 - protein, 9 aa, Artificial Sequence
  Anti-CD3 paratope 3 Kabat LCDR3
VLWYSNRWV

SEQ ID NO: 231 - protein, 125 aa, Artificial Sequence
  Anti-CD3 paratope 4 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 232 - protein, 5 aa, Artificial Sequence
  Anti-CD3 paratope 4 Kabat HCDR1
TYAMN

SEQ ID NO: 233 - protein, 19 aa, Artificial Sequence
  Anti-CD3 paratope 4 Kabat HCDR2
RIRSKYNNYATYYADSVKG

SEQ ID NO: 234 - protein, 14 aa, Artificial Sequence
  Anti-CD3 paratope 4 Kabat HCDR3
HGNFGNSYVSWFAY

SEQ ID NO: 235 - protein, 109 aa, Artificial Sequence
  Anti-CD3 paratope 4 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 236 - protein, 14 aa, Artificial Sequence
  Anti-CD3 paratope 4 Kabat LCDR1
GSSTGAVTTSNYAN

SEQ ID NO: 237 - protein, 7 aa, Artificial Sequence
  Anti-CD3 paratope 4 Kabat LCDR2
GTNKRAP

SEQ ID NO: 238 - protein, 9 aa, Artificial Sequence
  Anti-CD3 paratope 4 Kabat LCDR3
ALWYSNLWV

SEQ ID NO: 239 - protein, 121 aa, Artificial Sequence
  Anti-CD3 paratope 5 VH
QVQLVQSGAEVKKPGASVKVSCKASGYTFTRSTMHWVRQAPGQGLEWIGYINPSSAYTNYNQKFKDRVTITADKSTSTAYMELSSLRSEDTAVYYCASPQVHYDYNGFPYWGQGTLVTVSS

SEQ ID NO: 240 - protein, 5 aa, Artificial Sequence
  Anti-CD3 paratope 5 Kabat HCDR1
RSTMH

SEQ ID NO: 241 - protein, 17 aa, Artificial Sequence
  Anti-CD3 paratope 5 Kabat HCDR2
YINPSSAYTNYNQKFKD

SEQ ID NO: 242 - protein, 12 aa, Artificial Sequence
  Anti-CD3 paratope 5 Kabat HCDR3
PQVHYDYNGFPY

SEQ ID NO: 243 - protein, 106 aa, Artificial Sequence
  Anti-CD3 paratope 5 VL
DIQMTQSPSSLSASVGDRVTITCSASSSVSYMNWYQQKPGKAPKRLIYDSSKLASGVPSRFSGSGSGTEFTLTISSLQPEDFATYYCQQWSRNPPTFGGGTKVEIK

SEQ ID NO: 244 - protein, 10 aa, Artificial Sequence
  Anti-CD3 paratope 5 Kabat LCDR1
SASSSVSYMN

SEQ ID NO: 245 - protein, 7 aa, Artificial Sequence
  Anti-CD3 paratope 5 Kabat LCDR2
DSSKLAS

SEQ ID NO: 246 - protein, 9 aa, Artificial Sequence
  Anti-CD3 paratope 5 Kabat LCDR3
QQWSRNPPT

SEQ ID NO: 247 - protein, 101 aa, Artificial Sequence
  IgSF Member CD80 Residue 35-135
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVT

SEQ ID NO: 248 - protein, 99 aa, Artificial Sequence
  IgSF Member CD86 Residue 33-131
NETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELS

SEQ ID NO: 249 - protein, 109 aa, Artificial Sequence
  IgSF Member PD-L1 Residue 19-127
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRIT

SEQ ID NO: 250 - protein, 98 aa, Artificial Sequence
  IgSF Member PD-L2 Residue 21-118
FTVTVPKELYIIEHGSNVTLECNFDTGSHVNLGAITASLQKVENDTSPHRERATLLEEQLPLGKASFHIPQVQVRDEGQYQCIIIYGVAWDYKYLTLK

SEQ ID NO: 251 - protein, 102 aa, Artificial Sequence
  IgSF Member CTLA-4 Residue 39-140
HVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYY

SEQ ID NO: 252 - protein, 111 aa, Artificial Sequence
  IgSF Member PD-1 Residue 35-145
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVT

SEQ ID NO: 253 - protein, 110 aa, Artificial Sequence
  IgSF Member CD28 Residue 28-137
MLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKG

SEQ ID NO: 254 - protein, 109 aa, Artificial Sequence
  IgSF Member CD47 Residue 19-127
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETII

SEQ ID NO: 255 - protein, 106 aa, Artificial Sequence
  IgSF Member SIRPa Residue 32-137
EELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSG

SEQ ID NO: 256 - protein, 111 aa, Artificial Sequence
  IgSF Member ICOSL Residue 19-129
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVE

SEQ ID NO: 257 - protein, 103 aa, Artificial Sequence
  IgSF Member ICOS Residue 30-132
MFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYE

SEQ ID NO: 258 - protein, 111 aa, Artificial Sequence
  IgSF Member CD276 Residue 29-139
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVA

SEQ ID NO: 259 - protein, 112 aa, Artificial Sequence
  IgSF Member VTCN1 35-146 Residue 35-146
HSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYK

SEQ ID NO: 260 - protein, 136 aa, Artificial Sequence
  IgSF Member VISTA Residue 33-168
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV

SEQ ID NO: 261 - protein, 112 aa, Artificial Sequence
  IgSF Member NCR3LG1 Residue 27-138
KVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLE

SEQ ID NO: 262 - protein, 71 aa, Artificial Sequence
  IgSF Member HHLA2 Residue 61-131
IHWKYQDSYKVHSYYKGSDHLESQDPRYANRTSLFYNEIQNGNASLFFRRVSLLDEGIYTCYVGTAIQVIT

SEQ ID NO: 263 - protein, 107 aa, Artificial Sequence
  IgSF Member CD28H Residue 23-129
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNIT

SEQ ID NO: 264 - protein, 108 aa, Artificial Sequence
  IgSF Member NKp30 Residue 19-126
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLV

SEQ ID NO: 265 - protein, 5 aa, Artificial Sequence
  Mask linker
GPPPG

SEQ ID NO: 266 - protein, 7 aa, Artificial Sequence
  Mask linker
GGPPPGG

SEQ ID NO: 267 - protein, 6 aa, Artificial Sequence
  Mask linker
GPPPPG

SEQ ID NO: 268 - protein, 7 aa, Artificial Sequence
  Mask linker
GGPPPGG